(19) United States
(12) Patent Application Publication
(10) Pub. No.: US 2013/0164329 A1
(43) Pub. Date: Jun. 27, 2013

(54) CELL-BASED METHODS AND REAGENTS

(71) Applicants: Anthony Rossomando, Cambridge, MA (US); John M. Maraganore, Cambridge, MA (US); Stuart Pollard, Cambridge, MA (US); Muthiah Manoharan, Cambridge, MA (US)

(72) Inventors: Anthony Rossomando, Cambridge, MA (US); John M. Maraganore, Cambridge, MA (US); Stuart Pollard, Cambridge, MA (US); Muthiah Manoharan, Cambridge, MA (US)

(73) Assignee: Alnylam Pharmaceuticals, Inc., Cambridge, MA (US)

(21) Appl. No.: 13/578,886

(22) PCT Filed: Feb. 16, 2011
(86) PCT No.: PCT/US11/25010

§ 371 Date: Mar. 11, 2013

Related U.S. Application Data
(60) Provisional application No. 61/305,284, filed on Feb. 17, 2010.
(60) Provisional application No. 61/319,578, filed on Mar. 31, 2010.
(60) Provisional application No. 61/319,589, filed on Mar. 31, 2010.
(60) Provisional application No. 61/320,840, filed on Apr. 5, 2010.
(60) Provisional application No. 61/321,815, filed on Apr. 7, 2010.
(60) Provisional application No. 61/322,077, filed on Apr. 8, 2010.
(60) Provisional application No. 61/324,624, filed on Apr. 15, 2010.

(51) Int. Cl. C12N 7/02; C12N 7/00; G01N 33/569; C12N 1/20; C12N 1/10; C12Q 1/70; A61K 39/15

(52) U.S. Cl. CPC C12N 7/02; C12Q 1/701; C12N 7/00; A61K 39/15; C12N 1/20; C12N 1/10; G01N 33/56983
USPC 424/215.1; 435/5; 435/239; 435/235.1; 435/252.1; 435/258.1; 435/253.1; 435/258.2; 435/258.3

(57) ABSTRACT

The present invention relates to compositions and methods for isolating cells devoid of unwanted viral contaminants, and to methods for preparing a virus stock substantially devoid of viral contaminants. Virus stocks, cells, and immunogenic reagents produced using such methods are also provided.

### RELATED APPLICATIONS

This application claims the benefit of U.S. Provisional Patent Application No. 61/305,284, filed Feb. 17, 2010, entitled BIOPROCESSING; U.S. Provisional Patent Application No. 61/319,578, filed Mar. 31, 2010, entitled CELL-BASED BIOPROCESSING; U.S. Provisional Patent Application No. 61/319,589, filed Mar. 31, 2010, entitled CELL-BASED BIOPROCESSING; U.S. Provisional Patent Application No. 61/320,840, filed Apr. 5, 2010, entitled CELL-BASED BIOPROCESSING; U.S. Provisional Patent Application No. 61/321,815, filed Apr. 7, 2010, entitled CELL-BASED METHODS AND REAGENTS; U.S. Provisional Patent Application No. 61/322,077, filed Apr. 8, 2010, entitled CELL-BASED METHODS AND REAGENTS; and U.S. Provisional Patent Application No. 61/324,624, filed Apr. 15, 2010, entitled CELL-BASED METHODS AND REAGENTS; each of which is incorporated in their entirety herein by reference.

### FIELD OF THE INVENTION

The present invention relates to compositions and methods for isolating cells devoid of unwanted viral contaminants, and to methods for preparing a virus stock substantially devoid of viral contaminants. Virus stocks, cells, and immunogenic reagents produced using such methods are also provided.

### BACKGROUND

Cell culture techniques are used to manufacture a wide range of biological products, including biopharmaceuticals, vaccines biofuels, metabolites, vitamins, and nutraceuticals. Large-scale cell culture operations for biotechnology products use millions of liters of complex media and gases as well as huge quantities of organic and inorganic raw materials. A number of strategies have been developed to enhance productivity, yield, efficiency, and other aspects of cell-based bioprocesses in order to facilitate industrial scale production and meet applicable standards for product quality and consistency.

Endogenous risk factors, such as viral agents, remain a concern in the production of therapeutics and vaccines in cell culture. For example, latent DNA viruses (such as herpesviruses) and endogenous retroviruses (ERVs), or retroviral elements are likely present in all vertebrates. Endogenous retroviral sequences are an integral part of eukaryotic genomes, and although the majority of these sequences are defective, some can produce infectious virus, either spontaneously or upon long-term culture. ERV virus production can also be induced upon treatment with various chemical or other agents that may be part of the normal production system. Additionally, although many endogenous retroviruses do not readily re-infect their own cells, they can infect other species in vitro and in vivo. For example, two of three subgroups of pig ERVs (PERVs), can infect human cells in vitro.

There are at least twenty-six distinct groups of human endogenous retroviruses (HERVs); and mouse, cat, and pig harbor replication-competent ERVs that are capable of interacting with related exogenous virus. Retrovirus-induced tumorigenesis can involve the generation of a novel pathogenic virus by recombination between replication-competent and -defective sequences and/or activation of a cellular oncogene by a long terminal repeat (LTR) due to upstream or downstream insertion of retrovirus sequences. Thus, the activation of an endogenous, infectious retrovirus in a cell substrate that is used for the production of biologics is an important safety concern, especially in the case of live, viral vaccines, where minimal purification and inactivation steps are used in order to preserve high vaccine potency.

Adventitious viruses represent a major risk associated with the use of cell-substrate derived biologicals, including vaccines and antibodies, for human use. The possibility for viral contamination exists in primary cultures and established cultures, as well as Master Cell Banks, end-of-production cells, and bulk harvest fluids. For example, this is a major obstacle to the use of neoplastic-immortalized cells for which the mechanism of transformation is unknown, because these could have a higher risk of containing oncogenic viruses. Extensive testing for the presence of potential extraneous agents is therefore required to ensure the safety of the vaccines. The most common scenarios for adventitious viral contamination of biologics include bovine viral diarrhoea virus in foetal bovine serum; porcine parvovirus in porcine substrates; and murine minute virus, reovirus, vesivirus and Cache Valley virus in Chinese hamster cell-derived bulk harvests. The three last-named viral entities are believed to be introduced via bovine serum used during the manufacturing process (during scale-up or during the entire process).

During the production of live attenuated viral vaccines, removal of contaminating viral particles, nucleic acid, or proteins is problematic because any antiviral approach must leave the viral product intact and immunogenic. Indeed, endogenous avian viral particles have been found in commercially released human measles and mumps vaccines derived from chicken embryo fibroblasts. Moreover, endogenous viral proteins, particularly envelop proteins, often inhibit the efficiency of recombinant viral vectors used in creating transformed cell lines. Further, endogenous virus may aggravate the immune response of the host cell, often triggered during viral production or retroviral transduction. Hence, there remains a need for techniques to obtain reagents, e.g. virus stocks, or cells, that are substantially devoid of viral contaminants.

### SUMMARY

Embodiments of the present invention provide for methods and compositions for preparing a virus stock or viral sample that is substantially devoid of unwanted viral contaminants. In one aspect, the method comprises contacting a seed stock or sample containing desired virus with an agent (e.g., a receptor, an antibody, or an antigen binding fragment thereof) that binds unwanted viral contaminants, but does not bind the desired virus; and collecting the unbound desired virus. In one embodiment, the binding agent is a cell-surface receptor for the unwanted viral contaminant. In one embodiment, the cell-surface receptor for the unwanted viral contaminant is heparin. In another embodiment, expression of the cell-surface receptor in the cell has been increased using a RNA effector molecule(s). In a particular embodiment, the cell expression of heparin sulfate is increased using a RNA effector molecule that targets the heparanase gene. The cell can be a cell that does not express receptor(s) that bind the desired virus. In another embodiment, expression of the receptor(s) that bind to the desired virus in the cell has been decreased using an RNA effector molecule(s). In another embodiment of the invention, the unwanted virus-binding agent is an immobilized agent. The agent is capable of specifically binding the unwanted virus. Examples include heparin (heparin sulfate) or an antibody. In a specific embodiment, the binding agent is heparin sulfate, and the unwanted viral contaminant is porcine circovirus type 1(PCV1).

In a further embodiment of the invention, the unwanted virus-binding agent is a cell comprising a cell-surface receptor for the unwanted viral contaminant, for which expression has been increased using RNA effector molecule(s), and for which presence of a cell-surface receptor for the desired virus has been decreased. The decreased presence of the cell-surface receptor for the desired virus can be achieved using the RNA effector molecule(s) of the present invention, or by known techniques (e.g., enzymatic treatment). In a specific embodiment, the cell expression of heparin is increased using a RNA effector molecule that targets the heparanase gene, and the presence of cell-surface sialic acid is decreased by treatment of the cell with sialidase. In another specific embodiment, the cell expression of heparin is increased using a RNA effector molecule that targets the heparanase gene, and the presence of cell-surface sialic acid is decreased using a RNA effector molecule. In one embodiment, the RNA effector is selected to reduce cell-surface sialic acid content. In a particular embodiment, the RNA effector targets a gene selected from the group consisting of: UDP-GlcNAc 2-Epimerase (GNE), Sialic acid synthase (SAS), CMP-Neu5Ac synthetase (CMPNS), a Sialyltransferase, and N-acylneuraminate-9-phosphatase (3.1.3.29). In one embodiment, the unwanted viral contaminant is PCV1, and the desired virus is rotavirus.

Another aspect of the present invention provides for a method of enriching for a desired virus comprising propagating the virus in cells in which a receptor for a suspected viral contaminant has been inhibited; and collecting the desired virus. In one embodiment, the viral contaminant is PCV1 and the receptor is heparin, for which expression is decreased using a RNA effector molecule targeting the xylosyltransferase gene. In an additional embodiment, the method further comprises contacting the cells during propagation with an RNA effector molecule that targets a gene from PCV1 (for example PVC1 rep and/or cap genes), such that replication of PCV1 is inhibited. In another embodiment, the desired virus is rotavirus.

In additional aspects, the present invention provides for a method for isolating a cell that is substantially devoid of a target (e.g., contaminating) virus. The method comprises: (a) contacting a population of cells, a portion of which comprises the target virus with a RNA effector molecule that inhibits the growth or replication of the target virus; (b) detecting the presence of the target virus in each cell; and (c) isolating at least one cell that is substantially devoid of the target virus.

In one embodiment, the method further comprises repeating steps (a)-(c).

In another embodiment, the method further comprises detecting the presence or absence of target viral nucleic acid or protein (e.g., detect inhibition of transcription and/or translation). The presence or absence of the target virus, viral nucleic acid or protein can be detected, for example, by phenol emulsion DNA reassociation technique (PERT), polymerase chain reaction (PCR), Mass Spectrometry, reverse transcription polymerase chain reaction (RT-PCR), rapid amplification of cDNA ends (RACE), electrophoretic methods, immunofluorescence, dot blot hybridization, Northern hybridization, Southern hybridization, radioimmunoassay, competitive-binding assay, ELISA, Western blot, fluorescence activated cell sorting (FACS), immunohistochemistry, immunoprecipitation, proteomics, mass spectrometry, electrophoresis, immunofluoresence and the like.

In one embodiment, the target virus is a non-integrating virus.

In another embodiment, the target virus is selected from the group consisting of: hepatitis B virus (HBV), hepatitis C virus (HCV), Epstein Barr virus (EBV), human T-lymphotropic virus (HTLV), reovirus type 3 (Reo3), Sendai virus, feline sarcoma virus, human papillomavirus (HPV), hantavirus, Marburg virus, lactic dehydrogenase virus (DHM), lymphocytic choriomeningitis Virus (LCM), minute virus of mice (MVM), mouse adenovirus (MAV), mouse cytomegalovirus (MCMV), mouse rotavirus (EDIM), pneumonia virus of mice (PVM), pseudorabies virus (PRV), murine leukemia virus (MuLV), Semliki Forest virus (SFV), vesivirus, Kilham rat virus (KRV), Toolan virus (HI), bovine viral diarrhea virus (BVDV), infectious bovine rhinotracheitis virus, parainfluenza virus type 3, simian virus 40 (SV40), simian virus 5 (sv5), and simian virus 20 (SV20). In still another embodiment, the target virus is a porcine circovirus (PCV).

In another embodiment, the method further comprises (a) contacting the unbound virus stock with a host cell; (b) contacting the cell with an RNA effector molecule that inhibits growth or replication of the unwanted target virus; and (c) isolating the virus stock from the medium. The steps can be performed in any order. In one embodiment, the host cell does not have a receptor for the unwanted target virus.

The isolated cell is substantially devoid of a target virus. In one embodiment, the isolated cell contains at least 90%, for example at least 95%, at least 99%, at least 99.9%, or at least 99.99% reduced amount of detectable virus, viral particles or viral nucleic acid when compared with the initial cell population. In another embodiment, the isolated cell is free of the target virus.

In another embodiment, steps of the method are repeated as long as target viral DNA or protein is detected. In still another embodiment, the steps are repeated until the virus is no longer detectable.

The method contemplates the use of any method known in the art for detecting the presence of the target virus (contaminating virus) associated with the cell, including immunological methods (western blotting, immunofluorescence, fluorescence activated cell sorting (FACS), etc.). For example, an antibody that recognizes an epitope of the target virus can be used. In one such embodiment, the presence of the target virus is detected using an antibody against a capsid protein (Cap). Non-limiting examples of such antibodies useful for target virus detection include those described in Tischer et al., 140 Archiv. Virol. 1427-39 (1995).

In another embodiment, the cell further comprises a second virus. The second virus can be useful for production of an immunogenic agent. In one embodiment, the second virus is an attenuated virus. The method can further comprise detecting the presence of the second virus. The second virus can include any number of viruses that can be used in the production of an immunogenic agent. In one embodiment, the second virus is a rotavirus. The second virus can be detected by any method known in the art, including immunological methods to detect viral protein (western blotting, immunofluorescence, fluorescence activated cell sorting (FACS), etc), methods to detect viral nucleic acid (e.g., PERT, PCR, RT-PCR, RACE, etc). For example, an antibody that recognizes an epitope within the target virus can be used.

In one embodiment, the RNA effector molecule is an siRNA. In another embodiment, the RNA effector molecule is at least partly complementary to a portion of the target virus. In still another embodiment, the target virus is a porcine circovirus (for example, as described herein in Table 2, or as described in Sun et al., 123 Veterin. Microbiol. 203-09 (2007). In a further embodiment, the RNA effector molecule is detectably labeled. For example, the RNA effector molecule can be labeled with a fluorophore (e.g., Cy3, ALEXA FLUOR® dyes, fluorescein, etc). In another embodiment, the method further comprises detecting the presence of the RNA effector molecule in the cell. In one particular embodiment, cells with detectably labeled RNA effector molecules and without detectable target virus are isolated.

In another aspect, the invention provides for a cell that is substantially devoid of a target virus. The cell can be obtained by any of the processes described herein.

The cell thus isolated can be used in the manufacture of a biologic, for example, an immunogenic agent. Therefore, in another aspect, the present invention provides for a method of producing an immunogenic agent, comprising culturing the cell that is substantially devoid of a target virus and produced by any of the processes described herein under conditions that allow expression of the immunogenic agent; and, isolating the immunogenic agent.

The present invention provides for compositions and methods for producing an immunogenic agent in a host cell, comprising introducing into the host cell at least one oligonucleotide (e.g., a RNA effector molecule), a portion of which is complementary to at least one nucleic acid-based entity (e.g., a target gene); maintaining the host cell for a time sufficient to modulate expression of the at least one nucleic acid-based entity (e.g., a target gene), wherein the modulation of expression inhibits expression of latent, adventitious, and/or endogenous virus, and enhances production of an immunogenic agent in the host cell; and isolating the immunogenic agent from the host cell.

More specifically, some embodiments of the present invention relate to initiating RNA interference in a host cell, during or after microbial inoculation or vector transduction, to inhibit expression of endogenous, latent or adventitious virus that compromises the yield and/or quality of the harvested immunogenic agent. For example, an embodiment administers a siRNA or shRNA in naked, conjugated or formulated (e.g., lipid nanoparticle) form that targets an endogenous, latent or adventitious virus pathway (e.g., ev loci of endogenous avian leukosis virus (ALV-E) in avian cells; endogenous type C retrovirus-like particle genomes in Chinese Hamster Ovary (CHO) cells; or the rep gene of porcine circovirus type 1 (PCV-1) in Vero cells), and thereby increases quality and/or yield of the desired immunogenic agent.

In some embodiments of the invention, with regard to RNAi formulations, simple (naked siRNA in saline or similar solutions or formulations), conjugated (e.g., cholesterol or other targeting ligands) as well as LNP or alternate polymer formulations or delivery vehicles as well as plasmid or viral vectors for shRNA can be used.

In addition, the formulations can be co-formulated or incorporated into the infective seed or vectors themselves to facilitate delivery or stabilize RNAi materials to the relevant cell where the agent/vector can produce the desired product.

In some embodiments, the oligonucleotide (e.g., RNA effector molecule) can inhibit a target gene. In other embodiments, the RNA effector can activate a target gene. In particular embodiments, the target gene is associated with endogenous, adventitious or latent herpesviruses, polyomaviruses, hepadnaviruses, papillomaviruses, adenoviruses, poxviruses, bornaviruses, retroviruses, arenaviruse, orthomyxoviruses, paramyxoviruses, reoviruses, picornaviruses, flaviviruses, rabdoviruses, hantaviruses, circoviruses, or vesiviruses.

Particular endogenous and latent viruses that can be targeted by the methods of the present invention include Minute Virus of Mice (MVM), Murine leukemia/sarcoma (MLV), Circoviruses including porcine circovirus (PCV-1, PCV-2), Human herpesvirus 8 (HHV-8), arenavirus Lymphocytic choriomeningitis virus (LCMV), Lactate dehydrogenase virus (LDH or LDV), human species C adenoviruses, avian adeno-associated virus (AAV), primate endogenous retrovirus family K (ERV-K), and human endogenous retrovirus K (HERV-K).

Further regarding ERVs, in embodiments of the present invention the target genes of ERVs can be those of primate/human Class I Gamma ERVs pt01-Chr10r-17119458, pt01-Chr5-53871501, BaEV, GaLV, HERV-T, ERV-3, HERV-E, HERV-ADP, HERV-I, MER4like, HERV-FRD, HERV-W, HERVH-RTVLH2, HERVH-RGH2, HERV-Hconsensus, HERV-Fc1; primate/human Epsilon ERV hg15-chr3-152465283; primate/human Intermediate (epsilon-like) HERVL66; primate/human Class III Spuma-like ERVs HSRV, HFV, HERV-S, HERV-L, HERVL40, HERVL74; primate/human Delta ERVs HTLV-1, HTLV-2; primate/human Lenti ERVs HIV-1, HIV-2; primate/human Class II, Beta ERV MPMV, MMTV, HML1, HML2, HML3, HML4, HML7, HML8, HML5, HML10, HML6, or HML9.

In other embodiments of the present invention, the ERV is selected from rodent Class II, Beta ERV MMTV; rodent Class I Gamma ERV MLV; feline Class I Gamma ERV FLV; ungulate Class I Gamma ERV PERV; ungulate Delta ERV BLV; ungulate lentivirus Visna, EIAV; ungulate Class II, Beta ERV JSRV; avian Class III, Spuma-like ERVs gg01-chr7-7163462; gg01-chrU-52190725, gg01-Chr4-48130894; avian Alpha ERVs ALV, gg01-chr1-15168845; avian Intermediate Beta-like ERVs gg01-chr4-77338201; gg01-ChrU-163504869, gg01-chr7-5733782; Reptilian Intermediate Beta-like ERV Python-molurus; Fish Epsilon ERV WDSV; fish Intermediate (epsilon-like) ERV SnRV; Amphibian Epsilon ERV Xen1; Insect Errantivirus ERV Gypsy.

Other embodiments of the present invention target adventitious viruses of animal-origin, such as vesivirus, circovirus, hantaan virus, Marburg virus, SV40, SV20, Semliki Forest virus (SFV), simian virus 5 (sv5), lymphocytic choriomeningitis virus, feline sarcoma virus, porcine parvovirus, adenoassociated viruses (AAV), mouse hepatitis virus (MHV), murine leukemia virus (MuLV), pneumonia virus of mice (PVM), Theiler's encephalomyelitis virus (THEMV), murine minute virus (MMV or MVM), mouse adenovirus (MAV), mouse cytomegalovirus (MCMV), mouse rotavirus (EDIM), Kilham rat virus (KRV), Toolan's H-1 virus, Sendai virus (SeV, also know as murine parainfluenza virus type 1 or hemagglutinating virus of Japan (HVJ)), Parker's rat coronavirus (RCV or SDA), pseudorabies virus (PRV), reoviruses, Cache Valley virus, bovine viral diarrhoea virus, bovine parainfluenza virus type 3, bovine respiratory syncytial virus, bovine adenoviruses, bovine parvoviruses, bovine herpesvirus 1 (infectious bovine rhinotracheitis virus), other bovine herpesviruses, bovine reovirus, rabies virus, bluetongue viruses, bovine polyoma virus, bovine circovirus, and orthopoxviruses other than vaccinia, pseudocowpox virus (a widespread parapoxvirus that can infect humans), papillomavirus, herpesviruses, or leporipoxviruses.

Other embodiments target human-origin adventitious agents including HIV-1 and HIV-2; human T cell lymphotropic virus type I (HTLV-I) and HTLV-II; human hepatitis A, B, and C viruses; human cytomegalovirus; Epstein Barr virus (EBV or HHV-4); human herpesviruses 6, 7, and 8; human parvovirus B19; reoviruses; polyoma (JC/BK) viruses; SV40 virus; human coronaviruses; human papillomaviruses; influenza A, B, and C viruses; human enteroviruses; human parainfluenza viruses; and human respiratory syncytial virus.

Yet other embodiments of the present invention target host cell surface receptors or intracellular proteins to which endogenous, latent, or adventitious virus bind or which are required for viral replication. For example, in a particular embodiment, the target gene is a CHO cell MVM receptor gene, such as a gene associated with cellular sialic acid production.

In some embodiments, the immunogenic agent produced via the compositions and methods of the present invention is a viral immunogenic agent, from any type of animal virus, such as those of arenaviridae, orthomyxoviridae, paramyxoviridae, Filoviridae, rabdoviridae, birnaviridae, reoviridae, picornaviridae, coronaviridae, flaviviridae, togaviridae, adenoviridae, herpesviridae, papovaviridae, parvoviridae, circoviridae, poxyiridae, and retroviridae. In particular embodiments, the virus is poliovirus, hepatitis A virus (HAV), tick-borne encephalitis virus (TBEV), yellow fever virus, rubella virus, hepatitis C virus (HCV), hepatitis B virus (HBV), variola virus, mumps virus, measles virus, rubella virus, respiratory syncytial virus, vesicular stomatits virus (VSV), rabies virus, ebola virus, influenza virus, lassa virus, junin virus, reovirus, adenovirus type 1 to type 47, herpes simplex viruses (HSV 1, HSV 2), cytomegalo virus (CMV), varicella zoster vim (VZV), Epstein-Barr virus (EBV), human immunodeficiency virus (HIV), West Nile virus, rotavirus, or vaccinia virus.

In particular embodiments, the immunogenic agent is influenza, and can include attenuated influenza viruses, cold-adapted influenza viruses, temperature-sensitive influenza viruses, reassortant influenza viruses, high yield donor influenza viruses, wild-type influenza viruses isolated from throat swabs of infected mammals, and viruses that have been passaged in embryonated chicken eggs, or cell culture adapted strains of influenza viruses.

Thus, in some embodiments of the present invention, the immunogenic agent is a virus, including naturally occurring strains, variants, or mutants; mutagenized viruses (e.g., generated by exposure to mutagens, repeated passages and/or passage in non-permissive hosts); reassortants (in the case of segmented viral genomes); and/or genetically engineered viruses (e.g., using the “reverse genetics” techniques) having the desired phenotype; and other virus-based (viral) products. The viral immunogenic agent of the invention can be attenuated virus; i.e., virus that is infectious and can replicate in vivo, but generates low titers resulting in subclinical levels of infection that are non-pathogenic.

Additionally, in some embodiments the desired viral immunogenic agent is viral progeny that has been attenuated by targeting viral proteins associated with virulence (e.g., influenza NP, PA, PB1, PB2, M, and NS genes). The glycosylation pattern of biologic product of interest, such as the hemagglutinin (HA) and neuraminidase (NA) influenza proteins, can be attenuated to improve antigenicity or host adaptation. In essence, any aspect of the quality and attributes as well efficiency of bioprocessing can be modified by this approach.

Alternative embodiments of the present invention provide for production of an immunogenic bacterial agent, i.e., a bacterial immunogen, in a eukaryotic host cell. These bacteria include Shigella flexneri, Listeria monocytogenes, Rickettsiae tsutsugamushi, Rickettsiae rickettsiae, Mycobacterium leprae, Mycobacterium tuberculosis, Legionella pneumophila, Chlamydia ssp.

Additional embodiments of the present invention provide for production of an immunogenic protozoal agent, i.e., a protozoal immunogen, in a eukaryotic host cell. These protozoa include Plasmodium falciparum, Tripanosoma cruzi, and Leishmania donovani.

The method of the invention also can include the steps of monitoring the growth, infection and activation levels of the host cell culture, and as well as for varying the conditions of the host cell culture to maximize the growth, infection and activation levels of the host cells and desired agent, and for harvesting the immunogenic agent (e.g., virus) from the culture, preparing a vaccine with the harvested agent, and for the treatment and/or the prevention of infection or disease by administering to a subject a vaccine obtained by the method.

In some embodiments, the host cell is administered a plurality of different oligonuceotides (e.g., RNA effector molecules) to modulate expression of multiple target genes. The RNA effector molecules can be administered at different times or simultaneously, at the same frequency or different frequencies, at the same concentration or at different concentrations.

In further embodiments, the method further comprises administering to the host cell a second agent. The second agent can be a growth factor; an apoptosis inhibitor; a kinase inhibitor; a phosphatase inhibitor; a protease inhibitor; an inhibitor of pathogens (e.g., where a virus is the biological product, an agent that inhibits growth and/or propagation of other viruses or fungal or bacterial pathogens); or a histone demethylating agent. Where the virus being propagated is influenza, the second agent can be a protease that cleaves influenza hemagglutinin, such as pronase, thermolysin, subtilisin A, or a recombinant protease.

More specifically, in some embodiments the second target gene is a gene associated with host cell immune response, and the target gene selected from the group consisting of TLR3, TLR7, TLR21, RIG-1, LPGP2, RIG 1-like receptors, TRIM25, IFN-α, IFN-β, IFN-γ MAVS/VISA/IPS 1/Gardif, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF 9, IRF10, 2′,5′ oligoadenylate synthetase, RNaseL, dsRNA-dPKR, Mx, IFITM1, IFITM2, IFITM3, Proinflammatory cytokines, MYD88, TRIF, and a regulatory region of any of the foregoing.

In other specific embodiments, the second target gene is a gene associated with host cell viability, growth or cell cycle, and the target gene is selected from the group consisting of Bax, Bak, LDHA, LDHB, BIK, BAD, BIM, HRK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, BCL2L13, BCL2L14, FASLG, DPF2, AIFM2, AIFM3, STK17A, APITD1, SIVA1, FAS, TGFβ2, TGFBR1, LOC378902, or BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, a miRNA antagonist, host sialidase, NEU2 sialidase 2, NEU3 sialidase 3, Dicer, ISRE, and a regulatory region of any of the foregoing.

In some embodiments, the invention provides for a host cell that contains at least one RNA effector molecule provided herein. The host cell can be derived from an insect, amphibian, fish, reptile, bird, mammal, or human, or can be a hybridoma cell. For example, the cell can be a human Namalwa Burkitt lymphoma cell (BLc1-kar-Namalwa), baby hamster kidney fibroblast (BHK), CHO cell, Murine myeloma cell (NS0, SP2/0), hybridoma cell, human embryonic kidney cell (293 HEK), human retina-derived cell (PER.C6® cells), insect cell line (Sf9, derived from pupal ovarian tissue of Spodoptera frugiperda; or Hi-5, derived from Trichoplusia ni egg cell homogenates), Madin-Darby canine kidneycell (MDCK), primary mouse brain cells or tissue, primary calf lymph cells or tissue, primary monkey kidney cells, embryonated hens' egg, primary chicken embryo fibroblast (CEF), Rhesus fetal lung cell (FRhL-2), Human fetal lung cell (WI-38, MRC-5), African green monkey kidney epithelial cell (Vero, CV-1), Rhesus monkey kidney cell (LLC-MK2), or yeast cell.

In additional embodiments, the target gene encodes a protein that affects a physiological process of the host cell. In various embodiments, the physiological process is apoptosis, cell cycle progression, carbon metabolism or transport, lactate formation, or RNAi uptake and/or efficacy.

In other embodiments, the invention provides a composition for enhancing production of an immunogenic agent in a host cell by modulating the expression of a target gene in the cell. The composition typically includes one or more oligonuceotides, such as RNA effector molecules described herein, and a suitable carrier or delivery vehicle.

In other embodiments, a composition containing two or more different RNA effector molecules directed against different target genes for the same protein is used to enhance production of an immunogenic agent in a host cell by modulating expression of a the same protein gene in the host cell, wherein the first and second target gene can be associated with expression of the protein but, for example, the first target is a coding region and the second target is an untranslated region (UTR) of the protein gene.

Still another embodiment of the invention encompasses kits for enhancing production of an immunogenic agent in a host cell. In one aspect, a kit comprises a RNA effector molecule that modulates expression of a target gene to inhibit expression of a latent, adventitious, or endogenous virus and thus affect production of the desired immunogenic agent. In another embodiment, a kit comprises a host cell that expresses a RNA effector molecule that modulates expression of latent, adventitious, or endogenous virus that affects production of the desired immunogenic agent. Such kits can also comprise instructions for carrying out methods provided herein. The kits can also include at leat one reagent that facilitates RNA effector molecule-uptake, comprising a charged lipid, an emulsion, a liposome, a cationic or non-cationic lipid, an anionic lipid, a transfection reagent or a penetration enhancer. In a particular embodiment, the reagent that facilitates RNA effector molecule-uptake comprises a charged lipid.

Other embodiments of the present invention provide for methods of removing latent or adventitious virus from contaminated cells comprising: identifying a target gene that is required for viral replication or infectivity; designing at least one RNA effector molecule against that gene target; contacting the contaminated cells with the RNA effector molecule; screening the cells to identify those no longer expressing viral nucleic acids, proteins, or infective particles; and expanding those identified virus-free cells.

A particular embodiment of the present invention provides a method for producing an influenza virus and/or influenza viral proteins for use in a vaccine, the method comprising: providing a host cell with nucleic acid encoding an influenza virus or infecting the host cell with influenza virus; providing the host cell with at least one RNA effector molecule that inhibits latent, adventitious, or endogenous virus; culturing the host cell in a suitable medium and allowing for propagation of the influenza virus or production of the viral proteins; and harvesting the influenza virus and/or influenza viral proteins from the suitable medium and/or the host cell.

Other embodiments provides for a process for producing hemagglutinin proteins and/or neuraminidase proteins of an influenza virus, the process comprising: providing a host cell with nucleic acid encoding an influenza virus or infecting the cell with influenza virus; providing the host cell with at least one RNA effector molecule that targets latent, adventitious, or endogenous virus; culturing the hsot cell in a suitable medium and allowing for propagation of the influenza virus; harvesting the propagated influenza virus; disrupting the influenza virus; and isolating hemagglutinin proteins and/or neuraminidase proteins from the disrupted influenza virus.

An advantage of the present invention is the ability to substantially increase the yield of host cells and immunogenic agent-based products, i.e., vaccines, and thereby reduce production costs. Improved manufacturing logistics have the follow-on effect of enhancing quality control, as well as expand the vaccine supply, for example in epidemic and pandemic outbreaks of diseases such as influenza.

### DETAILED DESCRIPTION

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents, oligonucleotide sequences identified by gene identification numbers, and other publications identified herein are expressly incorporated by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. All gene identification numbers provided herein (GeneID) are those of the National Center for Biotechnology Information “Entrez Gene” web site unless identified otherwise. Although any known methods, devices, and materials can be used in the practice or testing of the invention, the methods, devices, and materials in this regard are described herein.

The present invention provides for the production of biological products devoid of contaminating viruses; biological products such as immunogenic agents, for example, viruses, viral antigens, bacteria, or protozoans, or recombinant proteins derived from a virus, viral expression vector, bacterium, or protozoan. Embodiments of the invention provide for biological products, cells (e.g., cell banks or production lines), and virus stock that are devoid of unwanted viral contaminants (e.g., virus particles, proteins, nucleic acids).

A current approach for producing viral-based vaccines involves the use of attenuated live virus vaccines, which are capable of replication but are not pathogenic, and, therefore, provide lasting immunity and afford greater protection against disease. The conventional methods for producing attenuated viruses involve the chance isolation of host range mutants, many of which are temperature sensitive, e.g., the virus is passaged through unnatural hosts, and progeny viruses which are immunogenic, yet not pathogenic, are selected. Efficient vaccine production requires the growth of large quantities of virus produced in high yields from a host system. Different types of virus require different growth conditions in order to obtain acceptable yields. The host in which the virus is grown is therefore of great significance. As a function of the virus type, a virus can be grown in embryonated eggs, primary tissue culture cells, or in established cell lines.

For example, since the introduction in the 1940s of an inactivated influenza vaccine containing inactivated virus material from infected incubated eggs, the risk and course of the infection as well as the mortality rates in the elderly have dropped. A particular advantage of inactivated influenza virus or influenza viral antigens obtained cell culture is the absence of egg-specific proteins that might trigger an allergic reaction against egg proteins. Therefore, the use according to the invention is especially suitable for the prophylaxis of influenza virus infections, particularly in populations that constitute higher-risk groups, such as asthmatics, those with allergies, and also people with suppressed immune systems and the elderly.

The cultivation conditions under which a virus strain is grown in cell culture also are of great significance with respect to achieving an acceptably high yield of the strain. In order to maximize the yield of a desired virus strain, both the host system and the cultivation conditions must be adapted specifically to provide an environment that is advantageous for the production of a desired virus strain. Many viruses are restricted to very specific host systems, some of which are very inefficient with regard to virus yields. Some of the mammalian cells which are used as viral host systems produce virus at high yields, but the tumorigenic nature of such cells invokes regulatory constraints against their use for vaccine production.

The problems arising from the use of serum in cell culture and/or protein additives derived from an animal or human source added to the culture medium, e.g., the varying quality and composition of different batches and the risk of contamination with mycoplasma, viruses or BSE-agent, are well-known. In general, serum or serum-derived substances like albumin, transferrin or insulin can contain unwanted agents that can contaminate the culture and the biological products produced therefrom. Furthermore, human serum derived additives have to be tested for all known viruses, like hepatitis or HIV, which can be transmitted by serum. Bovine serum and products derived therefrom, for example trypsin, bear the risk of bovine spongiform encephalitis-contamination. In addition, all serum-derived products can be contaminated by still unknown agents. Therefore, cells and culture conditions that do not require serum or other serum derived compounds are being pursued.

For example, the production of smallpox vaccine, modified vaccinia virus Ankara (MVA) is amplified in cell cultures of primary or secondary chicken embryo fibroblasts (CEF). The CEF are obtained from embryos of chicken eggs that have been incubated for 10 to 12 days, from which the cells are then dissociated and purified. These primary CEF cells can either be used directly or after one further cell passage as secondary CEF cells. Subsequently, the primary or secondary CEF cells are infected with the MVA. For the amplification of MVA the infected cells are incubated for 2 to 3 days at 37° C. See, e.g., Meyer et al., 72 J. Gen. Virol. 1031-38 (1991); Sutter et al., 12 Vaccine 1032-40 (1994). Many pox viruses replicate efficiently in CEF incubated at temperatures below 37° C., such as 30° C. See U.S. Pat. No. 6,924,137.

The use of established mammalian cell lines, such as Madin-Darby canine kidney (MDCK) line, has been successful in replicating some viral strains. Nevertheless, a number of virus strains will not replicate in the MDCK line. In addition, fears over possible adverse effects associated with employing cells with a tumorigenic potential for human vaccine production have precluded the use of MDCK, a highly transformed cell line, in this context.

Other attempts at developing alternative vaccine production methods have been undertaken. U.S. Pat. No. 4,783,411 discusses a method for preparing influenza vaccines in goldfish cell cultures. The virus particles for infecting the goldfish cell cultures, after their establishment, were obtained from chicken embryo cultures or from infected CD-I strain mice. The virus is passaged at least twice in the goldfish cell cultures, resulting in an attenuated influenza virus which can be used as a live vaccine. Additionally, African green monkey kidney epithelial cells (Vero) and chicken embryo cells (CEC) have been adapted to grow and produce influenzae virus and recombinant influenzae proteins in serum-free, protein-free media. See WO 96/015231.

Although the use of protein and serum free media limits the risk from adventitious virus contamination, it does not address the continued risk posed by latent viruses or endogenous retroviruses that exist in cell banks. The activation of an endogenous, infectious retrovirus in a cell substrate that is used for the production of biologics is an important safety concern, especially in the case of live, viral vaccines, where there are minimal purification and inactivation steps in order to preserve high vaccine potency.

Retroviruses replicate by reverse transcription, mediated by an RNA-dependent DNA polymerase (reverse transcriptase), encoded by the viral pol gene. Retroviruses also carry at least two additional genes: the gag gene encodes the proteins of the viral skeleton, matrix, nucleocapsid, and capsid; the env gene encodes the envelope glycoproteins. Additionally, retroviral transcription is regulated by promoter regions or “enhancers” situated in highly repeated regions (LTRs) which are present at both ends of the retroviral genome.

During the infection of a cell, reverse transcriptase makes a DNA copy of the RNA genome; this copy can then integrate into the host cell genome. Retroviruses can infect germ cells or embryos at an early stage and be transmitted by vertical Mendelian transmission. These endogenous retroviruses (ERVs) can degenerate during generations of the host organism and lose their initial properties. Some ERVs conserve all or part of their properties or of the properties of their constituent motifs, or acquire novel functional properties having an advantage for the host organism. These retroviral sequences can also undergo, over the generations, discrete modifications which will be able to trigger some of their potential and generate or promote pathological processes.

Human endogenous retroviral sequences (HERVs) represent a substantial part of the human genome. These retroviral regions exist in several forms: complete endogenous retroviral structures combining gag, pol and env motifs, flanked by repeat nucleic sequences which exhibit a significant analogy with the LTR-gag-pol-env-LTR structure of infectious retroviruses; truncated retroviral sequences, for example the retrotransposons lack their env domain; and the retroposons that lack the env and LTR regions. ERVs capable of shedding virus particles are often called type C ERVs.

Important ERVs include human teratocarcinoma retrovirus (HTDV), or HERV-K, an endogenous retrovirus known to produce viral particles from endogenous provirus. Lower et al., 68 J. Gen. Virol. 2807-15 (1987); Mold et al., 4 J. Biomed. Sci. 78082 (2005). HERV-R is another important ERV, because it has been found to be expressed in many tissues, including the adrenal cortex and various adrenal tumors such as cortical adenomas and pheochromocytomas. Katsumata et al., 66 Pathobiology 209-15 (1998). Murine leukemia virus (MLV) is another important ERV, that produces infective virus particles in rodent-derived cell culture upon induction. Khan & Sears, 106 Devel. Biol. 387-92 (2001). Indeed, cell culture changes that significantly alter the metabolic state of the cells and/or rates of protein expression (e.g., pH, temperature shifts, sodium butyrate addition) measurably increased the rate of endogenous retroviral synthesis in CHO cells. Brorson et al., 80 Biotech. Bioengin. 257-67 (2002).

An on-line database, called HERVd—Human Endogenous Retrovirus Database (NAR Molecular Biology Database Collection entry number 0495), has been compiled from the human genome nucleotide sequences, obtained mostly in the various ongoing Human Genome Projects. This provides a relatively simple and fast environment for screening HERVs, and makes it possible to continuously improve classification and characterization of retroviral families. The HERVd database now contains retroviruses from more than 90% of the human genome. Additionally, ERV sequences can be obtained readily through the National Institutes of Health's on-line “Entrez Gene” site.

Further regarding ERVs, embodiments of the present invention target at least one gene or LTR of primate/human Class I Gamma ERVs pt01-Chr10r-17119458, pt01-Chr5-53871501, BaEV, GaLV, HERV-T, HERV-R (HERV-3, ERV3 env gene, GeneID:2086), HERV-E (ERVE1, GeneID:85314), HERV-ADP, HERV-I, MER4like, HERV-FRD (ERVFRD1, Env protein, GeneID:405754; Pan troglodytes Env protein, GeneID:471856; Rattus norvegicus Herv-frd Env polyprotein, GeneID:290348), HERV-W (ERVWE2, ERV-W, env(C7), member 2, Pan troglodytes, GeneID:100190905; HERVWE1, ERV-W, env(C7), member 1, GeneID:30816), HERV-H(HHLA1, HERV-H LTR-associating protein 1, GeneID:10086, Pan troglodytes GeneID:736282; Hhla1, mouse GeneID:654498; HHLA2, HERV-H LTR-associating protein 2, GeneID:11148; HHLA3, HERV-H LTR-associating protein 3, GeneID:11147; Xenopus hhla2, GeneID:734131), HERVH-RTVLH2, HERVH-RGH2, HERV-Hconsensus, HERV-Fc1; primate/human Epsilon endogenous retrovirus hg15-chr3-152465283; primate/human Intermediate (epsilon-like) HERVL66; primate/human Class III Spuma-like ERVs HSRV, HFV, HERV-S, HERV-L, HERVL40, HERVL74; primate/human Delta ERV HTLV-1, HTLV-2; primate/human Lenti ERV (lentivirus) HIV-1, HIV-2; primate/human Class II, Beta ERVs MPMV, MMTV, HML1, HML2, HML3, HML4, HML7, HML8, HML5, HML10, HML6, HML9, human teratocarcinoma-derived retrovirus (HTDV/HERV-K), or HERV-V (HERV-V1 Env1, GeneID:147664; HERV-V2, HSV2, GeneID:100271846).

Additional primate ERV genes that can be targeted by the methods of the present invention include LOC471586 (similar to ERV-BabFcenv provirus ancestral Env polyprotein, Pan troglodytes GeneID:471586), LOC470639 (similar to ERV-BabFcenv provirus ancestral Env polyprotein, Pan troglodytes GeneID:470639); LOC100138322 (similar to HERV-K_{—}7p22.1 provirus ancestral Pol protein, Bos taurus GeneID:10013822; LOC110138431 (similar to HERV-K_{—}1q22 provirus ancestral Pol protein, Bos taurus GeneID:100138431; LOC100137757 (similar to HERV-K_{—}6q14.1 provirus ancestral Gag-Pol polyprotein, Bos taurus GeneID:100137757); LOC100141085 (similar to HERV-K_{—}8p23.1 provirus ancestral Pol protein, Bos taurus GeneID:100141085); LOC100138106 (similar to HERV-F(c)1_Xq21.33 provirus ancestral Gag polyprotein, Bos taurus GeneID: LOC100138106); LOC100140731 (similar to HERV-W_{—}3q26.32 provirus ancestral Gag polyprotein Bos taurus, GeneID:100140731); LOC100139657 (similar to HERV-W_{—}3q26.32 provirus ancestral Gag polyprotein Bos taurus GeneID:100139657).

In other embodiments of the present invention, the ERV is rodent Class II, Beta ERV mouse mammary tumor (MMTV, GeneID:2828729; MMTVgp7, GeneID:1491863; MMTV env GeneID:1491862; MMTVgp1, GeneID:1724724; MMTVgp2, GeneID:1724723; MMTV pol GeneID:1491865; MMTV pro, GeneID:1491865; MMTV gag, GeneID:1491864); rodent Class I Gamma ERV MLV (Mlv1, mouse GeneID:108317); feline Class I Gamma ERV FLV; ungulate Class I Gamma ERV PERV; ungulate Delta ERV BLV; ungulate lentivirus Visna, EIAV; ungulate Class II, Beta ERV JSRV; avian Class III, Spuma-like ERVs gg01-chr7-7163462; gg01-chrU-52190725, gg01-Chr4-48130894; avian Alpha ERVs ALV (ALV pol GeneID:1491910; ALVp2, GeneID:1491909; ALVp10, GeneID:1491908; ALV env, GeneID:1491907; ALV transmembrane protein, tm, GeneID:1491906; ALV trans-acting factor, GeneID:1491911), gg01-chr1-15168845; avian Intermediate Beta-like ERVs gg01-chr4-77338201; gg01-ChrU-163504869, gg01-chr7-5733782; Reptilian Intermediate Beta-like ERV Python-molurus; Fish Epsilon ERV WDSV; fish Intermediate (epsilon-like) ERV SnRV; Amphibian Epsilon ERV Xen1; Insect Errantivirus ERV Gypsy; or Ty1 in Saccharomyces cerevisiae, yeast ORF161 (ERV-1-like protein, Ectocarpus siliculosus virus 1, GeneID:920716).

Further regarding ERVs, as noted herein the HERV-K ERVs are particularly relevant because they can be activated by a variety a stimuli. Hence, aspects of the present invention target genes of the HERV-K family, including HERV-K3, GeneID:2088; HERV-K2, GeneID:2087; HERV-K_{—}11q22.1 provirus ancestral Pol protein, GeneID:100133495; HERV-K7, GeneID:449619; HERV-K6, GeneID:64006; HERV-K(1), ERVK4, GeneID:60359; and HERV-K(II), ERVK5, GeneID:60358; LOC100133495 (HERV-K_{—}11q22.1 provirus ancestral Pol protein, GeneID:100133495).

As described herein, in particular aspects of the present invention the target gene is an ERV env gene, for example eERV family W, env(C7), member 1 (ERVWE1), GeneID:30816; hypothetical LOC147664 (HERV-V1 or EnvV1), GeneID:147664; HERV-FRD provirus ancestral Env polyprotein (ERVFRDE1), GeneID:405754 and GeneID:471856; ERV sequence K, 6 (ERVK6 or HERV-K108), GeneID:64006; ERV sequence 3 envelop protein (ERV3), GeneID:2086 and GeneID:100190893; ALV Env protein, GeneID:1491907, or the Env protein of HERV-K18.

Additionally, Bornaviruses are genus of non-segmented, negative-sense, non-retroviral RNA viruses that establish persistent infection in the cell nucleus. Elements homologous to the bornavirus nucleoprotein (N) gene exist in the genomes of several mammalian species, and produce mRNA that encodes endogenous Borna-like N (EBLN) elements. Horie et al., 463 Nature 84-87 (2010). Hence, in some embodiments of the invention, the target gene is a bornaviral gene.

In addition to targeting ERV genes and regulatory sequences, some embodiments of the present invention target ERV receptors. For example, human solute carrier family 1 (neutral amino acid transporter), member 5 (SLC1A5, GeneID:6510) is a receptor for Simian type D retrovirus and feline endogenous RD-114 virus. Solute carrier family 1 (glutamate/neutral amino acid transporter), member 4 (Slc1a4, GeneID:55963) and member 5 (Slc1a5, GeneID: 20514) are mouse versions of related proteins. Human solute carrier family 1 (glutamate/neutral amino acid transporter), member 4 (SLC1A4, GeneID:6509), is used as receptor by HERV-W Env glycoprotein. Thus, inhibition of cellular viral receptors can decrease receptor interference, latent, endogenous or adventitious viral infection, and thus increase the production of immunogenic agent in the cell.

Latent DNA viruses that can be targeted by the methods of the present invention include adenoviruses. For example, species of C serotype adenovirus can establish latent infection in human tissues. See Garnett et al., 83 J. Virol. 2417-28 (2000). Avian adenovirus and adenovirus-associated virus (AAV) proteins have been produced by specific-pathogen-free chicks, indicating that avian AAV can exist as a latent infection in the germ line of chickens. Sadasiv et al., 33 Avian Dis. 125-33 (1989); see also Katano et al., 36 Biotechniq. 676-80 (2004). In some embodiments of the invention, the target gene is a latent DNA virus. For example, the target gene can be the latent membrane protein (LMP)-2A from HHV-4 (EBV), GeneID:3783751, which protein also transactivates the Env protein of HERV-K18.

Circoviridae are also DNA viruses that exhibit a latent phase. Porcine circoviridae type 1 (PCV1) was shown to contaminate Vero cell banks from which rotavirus vaccine was made, leading to an FDA hold on administration of the vaccine. Associated Press, Mar. 23, 2010. The rep gene of PCV1 is indispensable for replication of viral DNA. Mankertz & Hillenbrand, 279 Virol. 429-38 (2001). Hence, a particular embodiment of the present invention provides for a RNA effector molecule that inhibits a PCV1 rep gene. Example dsRNA molecules are provided herein.

Other embodiments of the present invention target the genes of adventitious animal viruses, including vesivirus, porcine circovirus, lymphocytic choriomeningitis virus, porcine parvovirus, adenoassociated viruses, reoviruses, rabies virus, papillomavirus, herpesviruses, leporipoxviruses, and leukosis virus (ALV), hantaan virus, Marburg virus, SV40, SV20, Semliki Forest virus (SFV), simian virus 5 (sv5), feline sarcoma virus, porcine parvovirus, adenoassociated viruses (AAV), mouse hepatitis virus (MHV), Moloney murine leukemia virus (MoMLV or MMLV, gag protein GeneID:1491870), murine leukemia virus (MuLV), pneumonia virus of mice (PVM), Theiler's encephalomyelitis virus (THEMV), murine minute virus (MMV or MVM, GeneID:2828495, vp1, GeneID:148592; vp, GeneID:1489591; nsl, GeneID:1489590), mouse adenovirus (MAV), mouse cytomegalovirus (MCMV), mouse rotavirus (EDIM), Kilham rat virus (KRV), Toolan's H-1 virus, Sendai virus (SeV, also known as murine parainfluenza virus type 1 or hemagglutinating virus of Japan (HVJ)), rat coronavirus (RCV or sialodacryoadenitis virus (SDA)), pseudorabies virus (PRV), Cache Valley virus, bovine diarrhea virus, bovine parainfluenza virus type 3, bovine respiratory syncytial virus, bovine adenoviruses, bovine parvoviruses, bovine herpesvirus 1 (infectious bovine rhinotracheitis virus), other bovine herpesviruses, bovine reovirus, other bovine herpesviruses, bovine reovirus, bluetongue viruses, bovine polyoma virus, bovine circovirus, and orthopoxviruses other than vaccinia, pseudocowpox virus (a widespread parapoxvirus that can infect humans), papillomavirus, herpesviruses, leporipoxviruses, or exogenous retroviruses.

Other embodiments target human-origin adventitious agents including HIV-1 and HIV-2; human T cell lymphotropic virus type I (HTLV-I) and HTLV-II; human hepatitis A, B, and C viruses; human cytomegalovirus (CMV); Epstein Barr virus (EBV); human herpesviruses 6, 7, and 8; human parvovirus B19; reoviruses; polyoma (JC/BK) viruses; 5V40 virus; human coronaviruses; human papillomaviruses; influenza A, B, and C viruses; various human enteroviruses; human parainfluenza viruses; and human respiratory syncytial virus.

Parvoviridae are single-stranded DNA viruses with genomes of about 4 to 5 kilobases. This family includes: Dependovirus such as human helper-dependent adeno-associated virus (AAV) serotypes 1 to 8, autonomous avian parvoviruse, and adeno associated viruses (AAV 1-8); Erythrovirus such as bovine, chipmunk, and autonomous primate parvoviruses, including human parvoviruses B19 (the cause of Fifth disease) and V9; and Parvovirus that includes parvoviruses of other animals and rodents, carnivores, and pigs, including MVM. These parvoviruses can infect several cell types and have been described in clinical samples. AAVs, in particular, have been implicated in decreased replication, propagation, and growth of other virus.

MVM gains cell entry by deploying a lipolytic enzyme, phospholipase A2 (PLA2), that is expressed at the N-terminus of virion protein 1 (VP1, also called MMVgp3), the MVM minor coat protein, GeneID:1489592. Farr et al., 102 P.N.A.S. 17148-53 (2005). Other MVM targets can be chosen from MVM VP (also called MMVgp2), GeneID:1489591; and MVM non-structural, initiator protein (NS1, also called MMVgp1), GeneID:1489590.

Polyomaviruses are double-stranded DNA viruses that can infect, for example, humans, primates, rodents, rabbits, and birds. Polyomaviruses (PyV) include SV40, JC and BK viruses, Murine pneumonotropic virus, hamster PyV, murine PyV virus, and Lymphotropic papovavirus (LPV, the African green monkey papovavirus). The sequences for these viruses are available via GenBank. See also U.S. Patent Pub. No. 2009/0220937. Because of their tumorigenic and oncogenic potential, it is important to eliminate these viruses in cell substrates used for vaccine production.

Papillomaviridae contains more that 150 known species representing varying host-specificity and sequence homology. They have been identified in mammals (humans, simians, bovines, canines, ovines) and in birds. Majority of the human Papillomaviruses (HPVs), including all HPV types traditionally called genital and mucosal HPVs belong to supergroup A. Within supergroup A, there are 11 groups; the most medically important of these are the human Papillomaviruses HPV 16, HPV 18, HPV 31, HPV 45, HPV 11, HPV 6 and HPV 2. Each of these has been reported as “high risk” viruses in the medical literature.

Exogenous retroviruses are known to cause various malignant and non-malignant diseases in animals over a wide range of species. These viruses infect most known animals and rodents. Examples include Deltaretroidvirus (HTLV-1, -2, -3, and -4, STLV-1, -2, and -3), Gammaretrovirus (MLV, PERV), Alpharetrovirus (Avian leucosis virus and Avian endogenous virus), and HIV 1 and 2.

Other viral families which are potential adventitious contaminants for which embodiments of the present invention are directed include: Bunyaviridae (LCMV, hantavirus), Herpesviridae (Human herpesviruses 1 through 8, Bovine herpesvirus, Canine herpesvirus and Simian cytomegalovirus), Hepadnaviridae (Hepatitis B virus), Hepeviridae (Hepatitis E virus), Deltavirus (Hepatitis delta virus), Adenoviridae (Human adenoviruses A-F and murine adenovirus), Coronaviridae, Flaviviridae (Bovine viral diarrhea virus, TBE, Yellow fever virus, Dengue viruses 1-4, WNV and hepatitis C virus), Orthomyxoviridae (influenza), Paramyxoviridae (parainfluenza, mumps, measles, RSV, Pneumonia virus of mice, Sendai virus, and Simian parainfluenza virus 5), Togaviridae (Western equine encephalomyelitis virus, rubella), Picornaviridae (Poliovirus types 1-13, coxsackie B, echovirus, rhinovirus, Human hepatitis A, Human coxsackievirus, Human cardiovirus, Human rhinovirus and Bovine rhinovirus), Reoviridae (Mouse rotavirus, reovirus type 3 and Colorado tick fever virus), and Rhabdoviridae (vesicular stomatitis virus).

For example, mouse or hamster cell banks can be infected with viruses known to be pathogenic to human. Mouse cell banks can carry lymphocytic choriomeningitis virus (LCM), sendai virus, hantaan virus, and/or lactic dehydrogenase virus. Hamster cell banks can carry LCM, sendai virus, and/or reovirus type 3. Indeed, commercially available monoclonal antibodies produced from transgenic mouse-derived cells are tested for virus including LCM, Ectromelia (MEV), mouse encephalomyelitis virus (GDVII), Hantaan, MVM, mouse adenovirus (MAV), mouse hepatitis (MHV), pneumonia virus of mice (PVM), Polyoma, Reovirus type 3 (REO-3), Sendai (SeV), virus of epizootic diarrhea of infant mice (EDIM), mouse cytomegalovirus (MCMV), papovavirus K, and LDVH viruses; Thymic Agent virus; bovine virus diarrhea (BVD), infectious bovine rhinotracheitis (IBR), respiratory parainfluenz-3 (PI-3), papillomavirus (BPV) and adenovirus-3 (BAV-3) viruses; and caprine (goat) adenovirus (CAV), herpesvirus (CHV), and arthritis encephalitis virus (CAEV) viruses. See Geigert, CHALLENGE OF CMC REGULATORY COMPLIANCE FOR BIOPHARMACEUTICALS, 109-11 (Springer, New York, N.Y., 2004); BLA reference No. 98-9912, Centocor, Infliximab Detailed Product Review (1997); BioProcessing J. (Fall, 2009).

As used herein an “adventitious virus” or “adventitious viral agent” refers to a virus contaminant present within a biological product, including, for example, vaccines, cell lines and other cell-derived products. Regarding vaccine products, for example, exogenous, adventitious ALV was found in commercial Marek's Disease vaccines propagated in CEF or DEF cell cultures by different manufacturers. Moreover, some of these vaccines were also contaminated with endogenous ALV. Fadly et al., 50 Avian Diseases 380-85 (2006); Zavala & Cheng, 50 Avian Diseases 209-15 (2006).

A conventional substrate for isolating and growing influenza viruses for vaccine purposes is the embryonated chicken egg. Influenza viruses are typically grown during 2 to 4 days at 37° C. in 10 to 11 day-old eggs. Although most of the human primary isolates of influenza A and B viruses grow better in the amniotic sac of the embryos, after two to three passages the viruses become adapted to grow in the cells of the allantoic cavity, which is accessible from the outside of the egg. Murphy & Webster, Orthomyxoviruses 1397-445, in FIELDS VIROLOGY (Lippincott-Raven pub., PA, 1996). Recombinant DNA technology and genetic engineering techniques, in theory, can afford a superior approach to producing an attenuated virus because specific mutations are deliberately engineered into the viral genome. The genetic alterations required for attenuation of viruses are not always predictable, however. In general, the attempts to use recombinant DNA technology to engineer viral vaccines have been directed to the production of subunit vaccines which contain only the protein subunits of the pathogen involved in the immune response, expressed in recombinant viral vectors such as vaccinia virus or baculovirus. More recently, recombinant DNA techniques have been utilized to produce herpes virus deletion mutants or polioviruses that mimic attenuated viruses found in nature or known host range mutants.

The yield of attenuated live influenza viruses produced in a host cell can be adversely affected by the immune response of the host cell, e.g., the interferon response of the host cell in which the virus is replicated. Additionally, the infected host cell(s) can become apoptotic before viral yield is maximized. Thus, although these attenuated viruses are immunogenic and non-pathogenic, they are often difficult to propagate in conventional cell substrates for the purposes of making vaccines. Hence, some embodiments of the present invention provide for compositions and methods using oligonucleotides (e.g., RNA effector molecules) to modulate the expression of adverse host cell responses and therefore increase viral yield. For example, some embodiments of the present invention relate to contacting an RNAi-based product siRNA into the cell prior to, during or after the viral or vector administration, to inhibit cellular and anti-viral processes that compromise the yield and quality of the viral/immunogenic product harvest.

As used herein, the term “RNA effector molecule” refers to an oligonucleotide agent capable of modulating the expression of a target gene, as defined herein, within a host cell, or a oligonucleotide agent capable of forming such an oligonucleotide, optionally, within a host cell. The RNA effector molecules described herein generally have a first strand and a second strand, one of which is substantially complementary to at least a portion of the target gene and modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and pre-translational mechanisms.

As used herein, the term “contacting” with respect to contacting cells refers to placing the cells in the presence of at least one RNA effector molecule. As used herein, the phrase “in the presence of at least one RNA effector molecule” encompasses exposure of the cell to a RNA effector molecule experessed within the cell, e.g., shRNA, or exposure by exogenous addition of the RNA effector molecule to the cell, e.g., delivery of the RNA effector molecule to the cell, optionally using an agent that facilitates uptake into the cell. In one embodiment, a cell is contacted by addition of the RNA effector, and optionally an agent that facilitates uptake of the RNA effector, to the cells growth or culture medium. A portion of a RNA effector molecule is substantially complementary to at least a portion of the target gene RNA (e.g. target (contaminating) virus), such as the coding region, the promoter region, the 3′ untranslated region (3′-UTR), or a long terminal repeat (LTR) of the target gene RNA. RNA effector molecules disclosed herein include a RNA strand (the antisense strand) having a region which is 30 nucleotides or less in length, e.g., 10 to 200 nucleotides in length, or 19 to 24 nucleotides in length, which region is substantially complementary to at least a portion of a target gene which encodes a protein that affects one or more aspects of the production of a biological product, such as the yield, purity, homogeneity, biological activity, or stability of the biological product. A RNA effector molecule interacts with RNA transcripts of a target gene and mediates its selective degradation or otherwise prevents its translation. In various embodiments of the present invention, the RNA effector molecule is at least one gapmer, or siRNA, miRNA, dsRNA, saRNA, shRNA, piRNA, tkRNAi, eiRNA, pdRNA, antagomir, or ribozyme.

In the context of this invention, the term “oligonucleotide” refers to a polymer or oligomer of nucleotide or nucleoside monomers comprising naturally occurring bases sugars and intersugar (backbone) linkages. The term “oligonucleotide” also includes polymers or oligomers comprising non-naturally occurring monomers, or portions thereof, which function similarly. Such modified or substituted oligonucleotides are often preferred over native forms because of properties such as, for example, enhanced cellular uptake, increased stability in the presence of nucleases, and the like.

Double-stranded and single-stranded oligonucleotides that are effective in inducing RNA interference are also referred to as siRNA, RNAi agent, or iRNA agent, herein. These RNA interference inducing oligonucleotides associate with a cytoplasmic multi-protein complex known as RNAi-induced silencing complex (RISC). Without being bound by theory, RNA interference leads to Argonaute-mediated post-transcriptional cleavage of target gene mRNA transcripts. In many embodiments, single-stranded and double-stranded RNAi agents are sufficiently long that they can be cleaved by an endogenous molecule, e.g. by Dicer, to produce smaller oligonucleotides that can enter the RISC machinery and participate in RISC mediated cleavage of a target sequence, e.g., a target mRNA.

As used herein, the term “region” or “portion,” when used in reference to an RNA effector molecule refers to a nucleic acid sequence of at least 3, at least 4, at least 5, at least 6, at least 7, at least 8, at least 9, at least 10 or more nucleotides up to and including the entire nucleic acid sequence of a strand of an RNA effector molecule. In some embodiments, the “region” or “portion” when used in reference to an RNA effecto molecule includes nucleic acid sequence one nucleotide shorter than the entire nucleic acid sequence of a strand of an RNA effector molecule. Thus, the term “portion” refers to a region of an RNA effector molecule having a desired length to effect complementary binding to a region of a target gene RNA or a desired length of a duplex region. One of skill in the art can vary the length of the “portion” that is complementary to the target gene or arranged in a duplex, such that an RNA effector molecule having desired characteristics (e.g., inhibition of a target gene or stability) is produced. While not wishing to be bound by theory, RNA effector molecules provided herein can modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and/or pre-translational mechanisms.

As defined herein, “substantially devoid” refers to a cell population, medium, or stock (e.g., a viral seed stock) that contains significantly reduced (e.g., at least 90%, at least 95%, at least 99%, at least 99.9%, or at least 99.99% reduced) amount of detectable unwanted virus, viral particles or viral nucleic acid when compared with the initial cell population, medium or stock prior to the method described herein.

Methods for isolating cells that are substantially devoid of target virus (e.g. contaminating virus) are provided. In one embodiment, the method comprises a) contacting a population of cells, a portion of which comprises the target virus, with an RNA effector molecule that inhibits the growth or replication of the target virus; b) detecting the presence of the target virus in each cell; and, c) isolating at least one cell that is substantially devoid of the target virus. Embodiments of the invention allow for removal of contaminating virus from established cell line banks that have been historically used for the production of immunogenic agents, e.g. vaccines.

In one embodiment, 100% of the cells in the population comprise the target virus. In one embodiment, at least 60%, at least 70%, at least 80%, at least 90%, or at least 95% of the cells in the population comprise the target virus.

In one embodiment, the method further comprises repeating steps a)-c). Through repetition, any given cell population can successively have a reduced viral load, thus facilitating the isolation of a cell that is free of contaminating virus

Contaminating virus can be removed from any cells that host replication of the contaminating virus. Host cell can be derived from a yeast, insect, amphibian, fish, reptile, bird, mammal or human, or can be a hybridoma cell. For example, the host cell can be a human Namalwa Burkitt lymphoma cell (BLc1-kar-Namalwa), baby hamster kidney fibroblast (BHK), CHO cell, Murine myeloma cell (NS0, SP2/0), hybridoma cell, human embryonic kidney cell (293 HEK), human retina-derived cell (PER.C6® cells, U.S. Pat. No. 7,550,284), insect cell line (Sf9, derived from pupal ovarian tissue of Spodoptera frugiperda; or Hi-5, derived from Trichoplusia ni egg cell homogenates; see also U.S. Pat. No. 7,041,500), Madin-Darby canine kidney cell (MDCK), primary mouse brain cells or tissue, primary calf lymph cells or tissue, primary monkey kidney cells, embryonated hens' egg, primary chicken embryo fibroblast (CEF), Rhesus fetal lung cell (FRhL-2), Human fetal lung cell (WI-38, MRC-5), African green monkey kidney epithelial cell (Vero, CV 1), Rhesus monkey kidney cell (LLC-MK2), or yeast cell.

As used herein, the phrase “inhibits the growth or replication of the target virus” with respect to an RNA effector refers to a reduction (inhibition) of viral replication in cells in the presence of the RNA effector by at least 10%, at least 20%, at least 30%, at least 40%, at least 50%, at least 60%, at least 70%, at least 80%, at least 90%, at least 95%, at least 98%, at least 99%, or even 100% (i.e. absent, not measurable) relative to the viral replication in control cells, which are not contacted with the RNA effector. Methods for quantitating viral replication are well known to those in the art. In one embodiment, viral replication is monitored by determining the viral titer present in the cells or in culture medium or by measuring the amount of viral protein in the cells or in culture medium e.g. by Western hybridization. More sensitive assays of viral replication include quantitation of viral mRNA, (e.g. capsid protein (e.g. Cap) mRNA or replication associated protein mRNA, (e.g.Rep) present within a population of cells. Any viral mRNA can be quatitated by means practiced in the art, e.g. RT-PCR.

The dose of RNA effector to be used in the methods can be determined by determining the inhibitory concentration (e.g. IC50) with respect to viral replication, e.g. as measured by mRNA quantitation. In one embodiment, the concentration of RNA effector in cell culture media of a population of cells is at least greater than the IC50 of the RNA effector. In one embodiment, the concentration is sufficient to inhibit at least 90%, at least 95%, at least 98%, at least 99%, up to 100% of viral replication. The step of contacting a population of cells with an RNA effector molecule(s) can be repeated more than once (e.g., twice, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more). In some embodiments, RNA effector is present at a concentration sufficient to inhibit viral replication through a period of time of at least one cell cycle division, at least two cell cycle division, at least three cell cycle divisions, at least four cell cycle divisions, at least five cell cycle divisions, or more, e.g. in a population of synchronized cells. In some embodiments, RNA effector is present at a concentration sufficient to inhibit viral replication through a period of time of at least 24 hours, at least 48 hours, at least 72 hours, or 148 hours.

In one embodiment, the RNA effector molecules are added to cells at a concentration from approximately 0.01 nM to 200 nM. In another embodiment, the RNA effector molecules are added at an amount of approximately 50 molecules per cell up to and including 500,000 molecules per cell. In another embodiment, the RNA effector molecules are added at a concentration from about 0.1 fmol/106 cells to about 1 μmol/106 cells. Alternative modes of delivery and dosages are described throughout the specification, herein.

In one embodiment the target virus is an episomal virus, i.e. a virus that does not integrate into host cell DNA, e.g. porcine circovirus (PCV). The inhibition of viral replication during cell growth and division of the host cell (e.g. Vero cells) can reduce viral load and subsequent infection of newly divided cells, e.g. that do not carry an epsiomal copy of target virus. Cells lacking a copy of target virus can then be isolated and identified, e.g. by dilution cloning or FACS analysis.

The presence of virus can be detected by monitoring the presence or absence target viral nucleic acid or protein in populations of sorted (and optionally expanded cells) using means that are well known to those of skill in the art. Methods to detect viral nucleic acid include, but are not limited to, polymerase chain reaction (PCR), reverse transcription polymerase chain reaction (RT-PCR), rapid amplification of cDNA ends (RACE), dot blot hybridization, Northern hybridization, and Southern hybridization (see, e.g. Current Protocols in Molecular Biology, Last Update: Jan. 11, 2011, Wiley Press, Edited by, Frederick M. Ausubel, Roger Brent, Fred Hutchinson, Robert E. Kingston, David D. Moore, J. G. Seidman, John A. Smith, Kevin Struhl).

Target virus protein can be detected, for example by radioimmunoassay, competitive-binding assay, ELISA, Western blot, fluorescent activated cell sorting (FACS), immunohistochemistry, immunoprecipitation, proteomics, mass spectrometry, electrophoresis, or immunofluoresence. As used herein, “immunologically detected” refers to detection of target virus using an antibody or antibody fragment. In one embodiment, the antibody selectively binds to a target viral protein. The term “selectively binds” refers to those antibodies that react with one or more antigenic determinants of the desired antigen, e.g., target virus or target viral protein, and do not react appreciably with other polypeptides. For example, in a competitive binding assay, preferably less than 5% of the antibody would bind another protein, more preferably less than 3%, still more preferably less than 2% and most preferably less than 1%. Antigenic determinants usually consist of chemically active surface groupings of molecules such as amino acids or sugar side chains and have specific three dimensional structural characteristics as well as specific charge characteristics.

Antibodies and antibody fragments of the present invention include, but are not limited to, monoclonal or polyconal antibodies, single chain antibodies, single chain Fv domains (scFv, also sometimes called sFv), Fab, Fab′, F(ab)₂, heavy chain single domain (dAb), humanized antibodies, human antibodies, and chimeric antibodies.

In one embodiment, target virus is detected using antibody directed against a viral capsid protein. Antibodies directed against virus proteins (e.g. capsid protein or virion specific antibodies) are commercially available in the art and can be obtained, for example from: Santa Cruz Biotechnology, Inc. Santa Cruz, Calif. 95060; or Rural Technologies, Inc. 1008 32nd Ave, Brookings, S. Dak. 57006; Abnova, Neihu District, Taipei City 114 Taiwan; Alternatively, antibodies can be made to virus or viral proteins using means well established in the art.

Some example commercially available antibodies against viral capsid proteins include, but are not limited to, a series of antiboes available from Santa Cruz Biotechnology, Inc. (Santa Cruz, Calif. 95060): catalog #(cat. #) sc-58123 EBV viral capsid antigen; cat. # sc-56999 VZV Major Capsid Protein (3H2); cat #sc-51945 EBV viral capsid antigen (4A8); cat. # sc-69945 Rotavirus capsid (0581); cat. # sc-69943 Rotavirus capsid (0531, cat #, sc-101363 Rotavirus capsid (2B4) Applications: WB, IF, 1HC(P), ELISA; cat. # sc-56989, HSV-1/2 ICPS Major Capsid Protein (3B6); cat. # sc-65935 Rubella Virus capsid protein (9B11); cat. # sc-65936 Rubella Virus capsid protein (10A1); cat. # sc-53559 Astrovirus (8E7); cat. # sc-58188 Rotavirus (0521); cat. # sc-52418 HIV-1 p24 (1A1); cat. # sc-34041 Hep C p19/p21 (vN-13); cat #sc-53558 Norwalk Virus (1D8); cat #sc-47699 HPV16 μl (CAMVIR-1); cat #sc-57824 HIV-1 p24 (39/6.14); cat #. sc-65624 FIV p24 (PAK3-2C1); cat #sc-52435 Rotavirus virus p42 (1-O-15); and cat. # sc-17526 Hep C NS3 (vG-20).

In one embodiment, the target virus is porcine circovirus (PCV) and the antibody is porcine circovirus (PCV) monoclonal antibody, e.g. catalog number-PCV2-A, available from Rural Technologies, Inc. 1008 32nd Ave, Brookings, S. Dak. 57006, U.S.A. DNA Cell Biol. 2009 January; 28(1):23-9. Additional antibodies to PCV include, but are not limited to, antibodies to PCV rep protein, or antibodies to PCV capsid. Monoclonal antibodies against PCV have been characterized in G. M. Allan et. al., Veterinary Immunology and Immunopathology Volume 43, Issue 4, November 1994, Pages 357-371. G. M. Allan et. al. supra further describes methods for producing mAbs to PCV. In addition, PCV rep antibodies are available, e.g. See Zhang X et al, characterization of monoclonal antibody against replication-associated protein of porcine circovirus, DNA Cell Biol. 2009 January; 28(1):23-9.

Cells that have no detectable virus can be continuously treated with RNA effector (prophalactically treated) and optionally resorted, e.g. to ensure that an isolated population of cells continues to be substantially free of contaminating virus. The prophalactically treated cells can then be used to produce immunogenic agents that are free of contaminationg virus.

In some embodiments, the RNA effector molecule is detectably labled and cells are further selected for the presence of the RNA effector molecule. RNA effector molecules can be labeled during synthesis or after synthesis with any molecule that is detectable, e.g. fluorophores such as Cy3™ (Glen Research) or radioacticve labels.

In one embodiment, binding of RNA effector to target is monitored by a fluorophore/quencher system, e.g. fluorescence resonance energy transfer (FRET). Upon binding of the oligomer sequence to its target ligand, the conformation of the oligomer changes, separating the fluorophore and quencher. Energy transfer pairs for fluorophore/quencher systems where both the donor and acceptor are covalently bound to the same nucleic acid are known to one skilled in the art. Such energy transfer pairs have been used to detect changes in oligonucleotide conformation, such as in e.g. Tyagi et al. (EP 0 745 690 A2 (1996)) and Pitner et al. (U.S. Pat. No. 5,691,145 (1997)).

In some embodiments, the isolated cells that are substantially free of contaminating virus are further tested for the presence of nucleic acid or protein from a second virus. For example, in the case where a target virus is removed from a previously established cell bank cell line that is used for production of a viral vaccine, the cell bank cell line may constitutively or inducibly express viral antigen, e.g. live or attenuated virus, or antigenic agent. Thus, in one embodiment, cells are selected for nucleic acid or protein from a second virus, e.g. immunogenic virus or viral antigen (e.g. rotavirus).

Several human, mammalian and avian viruses can infect cells, e.g. host cells. Infection results in the accumulation of immunogenic agent, such as live virus particles, which can be collected from cells or cell media after a suitable incubation period. The standard method of vaccine production consists of culturing cells, infecting with a live virus (e.g., rotavirus, influenza, yellow fever), incubation, harvesting of cells or cell media, downstream processing, and filling and finishing. For the classic inactived influenza vaccine, purification, inactivation, and stabilization of this harvested immunogenic agent yields vaccine product, which techniques are well known in the art. Host cells that are substantially devoid of contaminating virus (target virus) produced by methods described herein can be used to produce immugenic agents, e.g. vaccines, that are also devoid of contaminating virus.

Some embodiments of the invention include methods and compositions for depleting undesired viral contaminants in a virus seed stock or a sample of a virus suspected of being contaminated with an undesired virus by contacting the sample with a cell or matrix having a receptor for the unwanted virus. Generally, the virus seed stock or sample can be contacted with an agent that binds specifically to the unwanted virus (for example a cell surface receptor for the unwanted virus, an antibody or a portion or derivative thereof that specifically binds to the unwanted virus), but does not bind the desired virus. In one embodiment, the agent has at least a 3 fold higher affinity for the unwanted virus than the desired virus. For example, the agent has at least 5 fold, at least 10 fold, at least 20 fold, at least 50 fold, at least 100 fold higher affinity for the unwanted virus than the desired virus. The agent can be immobilized to a solid support to facilitate removal of adsorbed unwanted virus. In some embodiments, the receptor for unwanted virus is heparin. Heparin can be attached to an immobilized resin (for example Heparin HYPERD® resin, Pall Corp., Port Washington, N.Y.), or can be expressed on the surface of a cell. In some embodiments, the agent (e.g., heparin) can be up-regulated in cells using the methods of the present invention such that the cells adsorb and thereby deplete unwanted virus from a virus stock or sample. Alternatively, heparin matrices (see Hauk et al., 7 Mol. Ther. 419-25 (2003), describing heparin column purification of AAV2)), can be used in combination with the RNA effector molecule compositions and methods of the present invention.

For example, many viruses adhere to host cell-surface heparin, including PCV (Misinzo et al., 80 J. Virol. 3487-94 (2006); CMV (Compton et al., 193 Virology 834-41 (1993)); pseudorabies virus (Mettenleiter et al., 64 J. Virol. 278-86 (1990)); BHV-1 (Okazaki et al., 181 Virology 666-70 (1991)); swine vesicular disease virus (Escribano-Romero et al., 85 Gen. Virol. 653-63 (2004)); and HSV (WuDunn & Spear, 63 J. Virol. 52-58 (1989)). Additionally, enveloped viruses having infectivity associated with surface heparin binding include HIV-1 (Mondor et al., 72 J. Virol. 3623-34 (1998)); AAV-2 (Summerford & Samulski, 72J. Virol. 1438-45 (1998)); equine arteritis virus (Asagoe et al., 59 J. Vet. Med. Sci. 727-28 (1997)); Venezuelan equine encephalitis virus (Bernard et al., 276 Virology 93-103 (2000)); Sindbis virus (Byrnes & Griffin, 72 J. Virol. 7349-56 (1998); Chung et al., 72 J. Virol. 1577-85 (1998)); swine fever virus (Hulst et al., 75 J. Virol. 9585-95 (2001)); porcine reproductive and respiratory syndrome virus (Jusa et al., 62 Res. Vet. Sci. 261-64 (1997)); and RSV (Krusat & Streckert, 142 Arch. Virol. 1247-54 (1997)). A number of non-enveloped virus associate with cell surface heparin as well. Some picornaviridae family members associate with cell-surface heparin, including, foot-and-mouth disease virus (FMDV) (binds in in vitro culture) (Fry et al., 18 EMBO J. 543-54 (1999); Jackson et al., 70 J. Virol. 5282-87 (1996)); coxsackie virus B3 (CVB3) (Zautner et al., 77 J. Virol. 10071-77 (2003)); Theiler's murine encephalomyelitis virus (Reddi & Lipton, 76 J. Virol. 8400-07 (2002)); and certain echovirus serotypes (Goodfellow et al., 75 J. Virol. 4918-21 (2001)). In contrast, other picoraviridae, including poliovirus 3, coxsackie B2 viruses, and most echovirus serotypes (Goodfellow et al., 2001) do not adsorb to cell-surface heparin.

Hence, in particular embodiments of the present invention, a virus stock can be cleared of unwanted viral contamination, such as PCV1 contamination, by contacting the stock solution with cells expressing heparin or immobilized heparin (for example, using a heparin chromatography resin, see, e.g., Segura et al., 846 J. Chromatog. 124-31 (2007)), to obtain a virus stock that is devoid of contamination. In the instance where the expression of cell-surface heparin is modified, a RNA effector molecule can target genes associated with heparin degradation, such as genes encoding heparanase (hep) (e.g., mouse hep GeneID:15442, mouse hep 2 GeneID:545291, rat hep GeneID:64537, rat hep 2 GeneID:368128, human hep GeneID:10855, human hep 2 GeneID:60495, Xenopus hep GeneID:100145320, wild pig Sus scrofa hep GeneID:100271932, chicken hep GeneID:373981, chicken hep 2 GeneID:423834, dog hep GeneID:608707, bovine hep GeneID:8284471, Callithrix monkey hep Gene ID:100402671, Callithrix hep 2 GeneID:100407598), Pan troglodytes hep GeneID:461206, rabbit hep GeneID:100101601, Rhesus Macaque hep GeneID:707583, zebrafish hep GeneID:563020; Gingis-Velitski et al., 279 J. Biol. Chem. 44084-92 (2004)). In other embodiments, a RNA effector molecule can target genes associated with heparin synthesis or structure, such as epimerases, xylosyltransferases, galactosyltransferases, N-acetylglucosaminyl transferases, glucuronosyltransferases, or 2-O-sulfotransferases. See, e.g., Rostand & Esko, 65

As noted herein, the infectivity of influenza virus is dependent on the presence of sialic acid on the cell surface (Pedroso et al., 1236 Biochim. Biophys. Acta 323-30 (1995), as is the infectivity of rotaviruses (Isa et al., 23 Glycoconjugate J. 27-37 (2006); Fukudome et al., 172 Virol. 196-205 (1989)), other reoviruses (Paul et al., 172 Virol. 382-85 (1989)), and bovine coronaviruses (Schulze & Herrler, 73 J. Gen. Virol. 901-06 (1992)). Additional host cell-surface receptors include VACM-1 for encephalomyocarditis virus (Huberm 68 J. Virol. 3453-58 (1994); integrin VLA-2 for Echovirus (Bergelson et al., 1718-20 (1992); and members of the immunoglobulin super-family for poliovirus (Mendelson et al., 56 Cell 855-65 (1989).

In embodiments of the present invention relating to depleting undesired viral contaminants in a virus seed stock or a sample of a virus suspected of being contaminated with an undesired virus, different host cell receptors can be effected differently using RNA effector molecules. For example, if a rotavirus seed stock or sample is suspected of carrying PCV1 contamination, PCV1 can be depleted of by adsorption to (a) either cells that express cell surface heparin but not sialic acid (e.g., cells that naturally express cell-surface heparin but not sialic acid, or cells that express both heparin and sialic acid and are treated with sialidase to remove cell surface sialic acid, or cells in which the expression of heparin and/or sialic acid is manipulated through an effector RNA molecule), or (b) an immobilized heparin (e.g., a heparin chromatography resin) or an immobilized antibody. In this context, an “antibody” is any antigen-binding peptide derived from an antibody or a portion thereof, including a paratope, CDR, Fab, F_{V}, etc., as are well-known in the art. When a cell is used for the adsorption of unwanted virus, the cell can be a cell in which the unwanted virus does not replicate efficiently, or in which such replication is knocked down by the methods provided herein or by other techniques known in the art. For example, these methods can be combined with selective inhibition of circoviral replication by RNA effector molecule(s) that target the contaminating virus itself (e.g., PVC1 rep and/or cap, etc). Multiple absorptions can be performed to greatly and selectively reduce the PCV1 titer in the recovered fraction.

Conversely, embodiments of the present invention provide for the enrichment of the desired virus by expanding the virus in cells in which receptors for contaminating virus have been inhibited. For example, rotavirus can be propagated in cells that have been contacted with a RNA effector molecule that targets xylosyltransferase, thereby inhibiting heparin (a receptor for possible contaminating PCV). This result in more selective infection (and therefore amplification) of the desired virus, while reducing amplification of the unwanted virus. These methods can also be combined with selective inhibition of replication of circovirus by RNA

As used herein, immunogenic agent is an agent used to stimulate the immune system of a subject, so that one or more functions of the immune system are increased and directed towards the immunogenic agent. Immunogenic agents include vaccines Immunogenic agents can be used in the production of antibodies, both isolated polyclonal antibodies and monoclonal antibodies, using techniques known in the art. An antigen or immunogen is intended to mean a molecule containing one or more epitopes that can stimulate a host immune system to make a secretory, humoral and/or cellular immune response specific to that antigen.

As used herein, “vaccine” refers to an agent used to stimulate the immune system of a subject so that protection is provided against an antigen not recognized as a self-antigen by the subject's immune system. Immunization refers to the process of inducing a high level of antibody and/or cellular immune response in a subject, that is directed against a pathogen or antigen to which the organism has been exposed. Vaccines and immunogenic agents as used herein, refer to a subject's immune system: the anatomical features and mechanisms by which a subject produces antibodies and/or cellular immune responses against an antigenic material that invades the subject's cells or extra-cellular fluids. In the case of antibody production, the antibody so produced can belong to any of the immunological classes, such as immunoglobulins, A, D, E, G, or M. Vaccines that stimulate production of immunoglobulin A (IgA) are of interest, because IgA is the principal immunoglobulinof the secretory system in warm-blooded animals. Vaccines are likely to produce a broad range of other immune responses in addition to IgA formation, for example cellular and humoral immunity. Immune responses to antigens are well-studied and reported widely. See, e.g., Elgert, IMMUNOLOGY (Wiley Liss, Inc., 1996); Stites et al., BASIC & CLINICAL IMMUNOLOGY, 7th Ed. (Appleton & Lange, 1991). By contrast, the phrase “immune response of the host cell” refers to the responses of unicellular host organisms to the presence of foreign bodies.

The present invention provides for enhancing production of an immunogenic agent by introducing into the cell a RNA effector molecule to modulate expression of a target gene, optionally encoding a protein, that is involved with the expression of an adventitious, latent or endogenous virus. Thus, in some embodiments, the production of a viral product in a host cell is enhanced by introducing into the cell a RNA effector molecule that inhibits expression of a latent or endogenous viral protein such that the infectivity and/or load of the desired virus in the cell is increased. In some embodiments, the production of an immunogenic agent in a host cell is enhanced by introducing into the cell an additional RNA effector molecule that affects cell growth, cell division, cell viability, apoptosis, the immune response of the cells, nutrient handling, and/or other properties related to cell growth and/or division within the cell. In additional embodiments, production of an immunogenic agent in a cell is further enhanced by introducing a RNA effector molecule that modulates expression of a host cell protein involved in microbial infection or reproduction such that the infectivity and/or load of the microbe is increased. In further embodiments, production is enhanced by introducing into the cell a RNA effector molecule that transiently inhibits expression of viral proteins during the growth phase.

Thus, in some embodiments of the present invention, the immunogenic agent is a viral product, for example, naturally occurring viral strains, variants or mutants; mutagenized viruses (e.g., generated by exposure to mutagens, repeated passages and/or passage in non-permissive hosts), reassortants (in the case of segmented viral genomes), and/or genetically engineered viruses (e.g. using the “reverse genetics” techniques) having the desired phenotype. The viruses of these embodiments may be attenuated; i.e., they are infectious and can replicate in vivo, but generate low titers resulting in subclinical levels of infection that are generally non-pathogenic.

Additionally, the immunogenic agent of the present invention can be an intracellular parasite for which an immunogenic agent can be made using the compositions, cells, and/or methods of the present invention, e.g., using a RNA effector molecule. For example, alternative embodiments of the present invention provide for production of an immunogenic bacterial agent, i.e., a bacterial immunogen, in a eukaryotic cell. These bacteria include Shigella flexneri, Listeria monocytogenes, Rickettsiae tsutsugamushi, Rickettsiae rickettsiae, Mycobacterium leprae, Mycobacterium tuberculosis, Legionella pneumophila, Chlamydia ssp. Additional embodiments of the present invention provide for production of an immunogenic protozoan agent, i.e., a protozoan immunogen, in a eukaryotic cell. These protozoa include Plasmodium falciparum, Tripanosoma cruzi, and Leishmania donovani.

The modulation of expression (e.g., inhibition) of a target gene by a RNA effector molecule can be further alleviated by introducing a second RNA effector molecule, wherein at least a portion of the second RNA effector molecule is complementary to a target gene encoding a protein that mediates RNAi in the host cell. For example, the modulation of expression of a target gene can be alleviated by introducing into the cell a RNA effector molecule that inhibits expression of an Argonaute protein (e.g., argonaute-2) or other component of the RNAi pathway of the cell. In one embodiment, the immunogenic agent is a virus and expression of the virus is transiently inhibited by contacting the cell with a first RNA effector molecule targeted to a viral protein. The inhibition of expression of the viral product is then alleviated by introducing into the cell a second RNA effector molecule targeted against a gene encoding a protein of the RNAi pathway.

In another embodiment, the immunogenic agent is produced by a cell transfected with one or more retroviral vectors. Upon transfection with a first retroviral vector, expression of the retroviral vector Env and/or Gag molecule is transiently inhibited by contacting the cell with a first RNA effector molecule (i.e., targeting the env gene or gag gene), allowing more efficient transfection with a second retroviral vector. The inhibition of expression of the immunogenic agent can then be alleviated by introducing into the cell a second RNA effector molecule targeted against a gene encoding a protein of the RNAi pathway.

Additionally, the production of a desired immunogenic agent can be enhanced by introducing into the cell a RNA effector molecule during the production phase to modulate expression of a target gene encoding a protein that affects protein expression, post-translational modification, folding, secretion, and/or other processes related to production and/or recovery of the desired agent. Alternatively, the production of an immunogenic agent is enhanced by introducing into the cell a RNA effector molecule which inhibits cell growth and/or cell division during the production phase.

In some embodiments, the enhancement of production of an immunogenic agent, upon modulation of a target gene, is detected by monitoring one or more measurable bioprocess parameters, such as cell density, medium pH, oxygen levels, glucose levels, lactic acid levels, temperature, viral protein, or viral particle production. For example, viral protein production can be measured as specific productivity (SP) (the concentration of a product in solution) and can be expressed as mg/L or g/L; in the alternative, specific productivity can be expressed as pg/cell/day. An increase in SP can refer to an absolute or relative increase in the concentration of an immunogenic agent produced under two defined set of conditions. Alternatively, virus product can be titrated by well known plaque assays, measured as plaque forming units per mL (PFU/mL).

In some embodiments, the enhancement of production of an immunogenic agent is achieved by improving viability of the cells. As used herein, the term “improving cell viability” refers to an increase in cell density (e.g., as assessed by a Trypan Blue exclusion assay) or a decrease in apoptosis (e.g., as assessed using a TUNEL assay) of at least 10% in the presence of a RNA effector molecule(s), compared with the cell density or apoptosis levels in the absence of such a treatment. In some embodiments, the increase in cell density or decrease in apoptosis in response to treatment with a RNA effector molecule(s) is at least 20%, at least 30%, at least 40%, at least 50%, at least 60%, at least 70%, at least 80%, at least 90%, at least 95%, or even 100% compared to untreated cells. In some embodiments, the increase in cell density in response to treatment with a RNA effector molecule(s) is at least 2-fold, at least 5-fold, at least 10-fold, at least 20-fold, at least 50-fold, at least 100-fold, at least 1000-fold or higher than the cell density in the absence of the RNA effector molecule(s).

A “host cell,” as used herein, is any cell, cell culture, cellular biomass or tissue capable of being grown and maintained under conditions allowing for production and recovery of useful quantities of an immunogenic agent, including host cells contained within an egg. A host cell can be derived from a yeast, insect, amphibian, fish, reptile, bird, mammal or human, or can be a hybridoma cell. For example, the host cell can be a human Namalwa Burkitt lymphoma cell (BLc1-kar-Namalwa), baby hamster kidney fibroblast (BHK), CHO cell, Murine myeloma cell (NS0, SP2/0), hybridoma cell, human embryonic kidney cell (293 HEK), human retina-derived cell (PER.C6® cells, U.S. Pat. No. 7,550,284), insect cell line (Sf9, derived from pupal ovarian tissue of Spodoptera frugiperda; or Hi-5, derived from Trichoplusia ni egg cell homogenates; see also U.S. Pat. No. 7,041,500), Madin-Darby canine kidney cell (MDCK), primary mouse brain cells or tissue, primary calf lymph cells or tissue, primary monkey kidney cells, embryonated hens' egg, primary chicken embryo fibroblast (CEF), Rhesus fetal lung cell (FRhL-2), Human fetal lung cell (WI-38, MRC-5), African green monkey kidney epithelial cell (Vero, CV-1), Rhesus monkey kidney cell (LLC-MK2), or yeast cell.

Host cells can be unmodified or genetically modified (e.g., a cell from a transgenic animal). For example, CEFs from transgenic chicken eggs can have one or more genes essential for the IFN pathway, e.g. interferon receptor, STAT1, etc., has been disrupted, i.e., is a “knockout.” See, e.g., Sang, 12 Trends Biotech. 415 (1994); Perry et al., 2 Transgenic Res. 125 (1993); Stern, 212 Curr Top Micro. Immunol. 195-206 (1996); Shuman, 47 Experientia 897 (1991). Also, the cell can be modified to allow for growth under desired conditions, e.g., incubation at 30° C.

“G,” “C,” “A,” “T” and “U” each generally stand for a nucleotide that contains guanine, cytosine, adenine, thymine and uracil as a base, respectively. It should be understood that the term “deoxyribonucleotide,” “ribonucleotide,” or “nucleotide” can also refer to a modified nucleotide, as further detailed below, or a surrogate replacement moiety. The skilled person is well aware that ribonucleotide comprising a thymine base is also referred to as 5-methyl uridine and a deoxyribonucleotide comprising a uracil base is also referred to as deoxy-Uridine in the art. The skilled person is also well aware that guanine, cytosine, adenine, thymine and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

A “RNA effector molecule” includes a RNA agent capable of modulating the expression of a target gene, or a polynucleotide agent capable of forming such an RNA agent upon being introduced into a host cell. A portion of a RNA effector molecule is substantially complementary to at least a portion of the target gene, such as the coding region, the promoter region, the 3′ untranslated region (3′-UTR), and/or the 5′-UTR of the target gene. “Portion”, when used in reference to a RNA effector molecule refers to a nucleic acid sequence of at least four nucleotides up to and including nucleic acid sequences one nucleotide shorter than the entire RNA effector molecule. That is, a portion of a RNA effector molecule having a desired length to effect complementary binding to a region of a target gene. One of skill in the art can vary the length of the “portion” that is complementary to the target gene, such that an RNA effector molecule having desired characteristics (e.g., inhibition of a target gene) is produced. Although not bound by theory, RNA effector molecules provided herein can modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and/or pre-translational mechanisms.

As used herein, and unless otherwise indicated, the term “complementary”, when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as understood by the skilled artisan. Such conditions can, for example, be stringent conditions, where stringent conditions can include 400 mM NaCl, 40 mM PIPES pH 6.4, 1 mM EDTA, 50° C. or 70° C. for 12 to 16 hours followed by washing. Other conditions, such as physiologically relevant conditions as can be encountered inside an organism, can apply. The skilled artisan will be able to determine the set of conditions most appropriate for a test of complementarity of two sequences in accordance with the ultimate application of the hybridized nucleotides.

Complementary sequences within a RNA effector molecule, e.g., within a dsRNA (a double-stranded ribonucleic acid) as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. Where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. Where two oligonucleotides are designed to form, upon hybridization, one or more single-stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary”, “fully complementary” and “substantially complementary” herein can be used with respect to the base matching between the sense strand and the antisense strand of a dsRNA, or between the antisense strand of an RNA effector molecule agent and a target sequence, as will be understood from the context of their use.

As used herein, a polynucleotide that is “substantially complementary to at least part of a target gene refers to a polynucleotide that is substantially complementary to a contiguous portion of a target gene of interest (e.g., an mRNA encoded by a target gene, the target gene's promoter region or 3′ UTR, or ERV LTR). For example, a polynucleotide is complementary to at least a part of a target mRNA if the sequence is substantially complementary to a non-interrupted portion of a mRNA encoded by a target gene

RNA effector molecules can comprise a single strand or more than one strand, and can include, e.g., double stranded RNA (dsRNA), microRNA (miRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids and aptamers. The RNA effector molecule can be single-stranded or double-stranded. A single-stranded RNA effector molecule can have double-stranded regions and a double-stranded RNA effector can have single-stranded regions. The term “double-stranded RNA” or “dsRNA”, as used herein, refers to an oligonulceotide molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15 to 30 base pairs in length.

Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therebetween, including, but not limited to 10 to 11 base pairs, 10 to 12 base pairs, 10 to 13 base pairs, 10 to 14 base pairs, 10 to 15 base pairs, 15 to 30 base pairs, 15 to 26 base pairs, 15 to 23 base pairs, 15 to 22 base pairs, 15 to 21 base pairs, 15 to 20 base pairs, 15 to 19 base pairs, 15 to 18 base pairs, 15 to 17 base pairs, 18 to 30 base pairs, 18 to 26 base pairs, 18 to 23 base pairs, 18 to 22 base pairs, 18 to 21 base pairs, 18 to 20 base pairs, 19 to 30 base pairs, 19 to 26 base pairs, 19 to 23 base pairs, 19 to 22 base pairs, 19 to 21 base pairs, 19 to 20 base pairs, 20 to 30 base pairs, 20 to 26 base pairs, 20 to 25 base pairs, 20 to 24 base pairs, 20 to 23 base pairs, 20 to 22 base pairs, 20 to 21 base pairs, 21 to 30 base pairs, 21 to 26 base pairs, 21 to 25 base pairs, 21 to 24 base pairs, 21 to 23 base pairs, or 21 to 22 base pairs. dsRNAs generated in the cell by processing with Dicer and similar enzymes are generally in the range of 19 to 22 base pairs in length.

Double-stranded oligonucleotides, e.g., dsRNAs, generated in the cell by processing with Dicer and similar enzymes are generally in the range of 19-22 base pairs in length. One strand, antisense strand, of the duplex region of a double-stranded oligonucleotide comprises a sequence that is substantially complementary to a region of a target RNA. The two strands forming the duplex structure can be from a single oligonucleotide molecule having at least one self-complementary region, or can be formed from two or more separate oligonucleotide molecules. Where the duplex region is formed from two complementary regions of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 2, at least 3, at least 4, at least 5, at least 6, at least 7, at least 8, at least 9, at least 10, at least 20, at least 23 or more unpaired nucleotides. In some embodiments, the hairpin loop comprises 3, 4, 5, 6, or 7. Where the two substantially complementary strands of a double-stranded oligonucleotide are comprised by separate molecules, those molecules need not, but can be covalently connected. Where the two strands are connected covalently by means other than a hairpin loop, the connecting structure is referred to as a “linker.” The term “siRNA effector molecule” is also used herein to refer to a dsRNA as described herein.

As used herein, a “target gene” refers to a nucleic acid that encodes an RNA, for example, nucleic acid sequences including, but not limited to, genes encoding a polypeptide and genes encoding non coding RNAs. By “target gene RNA” or “target RNA” is meant RNA encoded by the target gene. The skilled person is well aware that a target gene RNA that encodes a polypeptide is more commonly known as messenger RNA (mRNA). The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, insect, protozoan, bacterium, or fungus. In some embodiments, the target gene encodes a protein that affects one or more aspects of the production of peptide glycosylation by a host cell, such that modulating expression of the gene permits production of a polypeptide comprising at least one terminal mannose.

The term “expression” as used herein is intended to mean the transcription to an RNA and/or translation to one or more polypeptides from a target gene coding for the sequence of the RNA and/or the polypeptide.

In some embodiments, the target gene encodes a non-coding RNA (ncRNA) that affects one or more aspects of the production of peptide glycosylation by a host cell, such that modulating expression of the gene permits production of a polypeptide comprising at least one terminal mannose. As used herein, a “non-coding RNA” refers to a target gene RNA that is not translated into a protein. The non-coding RNA is also referred to as non-protein-coding RNA (npcRNA), non-messenger RNA (mRNA), small non-messenger RNA (smRNA), and functional RNA (fRNA) in the art. The target gene from which a non-coding RNA is transcribed as the end product is also referred to as an RNA gene or non-coding RNA gene herein. Non-coding RNA genes include highly abundant and functionally important RNAs such as transfer RNA (tRNA) and ribosomal RNA (rRNA), as well as RNAs such as snoRNAs, microRNAs, siRNAs and piRNAs.

In some embodiments, the RNA effector molecule is a promoter-directed RNA (pdRNA) which is substantially complementary to at least a portion of a noncoding region of an mRNA transcript of a target gene. The pdRNA can be substantially complementary to at least a portion of the promoter region of a target gene mRNA at a site located upstream from the transcription start site, e.g., more than 100, more than 200, or more than 1,000 bases upstream from the transcription start site. Also, the pdRNA can substantially complementary to at least a portion of the 3′-UTR of a target gene mRNA transcript. For example, the pdRNA comprises dsRNA of 18 to 28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′MOE, 2′OMe, or Locked Nucleic Acid bases (LNA), which protect the gapmer from cellular nucleases.

pdRNA can be used to selectively increase, decrease, or otherwise modulate expression of a target gene. Without being limited to theory, pdRNAs can modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are known. See, e.g., WO 2009/046397.

Expressed interfering RNA (eiRNA) can be used to selectively increase, decrease, or otherwise modulate expression of a target gene. Typically, eiRNA, the dsRNA is expressed in the first transfected cell from an expression vector. In such a vector, the sense strand and the antisense strand of the dsRNA can be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Methods for making and using eiRNA effector molecules are known in the art. See, e.g., WO 2006/033756; U.S. Patent Pubs. No. 2005/0239728 and No. 2006/0035344.

In some embodiments, the RNA effector molecule comprises a small single-stranded Piwi-interacting RNA (piRNA effector molecule) which is substantially complementary to at least a portion of a target gene, as defined herein, and which selectively binds to proteins of the Piwi or Aubergine subclasses of Argonaute proteins. Without being limited to a particular theory, it is believed that piRNA effector molecules interact with RNA transcripts of target genes and recruit Piwi and/or Aubergine proteins to form a ribonucleoprotein (RNP) complex that induces transcriptional and/or post-transcriptional gene silencing of target genes. A piRNA effector molecule can be about 10 to 50 nucleotides in length, about 25 to 39 nucleotides in length, or about 26 to 31 nucleotides in length. See, e.g., U.S. Patent Pub. No. 2009/0062228.

In some embodiments, the RNA effector molecule is a siRNA or shRNA effector molecule introduced into a cell by introducing into the cell an invasive bacterium containing one or more siRNA or shRNA effector molecules or DNA encoding one or more siRNA or shRNA effector molecules (a process sometimes referred to as transkingdom RNAi (tkRNAi)). The invasive bacterium can be an attenuated strain of Listeria, Shigella, Salmonella, E. coli, or Bifidobacteriae, or a non-invasive bacterium that has been genetically modified to increase its invasive properties, e.g., by introducing one or more genes that enable invasive bacteria to access the cytoplasm of cells. Examples of such cytoplasm-targeting genes include listeriolysin O of Listeria and the invasin protein of Yersinia pseudotuberculosis. Methods for delivering RNA effector molecules to animal cells to induce transkingdom RNAi (tkRNAi) are known in the art. See, e.g., U.S. Patent Pubs. No. 2008/0311081 and No. 2009/0123426. In one embodiment, the RNA effector molecule is a siRNA molecule. In one embodiment, the RNA effector molecule is not a shRNA molecule.

MicroRNAs are a highly conserved class of small RNA molecules that are transcribed from DNA in the genomes of plants and animals, but are not translated into protein. Pre-microRNAs are processed into miRNAs. Processed microRNAs are single stranded ˜17-25 nucleotide (nt) RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. MicroRNAs cause post-transcriptional silencing of specific target genes, e.g., by inhibiting translation or initiating degradation of the targeted mRNA. In some embodiments, the miRNA is completely complementary with the target nucleic acid. In other embodiments, the miRNA has a region of noncomplementarity with the target nucleic acid, resulting in a “bulge” at the region of non-complementarity. In some embodiments, the region of noncomplementarity (the bulge) is flanked by regions of sufficient complementarity, e.g., complete complementarity, to allow duplex formation. For example, the regions of complementarity are at least 8 to 10 nucleotides long (e.g., 8, 9, or 10 nucleotides long).

miRNA can inhibit gene expression by, e.g., repressing translation, such as when the miRNA is not completely complementary to the target nucleic acid, or by causing target RNA degradation, when the miRNA binds its target with perfect or a high degree of complementarity. In further embodiments, the RNA effector molecule can include an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene. The oligonucleotide agent can include naturally occurring nucleobases, sugars, and covalent internucleotide (backbone) linkages and/or oligonucleotides having one or more non-naturally-occurring features that confer desirable properties, such as enhanced cellular uptake, enhanced affinity for the endogenous miRNA target, and/or increased stability in the presence of nucleases. In some embodiments, an oligonucleotide agent designed to bind to a specific endogenous miRNA has substantial complementarity, e.g., at least 70%, 80%, 90%, or 100% complementary, with at least 10, 20, or 25 or more bases of the target miRNA. Exemplary oligonucleiotde agents that target miRNAs and pre-miRNAs are described, for example, in U.S. Patent Pubs. No. 20090317907, No. 20090298174, No. 20090291907, No. 20090291906, No. 20090286969, No. 20090236225, No. 20090221685, No. 20090203893, No. 20070049547, No. 20050261218, No. 20090275729, No. 20090043082, No. 20070287179, No. 20060212950, No. 20060166910, No. 20050227934, No. 20050222067, No. 20050221490, No. 20050221293, No. 20050182005, and No. 20050059005.

A miRNA or pre-miRNA can be 10 to 200 nucleotides in length, for example from 16 to 80 nucleotides in length. Mature miRNAs can have a length of 16 to 30 nucleotides, such as 21 to 25 nucleotides, particularly 21, 22, 23, 24, or 25 nucleotides. miRNA precursors can have a length of 70 to 100 nucleotides and can have a hairpin conformation. In some embodiments, miRNAs are generated in vivo from pre-miRNAs by the enzymes cDicer and Drosha. miRNAs or pre-miRNAs can be synthesized in vivo by a cell-based system or can be chemically synthesized. miRNAs can comprise modifications which impart one or more desired properties, such as superior stability, hybridization thermodynamics with a target nucleic acid, targeting to a particular tissue or cell-type, and/or cell permeability, e.g., by an endocytosis-dependent or -independent mechanism. Modifications can also increase sequence specificity, and consequently decrease off-site targeting

In some embodiments, the RNA effector molecule comprises a single-stranded oligonucleotide that interacts with and directs the cleavage of RNA transcripts of a target gene. For example, single stranded RNA effector molecules comprise a 5′ modification including one or more phosphate groups or analogs thereof to protect the effector molecule from muclease degradation.

In some embodiments, the RNA effector molecule comprises an antagomir. Antagomirs are single stranded, double stranded, partially double stranded or hairpin structures that target a microRNA. An antagomir consists essentially of or comprises at least 10 or more contiguous nucleotides substantially complementary to an endogenous miRNA and more particularly a target sequence of an miRNA or pre-miRNA nucleotide sequence. Antagomirs preferably have a nucleotide sequence sufficiently complementary to a miRNA target sequence of about 12 to 25 nucleotides, such as about 15 to 23 nucleotides, to allow the antagomir to hybridize to the target sequence. More preferably, the target sequence differs by no more than 1, 2, or 3 nucleotides from the sequence of the antagomir. In some embodiments, the antagomir includes a non-nucleotide moiety, e.g., a cholesterol moiety, which can be attached, e.g., to the 3′ or 5′ end of the oligonucleotide agent.

In some embodiments, antagomirs are stabilized against nucleolytic degradation by the incorporation of a modification, e.g., a nucleotide modification. For example, in some embodiments, antagomirs contain a phosphorothioate comprising at least the first, second, and/or third internucleotide linkages at the 5′ or 3′ end of the nucleotide sequence. In further embodiments, antagomirs include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, antagomirs include at least one 2′-O-methyl-modified nucleotide

In some embodiments, the RNA effector molecule comprises an aptamer which binds to a non-nucleic acid ligand, such as a small organic molecule or protein, e.g., a transcription or translation factor, and subsequently modifies (e.g., inhibits) activity. An aptamer can fold into a specific structure that directs the recognition of a targeted binding site on the non-nucleic acid ligand. Aptamers can contain any of the modifications described herein.

In some embodiments, the RNA effector molecule is a single-stranded “antisense” nucleic acid having a nucleotide sequence that is complementary to at least a portion of a “sense” nucleic acid of a target gene, e.g., the coding strand of a double-stranded cDNA molecule or an RNA sequence, e.g., a pre-mRNA, mRNA, miRNA, or pre-miRNA. Accordingly, an antisense nucleic acid can form hydrogen bonds with a sense nucleic acid target. In an alternative embodiment, the RNA effector molecule comprises a duplex region of at least 9 nucleotides in length.

Given a coding strand sequence (e.g., the sequence of a sense strand of a cDNA molecule), antisense nucleic acids can be designed according to the rules of Watson-Crick base pairing. The antisense nucleic acid can be complementary to a portion of the coding or noncoding region of a RNA, e.g., the region surrounding the translation start site of a pre-mRNA or mRNA, e.g., the 5′ UTR. An antisense oligonucleotide can be, for example, about 10 to 25 nucleotides in length (e.g., 11, 12, 13, 14, 15, 16, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase its biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-β-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

In some embodiments, a plurality of RNA effector molecules is used to modulate expression of one or more target genes. A “plurality” refers to at least 2 or more RNA effector molecules e.g., 3, 4, 5, 6, 7, 8, 9, 10, 11, 12, 13, 14, 15, 16, 17, 18, 19, 20, 30, 40, 50, 60, 80, 100 RNA effector molecules or more. “Plurality” can also refer to at least 2 or more target genes, e.g., 3, 4, 5, 6, 7, 8, 9, 10, 11, 12, 13, 14, 15, 16, 17, 18, 19, 20, 30, 40, 50, 60, 70, 80, 90, 100 target genes or more.

The skilled artisan will recognize that the term “oligonucleotide” or “nucleic acid molecule” encompasses not only nucleic acid molecules as expressed or found in nature, but also analogs and derivatives of nucleic acids comprising one or more ribo- or deoxyribo-nucleotide/nucleoside analogs or derivatives as described herein or as known in the art. A “nucleoside” includes a nucleoside base and a ribose (2′-dooxy) sugar, and a “nucleotide” is a nucleoside with one, two or three phosphate moieties. The terms “nucleoside” and “nucleotide” can be considered to be equivalent as used herein. An oligonucleotide can be modified in the nucleobase structure or in the ribose-phosphate backbone structure, e.g., as described herein below, including the modification of a RNA nucleotide into a DNA nucleotide. The molecules comprising nucleoside analogs or derivatives must retain the ability to form a duplex.

As non-limiting examples, an oligonucleotide can also include at least one modified nucleoside including but not limited to a 2′-O-methyl modified nucleoside, a nucleoside comprising a 5′ phosphorothioate group, a terminal nucleoside linked to a cholesterol derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an oligonucleotide can comprise at least two modified nucleosides, at least 3, at least 4, at least 5, at least 6, at least 7, at least 8, at least 9, at least 10, at least 15, at least 20 or more, up to the entire length of the oligonucleotide. The modifications need not be the same for each of such a plurality of modified nucleosides in an oligonucleotide. When RNA effector molecule is double stranded, each strand can be independently modified as to number, type and/or location of the modified nucleosides. In one embodiment, modified oligonucleotides contemplated for use in methods and compositions described herein are peptide nucleic acids (PNAs) that have the ability to form the required duplex structure and that permit or mediate the specific degradation of a target RNA via a RISC pathway.

Similarly, the skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. The terms “ribonucleoside” and “ribonucleotide” can be considered to be equivalent as used herein. The RNA can be modified in the nucleobase structure or in the ribose-phosphate backbone structure, e.g., as described herein. The molecules comprising ribonucleoside analogs or derivatives must retain the ability to form a duplex. As non-limiting examples, a RNA molecule can also include at least one modified ribonucleoside including but not limited to a 2′-O-methyl modified nucleoside, a nucleoside comprising a 5′ phosphorothioate group, a terminal nucleoside linked to a cholesterol derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, a RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in a RNA molecule. In one embodiment, modified RNAs contemplated for use in methods and compositions described herein are peptide nucleic acids (PNAs) that have the ability to form the required duplex structure and that permit or mediate the specific degradation of a target RNA via a RISC pathway.

In one aspect, the oligonucleotides of the present invention can include a deoxyribonucleoside residue. In such an instance, a RNA effector molecule agent can comprise one or more deoxynucleosides, including, for example, a deoxynucleoside overhang(s), or one or more deoxynucleosides within the double stranded portion of a dsRNA. It is self evident that under no circumstances is a double stranded DNA molecule encompassed by the term “RNA effector molecule”.

A double-stranded oligonucleotide can include one or more single-stranded nucleotide overhangs. As used herein, the term “nucleotide overhang” refers to at least one unpaired nucleotide that protrudes from the terminus of a duplex structure of a double-stranded oligonucleotide, e.g., a dsRNA. For example, when a 3′-end of one strand of double-stranded oligonucleotide extends beyond the 5′-end of the other strand, or vice versa, there is a nucleotide overhang. A double-stranded oligonucleotide can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog. The overhang(s) can be on the sense strand, the antisense strand or any combination thereof. Furthermore, the nucleotide(s) of an overhang can be present on the 5′ end, 3′ end, or both ends of either an antisense or sense strand of a dsRNA.

The antisense strand of a double-stranded oligonucleotide has a 1 to 10 nucleotide overhang at the 3′ end and/or the 5′ end, such as a double-stranded oligonucleotide having a 1 to 10 nucleotide overhang at the 3′ end and/or the 5′ end. One or more of the internucloside linkages in the overhang can be replaced with a phosphorothioate. In some embodiments, the overhang comprises one or more deoxyribonucleoside or the overhang comprises the sequence 5′-dTdT-3. In some embodiments, overhang comprises the sequence 5′-dT*dT-3, wherein * is a phosphorothioate internucleoside linkage.

Without being bound theory, double-stranded oligonucleotides having at least one nucleotide overhang have unexpectedly superior inhibitory properties relative to their blunt-ended counterparts. Moreover, the presence of a nucleotide overhang on only one strand, at one end of a dsRNA, strengthens the interference activity of the double-stranded oligonucleotide, without affecting its overall stability.

dsRNA having only one overhang has proven particularly stable and effective in vivo, as well as in a variety of cells, cell culture media, blood, and serum. Generally, the single-stranded overhang is located at the 3′-terminal end of an antisense strand or, alternatively, at the 3′-terminal end of a sense strand. The dsRNA having an overhang on only one end will also have one blunt end, generally located at the 5′-end of the antisense strand. Such dsRNAs have superior stability and inhibitory activity, thus allowing administration at low dosages, i.e., less than 5 mg/kg body weight of the recipient per day. In one embodiment, the antisense strand of a dsRNA has a 1 to 10 nucleotide overhang at the 3′ end and/or the 5′ end. In one embodiment, the sense strand of a dsRNA has a 1 to 10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to double-stranded oligonucleotide mean that there are no unpaired nucleotides or nucleotide analogs at a given terminal end of a double-stranded oligonucleotide, i.e., no nucleotide overhang. One or both ends of a double-stranded oligonucleotide can be blunt. Where both ends are blunt, the oligonucleotide is said to be double-blunt ended. To be clear, a “double-blunt ended” oligonucleotide is a double-stranded oligonucleotide that is blunt at both ends, i.e., no nucleotide overhang at either end of the molecule. Most often such a molecule will be double-stranded over its entire length. When only one end of is blunt, the oligonucleotide is said to be single-blunt ended. To be clear, a “single-blunt ended” oligonucleotide is a double-stranded oligonucleotide that is blunt at only one end, i.e., no nucleotide overhang at one end of the molecule. Generally, a single-blunt ended oligonucleotide is blunt ended at the 5′-end of sense stand.

The term “antisense strand” refers to the strand of an RNA effector molecule, e.g., a dsRNA, which includes a region that is substantially complementary to a target sequence. The term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand,” as used herein, refers to the strand of an RNA effector molecule that includes a region that is substantially complementary to a region of the antisense strand as that term is defined herein.

The term “SNALP” refers to a stable nucleic acid-lipid particle. A SNALP represents a vesicle of lipids coating a reduced aqueous interior comprising a nucleic acid such as an RNA effector molecule or a plasmid from which an RNA effector molecule is transcribed. SNALPs are described, e.g., in U.S. Patent Pubs. No. 2006/0240093, No. 2007/0135372; No. 2009/0291131; U.S. patent application Ser. No. 12/343,342; No. 12/424,367.

In some embodiments, RNA effector molecule is a double-stranded oligonucleotide comprising a sense strand and an antisense strand, wherein the antisense strand has a region of complementary to at least part of a target gene RNA. The sense strand includes a region that is complementary to the antisense strand, such that the two strands hybridize and form a duplex structure when combined under suitable conditions. Typically, region of complementarity is 30 nucleotides or less in length, generally 10 to 26 nucleotides in length, preferably 18 to 25 nucleotides in length, and most preferably 19 to 24 nucleotides in length. Upon contact with a cell expressing the target gene, the RNA effector molecule inhibits the expression of the target gene by at least 10% as assayed by, for example, a PCR or branched DNA (bDNA)-based method, or by a protein-based method, such as by Western blot. Expression of a target gene in cell culture can be assayed by measuring target gene mRNA levels, e.g., by bDNA or TaqMan assay, or by measuring protein levels, e.g., by immunofluorescence analysis

Administering or introducing into the cell means that a cell is contacted with at least one RNA effector molecule, meaning the treatment of a cell with an agent e.g., at least one RNA effector molecule, often prepared in a composition comprising a delivery agent (e.g., LIPOFECTAMINE™ transfection reagent). The step of contacting a cell with an RNA effector molecule(s) can be repeated more than once (e.g., twice, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more). In one embodiment, the cell is contacted such that the target gene is modulated only transiently, e.g., by addition of a RNA effector molecule composition to the cell culture medium used for the production of an immunogenic agent where the presence of the RNA effector molecules dissipates over time, i.e., the RNA effector molecule is not constitutively expressed in the cell.

“Introducing into a cell,” when referring to a RNA effector molecule, means facilitating or effecting uptake or absorption into the cell, as is understood by those skilled in the art. Absorption or uptake of a RNA effector molecule can occur through unaided diffusive or active cellular processes, or by auxiliary agents or devices. Further approaches are described herein or known in the art.

For example, in some embodiments, a reagent that facilitates RNA effector molecule uptake comprises a charged lipid, an emulsion, a liposome, a cationic or non-cationic lipid, an anionic lipid, a transfection reagent or a penetration enhancer as described herein. In one embodiment, the reagent that facilitates RNA effector molecule uptake used herein comprises a charged lipid as described in U.S. Patent Application Ser. No. 61/267,419, filed Dec. 7, 2009.

The term “modulates expression of,” and the like, in so far as it refers to a target gene, herein refers to the modulation of expression of a target gene, as manifested by a change (e.g., an increase or a decrease) in the amount of target gene mRNA that can be isolated from or detected in a first cell or group of cells in which a target gene is transcribed and that has or have been treated such that the expression of a target gene is modulated, as compared to a second cell or group of cells substantially identical to the first cell or group of cells but that has or have not been so treated (control cells). The degree of modulation can be expressed in terms of:

$$\frac{( mRNA in control cells ) - ( mRNA in treated cells )}{( mRNA in control cells )} \cdot 100 \%$$

Alternatively, the degree of modulation can be given in terms of a parameter that is functionally linked to target gene expression, e.g., the amount of protein encoded by a target gene, or the number of cells displaying a certain phenotype, e.g., stabilization of microtubules. In principle, target gene modulation can be determined in any host cell expressing the target gene, either constitutively or by genomic engineering, and by any appropriate assay known in the art.

For example, in certain instances, expression of a target gene is inhibited. For example, expression of a target gene is inhibited by at least about 10%, 15%, 20%, 25%, 30%, 35%, 40%, 45%, or 50% by administration of an RNA effector molecule provided herein. In some embodiments, a target gene is inhibited by at least about 60%, 70%, or 80% by administration of a RNA effector molecule. In some embodiments, a target gene is inhibited by at least about 85%, 90%, or 95% or more by administration of a RNA effector molecule as described herein. In other instances, expression of a target gene is activated by at least about 10%, 20%, 25%, 50%, 100%, 200%, 400% or more by administration of a RNA effector molecule provided herein.

As used herein, a “target gene” refers to a gene that expresses a protein or regulates expression of a nucleic acid (i.e., non-encoding genes) that affects one or more aspects of the production of an immunogenic agent by a cell, such that modulating expression of the gene enhances production of the immunogenic agent. Target genes can be derived from the host cell, latent in the host cell, endogenous to the host cell (present in the host cell genome), transgenes (gene constructs inserted at ectopic sites in the host cell genome), or derived from a pathogen (e.g., a virus, fungus or bacterium) which is capable of infecting the cell or the subject who will use the an immunogenic agent or derivatives or products thereof (e.g., vaccines for humans).

In some embodiments, the target gene is a regulatory element or gene of an ERV of the cell. For example, in particular embodiments the target gene can encode a polypeptide or protein, such as an ERV LTR, env protein, or gag protein. In some embodiments, the target gene is a gene of a latent virus such as a herpesvirus or adenovirus. In particular embodiments, for example, the target gene can encode a polypeptide or protein, such as a latent HSV glycoprotein D or PCV-1 Rep protein. In some embodiments, the target gene is an endogenous cellular (i.e., non-viral) gene. For example, in particular embodiments the target gene can encode a polypeptide or protein. The target gene can also encode a host cell protein that directly or indirectly affects one or more aspects of the production of an immunogenic agent. Examples of target genes that affect the production of desired biological polypeptides include genes encoding proteins involved in the secretion, folding or post-translational modification of polypeptides (e.g., glycosylation, deamidation, disulfide bond formation, methionine oxidation, or pyroglutamation); genes encoding proteins that influence a property or phenotype of the host cell (e.g., growth, viability, cellular pH, cell cycle progression, apoptosis, carbon metabolism or transport, lactate formation, susceptibility to viral infection or RNAi uptake, activity or efficacy); and genes encoding proteins that impair the production of an immunogenic agent by the host cell (e.g., a protein that binds or co-purifies with an immunogenic agent).

Production of an immunogenic agent may be enhanced by reducing the expression of a protein that binds to the an immunogenic agent or vector. For example, in producing a viral protein it may be advantageous to reduce or inhibit expression of a receptor/ligand produced by a latent virus or ERV, so that its production in the cell does not inhibit super-infection by the desired virus or recombinant vector. Additionally, in producing a viral protein, it may be advantageous to reduce or inhibit expression of its natural receptor/ligand so that its production in the cell does not elicit a biological response (e.g., an immune response of the host cell). It will be known to a skilled artisan that a receptor can be a cell surface receptor or an internal (e.g., nuclear) receptor. The expression of the binding partner can be modulated by contacting the host cell with an RNA effector molecule directed at the receptor gene according to methods described herein.

The target gene may encode a host cell protein that indirectly affects the production of an immunogenic agent such that inhibiting expression of the target gene enhances production of the immunogenic agent. For example, the target gene can encode an abundantly expressed host cell protein that does not influence directly production of the immunogenic agent, but indirectly decreases its production, for example by utilizing cellular resources that could otherwise enhance production of the immunogenic agent.

For example, immunogenic agent such as proteins and viral products (including some live attenuated viruses) produced in cell culture on an industrial-scale are typically secreted by cultured cells and recovered and purified from the surrounding cell culture media. In general, the rate of protein production and the yield of recovered protein is directly related to the rate of protein folding and secretion by the host cells. For example, an accumulation of misfolded proteins in the endoplasmic reticulum (ER) of host cells can slow or stop secretion via the unfolded protein response (UPR) pathway. The UPR is triggered by stress-sensing proteins in the ER membrane which detect excess unfolded proteins. UPR activation leads to the upregulation of chaperone proteins (e.g., Bip) which bind to misfolded proteins and facilitate proper folding. UPR activation also upregulates the transcription factors XBP-1 and CHOP. CHOP generally functions as a negative regulator of cell growth, differentiation and survival, and its upregulation via the UPR causes cell cycle arrest and increases the rate of protein folding and secretion to clear excess unfolded proteins from the cell. Hence, cell cycle may be promoted initially, then repressed during virus production phase to increase viral product yield. An increase the rate of immunogenic protein secretion by the host cells can be measured by, e.g., monitoring the amount of protein present in the culture media over time

Proteins expressed in eukaryotic cells can undergo several post-translational modifications that may impair immunogenic agent production and/or the structure, biological activity, stability, homogeneity, and/or other properties of the immunogenic agent. Many of these modifications occur spontaneously during cell growth and polypeptide expression and can occur at several sites, including the peptide backbone, the amino acid side-chains, and the amino and/or carboxyl termini of a given polypeptide. In addition, a given polypeptide can comprise several different types of modifications. For example, proteins expressed in avian and mammalian cells can be subject to acetylation, acylation, ADP-ribosylation, amidation, ubiquitination, methionine oxidation, disulfide bond formation, methylation, demethylation, sulfation, formation of cysteine, formation of pyroglutamate, formylation, gamma-carboxylation, hydroxylation, iodination, myristoylation, oxidation, proteolytic processing, phosphorylation, prenylation, racemization, glycosylation, gluconoylation, sequence mutations, N-terminal glutamine cyclization and deamidation, and asparagine deamidation.

Post-translational modifications can require additional bioprocess steps to separate modified and unmodified polypeptides, increasing costs and reducing efficiency of immunogenic agent production. Accordingly, in some embodiments, in production of an immunogenic polypeptide agent in a cell is enhanced by modulating the expression of a target gene encoding a protein that affects post-translational modification. In additional embodiments, immunogenic agent production is enhanced by modulating the expression of a first target gene encoding a protein that affects a first post-translational modification, and modulating the expression of a second target gene encoding a protein that affects a second post-translational modification.

When the immunogenic agent comprise a glycoprotein, such as a viral product having viral surface membrane proteins, viral production may be enhanced by modulating expression of a target gene that encodes a protein involved in protein glycosylation. Glycosylation patterns are often important determinants of the structure and function of mammalian glycoproteins, and can influence the solubility, thermal stability, protease resistance, antigenicity, immunogenicity, serum half-life, stability, and biological activity of glycoproteins.

The use of cell-based bioprocesses for the manufacture of immunogenic viral agent is enhanced, in some embodiments, by modulating expression of a target gene affecting the host cell's reaction to viral infection. For example, in some embodiments the target gene is an avian cell interferon protein or a protein associated with interferon signaling. In particular, the gene may be an interferon gene such as IFN-α, Gene ID 396398; IFN-β, Gene ID 554219; or IFN-γ, Gene ID 396054. The gene may be an interferon receptor such as IFNAR1 (interferon alpha, beta and omega receptor 1), Gene ID: 395665; IFNAR2, Gene ID: 395664; IFNGR1 (interferon gamma receptor 1), Gene ID: 421685; or IFNGR2, Gene ID: 418502. The gene may be associated with interferon signaling such as STAT-1 (signal transducer and activator of transcription 1), Gene ID:424044; STAT-2, STAT-3, Gene ID:420027; STAT-4, Gene ID: 768406; STAT-5, Gene ID:395556; JAK-1 (Janus kinase 1), Gene ID: 395681; JAK-2, Gene ID: 374199; JAK-3, Gene ID:395845; IRF1, Gene ID: 396384; IRF2, Gene ID:396115; IRF3; IRF4, Gene ID:374179; IRF5, Gene ID: 430409; IRF6, Gene ID: 419863; IRF7, Gene ID:396330; IRF8, Gene ID:396385; IRF 9; or IRF10, Gene ID: 395243. The gene may encode an interferon-induced protein such as 2′,5′ oligoadenylate synthetases (2-5 OAS), an interferon induced antiviral protein; RNaseL (ribonuclease L (2′,5′-oligoisoadenylate synthetase-dependent), Gene ID: 424410 (Silverman et al., 14 J. Interferon Res. 101-04 (1994)); dsRNA-dependent protein kinase (PKR) aka: eukaryotic translation initiation factor 2-alpha kinase 2 (EIF2AK2) (Li et al., 106 PNAS 16410-05 (2009)); Mx (MX1 myxovirus (influenza virus) resistance 1, interferon-inducible protein p78), Gene ID: 395313 (Haller et al., 9 Microbes Infect. 1636-43 (2007)); IFITM1 (Brass et al., 139 Cell 1243-54 (2009)); IFITM2 (id.); IFITM3 (id.); Proinflammatory cytokines; MYD88 (myeloid differentiation primary response gene) up-regulated upon viral challenge, Gene ID: 420420 (Hghighi et al., Clin. Vaccine Immunol. (Jan. 13, 2010)); or TRIF (toll-like receptor adaptor molecule 1), Gene ID: 100008585(id.).

The use of bioprocesses for the manufacture of an immunogenic agent is enhanced, in some embodiments, by modulating expression of a target gene affecting the susceptibility of a cell to viral infection. For example, in some embodiments, the target gene is an avian cell protein that mediates viral infectivity, such as TLR3 that detects dsRNA, Gene ID: 422720 (all Gene IDs referring to the National Center for Biotechnology Information “Entrez Gene” web site); TLR7 that detects ssRNA, Gene ID: 418638; TLR21, that recognizes unmethylated DNA with CpG motifs, Gene ID: 415623, RIG-1 involved with viral sensing (Myong et al., 323 Science 1070-74 (2009)); LPGP2 and other RIG-1-like receptors, which are positive regulators of viral sensing (Satoh et al., 107 PNAS 1261-62 (2010); Nakhaei et al., 2009); TRIM25, Gene ID: 417401 (Gack et al., 5 Cell Host Microbe. 439-49 (2009)); or MAVS/VISA/IPS-1/Gardif, which interacts with RIG-1 to initiate an antiviral signaling cascade (Cui et al., 29 Mol. Cell. 169-79 (2008)); Kawai et al., 6 Nat. Immunol. 981-88 (2005)).

For optimal production of an immunogenic agent in cell-based bioprocesses described herein, it is desirable to maximize cell viability. Accordingly, in one embodiment, production of an immunogenic is enhanced by modulating expression of an avian cell protein that affects apoptosis or cell viability, such as Bax; Bak ((BCL2-antagonist/killer 1) Gene ID: 419912; LDHA (lactate dehydrogenase A), Gene ID: 396221; LDHB, Gene ID: 373997; BIK; BAD; BIM; HRK; BCLG; HR; NOXA; PUMA; BOK (BCL2-related ovarian killer), Gene ID: 395445; BOO; BCLB; CASP2 (apoptosis-related cysteine peptidase (neural precursor cell expressed, developmentally down-regulated 2)), Gene ID: 395857; CASP3 (apoptosis-related cysteine peptidase), Gene ID: 395476; CASP6, Gene ID: 395477; CASP7, Gene ID: 423901; CASP8, Gene ID: 395284; CASP9, Gene ID: 426970; CASP10, Gene ID: 424081; BCL2 (B-cell CLL/lymphoma 2), Gene ID: 396282; p53, Gene ID: 396200; APAF1; HSP70, Gene ID: 423504; TRAIL (TRAIL-LIKE TNF-related apoptosis inducing ligand-like), Gene ID: 395283; BCL2L1 (BCL2-like 1), Gene ID: 373954; BCL2L13 (BCL2-like 13 (apoptosis facilitator)), Gene ID: 418163; BCL2L14 (BCL2-like 14 (apoptosis facilitator)), Gene ID: 419096; FASLG (Fas ligand (TNF superfamily, member 6)), Gene ID: 429064; DPF2 (D4, zinc and double PHD fingers family 2), Gene ID: 429064; AIFM2 (apoptosis-inducing factor mitochondrion-associated 2), Gene ID: 423720; AIFM3, Gene ID: 416999; STK17A (serine/threonine kinase 17a (apoptosis-inducing)), Gene ID: 420775; APITD1 (apoptosis-inducing, TAF9-like domain 1), Gene ID: 771417; SIVA1 (apoptosis-inducing factor), Gene ID: 423493; FAS (TNF receptor superfamily member 6), Gene ID: 395274; TGFβ2 (transforming growth factor beta 2), Gene ID: 421352; TGFBR1 (transforming growth factor, beta receptor I), Gene ID: 374094; LOC378902 (death domain-containing tumor necrosis factor receptor superfamily member 23), Gene ID: 378902; or BCL2A1 (BCL2-related protein A1), Gene ID: 395673. For example, the BAK protein is known to down-regulate cell apoptosis pathways. Suyama et al., S1 Nucl. Acids. Res. 207-08 (2001). A particular embodiment thus provides for a RNA effector molecule that targets the BAK1 gene.

The production of an immunogenic agent in cell-based bioprocesses described herein may also be optimized by targeting genes that have been identified through screens. These include, for example, avian genes PUSL1 (pseudouridylate synthase-like 1) (Brass et al., 139 Cell 1243-54 (2009)); TPST1 (tyrosylprotein sulfotransferase 1), Gene ID: 417546 (id.); WDR33 (WD repeat domain 33) Gene ID 424753 (id.); Nod2 (Sabbah et al., 10 Nat. Immunol. 1973-80 (2009)); MCT4 (solute carrier family 16, member 3 (monocarboxylic acid transporter 4)), Gene ID: 395383 (Krishnan et al., (2008)); ACRC (acidic repeat containing) Gene ID: 422202 (id.); AMELY (id.); ATCAY (cerebellar, Cayman type (caytaxin)), Gene ID: 420094 (id); ANP32B (acidic (leucine-rich) nuclear phosphoprotein 32 family member), Gene ID: 420087(id); DEFA3 (id); DHRS10 (id); DOCK4 (dedicator of cytokinesis 4), Gene ID: 417779 (id); FAM106A (id); FKBP1B (FK506 binding protein 1B), Gene ID: 395254 (id); IRF3 (id); KBTBD8 (kelch repeat and BTB (POZ) domain containing 8), Gene ID: 416085 (id); KIAA0753 (homolog of KIAA0753 gene), Gene ID: 417681(id); LPGAT1 (lysophosphatidylglycerol acyltransferase 1), Gene ID: 421375 (id); MSMB (microseminoprotein, beta), Gene ID: 423773 (id); NFS1 (nitrogen fixation 1 homolog), Gene ID 419133 (id); NPIP (id); NPM3 (nucleophosmin/nucleoplasmin 3), Gene ID: 770430 (id); SCGB2A1 (id); SERPINB7 (id); SLC16A4 (solute carrier family 16, member 4 (monocarboxylic acid transporter 5)), Gene ID: 419809 (id); SPTBN4 (spectrin, beta, non-erythrocytic 4), Gene ID: 430775 (id); or TMEM146 (id).

Other target genes that may be affected to optimize immunogenic agents include genes associated with cell cycle and/or cell proliferation, such as avian cell CDKN1B (cyclin-dependent kinase inhibitor 1B, p27, kip1), Gene ID: 374106, a targert for which a siRNA against p27kip1 induces proliferation (Kikuchi et al. 47 Invest. Opthalmol. 4803-09 (2006)); or FOX01, a target for which a siRNA induces aortic endothelial cell proliferation (Fosbrink et al., J. Biol. Chem. 19009-18 (2006).

In some embodiments, for example when an immunogenic agent is viral, such as an influenza virus, target genes are those involved in reducing sialic acid from the host cell surface, which reduces virus binding, and therefore increases recovery of the virus in cell culture media (i.e., less virus remains stuck on cell membranes). These targets include: solute carrier family 35 (CMP-sialic acid transporter) member A1 (SLC35A1), CHO inferred from mouse GeneID:24060, avian target gene in Table 8; solute carrier family 35 (UDP-galactose transporter), member A2 (SLC35A2), CHO inferred from mouse GeneID:22232; UDP-N-acetylglucosamine 2-epimerase/N-acetylmannosamine kinase (GNE), CHO inferred from mouse GeneID:10090, avian target gene in Table 7; cytidine monophospho-N-acetylneuraminic acid synthetase (Cmas), CHO inferred from mouse Gene ID:12764, avian target gene in Table 6; UDP-Gal:betaGlcNAc beta 1,4-galactosyltransferase (B4GalT1), CHO inferred from mouse GeneID:14595, avian target gene in Table 4; and UDP-Gal:betaGlcNAc beta 1,4-galactosyltransferase, polypeptide 6 (B4GalT6), CHO inferred from mouse GeneID:56386,

Additional targets may include those involved in host sialidase in avian cells (see Wang et al., 10 BMC Genomics 512 (2009)), because influenzae binds to cell surface sialic acid residues, thus decreased sialidase may increase the rate of infection or purification: NEU2 sialidase 2 (cytosolic sialidase) Gene ID: 430542; NEU3 sialidase 3 (membrane sialidase) Gene ID: 68823. Additional target genes include miRNA antagonists that may be used to determine if this is the basis of some viruses not growing well in cells, for example Dicer (dicer 1, ribonuclease type III) because knock-down of Dicer leads to a modest increase in the rate of infection (Matskevich et al., 88 J. Gen. Virol. 2627-35 (2007)); or ISRE (interferon-stimulated response element), as a decoy titrate TFs away from ISRE-containing promoters.

Additionally, a plurality of different RNA effector molecules are introduced into the cell and permit modulation of one or more target genes. In one embodiment, the RNA effector molecules are administered during production of the viral product. In another embodiment, a plurality of different RNA effector molecules is contacted with the cells to permit modulation of PTEN, CDKN2A, BAK1, FN1, LDHA, IFN, or IFNAR1 gene expression. The effector molecules may be co-administered during the virus production and can optionally contains an additional gene or agent.

When a plurality of different RNA effector molecules are used to modulate expression of one or more target genes the plurality of RNA effector molecules are contacted with cells simultaneously or separately. In addition, each RNA effector molecule can have its own dosage regime. For example, one may prepare a composition comprising a plurality of RNA effector molecules are contacted with a cell. Alternatively, one may administer one RNA effector molecule at a time to the cell culture. In this manner, one can easily tailor the average percent inhibition desired for each target gene by altering the frequency of administration of a particular RNA effector molecule. For example, full inhibition (i.e., >80%) of lactate dehydrogenase (LDH) is not always necessary to significantly improve production of a viral product and under some conditions may be detrimental to cell viability. Thus, one may desire to contact a cell with an RNA effector molecule targeting LDH at a lower frequency (e.g., less often) or at a lower dosage (e.g., lower multiples over the IC₅₀) than the dosage for other RNA effector molecules. Contacting a cell with each RNA effector molecule separately can also prevent interactions between RNA effector molecules that can reduce efficiency of target gene modulation. For ease of use and to prevent potential contamination it may be preferred to administer a cocktail of different RNA effector molecules, thereby reducing the number of doses required and minimizing the chance of introducing a contaminant to the cell or cell culture.

Reactive oxygen species (ROS) are toxic to host cells and can mediate non-specific oxidation, degradation and/or cleavage and other structural modifications of the immunogenic agent that lead to increased heterogeneity, decreased biological activity, lower recoveries, and/or other impairments to of biological products produced by methods provided herein. Accordingly, production of an immunogenic agent is enhanced by modulating expression of a pro-oxidant enzyme, such as a protein selected from the group consisting of: NAD(p)H oxidase, peroxidase, myeloperoxidase, constitutive neuronal nitric oxide synthase (cnNOS), xanthine oxidase (XO) and myeloperoxidase (MPO), 15-lipoxygenase-1, NADPH cytochrome c reductase, NAPH cytochrome c reductase, NADH cytochrome b5 reductase, and cytochrome P4502E1.

Additionally, immunogenic protein production can be enhanced by modulating expression of a protein that affects the cell cycle of host cells, such as a cyclin (e.g., CDC2) or a cyclin dependent kinase (CDK). For example, the cyclin dependent kinase may be CDK2, CDK4, P10, P21, P27, p53, P57, p16INK4a, P14ARF, and CDK4. Thus, for example, the expression of one or more proteins that affect cell cycle progression can be transiently modulated during the growth and/or production phases of viral protein production in order to enhance expression and recovery of viral products. A particular embodiment provides for a RNA effector molecule that targets the CDKN1 gene.

It is known that production of lactic acid in cell cultures inhibits cell growth and influences metabolic pathways involved in glycolysis and glutaminolysis (Lao & Toth, 13 Biotech. Prog., 688-91 (1997)). The accumulation of lactate in cells is caused mainly by the incomplete oxidation of glucose to CO₂ and H₂O, in which most of the glucose is oxidized to pyruvate and finally converted to lactate by lactate dehydrogenase (LDH). The accumulation of lactic acid in cells is detrimental to achieving high cell density and viability. Accordingly, in one embodiment, immunogenic protein production is enhanced by modulating expression of a protein that affects lactate formation, such as lactate dehydrogenase A (LDHA). Hence, a particular embodiment provides for a RNA effector molecule that targets the LDHA1 gene.

In one embodiment, a cell culture is treated as described herein with RNA effector molecules that permit modulation of Bak and LDH expression. In another embodiment, the RNA effector molecules targeting Bak and LDH may be administered in combination with one or more additional RNA effector molecules and/or agents. Provided herein is a cocktail of RNA effector molecules targeting Bak and LDH expression, which can optionally be combined with additional RNA effector molecules or other bioactive agents as described herein.

In some embodiments, production of an immunogenic agent is enhanced by modulating expression of a protein that affects cellular pH, such as LDH or lysosomal V-type ATPase.

In some embodiments, production of an immunogenic agent is enhanced by modulating expression of a protein that affects carbon metabolism or transport, such as GLUT1, GLUT2, GLUT3, GLUT4, PTEN, or LDH.

In some embodiments, production of an immunogenic agent is enhanced by modulating expression of a protein that affects uptake or efficacy of an RNA effector molecule in host cells, such as ApoE, Mannose/Gal NAc-receptor, and Eril. In various embodiments, the expression of one or more proteins that affects RNAi uptake or efficacy in cells is modulated according to a method provided herein concurrently with modulation of one or more additional target genes, such as a target gene described herein, in order to enhance the degree and/or extent of modulation of the one or more additional target genes.

As used herein, a “RNA effector composition” includes an effective amount of a RNA effector molecule and an acceptable carrier. As used herein, “effective amount” refers to that amount of a RNA effector molecule effective to produce an effect on a bioprocess for the production of an immunogenic agent. In one embodiment, the RNA effector composition comprises a reagent that facilitates RNA effector molecule uptake (e.g., a transfection reagent).

The term “acceptable carrier” refers to a carrier for administration of a RNA effector molecule to cultured cells. Such carriers include, but are not limited to, saline, buffered saline, dextrose, water, glycerol, ethanol, and combinations thereof.

In some embodiments, RNA effector compositions include two or more RNA effector molecules, e.g., comprise two, three, four or more RNA effector molecules. In various embodiments, the two or more RNA effector molecules are capable of modulating expression of the same target gene and/or one or more additional target genes. Advantageously, certain compositions comprising multiple RNA effector molecules are more effective in enhancing production of an immunogenic agent, or one or more aspects of such production, than separate compositions comprising the individual RNA effector molecules.

A composition can comprise one or more RNA effector molecules capable of modulating expression of one or multiple genes relating to a common biological process or property of the cell, for example the interferon signaling pathway including IFN, STAT proteins or other proteins in the JAK-STAT signaling pathway, IFNRA1 and/or IFNRA2. For example, viral infection results in swift innate response in infected cells against potential lytic infection, transformation and/or apoptosis, which is characterized by the production of IFNα and IFNβ. This signaling results in activation of IFN-stimulates genes (ISGs) that mediate the effects of OFN. IFN regulatory factor (IRFs) are family of nine cellular factors that bind to consensus IFN-stimulated response elements (ISREs) and induce other ISGs. See Kirshner et al., 79 J. Virol. 9320-24 (2005). The IFNs increase the expression of intrinsic proteins including TRIM5α, Fv, Mx, eIF2α and 2′-5′ OAS, and induce apoptosis of virus-infected cells and cellular resistance to viral infection. Koyam et al., 43 Cytokine 336-41 (2008). Hence, a particular embodiment provides for a RNA effector molecule that targets a IFNR1 gene. Other embodiments target one or more genes in the IFN signaling pathway.

Inhibition of IFN signaling responses may be determined by measuring the phosphorylated state of components of the IFN pathway following viral infection, e.g., IRF-3, which is phosphorylated in response to viral dsRNA. In response to type I IFN, Jak1 kinase and TyK2 kinase, subunits of the IFN receptor, STAT1, and STAT2 are rapidly tyrosine phosphorylated. Thus, in order to determine whether the RNA effector molecule inhibits IFN responses, cells may be contacted with the RNA effector molecule, and following viral infection, the cells are lysed. IFN pathway components, such as Jak1 kinase or TyK2 kinase, are immunoprecipitated from the infected cell lysates, using specific polyclonal sera or antibodies, and the tyrosine phosphorylated state of the kinase determined by immunoblot assays with an anti-phosphotyrosine antibody. See, e.g., Krishnan et al., 247 Eur. J. Biochem. 298-305 (1997). A decreased phosphorylated state of any of the components of the IFN pathway following infection with the virus indicates decreased IFN responses by the virus in response to the RNA effector molecule(s).

Efficacy of IFN signaling inhibition can also be determined by measuring the ability to bind specific DNA sequences or the translocation of transcription factors induced in response to viral infection, and RNA effector molecule treatment, e.g., targeting IRF3, STAT1, STAT2, etc. In particular, STAT1 and STAT2 are phosphorylated and translocated from the cytoplasm to the nucleus in response to type I IFN. The ability to bind specific DNA sequences or the translocation of transcription factors can be measured by techniques known to skilled artisan, e.g., electromobility gel shift assays, cell staining, etc. Another approach to measuring inhibition of IFN induction determines whether an extract from the cell culture producing the desired viral product and contacted with a RNA effector molecule is capable of conferring protective activity against viral infection. More specifically, for example, cells are infected with the desired virus and contacted with a RNA effector. Approximately 15 to 20 hours post-infection, the cells or cell media are harvested and assayed for viral titer, or by quantitative product-enhanced reverse transcriptase (PERT) assay, immune assays, or in vivo challenge.

Described herein are RNA effector molecules that modulate expression of a target gene. In one embodiment, the RNA effector molecule agent includes double-stranded ribonucleic acid (dsRNA) molecules for inhibiting the expression of a target gene in a cell, where the dsRNA includes an antisense strand having a region of complementarity which is complementary to at least a part of a target gene formed in the expression of a target gene, and where the region of complementarity is 30 nucleotides or less in length, generally 19 to 24 nucleotides in length, and where the dsRNA, upon contact with an cell expressing the target gene, inhibits the expression of the target gene by at least 10% as assayed by, for example, a PCR, PERT or branched DNA (bDNA)-based method, or by a protein-based method, such as by Western blot. Expression of a target gene in an cell can be assayed by measuring target gene mRNA levels, e.g., by PERT, bDNA or TAQMAN® gene expression assay, or by measuring protein levels, e.g., by immunofluorescence analysis.

A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence, derived, for example, from the sequence of an mRNA formed during the expression of a target gene. The other strand (the sense strand) includes a region that is complementary to the antisense strand, such that the two strands hybridize and form a duplex structure when combined under suitable conditions. Generally, the duplex structure is between 10 and 30 inclusive, more generally between 18 and 25 inclusive, yet more generally between 19 and 24 inclusive, and most generally between 19 and 21 base pairs in length, inclusive. Similarly, the region of complementarity to the target sequence is between 10 and 30 inclusive, more generally between 18 and 25 inclusive, yet more generally between 19 and 24 inclusive, and most generally between 19 and 21 nucleotides in length, inclusive. In some embodiments, the dsRNA is between 10 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often a mRNA molecule. Where relevant, a “part” of a mRNA target is a contiguous sequence of a mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 10 nucleotides in length, such as 15 to 30 nucleotides in length.

One of skill in the art will also recognize that the duplex region is a primary functional portion of a dsRNA, e.g., a duplex region of 9 to 36, e.g., 15 to 30 base pairs. Thus, in one embodiment, to the extent that it becomes processed to a functional duplex of e.g., 15 to 30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, an RNA effector molecule is a dsRNA.

A dsRNA as described herein can further include one or more single-stranded nucleotide overhangs. The dsRNA can be synthesized by standard methods known in the art as further discussed below, e.g., by use of an automated DNA synthesizer, such as are commercially available from, for example, Biosearch Technologies (Novato, Calif.). In one embodiment, a target gene is a human target gene. In specific embodiments, the first sequence is a sense strand of a dsRNA that includes a sense sequence and the second sequence is a strand of a ds RNA that includes an antisense sequence. Alternative dsRNA agents that target elsewhere in the target sequence can readily be determined using the target sequence and the flanking target sequence. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand and the second oligonucleotide is described as the antisense strand. As described elsewhere herein and as known in the art, the complementary sequences of a dsRNA can also be contained as self-complementary regions of a single nucleic acid molecule, as opposed to being on separate oligonucleotides.

The skilled person is well aware that dsRNAs having a duplex structure of between 20 and 23, but specifically 21, base pairs have been hailed as particularly effective in inducing RNA interference. Elbashir et al., 20 EMBO 6877-88 (2001). In the embodiments described above, by virtue of the nature of the oligonucleotide sequences, dsRNAs described herein can include at least one strand of a length of 21 nucloetides. It can be reasonably expected that shorter duplexes having one of the sequences minus only a few nucleotides on one or both ends may be similarly effective as compared to the dsRNAs described in detail. Hence, dsRNAs having a partial sequence of at least 10, 11, 12, 13, 14, 15, 16, 17, 18, 19, 20, or more contiguous nucleotides from a given sequence, and differing in their ability to inhibit the expression of a target gene by not more than 5%, 10%, 15%, 20%, 25%, or 30% inhibition from a dsRNA comprising the full sequence, are contemplated according to the invention.

In addition, the RNAs provided herein identify a site in a target transcript that is susceptible to RISC-mediated cleavage. As such, the present invention further features RNA effector molecules that target within one of such sequences. As used herein, a RNA effector molecule is said to target within a particular site of a RNA transcript if the RNA effector molecule promotes cleavage of the transcript anywhere within that particular site. Such an RNA effector molecule will generally include at least 10 contiguous nucleotides from one of the sequences provided coupled to additional nucleotide sequences taken from the region contiguous to the selected sequence in a target gene.

Although a target sequence is generally 10 to 30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with a RNA effector molecule agent, mediate the best inhibition of target gene expression. Thus, while the sequences identified, for example, in Tables 2 to 7 represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified, e.g., in Table 2, further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of RNA effector molecules based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

A RNA effector molecule as described herein can contain one or more mismatches to the target sequence. For example, a RNA effector molecule as described herein contains no more than three mismatches. If the antisense strand of the RNA effector molecule contains mismatches to a target sequence, it is preferable that the area of mismatch not be located in the center of the region of complementarity. If the antisense strand of the RNA effector molecule contains mismatches to the target sequence, it is preferable that the mismatch be restricted to be within the last 5 nucleotides from either the 5′ or 3′ end of the region of complementarity. For example, for a 23-nucleotide RNA effector molecule agent RNA strand which is complementary to a region of a target gene, the RNA strand generally does not contain any mismatch within the central 13 nucleotides. The methods described herein, or methods known in the art, can be used to determine whether a RNA effector molecule containing a mismatch to a target sequence is effective in inhibiting the expression of a target gene. Consideration of the efficacy of RNA effector molecules with mismatches in inhibiting expression of a target gene is important, especially if the particular region of complementarity in a target gene is known to have polymorphic sequence variation within the population.

In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts. Moreover, the present inventors have discovered that the presence of a nucleotide overhang on only one strand, at one end of a dsRNA, strengthens the interference activity of the dsRNA, without affecting its overall stability. Such an overhang need not be a single nucleotide overhang; a dinucleotide overhang can also be present.

In yet another embodiment, an oligonucleotide (e.g., a RNA effector molecule) is chemically modified to enhance stability or other beneficial characteristics. Oligonucleotides can be modified to prevent rapid degradation of the oligonucleotides by endo- and exo-nucleases and avoid undesirable off-target effects. The nucleic acids featured in the invention can be synthesized and/or modified by methods well established in the art, such as those described in CURRENT PROTOCOLS IN NUCLEIC ACID CHEMISTRY (Beaucage et al., eds., John Wiley & Sons, Inc., NY). Modifications include, for example, (a) end modifications, e.g., 5′ end modifications (phosphorylation, conjugation, inverted linkages, etc.), or 3′ end modifications (conjugation, DNA nucleotides, inverted linkages, etc.); (b) base modifications, e.g., replacement with stabilizing bases, destabilizing bases, or bases that base pair with an expanded repertoire of partners, removal of bases (abasic nucleotides), or conjugated bases; (c) sugar modifications (e.g., at the 2′ position or 4′ position) or replacement of the sugar; as well as (d) internucleoside linkage modifications, including modification or replacement of the phosphodiester linkages. Specific examples of oligonucleotide compounds useful in this invention include, but are not limited to RNAs containing modified backbones or no natural internucleoside linkages. RNAs having modified backbones include, among others, those that do not have a phosphorus atom in the backbone. Specific examples of oligonucleotide compounds useful in this invention include, but are not limited to oligonucleotides containing modified or non-natural internucleoside linkages. Oligonucleotides having modified internucloside linkages include, among others, those that do not have a phosphorus atom in the internucleoside linkage. For the purposes of this specification, and as sometimes referenced in the art, modified oligonucleotides that do not have a phosphorus atom in their internucleoside linkage(s) can also be considered to be oligonucleosides. In particular embodiments, the modified oligonucleotides will have a phosphorus atom in its internucleoside linkage(s). For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified internucleoside linkages include (e.g., RNA backbones) include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative patents that teach the preparation of the above phosphorus-containing linkages include, but are not limited to, U.S. Pat. No. 3,687,808; U.S. Pat. No. 4,469,863; U.S. Pat. No. 4,476,301; U.S. Pat. No. 5,023,243; U.S. Pat. No. 5,177,195; U.S. Pat. No. 5,188,897; U.S. Pat. No. 5,264,423; U.S. Pat. No. 5,276,019; U.S. Pat. No. 5,278,302; U.S. Pat. No. 5,286,717; U.S. Pat. No. 5,321,131; U.S. Pat. No. 5,399,676; U.S. Pat. No. 5,405,939; U.S. Pat. No. 5,453,496; U.S. Pat. No. 5,455,233; U.S. Pat. No. 5,466,677; U.S. Pat. No. 5,476,925; U.S. Pat. No. 5,519,126; U.S. Pat. No. 5,536,821; U.S. Pat. No. 5,541,316; U.S. Pat. No. 5,550,111; U.S. Pat. No. 5,563,253; U.S. Pat. No. 5,571,799; U.S. Pat. No. 5,587,361; U.S. Pat. No. 5,625,050; U.S. Pat. No. 6,028,188; U.S. Pat. No. 6,124,445; U.S. Pat. No. 6,160,109; U.S. Pat. No. 6,169,170; U.S. Pat. No. 6,172,209; U.S. Pat. No. 6,239,265; U.S. Pat. No. 6,277,603; U.S. Pat. No. 6,326,199; U.S. Pat. No. 6,346,614; U.S. Pat. No. 6,444,423; U.S. Pat. No. 6,531,590; U.S. Pat. No. 6,534,639; U.S. Pat. No. 6,608,035; U.S. Pat. No. 6,683,167; U.S. Pat. No. 6,858,715; U.S. Pat. No. 6,867,294; U.S. Pat. No. 6,878,805; U.S. Pat. No. 7,015,315; U.S. Pat. No. 7,041,816; U.S. Pat. No. 7,273,933; U.S. Pat. No. 7,321,029; and No. RE39464.

Modified oligonucleotide internucleoside linakges (e.g., RNA backbones) that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative patents that teach the preparation of the above oligonucleosides include, but are not limited to, U.S. Pat. No. 5,034,506; U.S. Pat. No. 5,166,315; U.S. Pat. No. 5,185,444; U.S. Pat. No. 5,214,134; U.S. Pat. No. 5,216,141; U.S. Pat. No. 5,235,033; U.S. Pat. No. 5,64,562; U.S. Pat. No. 5,264,564; U.S. Pat. No. 5,405,938; U.S. Pat. No. 5,434,257; U.S. Pat. No. 5,466,677; U.S. Pat. No. 5,470,967; U.S. Pat. No. 5,489,677; U.S. Pat. No. 5,541,307; U.S. Pat. No. 5,561,225; U.S. Pat. No. 5,596,086; U.S. Pat. No. 5,602,240; U.S. Pat. No. 5,608,046; U.S. Pat. No. 5,610,289; U.S. Pat. No. 5,618,704; U.S. Pat. No. 5,623,070; U.S. Pat. No. 5,663,312; U.S. Pat. No. 5,633,360; U.S. Pat. No. 5,677,437; and U.S. Pat. No. 5,677,439.

In other modified oligonucleotides suitable or contemplated for use in RNA effector molecules, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative patents that teach the preparation of PNA compounds include, but are not limited to, U.S. Pat. No. 5,539,082; U.S. Pat. No. 5,714,331; and U.S. Pat. No. 5,719,262. Further teaching of PNA compounds can be found, for example, in Nielsen et al., 254 Science 1497-1500 (1991).

Some embodiments featured in the invention include oligonucleotides with phosphorothioate internucleoside linkages and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester internucleoside linkage is represented as —O—P—O—CH₂—] (see U.S. Pat. No. 5,489,677), and amide backbones (see U.S. Pat. No. 5,602,240). In some embodiments, the oligonucleotides featured herein have morpholino backbone structures (see U.S. Pat. No. 5,034,506).

Modified oligonucleotides can also contain one or more substituted sugar moieties. The RNA effector molecules, e.g., dsRNAs, featured herein can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙNH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to 10, inclusive. In some embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide (e.g., a RNA effector molecule), or a group for improving the pharmacodynamic properties of an oligonucleotide (e.g., a RNA effector molecule), and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., 78 Helv. Chim. Acta 486-504 (1995)), i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the oligonucleotide, particularly the 3′ position of the sugar on the 3′ terminal nucleotide or in 2′-5′ linked oligonucleotide and the 5′ position of 5′ terminal nucleotide. Oligonucletodides can also have sugar mimetics such as cyclobutyl moieties in place of the pentofuranosyl sugar. Representative patents that teach the preparation of such modified sugar structures include, but are not limited to, U.S. Pat. No. 4,981,957; U.S. Pat. No. 5,118,800; No. 5,319,080; U.S. Pat. No. 5,359,044; U.S. Pat. No. 5,393,878; U.S. Pat. No. 5,446,137; U.S. Pat. No. 5,466,786; U.S. Pat. No. 5,514,785; No. 5,519,134; U.S. Pat. No. 5,567,811; U.S. Pat. No. 5,576,427; U.S. Pat. No. 5,591,722; U.S. Pat. No. 5,597,909; U.S. Pat. No. 5,610,300; No. 5,627,053; U.S. Pat. No. 5,639,873; U.S. Pat. No. 5,646,265; U.S. Pat. No. 5,658,873; U.S. Pat. No. 5,670,633; and U.S. Pat. No. 5,700,920, certain of which are commonly owned with the instant application.

An oligonucleotide (e.g., a RNA effector molecule) can also include nucleobase (often referred to in the art simply as “base”) modifications or substitutions. As used herein, “unmodified” or “natural” nucleobases include the purine bases adenine (A) and guanine (G), and the pyrimidine bases cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil,4 (thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylamino-carbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, O6-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleobases also include natural bases that comprise conjugated moieties, e.g., a ligand.

Further nucleobases include those disclosed in U.S. Pat. No. 3,687,808; MODIFIED NUCLEOSIDES BIOCHEM., BIOTECH. & MEDICINE (Herdewijn, ed., Wiley-VCH, 2008); PCT/US09/038,425; CONCISE ENCYCLOPEDIA OF POLYMER SCIENCE & ENGIN. 858-59 (Kroschwitz ed., John Wiley & Sons, 1990); Englisch et al., 30 Angewandte Chemie, Int'Ed. 613 (1991); Sanghvi, 15 DsRNA RESEARCH & APPLICATIONS 289-302 (Crooke & Lebleu, eds., CRC Press, Boca Raton, Fla., 1993). Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, 276-78 (1993)), and are exemplary base substitutions, even more particularly when combined with 2′-O-methoxyethyl sugar modifications.

Representative patents that teach the preparation of certain of the above noted modified nucleobases as well as other modified nucleobases include, but are not limited to, the above noted U.S. Pat. No. 3,687,808; U.S. Pat. No. 4,845,205; U.S. Pat. No. 5,130,30; U.S. Pat. No. 5,134,066; U.S. Pat. No. 5,175,273; U.S. Pat. No. 5,367,066; U.S. Pat. No. 5,432,272; U.S. Pat. No. 5,457,191 U.S. Pat. No. 5,457,187; U.S. Pat. No. 5,459,255; U.S. Pat. No. 5,484,908; U.S. Pat. No. 5,502,177; U.S. Pat. No. 5,525,711; U.S. Pat. No. 5,552,540; U.S. Pat. No. 5,587,469; U.S. Pat. No. 5,594,121, U.S. Pat. No. 5,596,091; U.S. Pat. No. 5,614,617; U.S. Pat. No. 5,681,941; U.S. Pat. No. 6,015,886; U.S. Pat. No. 6,147,200; U.S. Pat. No. 6,166,197; U.S. Pat. No. 6,222,025; U.S. Pat. No. 6,235,887; U.S. Pat. No. 6,380,368; U.S. Pat. No. 6,528,640; U.S. Pat. No. 6,639,062; U.S. Pat. No. 6,617,438; U.S. Pat. No. 7,045,610; U.S. Pat. No. 7,427,672; and U.S. Pat. No. 7,495,088; and U.S. Pat. No. 5,750,692.

The oligonucleotides can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to oligonucleotide molecules has been shown to increase oligonucleotide molecule stability in serum, and to reduce off-target effects. Elmen et al., 33 Nucl. Acids Res. 439-47 (2005); Mook et al., 6 Mol. Cancer. Ther. 833-43 (2007); Grunweller et al., 31 Nucl. Acids Res. 3185-93 (2003); U.S. Pat. No. 6,268,490; U.S. Pat. No. 6,670,461; U.S. Pat. No. 6,794,499; U.S. Pat. No. 6,998,484; U.S. Pat. No. 7,053,207; U.S. Pat. No. 7,084,125; and U.S. Pat. No. 7,399,845.

Another modification of the oligonucleotides (e.g., of a RNA effector molecule) featured in the invention involves chemically linking to the oligonucleotide one or more ligands, moieties or conjugates that enhance the activity, cellular distribution or cellular uptake of the oligonucleotide. Such moieties include but are not limited to lipid moieties such as a cholesterol moiety (Letsinger et al., 86 PNAS 6553-56 (1989); cholic acid (Manoharan et al., 4 Biorg. Med. Chem. Let. 1053-60 (1994)); a thioether, e.g., beryl-5-tritylthiol (Manoharan et al., 660 Ann. N.Y. Acad. Sci. 306309 (1992); Manoharan et al., 3 Biorg. Med. Chem. Let. 2765-70 (1993)); a thiocholesterol (Oberhauser et al., 20 Nucl. Acids Res. 533-38 (1992)); an aliphatic chain, e.g., dodecandiol or undecyl residues (Saison-Behmoaras et al., 10 EMBO J. 1111-18 (1991); Kabanov et al., 259 FEBS Lett. 327-30 (1990); Svinarchuk et al., 75 Biochimie 49-54 (1993)); a phospholipid, e.g., di-hexadecyl-rac-glycerol or triethyl-ammonium 1,2-di-O-hexadecyl-rac-glycero-3-phosphonate (Manoharan et al., 36 Tetrahedron Lett. 3651-54 (1995); Shea et al., 18 Nucl. Acids Res. 3777-83 (1990)); a polyamine or a polyethylene glycol chain (Manoharan et al., 14 Nucleosides & Nucleotides 969-73 (1995)); or adamantane acetic acid (Manoharan et al., Tetrahedron Lett., 1995); a palmityl moiety (Mishra et al., 1264 Biochim. Biophys. Acta 229-37 (1995)); or an octadecylamine or hexylamino-carbonyloxycholesterol moiety (Crooke et al., 227 J. Pharmacol. Exp. Ther. 923-37 (1996)).

In one embodiment, a ligand alters the distribution, targeting or lifetime of a RNA effector molecule agent into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Ideally, ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example polyamines include polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA effector molecule agent into the cell, for example, by disrupting the cell's cytoskeleton, e.g., by disrupting the cell's microtubules, microfilaments, and/or intermediate filaments. The drug can be, for example, taxol, vincristine, vinblastine, cytochalasin, nocodazole, japlakinolide, latrunculin A, phalloidin, swinholide A, indanocine, or myoservin.

An example ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, Naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the embryo. A lipid or lipid-based ligand that binds to HSA less strongly can be used to target the conjugate to the kidney. For example, the lipid based ligand binds HSA, or it binds HSA with a sufficient affinity such that the conjugate will be distributed to a non-kidney tissue but also be reversible. Alternatively, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, that is taken up by an embryonic cell, e.g., a proliferating cell. Exemplary vitamins include vitamin A, E, and K. Other exemplary vitamins include are B vitamin, e.g., folic acid, B12, riboflavin, biotin, pyridoxal or other vitamins or nutrients taken up by embryonic cells. Also included are HSA and low density lipoproteins.

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent may be an α-helical agent, and may include a lipophilic and a lipophobic phase.

The ligand can be a peptide or peptidomimetic. A peptidomimetic (also referred to herein as an oligopeptidomimetic) is a molecule capable of folding into a defined 3-dimensional structure similar to a natural peptide. The attachment of peptide and peptidomimetics to RNA effector molecule agents can affect pharmacokinetic distribution of the RNA effector molecule, such as by enhancing cellular recognition and absorption. The peptide or peptidomimetic moiety can be about 5 to 50 amino acids long, e.g., about 5, 10, 15, 20, 25, 30, 35, 40, 45, or 50 amino acids long (see Table 1, for example).

TABLE 1
Exemplary Cell Permeation Peptides

| Cell Permeation Peptide | Amino acid Sequence | Reference |
|---|---|---|
| Penetratin | RQIKIWFQNRRMKWKK | Derossi et al., 269 J. Biol. |
| | (SEQ ID NO: 1) | Chem. 10444 (1994) |
| Tat fragment (48-60) | GRKKRRQRRRPPQC | Vives et al., 272 J. Biol. |
| | (SEQ ID NO: 2) | Chem. 16010 (1997) |
| Signal Sequence-based | GALFLGWLGAAGSTMGAWSQPKKKRKV | Chaloin et al., 243 |
| peptide | (SEQ ID NO: 3) | Biochem. Biophys. Res. |
| | | Commun 601 (1998) |
| PVEC | LLIILRRRIRKQAHAHSK | Elmquist et al., 269 Exp. |
| | (SEQ ID NO: 4) | Cell Res. 237 (2001) |
| Transportan | GWTLNSAGYLLKINLKALAALAKKIL | Pooga et al., 12 FASEB |
| | (SEQ ID NO: 5) | J. 67 (1998) |
| Amphiphilic model | KLALKLALKALKAALKLA | Oehlke et al., 2 Mol. |
| peptide | (SEQ ID NO: 6) | Ther. 339 (2000) |
| Arg₉ | RRRRRRRRR | Mitchell et al., 56 J. Pept. |
| | (SEQ ID NO: 7) | Res. 318 (2000) |
| Bacterial cell wall | KFFKFFKFFK | |
| permeating | (SEQ ID NO: 8) | |
| LL-37 | LLGDFFRKSKEKIGKEFKRIVQRIKDFLRNLVP | |
| | RTES | |
| | (SEQ ID NO: 9) | |
| Cecropin P1 | SWLSKTAKKLENSAKKRISEGIAIAIQGGPR | |
| | (SEQ ID NO: 10) | |
| α-defensin | ACYCRIPACIAGERRYGTCIYQGRLWAFCC | |
| | (SEQ ID NO: 11) | |
| b-defensin | DHYNCVSSGGQCLYSACPIFTKIQGTCYRGKAK | |
| | CCK | |
| | (SEQ ID NO: 12) | |
| Bactenecin | RKCRIVVIRVCR | |
| | (SEQ ID NO: 13) | |
| PR-39 | RRRPRPPYLPRPRPPPFFPPRLPPRIPPGFPPR | |
| | FPPRFPGKR-NH2 | |
| | (SEQ ID NO: 14) | |
| Indolicidin | ILPWKWPWWPWRR-NH2 | |
| | (SEQ ID NO: 15) | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:1441 RFGF analogue (e.g., amino acid sequence AALLPVLLAAP (SEQ ID NO:1442) containing a hydrophobic MTS can also be a targeting moiety. The peptide moiety can be a “delivery” peptide that carres large polar molecules including peptides, oligonucleotides, and protein across cell membranes. For example, sequences from the HIV Tat protein (GRKKRRQRRRPPQ (SEQ ID NO:1443)) and the Drosophila antennapedia protein (RQIKIWFQNRRMKWKK (SEQ ID NO:1444)) can function as delivery peptides. A peptide or peptidomimetic can be encoded by a random sequence of DNA, such as a peptide identified from a phage-display library, or one-bead-one-compound (OBOC) combinatorial library. Lam et al., 354 Nature 82-84 (1991). The peptide or peptidomimetic can be tethered to a dsRNA agent via an incorporated monomer unit is a cell targeting peptide such as an arginine-glycine-aspartic acid (RGD)-peptide, or RGD mimic. As noted, the peptide moieties can have a structural modification, such as to increase stability or direct conformational properties. Any of the structural modifications described herein can be utilized.

An RGD peptide moiety can be used to target a tumor cell, such as an endothelial tumor cell or a breast cancer tumor cell. Zitzmann et al., 62 Cancer Res. 5139-43 (2002). An RGD peptide can facilitate targeting of an dsRNA agent to tumors of a variety of other tissues, including the lung, kidney, spleen, or liver. Aoki et al., 8 Cancer Gene Ther. 783-87 (2001). Preferably, the RGD peptide will facilitate targeting of an RNA effector molecule agent to the kidney. The RGD peptide can be linear or cyclic, and can be modified, e.g., glycosylated or methylated to facilitate targeting to specific tissues. For example, a glycosylated RGD peptide can deliver a RNA effector molecule agent to a tumor cell expressing αVβ3. Haubner et al., 42 J. Nucl. Med. 326-36 (2001).

A “cell permeation peptide” is capable of permeating a cell, e.g., an avian cell. It may be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen. Simeoni et al., 31 Nucl. Acids Res. 2717-24 (2003).

Representative patents that teach the preparation of oligonucleotide conjugates include, but are not limited to, U.S. Pat. No. 4,828,979; U.S. Pat. No. 4,948,882; U.S. Pat. No. 5,218,105; U.S. Pat. No. 5,525,465; U.S. Pat. No. 5,541,313; U.S. Pat. No. 5,545,730; U.S. Pat. No. 5,552,538; U.S. Pat. No. 5,578,717, U.S. Pat. No. 5,580,731; U.S. Pat. No. 5,591,584; U.S. Pat. No. 5,109,124; U.S. Pat. No. 5,118,802; U.S. Pat. No. 5,138,045; U.S. Pat. No. 5,414,077; U.S. Pat. No. 5,486,603; U.S. Pat. No. 5,512,439; U.S. Pat. No. 5,578,718; U.S. Pat. No. 5,608,046; U.S. Pat. No. 4,587,044; U.S. Pat. No. 4,605,735; U.S. Pat. No. 4,667,025; U.S. Pat. No. 4,762,779; U.S. Pat. No. 4,789,737; U.S. Pat. No. 4,824,941; U.S. Pat. No. 4,835,263; U.S. Pat. No. 4,876,335; U.S. Pat. No. 4,904,582; U.S. Pat. No. 4,958,013; U.S. Pat. No. 5,082,830; U.S. Pat. No. 5,112,963; U.S. Pat. No. 5,214,136; U.S. Pat. No. 5,082,830; U.S. Pat. No. 5,112,963; U.S. Pat. No. 5,214,136; U.S. Pat. No. 5,245,022; U.S. Pat. No. 5,254,469; U.S. Pat. No. 5,258,506; U.S. Pat. No. 5,262,536; U.S. Pat. No. 5,272,250; U.S. Pat. No. 5,292,873; U.S. Pat. No. 5,317,098; U.S. Pat. No. 5,371,241, U.S. Pat. No. 5,391,723; U.S. Pat. No. 5,416,203, U.S. Pat. No. 5,451,463; U.S. Pat. No. 5,510,475; U.S. Pat. No. 5,512,667; U.S. Pat. No. 5,514,785; U.S. Pat. No. 5,565,552; U.S. Pat. No. 5,567,810; U.S. Pat. No. 5,574,142; U.S. Pat. No. 5,585,481; U.S. Pat. No. 5,587,371; U.S. Pat. No. 5,595,726; U.S. Pat. No. 5,597,696; U.S. Pat. No. 5,599,923; U.S. Pat. No. 5,599,928; U.S. Pat. No. 5,688,941; U.S. Pat. No. 6,294,664; U.S. Pat. No. 6,320,017; U.S. Pat. No. 6,576,752; U.S. Pat. No. 6,783,931; U.S. Pat. No. 6,900,297; and U.S. Pat. No. 7,037,646.

It is not necessary for all positions in a given compound to be uniformly modified, and in fact more than one of the aforementioned modifications can be incorporated in a single compound or even at a single nucleoside within sn oligonucleotide. The present invention also includes oligonucleotide molecule compounds which are chimeric compounds. “Chimeric” RNA effector molecule compounds or “chimeras,” in the context of this invention, are oligonucleotide compounds, such as dsRNAs, that contain two or more chemically distinct regions, each made up of at least one monomer unit, i.e., a nucleotide in the case of a dsRNA compound. These RNA effector molecules typically contain at least one region wherein the RNA is modified so as to confer upon the RNA effector molecule increased resistance to nuclease degradation, increased cellular uptake, and/or increased binding affinity for the target nucleic acid. An additional region of the oligonucleotide can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of RNA effector molecule inhibition of gene expression. Consequently, comparable results can often be obtained with shorter RNA effector molecules when chimeric dsRNAs are used, compared to phosphorothioate deoxydsRNAs hybridizing to the same target region. Cleavage of the oligonucleotide can be routinely detected by gel electrophoresis and, if necessary, associated nucleic acid hybridization techniques known in the art.

In certain instances, the oligonucleotides of a RNA effector molecule can be modified by a non-ligand group. A number of non-ligand molecules have been conjugated to oligonucleotides in order to enhance the activity, cellular distribution or cellular uptake of the oligonucleotides, and procedures for performing such conjugations are available in the scientific literature. Such non-ligand moieties have included lipid moieties, such as cholesterol (Kubo et al., 365 Biochem. Biophys. Res. Comm. 54-61 (2007)); Letsinger et al., 86 PNAS 6553 (1989)); cholic acid (Manoharan et al., 1994); a thioether, e.g., hexyl-5-tritylthiol (Manoharan et al., 1992; Manoharan et al., 1993); a thiocholesterol (Oberhauser et al., 1992); an aliphatic chain, e.g., dodecandiol or undecyl residues (Saison-Behmoaras et al., 1991; Kabanov et al., 259 FEBS Lett. 327 (1990); Svinarchuk et al., 75 Biochimie 75 (1993)); a phospholipid, e.g., di-hexadecyl-rac-glycerol or triethylammonium 1,2-di-O-hexadecyl-rac-glycero-3-H-phosphonate (Manoharan et al., 1995); Shea et al., 18 Nucl. Acids Res. 3777 (1990)); a polyamine or a polyethylene glycol chain (Manoharan et al., Nucleosides & Nucleotides, 1995); or adamantane acetic acid (Manoharan et al., Tetrahedron Lett., 1995); a palmityl moiety (Mishra et al., 1995); or an octadecylamine or hexylamino-carbonyl-oxycholesterol moiety (Crooke et al., 1996). Representative United States patents that teach the preparation of such RNA conjugates have been listed herein. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

The delivery of an oligonucleotide (e.g., a RNA effector molecule) to cells according to methods provided herein can be achieved in a number of different ways. For example, delivery can be performed directly by administering a composition comprising a RNA effector molecule, e.g. a dsRNA, into cell culture. Alternatively, delivery can be performed indirectly by administering into the cell one or more vectors that encode and direct the expression of the RNA effector molecule. These alternatives are discussed further.

Oligonucleotides can be modified to prevent rapid degradation of the dsRNA by endo- and exo-nucleases and avoid undesirable off-target effects. For example, RNA effector molecules can be modified by chemical conjugation to lipophilic groups such as cholesterol to enhance cellular uptake and prevent degradation. In an alternative embodiment, RNA effector molecules can be delivered using a drug delivery system such as a nanoparticle, a dendrimer, a polymer, a liposome, or a cationic delivery system. Positively charged cationic delivery systems facilitate binding of an RNA effector molecule (negatively charged) and also enhance interactions at the negatively charged cell membrane to permit efficient cellular uptake. Cationic lipids, dendrimers, or polymers can either be bound to RNA effector molecules, or induced to form a vesicle or micelle that encases the RNA effector molecule. See, e.g., Kim et al., 129 J. Contr. Release 107-16 (2008). Methods for making and using cationic-RNA effector molecule complexes are well within the abilities of those skilled in the art. See e.g., Sorensen et al 327 J. Mol. Biol. 761-66 (2003); Verma et al., 9 Clin. Cancer Res. 1291-1300 (2003); Arnold et al., 25 J. Hypertens. 197-205 (2007).

Where the RNA effector molecule is a double-stranded molecule, such as a small interfering RNA (siRNA), comprising a sense strand and an antisense strand, the sense strand and antisense strand can be separately and temporally exposed to a cell, cell lysates, tissue, or cell culture. The phrase “separately and temporally” refers to the introduction of each strand of a double-stranded RNA effector molecule to a cell, cell lysates, tissue or cell culture in a single-stranded form, e.g., in the form of a non-annealed mixture of both strands or as separate, i.e., unmixed, preparations of each strand. In some embodiments, there is a time interval between the introduction of each strand which can range from seconds to several minutes to about an hour or more, e.g., 12, 24, 48, 72, 84, 96, or 108 hours or more. Separate and temporal administration can be performed with canonical or non-canonical RNA effector molecules.

It is also contemplated herein that a plurality of RNA effector molecules are administered in a separate and temporal manner. Thus, each of a plurality of RNA effector molecules can be administered at a separate time or at a different frequency interval to achieve the desired average percent inhibition for the target gene. For example, RNA effector molecules targeting Bak can be administered more frequently than RNA effector molecule targeting LDH, as the expression of Bak recovers faster following treatment with a Bak RNA effector molecule. In one embodiment, the RNA effector molecules are added at a concentration from approximately 0.01 nM to 200 nM. In another embodiment, the RNA effector molecules are added at an amount of approximately 50 molecules per cell up to and including 500,000 molecules per cell. In another embodiment, the RNA effector molecules are added at a concentration from about 0.1 fmol/10⁶ cells to about 1 pmol/10⁶ cells.

In another aspect, a RNA effector molecule for modulating expression of a target gene can be expressed from transcription units inserted into DNA or RNA vectors. See, e.g., Couture et al., 12 TIG 5-10 (1996); WO 00/22113; WO 00/22114; U.S. Pat. No. 6,054,299. Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extra chromosomal plasmid. Gassmann, et al., 92 P.N.A.S. 1292 (1995).

The individual strand or strands of a RNA effector molecule can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as an inverted repeat joined by a linker polynucleotide sequence such that the dsRNA has a stem and loop structure.

RNA effector molecule expression vectors are generally DNA plasmids or viral vectors. Expression vectors compatible with eukaryotic cells, such as those compatible with vertebrate cells, insect cells, or yeast cells can be used to produce recombinant constructs for the expression of an RNA effector molecule as described herein. Eukaryotic cell expression vectors are well known in the art and are available from a number of commercial sources. Typically, such vectors are provided containing convenient restriction sites for insertion of the desired nucleic acid segment. RNA effector molecule expressing vectors can be delivered directly to target cells using standard transfection and transduction methods.

RNA effector molecule expression plasmids can be transfected into target cells as a complex with cationic lipid carriers (e.g., Oligofectamine™ reagent) or non-cationic lipid-based carriers (e.g., TRANSIT-TKO® transfection reagent, Mims Bio LLC, Madison, Wis.). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance. RNA effector molecule expression plasmids can be transfected into target cells as a complex with cationic lipid carriers (e.g., Oligofectamine™ reagent) or non-cationic lipid-based carriers (e.g., TRANSIT-TKO® transfection reagent). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as GFP. Stable transfection of cells ex vivo can be ensured using markers that provide the transfected cell with resistance to specific environmental factors (e.g., antibiotics and drugs), such as hygromycin B resistance

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g., EPV and EBV vectors. Constructs for the recombinant expression of an RNA effector molecule will generally require regulatory elements, e.g., promoters, enhancers, etc., to ensure the expression of the RNA effector molecule in target cells. Other aspects to consider for vectors and constructs are further described herein.

Vectors useful for the delivery of an RNA effector molecule will include regulatory elements (promoter, enhancer, etc.) sufficient for expression of the RNA effector molecule in the desired target cell or tissue. The regulatory elements can be chosen to provide either constitutive or regulated/inducible expression.

Expression of the RNA effector molecule can be precisely regulated, for example, by using an inducible regulatory sequence that is sensitive to certain physiological regulators, e.g., glucose levels. Docherty et al., 8 FASEB J. 20-24 (1994). Such inducible expression systems, suitable for the control of dsRNA expression in cells include, for example, regulation by ecdysone, estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the RNA effector molecule transgene.

In a specific embodiment, viral vectors that contain nucleic acid sequences encoding an RNA effector molecule can be used. For example, a retroviral vector can be used. See Miller et al., 217 Meth. Enzymol. 581-99 (1993); U.S. Pat. No. 6,949,242. Retroviral vectors contain the components necessary for the correct packaging of the viral genome and integration into the host cell DNA. The nucleic acid sequences encoding an RNA effector molecule are cloned into one or more vectors, which facilitates delivery of the nucleic acid into a cell. More detail about retroviral vectors can be found, for example, in Boesen et al., 6 Biotherapy 291-302 (1994), which describes the use of a retroviral vector to deliver the mdr1 gene to hematopoietic stem cells in order to make the stem cells more resistant to chemotherapy. Other references illustrating the use of retroviral vectors in gene therapy include Clowes et al., 93 J. Clin. Invest. 644-651 (1994); Kiem et al., 83 Blood 1467-73 (1994); Salmons & Gunzberg, 4 Human Gene Ther. 129-11 (1993); Grossman & Wilson, 3 Curr. Opin. Genetics Devel. 110-14 (1993). Lentiviral vectors contemplated for use include, for example, the HIV based vectors described in U.S. Pat. No. 6,143,520; U.S. Pat. No. 5,665,557; and No. 5,981,276.

It should be noted, as discussed herein, that host cell-surface receptors for retroviral entry may be inhabited by ERV Env proteins (virus interference). See Miller, 93 P.N.A.S. 11407-13 (1996). The retroviral envelope (Env) protein mediates the binding of virus particles to their cellular receptors, enabling virus entry: the first step in a new replication cycle. If an ERV is expressed in a cell, re-infection by a related exogenous retrovirus is prevented through interference (also called receptor interference). The Env protein of an ERV that is inserted into the cell membrane will interfere with the corresponding exogenous virus by receptor competition. This protects the cell from being overloaded with retroviruses. For example, enJSRVs can block the entry of exogenous JSRVs because they all utilize the cellular hyaluronidase-2 as a receptor. Spencer et al., 77 J. Virol. 5749-53 (2003). It is noteworthy that defective ERVs are no less interfering. Two enJSRVs, enJS56A1 and enJSRV-20, contain a mutant gag polyprotein that can interfere with the late stage replication of exogenous JSRVs. Arnaud et al., 2 PLoS e170 (2007). Thus interference between defective and replication-competent retroviruses provides an important mechanism of ERV copy number control. Thus, receptor interference by ERV-expressed Env molecules (e.g., expressed by the HERV-H family) may hinder infection or re-infection of cells intended to produce recombinant proteins. Such effects may explain low copy number or low expression in retroviral vector-mediated recombinant host cells. Hence, a target gene of the present embodiments that inhibits expression of ERV Env protein(s) provides for increasing retroviral vector multiplicity in host cells and increased yield of immunogenic agent.

Adenoviruses are also contemplated for use in delivery of RNA effector molecules. A suitable AV vector for expressing an RNA effector molecule featured in the invention, a method for constructing the recombinant AV vector, and a method for delivering the vector into target cells, are described in Xia et al., 20 Nature Biotech. 1006-10 (2002).

Use of Adeno-associated virus (AAV) vectors is also contemplated (Walsh et al., 204 Proc. Soc. Exp. Biol. Med. 289-300 (1993); U.S. Pat. No. 5,436,146. In one embodiment, the RNA effector molecule can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski et al., 61 J. Virol. 61: 3096-101 (1987); Fisher et al., 70 J. Virol, 70: 520-32 (1996); Samulski et al., 63 J. Virol. 3822-26 (1989); U.S. Pat. No. 5,252,479 and No. 5,139,941; WO 94/13788; WO 93/24641.

Another viral vector is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, Baculovirus, and the like. Mononegavirales, e.g., VSV or respiratory syncytial virus (RSV) may be pseudotyped with Baculovirus. U.S. Pat. No. 7,041,489. AAV vectors can be made to target different cells by engineering the vectors to express different capsid protein serotypes. See, e.g., Rabinowitz et al., 76 J. Virol. 791-801 (2002).

In one embodiment, the invention provides compositions containing a RNA effector molecule, as described herein, and an acceptable carrier. The composition containing the RNA effector molecule is useful for enhancing the production of an immunogenic agent by a cell by modulating the expression or activity of a target gene in the cell. Such compositions are formulated based on the mode of delivery. Provided herein are exemplary RNA effector molecules useful in improving the production of an immunogenic agent. In one embodiment, the RNA effector molecule is a siRNA. Alternatively, the RNA effector molecule is not a siRNA.

In another embodiment, a composition is provided herein comprising a plurality of RNA effector molecules that permit inhibition of expression of an immune response pathway and a cellular process; such as INFRA1 or CDKNA2 genes, and PTEN, BAK, FN1, or LDHA genes. The composition can optionally be combined (or administered) with at least one additional RNA effector molecule targeting an additional cellular process including, but not limited to: carbon metabolism and transport, apoptosis, RNAi uptake and/or efficiency, reactive oxygen species production, cell cycle control, protein folding, pyroglutamation protein modification, deamidase, glycosylation, disulfide bond formation, protein secretion, gene amplification, viral replication, viral infection, viral particle release, control of pH, and protein production.

In one embodiment, the compositions described herein comprise a plurality of RNA effector molecules. In one embodiment of this aspect, each of the plurality of RNA effector molecules is provided at a different concentration. In another embodiment of this aspect, each of the plurality of RNA effector molecules is provided at the same concentration. In another embodiment of this aspect, at least two of the plurality of RNA effector molecules are provided at the same concentration, while at least one other RNA effector molecule in the plurality is provided at a different concentration. It is appreciated one of skill in the art that a variety of combinations of RNA effector molecules and concentrations can be provided to a cell in culture to produce the desired effects described herein.

The compositions featured herein are administered in amounts sufficient to inhibit expression of target genes. In general, a suitable dose of RNA effector molecule will be in the range of 0.001 to 200.0 milligrams per unit volume per day. In another embodiment, the RNA effector molecule is provided in the range of 0.001 nM to 200 mM per day, generally in the range of 0.1 nM to 500 nM, inclusive. For example, the dsRNA can be administered at 0.01 nM, 0.05 nM, 0.1 nM, 0.5 nM, 0.75 nM, 1 nM, 1.5 nM, 2 nM, 3 nM, 10 nM, 20 nM, 30 nM, 40 nM, 50 nM, 100 nM, 200 nM, 400 nM, or 500 nM per single dose.

The composition can be administered once daily, or the RNA effector molecule can be administered as two, three, or more sub-doses at appropriate intervals throughout the day or delivery through a controlled release formulation. In that case, the RNA effector molecule contained in each sub-dose must be correspondingly smaller in order to achieve the total daily dosage. The dosage unit can also be compounded for delivery over several days, e.g., using a conventional sustained release formulation, which provides sustained release of the RNA effector molecule over a several-day-period. Sustained release formulations are well known in the art and are particularly useful for delivery of agents to a particular site, such as could be used with the agents of the present invention. It should be noted that when administering a plurality of RNA effector molecules, one should consider that the total dose of RNA effector molecules will be higher than when each is administered alone. For example, administration of three RNA effector molecules each at 1 nM (e.g., for effective inhibition of target gene expression) will necessarily result in a total dose of 3 nM to the cell. One of skill in the art can modify the necessary amount of each RNA effector molecule to produce effective inhibition of each target gene while preventing any unwanted toxic effects to the embryo resulting from high concentrations of either the RNA effector molecules or delivery agent.

The effect of a single dose on target gene transcript levels can be long-lasting, such that subsequent doses are administered at not more than 3-, 4-, or 5-day intervals, or at not more than 1-, 2-, 3-, or 4-week intervals.

It is also noted that, in certain embodiments, it can be beneficial to contact the cells in culture with an RNA effector molecule such that a constant number (or at least a minimum number) of RNA effector molecules per each cell is maintained. Maintaining the levels of the RNA effector molecule as such can ensure that modulation of target gene expression is maintained even at high cell densities.

Alternatively, the amount of an RNA effector molecule can be administered according to the cell density. In such embodiments, the RNA effector molecule(s) is added at a concentration of at least 0.01 fmol/10⁶ cells, at least 0.1 fmol/10⁶ cells, at least 0.5 fmol/10⁶ cells, at least 0.75 fmol/10⁶ cells, at least 1 fmol/10⁶ cells, at least 2 fmol/10⁶ cells, at least 5 fmol/10⁶ cells, at least 10 fmol/10⁶ cells, at least 20 fmol/10⁶ cells, at least 30 fmol/10⁶ cells, at least 40 fmol/10⁶ cells, at least 50 fmol/10⁶ cells, at least 60 fmol/10⁶ cells, at least 100 fmol/10⁶ cells, at least 200 fmol/10⁶ cells, at least 300 fmol/10⁶ cells, at least 400 fmol/10⁶ cells, at least 500 fmol/10⁶ cells, at least 700 fmol/10⁶ cells, at least 800 fmol/10⁶ cells, at least 900 fmol/10⁶ cells, or at least 1 pmol/10⁶ cells, or more.

In an alternate embodiment, the RNA effector molecule is administered at a dose of at least 10 molecules per cell, at least 20 molecules per cell (molecules/cell), at least 30 molecules/cell, at least 40 molecules/cell, at least 50 molecules/cell, at least 60 molecules/cell, at least 70 molecules/cell, at least 80 molecules/cell, at least 90 molecules/cell at least 100 molecules/cell, at least 200 molecules/cell, at least 300 molecules/cell, at least 400 molecules/cell, at least 500 molecules/cell, at least 600 molecules/cell, at least 700 molecules/cell, at least 800 molecules/cell, at least 900 molecules/cell, at least 1000 molecules/cell, at least 2000 molecules/cell, at least 5000 molecules/cell or more.

In some embodiments, the RNA effector molecule is administered at a dose within the range of 10-100 molecules/cell, 10-90 molecules/cell, 10-80 molecules/cell, 10-70 molecules/cell, 10-60 molecules/cell, 10-50 molecules/cell, 10-40 molecules/cell, 10-30 molecules/cell, 10-20 molecules/cell, 90-100 molecules/cell, 80-100 molecules/cell, 70-100 molecules/cell, 60-100 molecules/cell, 50-100 molecules/cell, 40-100 molecules/cell, 30-100 molecules/cell, 20-100 molecules/cell, 30-60 molecules/cell, 30-50 molecules/cell, 40-50 molecules/cell, 40-60 molecules/cell, or any range therebetween.

In one embodiment of the methods described herein, the RNA effector molecule is provided to the cells in a continuous infusion. The continuous infusion can be initiated at day zero (e.g., the first day of cell culture or day of inoculation with an RNA effector molecule) or can be initiated at any time period during the biological production process. Similarly, the continuous infusion can be stopped at any time point during the biological production process. Thus, the infusion of a RNA effector molecule or composition can be provided and/or removed at a particular phase of cell growth, a window of time in the production process, or at any other desired time point. The continuous infusion can also be provided to achieve a “desired average percent inhibition” for a target gene, as that term is used herein.

In one embodiment, a continuous infusion can be used following an initial bolus administration of an RNA effector molecule to a cell culture. In this embodiment, the continuous infusion maintains the concentration of RNA effector molecule above a minimum level over a desired period of time. The continuous infusion can be delivered at a rate of 0.03 pmol/liter of culture/hour to 3 pmol/liter of culture/hour, for example, at 0.03 pmol/l/hour, 0.05 pmol/l/hour, 0.08 pmol/l/hour, 0.1 pmol/l/hour, 0.2 pmol/l/hour, 0.3 pmol/l/hour, 0.5 pmol/l/hour, 1.0 pmol/l/hour, 2 pmol/l/hour, or 3 pmol/l/hour, or any value therebetween.

In one embodiment, the RNA effector molecule is administered as a sterile aqueous solution. In another embodiment, the RNA effector molecule is formulated in a cationic or non-cationic lipid formulation. In still another embodiment, the RNA effector molecule is formulated in a cell medium suitable for culturing a host cell (e.g., a serum-free medium). In one embodiment, an initial concentration of RNA effector molecule(s) is supplemented with a continuous infusion of the RNA effector molecule to maintain modulation of expression of a target gene. In another embodiment, the RNA effector molecule is applied to cells in culture at a particular stage of cell growth (e.g., early log phase) in a bolus dosage to achieve a certain concentration (e.g., 1 nM), and provided with a continuous infusion of the RNA effector molecule.

The RNA effector molecule(s) can be administered once daily, or the RNA effector molecule treatment can be repeated (e.g., two, three, or more doses) by adding the composition to the culture medium at appropriate intervals/frequencies throughout the production of the biological product. As used herein the term “frequency” refers to the interval at which transfection of the cell culture occurs and can be optimized by one of skill in the art to maintain the desired level of inhibition for each target gene. In one embodiment, RNA effector molecules are contacted with cells in culture at a frequency of every 48 hours. In other embodiments, the RNA effector molecules are administered at a frequency of e.g., every 4 hours, every 6 hour, every 12 hours, every 18 hours, every 24 hours, every 36 hours, every 72 hours, every 84 hours, every 96 hours, every 5 days, every 7 days, every 10 days, every 14 days, every 3 weeks, or more during the production of the biological product. The frequency can also vary, such that the interval between each dose is different (e.g., first interval 36 hour, second interval 48 hour, third interval 72 hour, etc).

The term “frequency” can be similarly applied to nutrient feeding of a cell culture during the production of a biological product. The frequency of treatment with RNA effector molecule(s) and nutrient feeding need not be the same. To be clear, nutrients can be added at the time of RNA effector treatment or at an alternate time. The frequency of nutrient feeding can be a shorter interval or a longer interval than RNA effector molecule treatment. As but one example, the dose of RNA effector molecule can be applied at a 48-hour-interval while nutrient feeding may be applied at a 24-hour-interval. During the entire length of the interval for producing the biological product (e.g., 3 weeks) there can be more doses of nutrients than RNA effector molecules or less doses of nutrients than RNA effector molecules. Alternatively, the amount of treatments with RNA effector molecule(s) is equal to that of nutrient feedings.

The frequency of RNA effector molecule treatment can be optimized to maintain an “average percent inhibition” of a particular target gene. As used herein, the term “desired average percent inhibition” refers to the average degree of inhibition of target gene expression over time that is necessary to produce the desired effect and which is below the degree of inhibition that produces any unwanted or negative effects. For example, the desired inhibition of Bak is typically >80% inhibition to effect a decrease in apoptosis, while the desired average inhibition of LDH may be less (e.g., 70%) as high degrees of LDH average inhibition (e.g., 90%) decrease cell viability. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). One of skill in the art can also use methods to detect target gene expression (e.g., PERT) to determine an amount of an RNA effector molecule that produces gene modulation. See Zhang et al., 102 Biotech. Bioengin. 1438-47 (2009). The percent inhibition is described herein as an average value over time, since the amount of inhibition is dynamic and can fluctuate slightly between doses of the RNA effector molecule.

In one embodiment of the methods described herein, the RNA effector molecule is added to the culture medium of the cells in culture. The methods described herein can be applied to any size of cell culture flask and/or bioreactor. For example, the methods can be applied in bioreactors or cell cultures of 1 L, 3 L, 5 L, 10 L, 15 L, 40 L, 100 L, 500 L, 1000 L, 2000 L, 3000 L, 4000 L, 5000 L or larger. In some embodiments, the cell culture size can range from 0.01 L to 5000 L, from 0.1 L to 5000 L, from 1 L to 5000 L, from 5 L to 5000 L, from 40 L to 5000 L, from 100 L to 5000 L, from 500 L to 5000 L, from 1000 to 5000 L, from 2000 to 5000 L, from 3000 to 5000 L, from 4000 to 5000 L, from 4500 to 5000 L, from 0.01 L to 1000 L, from 0.01 to 500 L, from 0.01 to 100 L, from 0.01 to 40 L, from 15 to 2000 L, from 40 to 1000 L, from 100 to 500 L, from 200 to 400 L, or any integer or culture size therebetween.

The RNA effector molecule(s) can be added during any phase of cell growth including, but not limited to, lag phase, stationary phase, early log phase, mid-log phase, late-log phase, exponential phase, or death phase. It is preferred that the cells are contacted with the RNA effector molecules prior to their entry into the death phase. In some embodiments, such as when targeting an apoptotic pathway, it may be desired to contact the cell in an earlier growth phase such as the lag phase, early log phase, mid-log phase or late-log phase (e.g., Bax/Bak inhibition). In other embodiments, it may be desired or acceptable to inhibit target gene expression at a later phase in the cell growth cycle (e.g., late-log phase or stationary phase), for example when growth-limiting products such as lactate are formed (e.g., LDH inhibition).

As noted, the RNA effector molecules featured in the invention can be encapsulated within liposomes or can form complexes thereto, in particular to cationic liposomes. Alternatively, RNA effector molecules can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride, or acceptable salts thereof.

In one embodiment, a RNA effector molecules featured in the invention are fully encapsulated in the lipid formulation (e.g., to form a SPLP, pSPLP, SNALP, or other nucleic acid-lipid particle). As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SPLPs include “pSPLP,” which include an encapsulated condensing agent-nucleic acid complex as set forth in WO 00/03683. The particles in this embodiment typically have a mean diameter of about 50 nm to about 150 nm, or about 60 nm to about 130 nm, or about 70 nm to about 110 nm, or typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. No. 5,976,567; U.S. Pat. No. 5,981,501; U.S. Pat. No. 6,534,484; U.S. Pat. No. 6,586,410; U.S. Pat. No. 6,815,432; WO 96/40964.

The lipid to drug ratio (mass/mass ratio) (e.g., lipid to dsRNA ratio) can be in ranges of from about 1:1 to about 50:1, from about 1:1 to about 25:1, from about 3:1 to about 15:1, from about 4:1 to about 10:1, from about 5:1 to about 9:1, or about 6:1 to about 9:1, inclusive.

A cationic lipid of the formulation may comprise at least one protonatable group having a pKa of from 4 to 15. The cationic lipid can be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), 2,2-Dilinoleyl-4-dimethylaminoethyl-[1,3]-dioxolane, or a mixture thereof. The cationic lipid can comprise from about 20 mol % to about 70 mol %, inclusive, or about 40 mol % to about 60 mol %, inclusive, of the total lipid present in the particle. In one embodiment, cationic lipid can be further conjugated to a ligand.

A non-cationic lipid can be an anionic lipid or a neutral lipid, such as distearoyl-phosphatidylcholine (DSPC), dioleoylphosphatidylcholine (DOPC), dipalmitoyl-phosphatidylcholine (DPPC), dioleoylphosphatidylglycerol (DOPG), dipalmitoyl-phosphatidylglycerol (DPPG), dioleoyl-phosphatidylethanolamine (DOPE), palmitoyloleoyl-phosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, inclusive, of about 10 mol %, to about 58 mol %, inclusive, if cholesterol is included, of the total lipid present in the particle.

The lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA may be, for example, a PEG-dilauryloxypropyl (Ci2), a PEG-dimyristyloxypropyl (C14), a PEG-dipalmityloxypropyl (C16), or a PEG-distearyloxypropyl (C 18). The lipid that prevents aggregation of particles may be from 0 mol % to about 20 mol % or about 2 mol % of the total lipid present in the particle. In one embodiment, PEG lipid can be further conjugated to a ligand.

In some embodiments, the nucleic acid-lipid particle further includes a steroid such as, cholesterol at, e.g., about 10 mol % to about 60 mol %, inclusive, or about 48 mol % of the total lipid present in the particle.

In one embodiment, the lipid particle comprises a steroid, a PEG lipid and a cationic lipid of formula (I):

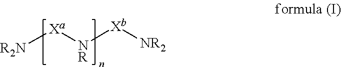

wherein each Xa and Xb, for each occurrence, is independently C1-6 alkylene; n is 0, 1, 2, 3, 4, or 5; each R is independently H,

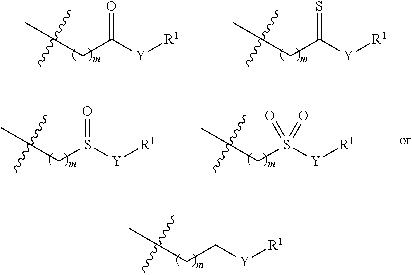

m is 0, 1, 2, 3 or 4; Y is absent, O, NR², or S; R¹ is alkyl alkenyl or alkynyl; each of which is optionally substituted with one or more substituents; and R² is H, alkyl alkenyl or alkynyl; each of which is optionally substituted each of which is optionally substituted with one or more substituents.

In one example, the lipidoid ND98.4HCl(MW 1487) (Formula 2), Cholesterol (Sigma-Aldrich), and PEG-Ceramide C16 (Avanti Polar Lipids) can be used to prepare lipid RNA effector molecule nanoparticles (e.g., LNP01 particles). Stock solutions of each in ethanol can be prepared as follows: ND98, 133 mg/mL; Cholesterol, 25 mg/mL, PEG-Ceramide C16, 100 mg/mL. The ND98, Cholesterol, and PEG-Ceramide C16 stock solutions can then be combined in a, e.g., 42:48:10 molar ratio. The combined lipid solution can be mixed with aqueous RNA effector molecule (e.g., in sodium acetate pH 5) such that the final ethanol concentration is about 35% to 45% and the final sodium acetate concentration is about 100 mM to 300 mM, inclusive. Lipid RNA effector molecule nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

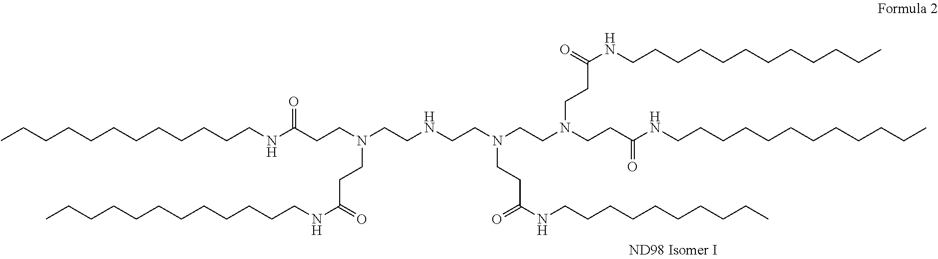 [SMILES: CCCCCCCCCCCCNC(=O)CCN(CCNCCN(CCC(=O)NCCCCCCCCCC)CCN(CCC(=O)NCCCCCCCCCCCC)CCC(=O)NCCCCCCCCCCCC)CCC(=O)NCCCCCCCCCCCC]

LNP01 formulations are described elsewhere, e.g., WO 2008/042973. Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as 40-100 nm in size. The particle size distribution should be unimodal. The total sRNA effector molecule concentration in the formulation, as well as the entrapped fraction, is estimated using a dye exclusion assay. A sample of the formulated RNA effector molecule can be incubated with an RNA-binding dye, such as Ribogreen (Molecular Probes) in the presence or absence of a formulation disrupting surfactant, e.g., 0.5% Triton-X100. The total RNA effector molecule in the formulation can be determined by the signal from the sample containing the surfactant, relative to a standard curve. The entrapped fraction is determined by subtracting the “free” RNA effector molecule content (as measured by the signal in the absence of surfactant) from the total RNA effector molecule content. Percent entrapped RNA effector molecule is typically >85%. For lipid nanoparticle formulation, the particle size is at least 30 nm, at least 40 nm, at least 50 nm, at least 60 nm, at least 70 nm, at least 80 nm, at least 90 nm, at least 100 nm, at least 110 nm, or at least 120 nm. The suitable range is typically about at least 50 nm to about at least 110 nm, about at least 60 nm to about at least 100 nm, or about at least 80 nm to about at least 90 nm, inclusive.

In some embodiments, RNA effector molecules featured in the invention are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

The compositions of the present invention may be prepared and formulated as emulsions. Emulsions are typically heterogenous systems of one liquid dispersed in another in the form of droplets usually exceeding 0.1 μm in diameter. See, e.g., Ansel's PHARM. DOSAGE FORMS & DRUG DELIV. SYS. (8th ed. Allen et al., eds., Lippincott Williams & Wilkins, NY, 2004); Idson, in 1 PHARM. DOSAGE FORMS 199 (Lieberman et al., eds., Marcel Dekker, Inc., NY, 1988); Rosoff, in 1 PHARM. DOSAGE FORMS 245 (Lieberman et al., eds., Marcel Dekker, Inc., NY, 1988); Block in 2 PHARM. DOSAGE FORMS 335 (Lieberman et al., eds., Marcel Dekker, Inc., NY, 1988); Higuchi et al., in REMINGTON'S PHARM. SCI. 301 (Mack Publishing Co., Easton, Pa., 1985). Emulsions are often biphasic systems comprising two immiscible liquid phases intimately mixed and dispersed with each other.

In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids. See, e.g., ANSEL'S PHARM. DOSAGE FORMS & DRUG DELIV. SYS., 2004; Idson, in PHARM. DOSAGE FORMS, 1988.

Synthetic surfactants, also known as surface active agents, have found wide applicability in the formulation of emulsions and have been reviewed in the literature. See, e.g., ANSEL'S PHARM. DOSAGE FORMS & DRUG DELIV. SYS., 2004; Idson, in PHARM. DOSAGE FORMS, 1988; Rieger, in PHARM. DOSAGE FORMS, 1988. Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric. See, e.g., ANSEL'S PHARM. DOSAGE FORMS & DRUG DELIV. SYS., 2004; Idson, in PHARM. DOSAGE FORMS, 1988; Rieger, in PHARM. DOSAGE FORMS, 1988.

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants. Block, in 1 PHARM. DOSAGE FORMS 335 (Lieberman et al., eds., Marcel Dekker, Inc., NY, 1988); Idson, in PHARM. DOSAGE FORMS (1988).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Because emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of RNA effector molecules and nucleic acids are formulated as microemulsions. A microemulsion may be defined as a system of water, oil and amphiphile which is a single optically isotropic and thermodynamically stable liquid solution. See, e.g., ANSEL'S PHARM. DOSAGE FORMS & DRUG DELIV. SYS. (8th ed., Allen et al, eds., Lippincott Williams & Wilkins, NY, 2004); Rosoff, in PHARM. DOSAGE FORMS, 1988. Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules. Leung & Shah, in CONTROLLED RELEASE DRUGS: POLYMERS & AGGREGATE SYS. 185-215 (Rosoff, ed., VCH Publishers, NY, 1989). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules. Schott, in REMINGTON'S PHARM. SCI. 271 (1985).

The phenomenological approach utilizing phase diagrams has been extensively studied and has yielded a comprehensive knowledge, to one skilled in the art, of how to formulate microemulsions. See, e.g., ANSEL'S PHARM. DOSAGE FORMS & DRUG DELIV. SYS. (8th ed., Allen et al, eds., Lippincott Williams & Wilkins, NY, 2004); Rosoff, 1988; Block, 1988. Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions afford advantages of better drug solubilization, protection of drug from enzymatic hydrolysis, possible enhancement of drug absorption due to surfactant-induced alterations in membrane fluidity and permeability, ease of preparation, and decreased toxicity. See, e.g., U.S. Pat. No. 6,191,105; U.S. Pat. No. 7,063,860; U.S. Pat. No. 7,070,802; U.S. Pat. No. 7,157,099; Constantinides et al., 11 Pharm. Res. 1385 (1994); Ho et al., 85 J. Pharm. Sci. 138-43 (1996). Often, microemulsions may form spontaneously when their components are brought together at ambient temperature. This may be particularly advantageous when formulating thermolabile drugs, peptides or RNA effector molecules.

Microemulsions of the present invention may also contain additional components and additives such as sorbitan monostearate (Grill 3), Labrasol, and penetration enhancers to improve the properties of the formulation and to enhance the absorption of the RNA effector molecules and nucleic acids of the present invention. Penetration enhancers used in the microemulsions of the present invention may be classified as belonging to one of five broad categories—surfactants, fatty acids, bile salts, chelating agents, and non-chelating non-surfactants. Lee et al., Crit. Rev. Therapeutic Drug Carrier Sys. 92 (1991).

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. In order to cross intact cell membranes, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include: liposomes obtained from natural phospholipids are biocompatible and biodegradable; liposomes can incorporate a wide range of water and lipid soluble drugs; and liposomes can protect encapsulated drugs in their internal compartments from metabolism and degradation. See, e.g., Wang et al., DRUG DELIV. PRINCIPLES & APPL. (John Wiley & Sons, Hoboken, N.J., 2005); Rosoff, 1988. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act. Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Liposomes fall into two broad classes. Cationic liposomes are positively charged liposomes which interact with the negatively charged polynucleotide molecules to form a stable complex. The positively charged polynucleotide/liposome complex binds to the negatively charged cell surface and is internalized in an endosome. Due to the acidic pH within the endosome, the liposomes are ruptured, releasing their contents into the cell cytoplasm. Wang et al., 147 Biochem. Biophys. Res. Commun. 980-85 (1987).

Liposomes which are pH-sensitive or negatively-charged, entrap polynucleotide rather than complex with it. Because both the polynucleotide and the lipid are similarly charged, repulsion rather than complex formation occurs. Nevertheless, some polynucleotide is entrapped within the aqueous interior of these liposomes. pH-sensitive liposomes have been used to deliver DNA encoding the thymidine kinase gene to cell monolayers in culture. Expression of the exogenous gene was detected in the target cells. Zhou et al., 19 J. Controlled Rel. 269-74 (1992).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES). Allen et al., 223 FEBS Lett. 42 (1987); Wu et al., 53 Cancer Res. 3765 (1993).

Various liposomes comprising one or more glycolipids are known in the art. Papahadjopoulos et al. (507 Ann. N.Y. Acad. Sci. 64 (1987)), reported the ability of monosialoganglioside GM1, galactocerebroside sulfate and phosphatidylinositol to improve blood half-lives of liposomes. These findings were expounded upon by Gabizon et al. (85 PNAS 6949 (1988)). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (53 Bull. Chem. Soc. Jpn. 2778 (1980)) described liposomes comprising a nonionic detergent, 2C1215G, that contains a PEG moiety. Illum et al. (167 FEBS Lett. 79 (1984)), noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. No. 4,426,330 and No. 4,534,899). In addition, antibodies can be conjugated to a polyakylene derivatized liposome (see e.g., PCT Application US 2008/0014255). Klibanov et al. (268 FEBS Lett. 235 (1990)), described experiments demonstrating that liposomes comprising phosphatidylethanolamine (PE) derivatized with PEG or PEG stearate have significant increases in blood circulation half-lives. Blume et al. (1029 Biochim. Biophys. Acta 1029, (1990)), extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. 0 445 131 B1 and

Liposome compositions containing 1-20 mole percent of PE derivatized with PEG, and methods of use thereof, are described by Woodle et al. (U.S. Pat. No. 5,013,556; No. 5,356,633) and Martin et al. (U.S. Pat. No. 5,213,804; European Patent No. 0 496813 B1). Liposomes comprising a number of other lipid-polymer conjugates are disclosed in WO 91/05545 and U.S. Pat. No. 5,225,212 and in WO 94/20073. Liposomes comprising PEG-modified ceramide lipids are described in WO 96/10391. U.S. Pat. No. 5,540,935 and U.S. Pat. No. 5,556,948 describe PEG-containing liposomes that can be further derivatized with functional moieties on their surfaces. Methods and compositions relating to liposomes comprising PEG can be found in, e.g., U.S. Pat. No. 6,049,094; U.S. Pat. No. 6,224,903; U.S. Pat. No. 6,270,806; U.S. Pat. No. 6,471,326; U.S. Pat. No. 6,958,241.

As noted above, liposomes may optionally be prepared to contain surface groups, such as antibodies or antibody fragments, small effector molecules for interacting with cell-surface receptors, antigens, and other like compounds, and these groups can facilitate delivery of liposomes and their contents to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

Lipids can be derivatized using a variety of targeting moieties, such as ligands, cell surface receptors, glycoproteins, vitamins (e.g., riboflavin) and monoclonal antibodies by covalently attaching the ligand to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and as noted above, the hydrophilic polymer polyethyleneglycol (PEG) has been studied widely. Allen et al., 1237 Biochem. Biophys. Acta 99-108 (1995); Zalipsky, 4 Bioconj. Chem. 296-99 (1993); Zalipsky et al., 353 FEBS Lett. 1-74 (1994); Zalipsky et al., Bioconj. Chem. 705-08 (1995); Zalipsky, in STEALTH LIPOSOMES (Lasic & Martin, eds. CRC Press, Boca Raton, Fla., 1995).

A number of liposomes comprising nucleic acids are known in the art, such as methods for encapsulating high molecular weight nucleic acids in liposomes. WO 96/40062. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene. In addition, methods for preparing a liposome composition comprising a nucleic acid can be found in, e.g., U.S. Pat. No. 6,011,020; U.S. Pat. No. 6,074,667; U.S. Pat. No. 6,110,490; U.S. Pat. No. 6,147,204; U.S. Pat. No. 6,271,206; U.S. Pat. No. 6,312,956; U.S. Pat. No. 6,465,188; U.S. Pat. No. 6,506,564; U.S. Pat. No. 6,750,016; U.S. Pat. No. 7,112,337.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing, self-repairing, frequently reach their targets without fragmenting, and often self-loading. To make transfersomes it is possible to add surface edge-activators, usually surfactants, to a standard liposomal composition.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations. See e.g., Malmsten, SURFACTANTS & POLYMERS IN DRUG DELIV. (Informa Health Care, NY, 2002); Rieger, in PHARM. DOSAGE FORMS 285 (Marcel Dekker, Inc., NY, 1988).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class. If the surfactant molecule has the ability to carry either a positive or negative charge, the surfactant is classified as amphoteric. Amphoteric surfactants include acrylic acid derivatives, substituted alkylamides, N-alkylbetaines and phosphatides.

In one embodiment, the present invention employs various penetration enhancers to effect the efficient delivery of nucleic acids, particularly RNA effector molecules, to the cell. Most drugs are present in solution in both ionized and nonionized forms. Usually, only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories: surfactants, fatty acids, bile salts, chelating agents, and non-chelating non-surfactants. See, e.g., Malmsten, 2002; Lee et al., Crit. Rev. Therapeutic Drug Carrier Sys. 92 (1991).

In connection with the present invention, penetration enhancers include surfactants (or “surface-active agents”), which are chemical entities that, when dissolved in an aqueous solution, reduce the surface tension of the solution or the interfacial tension between the aqueous solution and another liquid, with the result that absorption of RNA effector molecules through cellular membranes and other biological barriers is enhanced. In addition to bile salts and fatty acids, these penetration enhancers include, for example, sodium lauryl sulfate, polyoxyethylene-9-lauryl ether and polyoxyethylene-20-cetyl ether) (see, e.g., Malmsten, 2002; Lee et al., 1991); and perfluorochemical emulsions, such as FC-43 (Takahashi et al., 40 J. Pharm. Pharmacol. 252 (1988)).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacyclo-heptan-2-one, acylcarnitines, acylcholines, C1-20 alkyl esters thereof (e.g., methyl, isopropyl and t-butyl), and mono- and di-glycerides thereof (i.e., oleate, laurate, caprate, myristate, palmitate, stearate, linoleate, etc.). See, e.g., Touitou et al., ENHANCEMENT IN DRUG DELIV. (CRC Press, Danvers, Mass., 2006); Lee et al., 1991; Muranishi, 7 Crit. Rev. Therapeutic Drug Carrier Sys. 1-33 (1990); El Hariri et al., 44 J. Pharm. Pharmacol. 651-54 (1992).

The physiological role of bile includes the facilitation of dispersion and absorption of lipids and fat-soluble vitamins. See, e.g., Malmsten, 2002; Brunton, Chapt. 38 in GOODMAN & GILMAN'S PHARMACOLOGICAL BASIS THERAPEUTICS, 9TH ED. 934-35 (Hardman et al., eds., McGraw-Hill, NY, 1996). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, 2002; Lee et al., 1991; Swinyard, Chapt. 39 in REMINGTON'S PHARM. SCI., 18th Ed. 782-83 (Gennaro, ed., Mack Publishing Co., Easton, Pa., 1990); Muranishi, 1990; Yamamoto et al., 263 J. Pharm. Exp. Ther. 25 (1992); Yamashita et al., 79 J. Pharm. Sci. 579-83 (1990).

Chelating agents, as used in connection with the present invention, can be defined as compounds that remove metallic ions from solution by forming complexes therewith, with the result that absorption of RNA effector molecules through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents. Jarrett, 618 J. Chromatogr. 315-39 (1993). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines). See, e.g., Katdare et al., EXCIPIENT DEVEL. PHARM. BIOTECH. & DRUG DELIV. (CRC Press, Danvers, Mass., 2006); Lee et al., 1991; Muranishi, 1990; Buur et al., 14 J. Control Rel. 43-51 (1990).

As used herein, non-chelating non-surfactant penetration enhancing compounds can be defined as compounds that demonstrate insignificant activity as chelating agents or as surfactants but that nonetheless enhance absorption of RNA effector molecules through the alimentary mucosa. See e.g., Muranishi, 1990. This class of penetration enhancers include, for example, unsaturated cyclic ureas, 1-alkyl- and 1-alkenylazacyclo-alkanone derivatives (Lee et al., 1991); and non-steroidal anti-inflammatory agents such as diclofenac sodium, indomethacin and phenylbutazone (Yamashita et al., 1987).

Agents that enhance uptake of RNA effector molecules at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), are also known to enhance the cellular uptake of dsRNAs. Examples of commercially available transfection reagents include, for example LIPOFECTAMINE™ (Invitrogen; Carlsbad, Calif.), LIPOFECTAMINE 2000™ (Invitrogen; Carlsbad, Calif.), 293 FECTIN™ (Invitrogen), CELLFECTIN™ (Invitrogen), DMRIE-C™ (Invitrogen), FREESTYLE™ MAX (Invitrogen), LIPOFECTAMINE™ 2000 CD (Invitrogen), LIPOFECTAMINE™ (Invitrogen), RNAiMAX (Invitrogen), OLIGOFECTAMINE™ (Invitrogen), and OPTIFECT™ (Invitrogen) transfection reagents; and X-tremeGENE Q2 Transfection Reagent (Roche Applied Science; Grenzacherstrasse, Switzerland), DOTAP Liposomal Transfection Reagent (Avante Polar Lipids, Inc., Alabaster, Ala.), DOSPER Liposomal Transfection Reagent (Roche), or FuGENE® (Promega; Madison, Wis.) or TRANSFECTAM® Reagent (Promega), TRANSFAST™ Transfection Reagent (Promega), TFX™-20 Reagent (Promega), TFX™-50 Reagent (Promega); DREAMFECT™ (OZ Biosciences; Marseille, France), EcoTransfect (OZ Biosciences); TRANSPASS® D1 Transfection Reagent (New England Biolabs; Ipswich, Mass.); LYOVEC™/LIPOGENT™ (InvivoGen; San Diego, Calif.); PerFectin Transfection Reagent (Genlantis; San Diego, Calif.), NEUROPORTER Transfection Reagent (Genlantis), GENEPORTER Transfection reagent (Genlanti), GENEPORTER 2 Transfection reagent (Genlantis), CYTOFECTIN Transfection Reagent (Genlantis), BACULOPORTER Transfection Reagent (Genlantis), TROGANPORTERT™ transfection reagent (Genlantis); RIBOFECT (Bioline; Taunton, Mass., U.S.), PLASFECT (Bioline), UNIFECTOR (B-Bridge International; Mountain View, Calif.), SUREFECTOR (B-Bridge International), or HIFECT™ (B-Bridge Int'l), among others.

Other agents can be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal.

The compositions of the present invention can additionally contain other adjunct components so long as such materials, when added, do not unduly interfere with the biological activities of the components of the compositions of the present invention. The formulations can be sterilized and, if desired, mixed with auxiliary agents that do not deleteriously interact with the RNA effector molecules of the formulation.

Aqueous suspensions can contain substances which increase the viscosity of the suspension including, for example, sodium carboxymethylcellulose, sorbitol and/or dextran. The suspension can also contain stabilizers.

Toxicity and therapeutic efficacy of such compounds can be determined by standard pharmaceutical procedures in cell cultures or in cells, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are particularly useful. The data obtained from cell culture assays and animal studies can be used in formulating a range of dosages for use in the instant methods. The dosage of compositions featured in the invention lies generally within a range of concentrations that includes the ED₅₀ with little or no toxicity. The dosage can vary within this range depending upon the dosage form employed and the route of administration utilized.

In yet another aspect, the invention provides a method for inhibiting the expression of a target gene in a host cell by administering a composition featured in the invention to the host cell such that expression of the target gene is decreased for an extended duration, e.g., at least two, three, four days or more, e.g., one week, two weeks, three weeks, or four weeks or longer. The effect of the decreased expression of the target gene preferably results in a decrease in levels of the protein encoded by the target gene by at least 10%, at least 15%, at least 20%, at least 25%, at least 30%, at least 40%, at least 50%, or at least 60%, or more, as compared to pretreatment levels.

In some embodiments, kits are provided for testing the effect of a RNA effector molecule or a series of RNA effector molecules on the production of an immunogenic agent by the cell, where the kits comprise a substrate having one or more assay surfaces suitable for culturing cells under conditions that allow production of an immunogenic agent. In some embodiments, the exterior of the substrate comprises wells, indentations, demarcations, or the like at positions corresponding to the assay surfaces. In some embodiments, the wells, indentations, demarcations, or the like retain fluid, such as cell culture media, over the assay surfaces.

In some embodiments, the assay surfaces on the substrate are sterile and are suitable for culturing host cells under conditions representative of the culture conditions during large-scale (e.g., industrial scale) production of the immunogenic agent. Advantageously, kits provided herein offer a rapid, cost-effective means for testing a wide-range of agents and/or conditions on the production of an immunogenic agent, allowing the cell culture conditions to be established prior to full-scale production of the immunogenic agent.

In some embodiments, one or more assay surfaces of the substrate comprise a concentrated test agent, such as a RNA effector molecule, such that the addition of suitable media to the assay surfaces results in a desired concentration of the RNA effector molecule surrounding the assay surface. In some embodiments, the RNA effector molecules can be printed or ingrained onto the assay surface, or provided in a lyophilized form, e.g., within wells, such that the effector molecules can be reconstituted upon addition of an appropriate amount of media. In some embodiments, the RNA effector molecules are reconstituted by plating cells onto assay surfaces of the substrate.

In some embodiments, kits provided herein further comprise cell culture media suitable for culturing a cell under conditions allowing for the production of an immunogenic agent of interest. The media can be in a ready to use form or can be concentrated (e.g., as a stock solution), lyophilized, or provided in another reconstitutable form.

In further embodiments, kits provided herein further comprise one or more reagents suitable for detecting production of the immunogenic agent by the cell, cell culture, or tissue culture. In further embodiments, the reagent(s) are suitable for detecting a property of the cell, such as maximum cell density, cell viability, or the like, which is indicative of production of the desired immunogenic agent. In some embodiments, the reagent(s) are suitable for detecting the immunogenic agent or a property thereof, such as the biological activity, homogeneity, or structure of the immunogenic agent.

In some embodiments, one or more assay surfaces of the substrate further comprise a carrier for which facilitates uptake of RNA effector molecules by cells. Carriers for RNA effector molecules are known in the art and are described herein. For example, in some embodiments, the carrier is a lipid formulation such as LIPOFECTAMINE™ transfection reagent (Invitrogen; Carlsbad, Calif.) or a related formulation. Examples of such carrier formulations are described herein.

In some embodiments, one or more assay surfaces of the substrate comprise a RNA effector molecule or series of RNA effector molecules and a carrier, each in concentrated form, such that plating test cells onto the assay surface(s) results in a concentration the RNA effector molecule(s) and the carrier effective for facilitating uptake of the RNA effector molecule(s) by the cells and modulation of the expression of one or more genes targeted by the RNA effector molecules.

In some embodiments, the substrate further comprises a matrix which facilitates three-dimensional (3-D) cell growth and/or production of the immunogenic agent by the cells. In further embodiments, the matrix facilitates anchorage-dependent growth of cells. Non-limiting examples of matrix materials suitable for use with various kits described herein include agar, agarose, methylcellulose, alginate hydrogel (e.g., 5% alginate +5% collagen type I), chitosan, hydroactive hydrocolloid polymer gels, polyvinyl alcohol-hydrogel (PVA-H), polylactide-co-glycolide (PLGA), collagen vitrigel, PHEMA (poly(2-hydroxylmethacrylate)) hydrogels, PVP/PEO hydrogels, BD PURAMATRIX™ hydrogels, and copolymers of 2-methacryloyloxyethyl phosphorylcholine (MPC).

In some embodiments, the substrate comprises a microarray plate, a biochip, or the like which allows for the high-throughput, automated testing of a range of test agents, conditions, and/or combinations thereof on the production of an immunogenic agent by cultured cells. For example, the substrate can comprise a 2-dimensional microarray plate or biochip having m columns and n rows of assay surfaces (e.g., residing within wells) which allow for the testing of m×n combinations of test agents and/or conditions (e.g., on a 24, 96 or 384-well microarray plate). The microarray substrates are preferably designed such that all necessary positive and negative controls can be carried out in parallel with testing of the agents and/or conditions.

In further embodiments, kits are provided comprising one or more microarray substrates seeded with a set of RNA effector molecules designed to modulate a particular pathway, function, or property of a cell which affects the production of the immunogenic agent. For example, in some embodiments, the RNA effector molecules are directed against target genes comprising a pathway involved in the expression, folding, secretion, or post-translational modification of a recombinant protein immunogenic agent by the cell.

In further embodiments, kits are provided herein comprising one or more microarray substrates seeded with a set of RNA effector molecules designed to address a particular problem or class of problems associated with the production of an immunogenic agent in cell-based systems. For example, in some embodiments, the RNA effector molecules are directed against target genes expressed by latent or endogenous viruses; or involved in cell processes, such as cell cycle progression, cell metabolism or apoptosis which inhibit or interfere production or purification of the immunogenic agent. In further embodiments, the RNA effector molecules are directed against target genes that mediate enzymatic degradation, aggregation, misfolding, or other processes that reduce the activity, homogeneity, stability, and/or other qualities of the immunogenic agent. In yet further embodiments, the effector molecules are directed against target genes that affect the infectivity of exogenous or adventitious contaminating microbes.

In one embodiment, the immunogenic agent includes a glycoprotein, and the RNA effector molecules are directed against target genes involved in glycosylation (e.g., fucosylation) and/or proteolytic processing of glycoproteins by the host cell.

In another embodiment, the immunogenic agent is a multi-subunit recombinant protein and the RNA effector molecules are directed against target genes involved in the folding and/or secretion of the protein by the host cell. In another embodiment, the RNA effector molecules are directed against target genes involved in post-translation modification of the immunogenic protein in the cells, such as methionine oxidation, glycosylation, disulfide bond formation, pyroglutamation and/or protein deamidation.

In some embodiments, kits provided herein allow for the selection or optimization of at least one factor for enhancing production of the immunogenic agent. For example, the kits can allow for the selection of an RNA effector molecule from among a series of candidate RNA effector molecules, or for the selection of a concentration or concentration range from a wider range of concentrations of a given RNA effector molecule. In some embodiments, the kits allow for selection of one or more RNA effector molecules from a series of candidate RNA effector molecules directed against a common target gene. In further embodiments, the kits allow for selection of one or more RNA effector molecules from a series of candidate RNA effector molecules directed against two or more functionally related target genes or two or more target genes of a common host cell pathway.

In some embodiments, kits provided herein allow for the selection or optimization of a combination of two or more factors in the production of an immunogenic agent. For example, the kits can allow for the selection of a suitable RNA effector molecule from among a series of candidate RNA effector molecules as well as a concentration of the RNA effector molecule. In further embodiments, kits provided herein allow for the selection of a first RNA effector molecule from a first series of candidate RNA effector molecules and a second RNA effector molecule from a second series of candidate RNA effector molecules. In some embodiments, the first and/or second series of candidate RNA effector molecules are directed against a common target gene. In further embodiments, the first and/or second series of RNA effector molecules are directed against two or more functionally related target genes or two or more target genes of a common host cell pathway.

In another embodiment, a kit for enhancing production of an immunogenic agent in a cell, comprising at least a first RNA effector molecule, a portion of which is complementary to at least a first target gene of a latent or endogenous virus; a second RNA effector molecule, a portion of which is complementary to at least a secon target gene of the cellular immune response; and, optionally, a third RNA effector molecule, a portion of which is complementary to at least a third target gene of a cellular process. For example, the first target gene is an ERV env gene, the second target gene is a IFNAR1 or CDKN2A gene, and the third target gene is a PTEN, BAK1, FN1, or LDHA gene. The kit can further comprise at least additional RNA effector molecule that targets a cellular process including, but not limited to, carbon metabolism and transport, apoptosis, RNAi uptake and/or efficiency, reactive oxygen species production, cell cycle control, protein folding, pyroglutamation protein modification, deamidase, glycosylation, disulfide bond formation, protein secretion, gene amplification, viral replication, viral infection, viral particle release, control of cellular pH, and protein production.

In yet another aspect, the invention provides a method for inhibiting the expression of a target gene in a cell. The method includes administering a composition featured in the invention to the cell such that expression of the target gene is decreased, such as for an extended duration, e.g., at least two, three, four days or more. The RNA effector molecules useful for the methods and compositions featured in the invention specifically target RNAs (primary or processed) of the target gene. Compositions and methods for inhibiting the expression of these target genes using RNA effector molecules can be prepared and performed as described herein.

The invention encompasses vaccine formulations comprising the immunogenic agent and a suitable excipient. The terms “vaccine,” is well-understood. For example, the terms vaccine, vaccination or immunization can be understood to be a process or composition that increases a subject's immune reaction to an immunogen (e.g., by providing an active immune response), and therefore its ability to resist, overcome and/or recover from infection (i.e., a protective immune response).

The immunogenic agent used in the vaccine formulation can be a virus selected from naturally occurring mutants or variants, mutagenized viruses or genetically engineered viruses. Attenuated strains of segmented RNA viruses can also be generated via reassortment techniques, or by using a combination of the reverse genetics approach and reassortment techniques. Naturally occurring variants include viruses isolated from nature as well as spontaneous occurring variants generated during virus propagation, having, for example, an impaired ability to antagonize the cellular IFN response. The attenuated virus can itself be used as the active ingredient in the vaccine formulation. For example, the virus can include attenuated influenza viruses, cold-adapted influenza viruses, temperature-sensitive influenza viruses, reassortant influenza viruses, high yield donor influenza viruses, wild-type influenza viruses isolated from throat swabs of infected mammals, and viruses that have been passaged in embryonated chicken eggs or cell culture adapted strains of influenza viruses

Virus or viral-based immunogenic agents of the embodiments of the invention can be any type of animal virus, such as those of arenaviridae, orthomyxoviridae, paramyxoviridae, filoviridae, rabdoviridae, birnaviridae, reoviridae, picornaviridae, coronaviridae, flaviviridae, togaviridae, adenoviridae, herpesviridae, papovaviridae, parvoviridae, circoviridae, poxyiridae, and retroviridae. Particular viruses include poliovirus, hepatitis A virus (HAV), tick-borne encephalitis virus (TBEV), yellow fever virus, rubella virus, hepatitis C virus (HCV), hepatitis B virus (HBV), variola virus, mumps virus, measles virus, rubella virus, respiratory syncytial virus, vesicular stomatits virus (VSV), rabies virus, ebola virus, influenza virus, lassa virus, junin virus, reovirus, adenovirus type 1 to type 47, herpes simplex viruses (HSV 1, HSV 2), cytomegalo virus (CMV), varicella zoster vim (VZV), Epstein-Barr virus (EBV), human immunodeficiency virus (HIV), West Nile virus, rotavirus, or vaccinia virus.

Additionally, immunogenic agents of the embodiments of the present invention can be an antigenic composition or vaccine against the obligate intracellular parasite, Chlamydiae. For example, the chlamydiae can be Chlamydiae trachomatis, C. trachomatis, C. suis, C. pneumoniae, C. pecorum, C. psittaci, C. abortus, C. felis, or C. caviae. C. pneumoniae is a major cause of pneumonia in humans. C. trachomatis causes sexually transmitted diseases and eye infections in humans, is responsible for nearly 2.3 million cases of infection in the U.S. population, and includes the biovars trachoma (serovars A, B, Ba or C), urethritis (serovars D-K), and lymphogranuloma venereum (LGV, serovars L1, 2 and 3). See, e.g., Ryan & Ray, SHERRIS MED. MICRO. 463-70 (4th ed., McGraw Hill, 2004). Modified live C. psittaci vaccine of chicken embryo origin and inactivated chlamydial vaccines have been used. See, e.g., U.S. Pat. No. 4,271,146. Recombinant chlamydial antigens, such as chlamydial protease-like activity factor (CPAF) protein, have been described also. See, e.g., U.S. Patent Pubs. No. 2009/0098165, No. 2009/0155350; EP 0293079.

Additionally, the embodiments of the present invention provide are also applicable to production of an immunogenic bacterial agent in a eukaryotic cell. These include Shigella flexneri, Listeria monocytogenes, Rickettsiae tsutsugamushi, Rickettsiae rickettsiae, Mycobacterium leprae, Mycobacterium tuberculosis, Legionella pneumophila, Chlamydia ssp. Additionally, the embodiments of the present invention provide are also applicable to production of an immunogenic protozoal agent in a eukaryotic cell. These include Plasmodium falciparum, Trpanosoma cruzi, and Leishmania donovani.

Alternatively, the attenuated virus, bacteria, or protozoa can be used as the vector or “backbone” of recombinantly produced vaccines. To this end, recombinant techniques such as reverse genetics (or, for segmented viruses, combinations of the reverse genetics and reassortment techniques) can be used to engineer mutations or introduce foreign antigens into the attenuated virus used in the vaccine formulation. In this way, vaccines can be designed for immunization against strain variants, or in the alternative, against completely different infectious agents or disease antigens.

Any practical heterologous gene sequence can be constructed into the immunogenic agents (e.g., viruses, bacteria, or protozoa) of the invention for use in vaccines. Epitopes that induce a protective immune response to any of a variety of pathogens, or antigens that bind neutralizing antibodies can be expressed by or as part of the viruses. For example, heterologous gene sequences that can be constructed into the viruses of the invention for use in vaccines include but are not limited to epitopes of human immunodeficiency virus (HIV) such as gp120; hepatitis B virus surface antigen (HBsAg); the glycoproteins of herpes virus (e.g., gD, gE, gH, gL); VP1 of poliovirus; antigenic determinants of non-viral pathogens such as bacteria and parasites, to name but a few. In another embodiment, all or portions of immunoglobulin genes can be expressed. For example, variable regions of anti-idiotypic immunoglobulins that mimic such epitopes can be constructed into the viruses of the invention. In yet another embodiment, tumor associated antigens can be expressed.

Either a live recombinant vaccine or an inactivated recombinant vaccine can be formulated. A live vaccine may be preferred because multiplication in the host leads to a prolonged stimulus of similar kind and magnitude to that occurring in natural infections, and therefore, confers substantial, long-lasting immunity. Production of such live recombinant vaccine formulations can be accomplished using conventional methods involving propagation of the microbe or recombant vector in cell culture followed by purification.

Regarding influenza, these vaccine formulations can include genetically engineered negative strand RNA viruses that have mutations in the NS1 or analogous gene including but not limited to the truncated NS1 influenza mutants described in the working examples. They can also be formulated using natural variants, such as the A/turkey/Ore/71 natural variant of influenza A, or B/201, and B/AWBY-234, which are natural variants of influenza B. When formulated as a live virus vaccine, a range of about 10⁴ pfu to about 5×10⁶ pfu per dose can be used.

Vaccine formulations comprising the immunogenic agents of the invention and a pharmaceutically acceptable carrier are also provided, which can be formulated for administration in a pharmaceutical carrier in accordance with known techniques. See, e.g., Remington, SCIENCE & PRACTICE OF PHARMACY (latest edition); U.S. Patent Pub. No. 2009/0098165.

Many methods can be used to introduce the vaccine formulations described herein, these include but are not limited to intranasal, intratracheal, oral, intradermal, intramuscular, intraperitoneal, intravenous, and subcutaneous routes. It may be preferable to introduce the virus vaccine formulation via the natural route of infection of the pathogen for which the vaccine is designed, or via the natural route of infection of the parental attenuated virus. Where a live influenza virus vaccine preparation is used, it may be preferable to introduce the formulation via the natural route of infection for influenza virus. The ability of influenza virus to induce a vigorous secretory and cellular immune response can be used advantageously. For example, infection of the respiratory tract by influenza viruses may induce a strong secretory immune response, for example in the urogenital system, with concomitant protection against a particular disease causing agent.

A vaccine of the present invention could be administered once, or twice or three times with an interval of 2 to 6 months between doses. Alternatively, a vaccine of the present invention, comprising could be administered as often as needed to an animal or a human being.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The present invention may be as defined in any one of the following numbered paragraphs.

Paragraph 1. A method of isolating a cell that is substantially devoid of a target virus, comprising: a. contacting a population of cells a portion of which comprises the target virus with an RNA effector molecule that inhibits the growth or replication of the target virus; b.

detecting the presence of the target virus in each cell; and, c. isolating at least one cell that is substantially devoid of the target virus.

Paragraph 2. The method of paragraph 1, further comprising repeating steps a-c.

Paragraph 3. The method of any of paragraphs 1-2, further comprising detecting the presence or absence of target viral nucleic acid or protein.

Paragraph 4. The method of any of paragraphs 1-3, wherein the isolated cell is free of the target virus.

Paragraph 5. The method of any of paragraphs 1-4, wherein steps a-c are repeated when target viral nucleic acid or protein is detected.

Paragraph 6. The method any paragraphs 1-5, wherein the target viral nucleic acid is detected by a method selected from the group consisting of: polymerase chain reaction (PCR), reverse transcription polymerase chain reaction (RT-PCR), rapid amplification of cDNA ends (RACE), dot blot hybridization, Northern hybridization, and Southern hybridization.

Paragraph 7. The method any of paragraphs 1-5, wherein the target viral protein is detected by a method selected from the group consisting of: radioimmunoassay, competitive-binding assay, ELISA, Western blot, FACS, immunohistochemistry, immunoprecipitation, proteomics, mass spectrometry, electrophoresis, and immunofluoresence.

Paragraph 8. The method of any of paragraphs 1-7, wherein the presence of the target virus of step b) is immunologically detected.

Paragraph 9. The method of paragraph 8, wherein the presence of the target virus is detected using an antibody against a capsid protein (Cap).

Paragraph 10. The method of any of paragraphs 1-9, wherein the cell further comprises a second virus.

Paragraph 11. The method of any of paragraphs 1-10, further comprising detecting the presence of the second virus.

Paragraph 12. The method of any of paragraphs 1-11, wherein the presence of the second virus is detected by detection of viral nucleic acid or viral protein of the second virus.

Paragraph 13. The method of paragraph 12, wherein the viral nucleic acid is detected by a method selected from the group consisting of: polymerase chain reaction (PCR), reverse transcription polymerase chain reaction (RT-PCR), rapid amplification of cDNA ends (RACE), dot blot hybridization, Northern hybridization, and Southern hybridization.

Paragraph 14. The method of paragraph 12, wherein the viral protein is detected by a method selected from the group consisting of: radioimmunoassay, competitive-binding assay, ELISA, Western blot, FACS, immunohistochemistry, immunoprecipitation, proteomics, mass spectrometry, electrophoresis, and immunofluoresence.

Paragraph 15. The method of paragraph 10, wherein the second virus is rotavirus.

Paragraph 16. The method of any of paragraphs 1-15, wherein the target virus is a non-integrating virus.

Paragraph 17. The method of paragraph 16, wherein the target virus is porcine circovirus.

Paragraph 18. The method of any of paragraphs 1-17, wherein the RNA effector molecule is detectably labeled.

Paragraph 19. The method of any of paragraphs 1-18, further comprising detecting the presence of the RNA effector molecule in the cell.

Paragraph 20. A cell that is substantially devoid of a target virus, obtained by the process of any of paragraphs 1-19.

Paragraph 21. A method of producing an immunogenic agent, comprising: a.

culturing the cell of paragraph 15 under conditions that allow expression of the immunogenic agent; and, b. isolating the immunogenic agent.

Paragraph 22. A method of preparing a virus stock that is substantially devoid of an unwanted target virus, comprising: (a) contacting a virus stock that contains unwanted target virus with an agent that binds specifically with the unwanted target virus; and (b) collecting the unbound virus stock.

Paragraph 23. The method of paragraph 22, further comprising repeating steps (a) and (b).

Paragraph 24. The method of any of paragraphs 22-23, further comprising detecting the presence or absence of target viral nucleic acid or protein from the virus stock.

Paragraph 25. The method of any of paragraphs 22-24, wherein the agent is immobilized.

Paragraph 26. The method of any of paragraphs 22-25, wherein the agent is a receptor, an antibody, or an antigen binding fragment thereof.

Paragraph 27. The method of any of paragraphs 22-26, wherein the agent is an antibody that binds to the porcine circovirus-1 (PCV-1)

Paragraph 28. The method of paragraph 27, wherein the antibody binds to the porcine circovirus-1 (PCV-1) capsid (Cap) protein.

Paragraph 29. The method of paragraph 26, wherein the receptor is heparin.

Paragraph 30. The method of any of paragraphs 22-29, wherein the agent is a receptor on a cell surface.

Paragraph 31. The method of paragraph 30, wherein the receptor on a cell surface is heparin.

Paragraph 32. The method of any of paragraphs 22-31, further comprising contacting the cell with a RNA effector molecule that increases the presence of the receptor on the cell surface.

Paragraph 33. The method of paragraph 32, wherein the RNA effector molecule targets the heparanase gene.

Paragraph 34. The method of paragraph 30, wherein the cell does not have a receptor that binds the desired virus of the virus stock.

Paragraph 35. The method of paragraph 34, wherein the receptor is sialic acid.

Paragraph 36. The method of paragraph 30, wherein the cell is treated to have decreased level of cell receptor to the desired virus stock.

Paragraph 37. The method of paragraph 36, wherein the cell is treated by physical, chemical, or enzymatic means.

Paragraph 38. The method of paragraph 34 or paragraph 36, wherein the presence of a cell receptor to the desired viral stock has been decreased by contacting the cell with a RNA effector molecule that inhibits expression of the cell receptor.

Paragraph 39. The method of any one of paragraphs 30 to 38, further comprising (a) contacting the unbound virus stock with a host cell; (b) contacting the cell with an RNA effector molecule that inhibits growth or replication of the unwanted target virus; and (c) isolating the virus stock from the medium.

Paragraph 40. The method of paragraph 39, wherein the host cell does not have a receptor for the unwanted target virus.

Paragraph 41. A virus stock that is substantially devoid of unwanted target virus prepared by the methods of any one of paragraphs 22 to 40.

Paragraph 42. The virus stock of paragraph 41, wherein the virus stock is rotavirus.

Paragraph 43. The virus stock of paragraph 41 or paragraph 42, wherein the unwanted target virus is PCV1.

Paragraph 44. A biological product made using the virus stock of paragraph 41.

Paragraph 45. The biological product of paragraph 44, wherein the product is rotavirus vaccine.

Paragraph 46. A method of preparing a virus stock that is substantially devoid of an unwanted target virus, comprising: (a) propagating a virus stock in a host cell in which expression of a receptor for a suspected viral contaminant has been inhibited; and (b) collecting the virus stock.

Paragraph 47. The method of paragraph 46, wherein the receptor is heparin.

Paragraph 48. The method of paragraph 47, wherein the heparin is inhibited by contacting the host cell with a RNA effector molecule that targets an epimerase, a xylosyltransferase, a galactosyltransferase, a N-acetylglucosaminyl transferase, a glucuronosyl transferase, or a 2 O sulfotransferase gene.

Paragraph 49. The method of any one of paragraphs 46 to 48, further comprising contacting the host cell with a RNA effector molecule that inhibits the growth or replication of the unwanted target virus.

Paragraph 50. The method of any of paragraphs 46 to 49, wherein the unwanted target virus is PCV1.

Paragraph 51. A virus stock that is substantially devoid of an unwanted target virus, prepared by any of the paragraphs of paragraphs 46 to 50.

Paragraph 52. The virus stock of paragraph 51, wherein the virus stock is rotavirus.

Paragraph 53. A biological product made using the virus stock of paragraph 51.

Paragraph 54. The biological product of paragraph 53, wherein the product is rotavirus vaccine.

Paragraph 55. A virus stock that is substantially devoid of an unwanted target virus, prepared by the process comprising: (a) contacting a virus stock that contains unwanted target virus with an agent that binds specifically with the unwanted target virus; and (b) collecting the unbound virus stock.

Paragraph 56. A virus stock that is substantially devoid of an unwanted target virus, prepared by the process comprising: (a) propagating a virus stock in a host cell in which expression of a receptor for a suspected viral contaminant has been inhibited; and (b) collecting the stock virus.

### EXAMPLES

### Example 1

### dsRNAs for Target Gene PCV1 Rep Coding Sequence

Where the source of a reagent is not specifically given herein, such reagent can be obtained from any supplier of reagents for molecular biology at a quality/purity standard for application in molecular biology. Oligonucleotides are synthesized as described elsewhere, see, e.g., U.S. Application Ser. No. 61/223,370.

Duplexes against the Rep/Rep′ coding sequence portion of the porcine circovirus genome (reference strain, genbank accession AY193712.1, subsequence from position 47 to 985) were designed as shown in Table 2. Duplexes designed against Porcine Circovirus 1 (PCV1) Cap gene are shown in Table 21. In one embodiment, the dsRNA for targeting PCV1 rep is a duplex designed from start position 38, 39, 41, 61, 90, 121, 126, 133, 136, 163, 169, 184, 185, 204, 233, 253, 254, 258, 346, 353, 378, 409, 412, 438, 489, 534, 560, 573, 594, 600, 613, 614, 622, 648, 651, 676, 684, 691, 733, 740, 769, 797, 808, 819, 822, 827, 843, or 908 as shown in Table 2 (wherein # indicates start position):

TABLE 2
Target gene: PCV1 rep

| SEQ ID NO: | # | Sense (5′ to 3′) | Antisense (5′ to 3′) | SEQ ID NO: |
|---|---|---|---|---|
| 16 | 1 | AUGCCAAGCAAGAAAAGCG | CGCUUUUCUUGCUUGGCAU | 678 |
| 17 | 2 | UGCCAAGCAAGAAAAGCGG | CCGCUUUUCUUGCUUGGCA | 679 |
| 18 | 3 | GCCAAGCAAGAAAAGCGGC | GCCGCUUUUCUUGCUUGGC | 680 |
| 19 | 4 | CCAAGCAAGAAAAGCGGCC | GGCCGCUUUUCUUGCUUGG | 681 |
| 20 | 5 | CAAGCAAGAAAAGCGGCCC | GGGCCGCUUUUCUUGCUUG | 682 |
| 21 | 6 | AAGCAAGAAAAGCGGCCCG | CGGGCCGCUUUUCUUGCUU | 683 |
| 22 | 9 | CAAGAAAAGCGGCCCGCAA | UUGCGGGCCGCUUUUCUUG | 684 |
| 23 | 10 | AAGAAAAGCGGCCCGCAAC | GUUGCGGGCCGCUUUUCUU | 685 |
| 24 | 22 | CCGCAACCCCAUAAGAGGU | ACCUCUUAUGGGGUUGCGG | 686 |
| 25 | 23 | CGCAACCCCAUAAGAGGUG | CACCUCUUAUGGGGUUGCG | 687 |
| 26 | 24 | GCAACCCCAUAAGAGGUGG | CCACCUCUUAUGGGGUUGC | 688 |
| 27 | 25 | CAACCCCAUAAGAGGUGGG | CCCACCUCUUAUGGGGUUG | 689 |
| 28 | 26 | AACCCCAUAAGAGGUGGGU | ACCCACCUCUUAUGGGGUU | 690 |
| 29 | 27 | ACCCCAUAAGAGGUGGGUG | CACCCACCUCUUAUGGGGU | 691 |
| 30 | 28 | CCCCAUAAGAGGUGGGUGU | ACACCCACCUCUUAUGGGG | 692 |
| 31 | 29 | CCCAUAAGAGGUGGGUGUU | AACACCCACCUCUUAUGGG | 693 |
| 32 | 30 | CCAUAAGAGGUGGGUGUUC | GAACACCCACCUCUUAUGG | 694 |
| 33 | 31 | CAUAAGAGGUGGGUGUUCA | UGAACACCCACCUCUUAUG | 695 |
| 34 | 32 | AUAAGAGGUGGGUGUUCAC | GUGAACACCCACCUCUUAU | 696 |
| 35 | 33 | UAAGAGGUGGGUGUUCACC | GGUGAACACCCACCUCUUA | 697 |
| 36 | 34 | AAGAGGUGGGUGUUCACCC | GGGUGAACACCCACCUCUU | 698 |
| 37 | 35 | AGAGGUGGGUGUUCACCCU | AGGGUGAACACCCACCUCU | 699 |
| 38 | 36 | GAGGUGGGUGUUCACCCUU | AAGGGUGAACACCCACCUC | 700 |
| 39 | 37 | AGGUGGGUGUUCACCCUUA | UAAGGGUGAACACCCACCU | 701 |
| 40 | 38 | GGUGGGUGUUCACCCUUAA | UUAAGGGUGAACACCCACC | 702 |
| 41 | 39 | GUGGGUGUUCACCCUUAAU | AUUAAGGGUGAACACCCAC | 703 |
| 42 | 40 | UGGGUGUUCACCCUUAAUA | UAUUAAGGGUGAACACCCA | 704 |
| 43 | 41 | GGGUGUUCACCCUUAAUAA | UUAUUAAGGGUGAACACCC | 705 |
| 44 | 42 | GGUGUUCACCCUUAAUAAU | AUUAUUAAGGGUGAACACC | 706 |
| 45 | 43 | GUGUUCACCCUUAAUAAUC | GAUUAUUAAGGGUGAACAC | 707 |
| 46 | 44 | UGUUCACCCUUAAUAAUCC | GGAUUAUUAAGGGUGAACA | 708 |
| 47 | 45 | GUUCACCCUUAAUAAUCCU | AGGAUUAUUAAGGGUGAAC | 709 |
| 48 | 46 | UUCACCCUUAAUAAUCCUU | AAGGAUUAUUAAGGGUGAA | 710 |
| 49 | 47 | UCACCCUUAAUAAUCCUUC | GAAGGAUUAUUAAGGGUGA | 711 |
| 50 | 48 | CACCCUUAAUAAUCCUUCC | GGAAGGAUUAUUAAGGGUG | 712 |
| 51 | 49 | ACCCUUAAUAAUCCUUCCG | CGGAAGGAUUAUUAAGGGU | 713 |
| 52 | 50 | CCCUUAAUAAUCCUUCCGA | UCGGAAGGAUUAUUAAGGG | 714 |
| 53 | 51 | CCUUAAUAAUCCUUCCGAG | CUCGGAAGGAUUAUUAAGG | 715 |
| 54 | 52 | CUUAAUAAUCCUUCCGAGG | CCUCGGAAGGAUUAUUAAG | 716 |
| 55 | 53 | UUAAUAAUCCUUCCGAGGA | UCCUCGGAAGGAUUAUUAA | 717 |
| 56 | 54 | UAAUAAUCCUUCCGAGGAG | CUCCUCGGAAGGAUUAUUA | 718 |
| 57 | 55 | AAUAAUCCUUCCGAGGAGG | CCUCCUCGGAAGGAUUAUU | 719 |
| 58 | 56 | AUAAUCCUUCCGAGGAGGA | UCCUCCUCGGAAGGAUUAU | 720 |
| 59 | 57 | UAAUCCUUCCGAGGAGGAG | CUCCUCCUCGGAAGGAUUA | 721 |
| 60 | 58 | AAUCCUUCCGAGGAGGAGA | UCUCCUCCUCGGAAGGAUU | 722 |
| 61 | 59 | AUCCUUCCGAGGAGGAGAA | UUCUCCUCCUCGGAAGGAU | 723 |
| 62 | 60 | UCCUUCCGAGGAGGAGAAA | UUUCUCCUCCUCGGAAGGA | 724 |
| 63 | 61 | CCUUCCGAGGAGGAGAAAA | UUUUCUCCUCCUCGGAAGG | 725 |
| 64 | 78 | AAACAAAAUACGGGAGCUU | AAGCUCCCGUAUUUUGUUU | 726 |
| 65 | 79 | AACAAAAUACGGGAGCUUC | GAAGCUCCCGUAUUUUGUU | 727 |
| 66 | 80 | ACAAAAUACGGGAGCUUCC | GGAAGCUCCCGUAUUUUGU | 728 |
| 67 | 81 | CAAAAUACGGGAGCUUCCA | UGGAAGCUCCCGUAUUUUG | 729 |
| 68 | 82 | AAAAUACGGGAGCUUCCAA | UUGGAAGCUCCCGUAUUUU | 730 |
| 69 | 83 | AAAUACGGGAGCUUCCAAU | AUUGGAAGCUCCCGUAUUU | 731 |
| 70 | 84 | AAUACGGGAGCUUCCAAUC | GAUUGGAAGCUCCCGUAUU | 732 |
| 71 | 85 | AUACGGGAGCUUCCAAUCU | AGAUUGGAAGCUCCCGUAU | 733 |
| 72 | 86 | UACGGGAGCUUCCAAUCUC | GAGAUUGGAAGCUCCCGUA | 734 |
| 73 | 87 | ACGGGAGCUUCCAAUCUCC | GGAGAUUGGAAGCUCCCGU | 735 |
| 74 | 89 | GGGAGCUUCCAAUCUCCCU | AGGGAGAUUGGAAGCUCCC | 736 |
| 75 | 90 | GGAGCUUCCAAUCUCCCUU | AAGGGAGAUUGGAAGCUCC | 737 |
| 76 | 91 | GAGCUUCCAAUCUCCCUUU | AAAGGGAGAUUGGAAGCUC | 738 |
| 77 | 92 | AGCUUCCAAUCUCCCUUUU | AAAAGGGAGAUUGGAAGCU | 739 |
| 78 | 109 | UUUGAUUAUUUUGUUUGCG | CGCAAACAAAAUAAUCAAA | 740 |
| 79 | 110 | UUGAUUAUUUUGUUUGCGG | CCGCAAACAAAAUAAUCAA | 741 |
| 80 | 111 | UGAUUAUUUUGUUUGCGGA | UCCGCAAACAAAAUAAUCA | 742 |
| 81 | 112 | GAUUAUUUUGUUUGCGGAG | CUCCGCAAACAAAAUAAUC | 743 |
| 82 | 113 | AUUAUUUUGUUUGCGGAGA | UCUCCGCAAACAAAAUAAU | 744 |
| 83 | 114 | UUAUUUUGUUUGCGGAGAG | CUCUCCGCAAACAAAAUAA | 745 |
| 84 | 115 | UAUUUUGUUUGCGGAGAGG | CCUCUCCGCAAACAAAAUA | 746 |
| 85 | 116 | AUUUUGUUUGCGGAGAGGA | UCCUCUCCGCAAACAAAAU | 747 |
| 86 | 117 | UUUUGUUUGCGGAGAGGAA | UUCCUCUCCGCAAACAAAA | 748 |
| 87 | 118 | UUUGUUUGCGGAGAGGAAG | CUUCCUCUCCGCAAACAAA | 749 |
| 88 | 119 | UUGUUUGCGGAGAGGAAGG | CCUUCCUCUCCGCAAACAA | 750 |
| 89 | 120 | UGUUUGCGGAGAGGAAGGU | ACCUUCCUCUCCGCAAACA | 751 |
| 90 | 121 | GUUUGCGGAGAGGAAGGUU | AACCUUCCUCUCCGCAAAC | 752 |
| 91 | 122 | UUUGCGGAGAGGAAGGUUU | AAACCUUCCUCUCCGCAAA | 753 |
| 92 | 123 | UUGCGGAGAGGAAGGUUUG | CAAACCUUCCUCUCCGCAA | 754 |
| 93 | 124 | UGCGGAGAGGAAGGUUUGG | CCAAACCUUCCUCUCCGCA | 755 |
| 94 | 125 | GCGGAGAGGAAGGUUUGGA | UCCAAACCUUCCUCUCCGC | 756 |
| 95 | 126 | CGGAGAGGAAGGUUUGGAA | UUCCAAACCUUCCUCUCCG | 757 |
| 96 | 127 | GGAGAGGAAGGUUUGGAAG | CUUCCAAACCUUCCUCUCC | 758 |
| 97 | 128 | GAGAGGAAGGUUUGGAAGA | UCUUCCAAACCUUCCUCUC | 759 |
| 98 | 129 | AGAGGAAGGUUUGGAAGAG | CUCUUCCAAACCUUCCUCU | 760 |
| 99 | 130 | GAGGAAGGUUUGGAAGAGG | CCUCUUCCAAACCUUCCUC | 761 |
| 100 | 131 | AGGAAGGUUUGGAAGAGGG | CCCUCUUCCAAACCUUCCU | 762 |
| 101 | 132 | GGAAGGUUUGGAAGAGGGU | ACCCUCUUCCAAACCUUCC | 763 |
| 102 | 133 | GAAGGUUUGGAAGAGGGUA | UACCCUCUUCCAAACCUUC | 764 |
| 103 | 134 | AAGGUUUGGAAGAGGGUAG | CUACCCUCUUCCAAACCUU | 765 |
| 104 | 135 | AGGUUUGGAAGAGGGUAGA | UCUACCCUCUUCCAAACCU | 766 |
| 105 | 136 | GGUUUGGAAGAGGGUAGAA | UUCUACCCUCUUCCAAACC | 767 |
| 106 | 137 | GUUUGGAAGAGGGUAGAAC | GUUCUACCCUCUUCCAAAC | 768 |
| 107 | 138 | UUUGGAAGAGGGUAGAACU | AGUUCUACCCUCUUCCAAA | 769 |
| 108 | 139 | UUGGAAGAGGGUAGAACUC | GAGUUCUACCCUCUUCCAA | 770 |
| 109 | 141 | GGAAGAGGGUAGAACUCCU + C57 | GGAGUUCUACCCUCUUCCA | 771 |
| 110 | 142 | GAAGAGGGUAGAACUCCUC | AGGAGUUCUACCCUCUUCC | 772 |
| 111 | 143 | AAGAGGGUAGAACUCCUCA | GAGGAGUUCUACCCUCUUC | 773 |
| 112 | 144 | AGAGGGUAGAACUCCUCAC | UGAGGAGUUCUACCCUCUU | 774 |
| 113 | 145 | GAGGGUAGAACUCCUCACC | GUGAGGAGUUCUACCCUCU | 775 |
| 114 | 146 | AGGGUAGAACUCCUCACCU | GGUGAGGAGUUCUACCCUC | 776 |
| 115 | 147 | GGGUAGAACUCCUCACCUC | AGGUGAGGAGUUCUACCCU | 777 |
| 116 | 148 | GGUAGAACUCCUCACCUCC | GAGGUGAGGAGUUCUACCC | 778 |
| 117 | 149 | GUAGAACUCCUCACCUCCA | GGAGGUGAGGAGUUCUACC | 779 |
| 118 | 150 | UAGAACUCCUCACCUCCAG | UGGAGGUGAGGAGUUCUAC | 780 |
| 119 | 151 | AGAACUCCUCACCUCCAGG | CUGGAGGUGAGGAGUUCUA | 781 |
| 120 | 156 | UCCUCACCUCCAGGGGUUU | CCUGGAGGUGAGGAGUUCU | 782 |
| 121 | 161 | ACCUCCAGGGGUUUGCGAA | AAACCCCUGGAGGUGAGGA | 783 |
| 122 | 162 | CCUCCAGGGGUUUGCGAAU | UUCGCAAACCCCUGGAGGU | 784 |
| 123 | 163 | CUCCAGGGGUUUGCGAAUU | AUUCGCAAACCCCUGGAGG | 785 |
| 124 | 164 | UCCAGGGGUUUGCGAAUUU | AAUUCGCAAACCCCUGGAG | 786 |
| 125 | 169 | GGGUUUGCGAAUUUUGCUA | AAAUUCGCAAACCCCUGGA | 787 |
| 126 | 170 | GGUUUGCGAAUUUUGCUAA | UAGCAAAAUUCGCAAACCC | 788 |
| 127 | 171 | GUUUGCGAAUUUUGCUAAG | UUAGCAAAAUUCGCAAACC | 789 |
| 128 | 172 | UUUGCGAAUUUUGCUAAGA | CUUAGCAAAAUUCGCAAAC | 790 |
| 129 | 173 | UUGCGAAUUUUGCUAAGAA | UCUUAGCAAAAUUCGCAAA | 791 |
| 130 | 174 | UGCGAAUUUUGCUAAGAAG | UUCUUAGCAAAAUUCGCAA | 792 |
| 131 | 175 | GCGAAUUUUGCUAAGAAGC | CUUCUUAGCAAAAUUCGCA | 793 |
| 132 | 176 | CGAAUUUUGCUAAGAAGCA | GCUUCUUAGCAAAAUUCGC | 794 |
| 133 | 177 | GAAUUUUGCUAAGAAGCAG | UGCUUCUUAGCAAAAUUCG | 795 |
| 134 | 178 | AAUUUUGCUAAGAAGCAGA | CUGCUUCUUAGCAAAAUUC | 796 |
| 135 | 179 | AUUUUGCUAAGAAGCAGAC | UCUGCUUCUUAGCAAAAUU | 797 |
| 136 | 180 | UUUUGCUAAGAAGCAGACU | GUCUGCUUCUUAGCAAAAU | 798 |
| 137 | 181 | UUUGCUAAGAAGCAGACUU | AGUCUGCUUCUUAGCAAAA | 799 |
| 138 | 182 | UUGCUAAGAAGCAGACUUU | AAGUCUGCUUCUUAGCAAA | 800 |
| 139 | 183 | UGCUAAGAAGCAGACUUUU | AAAGUCUGCUUCUUAGCAA | 801 |
| 140 | 184 | GCUAAGAAGCAGACUUUUA | AAAAGUCUGCUUCUUAGCA | 802 |
| 141 | 185 | CUAAGAAGCAGACUUUUAA | UAAAAGUCUGCUUCUUAGC | 803 |
| 142 | 186 | UAAGAAGCAGACUUUUAAC | UUAAAAGUCUGCUUCUUAG | 804 |
| 143 | 187 | AAGAAGCAGACUUUUAACA | GUUAAAAGUCUGCUUCUUA | 805 |
| 144 | 188 | AGAAGCAGACUUUUAACAA | UGUUAAAAGUCUGCUUCUU | 806 |
| 145 | 189 | GAAGCAGACUUUUAACAAG | UUGUUAAAAGUCUGCUUCU | 807 |
| 146 | 190 | AAGCAGACUUUUAACAAGG | CUUGUUAAAAGUCUGCUUC | 808 |
| 147 | 191 | AGCAGACUUUUAACAAGGU | CCUUGUUAAAAGUCUGCUU | 809 |
| 148 | 192 | GCAGACUUUUAACAAGGUG | ACCUUGUUAAAAGUCUGCU | 810 |
| 149 | 193 | CAGACUUUUAACAAGGUGA | CACCUUGUUAAAAGUCUGC | 811 |
| 150 | 194 | AGACUUUUAACAAGGUGAA | UCACCUUGUUAAAAGUCUG | 812 |
| 151 | 195 | GACUUUUAACAAGGUGAAG | UUCACCUUGUUAAAAGUCU | 813 |
| 152 | 196 | ACUUUUAACAAGGUGAAGU | CUUCACCUUGUUAAAAGUC | 814 |
| 153 | 197 | CUUUUAACAAGGUGAAGUG | ACUUCACCUUGUUAAAAGU | 815 |
| 154 | 198 | UUUUAACAAGGUGAAGUGG | CACUUCACCUUGUUAAAAG | 816 |
| 155 | 199 | UUUAACAAGGUGAAGUGGU | CCACUUCACCUUGUUAAAA | 817 |
| 156 | 200 | UUAACAAGGUGAAGUGGUA | ACCACUUCACCUUGUUAAA | 818 |
| 157 | 201 | UAACAAGGUGAAGUGGUAU | UACCACUUCACCUUGUUAA | 819 |
| 158 | 202 | AACAAGGUGAAGUGGUAUU | AUACCACUUCACCUUGUUA | 820 |
| 159 | 203 | ACAAGGUGAAGUGGUAUUU | AAUACCACUUCACCUUGUU | 821 |
| 160 | 204 | CAAGGUGAAGUGGUAUUUU | AAAUACCACUUCACCUUGU | 822 |
| 161 | 205 | AAGGUGAAGUGGUAUUUUG | AAAAUACCACUUCACCUUG | 823 |
| 162 | 206 | AGGUGAAGUGGUAUUUUGG | CAAAAUACCACUUCACCUU | 824 |
| 163 | 207 | GGUGAAGUGGUAUUUUGGU | CCAAAAUACCACUUCACCU | 825 |
| 164 | 208 | GUGAAGUGGUAUUUUGGUG | ACCAAAAUACCACUUCACC | 826 |
| 165 | 209 | UGAAGUGGUAUUUUGGUGC | CACCAAAAUACCACUUCAC | 827 |
| 166 | 210 | GAAGUGGUAUUUUGGUGCC | GCACCAAAAUACCACUUCA | 828 |
| 167 | 211 | AAGUGGUAUUUUGGUGCCC | GGCACCAAAAUACCACUUC | 829 |
| 168 | 212 | AGUGGUAUUUUGGUGCCCG | GGGCACCAAAAUACCACUU | 830 |
| 169 | 213 | GUGGUAUUUUGGUGCCCGC | GCGGGCACCAAAAUACCAC | 831 |
| 170 | 214 | UGGUAUUUUGGUGCCCGCU | AGCGGGCACCAAAAUACCA | 832 |
| 171 | 215 | GGUAUUUUGGUGCCCGCUG | CAGCGGGCACCAAAAUACC | 833 |
| 172 | 216 | GUAUUUUGGUGCCCGCUGC | GCAGCGGGCACCAAAAUAC | 834 |
| 173 | 217 | UAUUUUGGUGCCCGCUGCC | GGCAGCGGGCACCAAAAUA | 835 |
| 174 | 218 | AUUUUGGUGCCCGCUGCCA | UGGCAGCGGGCACCAAAAU | 836 |
| 175 | 228 | CCGCUGCCACAUCGAGAAA | UUUCUCGAUGUGGCAGCGG | 837 |
| 176 | 229 | CGCUGCCACAUCGAGAAAG | CUUUCUCGAUGUGGCAGCG | 838 |
| 177 | 230 | GCUGCCACAUCGAGAAAGC | GCUUUCUCGAUGUGGCAGC | 839 |
| 178 | 231 | CUGCCACAUCGAGAAAGCG | CGCUUUCUCGAUGUGGCAG | 840 |
| 179 | 232 | UGCCACAUCGAGAAAGCGA | UCGCUUUCUCGAUGUGGCA | 841 |
| 180 | 233 | GCCACAUCGAGAAAGCGAA | UUCGCUUUCUCGAUGUGGC | 842 |
| 181 | 234 | CCACAUCGAGAAAGCGAAA | UUUCGCUUUCUCGAUGUGG | 843 |
| 182 | 235 | CACAUCGAGAAAGCGAAAG | CUUUCGCUUUCUCGAUGUG | 844 |
| 183 | 236 | ACAUCGAGAAAGCGAAAGG | CCUUUCGCUUUCUCGAUGU | 845 |
| 184 | 237 | CAUCGAGAAAGCGAAAGGA | UCCUUUCGCUUUCUCGAUG | 846 |
| 185 | 238 | AUCGAGAAAGCGAAAGGAA | UUCCUUUCGCUUUCUCGAU | 847 |
| 186 | 239 | UCGAGAAAGCGAAAGGAAC | GUUCCUUUCGCUUUCUCGA | 848 |
| 187 | 240 | CGAGAAAGCGAAAGGAACC | GGUUCCUUUCGCUUUCUCG | 849 |
| 188 | 241 | GAGAAAGCGAAAGGAACCG | CGGUUCCUUUCGCUUUCUC | 850 |
| 189 | 242 | AGAAAGCGAAAGGAACCGA | UCGGUUCCUUUCGCUUUCU | 851 |
| 190 | 243 | GAAAGCGAAAGGAACCGAC | GUCGGUUCCUUUCGCUUUC | 852 |
| 191 | 244 | AAAGCGAAAGGAACCGACC | GGUCGGUUCCUUUCGCUUU | 853 |
| 192 | 245 | AAGCGAAAGGAACCGACCA | UGGUCGGUUCCUUUCGCUU | 854 |
| 193 | 246 | AGCGAAAGGAACCGACCAG | CUGGUCGGUUCCUUUCGCU | 855 |
| 194 | 248 | CGAAAGGAACCGACCAGCA | UGCUGGUCGGUUCCUUUCG | 856 |
| 195 | 249 | GAAAGGAACCGACCAGCAG | CUGCUGGUCGGUUCCUUUC | 857 |
| 196 | 250 | AAAGGAACCGACCAGCAGA | UCUGCUGGUCGGUUCCUUU | 858 |
| 197 | 251 | AAGGAACCGACCAGCAGAA | UUCUGCUGGUCGGUUCCUU | 859 |
| 198 | 252 | AGGAACCGACCAGCAGAAU | AUUCUGCUGGUCGGUUCCU | 860 |
| 199 | 253 | GGAACCGACCAGCAGAAUA | UAUUCUGCUGGUCGGUUCC | 861 |
| 200 | 254 | GAACCGACCAGCAGAAUAA | UUAUUCUGCUGGUCGGUUC | 862 |
| 201 | 255 | AACCGACCAGCAGAAUAAA | UUUAUUCUGCUGGUCGGUU | 863 |
| 202 | 256 | ACCGACCAGCAGAAUAAAG | CUUUAUUCUGCUGGUCGGU | 864 |
| 203 | 257 | CCGACCAGCAGAAUAAAGA | UCUUUAUUCUGCUGGUCGG | 865 |
| 204 | 258 | CGACCAGCAGAAUAAAGAA | UUCUUUAUUCUGCUGGUCG | 866 |
| 205 | 259 | GACCAGCAGAAUAAAGAAU | AUUCUUUAUUCUGCUGGUC | 867 |
| 206 | 260 | ACCAGCAGAAUAAAGAAUA | UAUUCUUUAUUCUGCUGGU | 868 |
| 207 | 261 | CCAGCAGAAUAAAGAAUAC | GUAUUCUUUAUUCUGCUGG | 869 |
| 208 | 262 | CAGCAGAAUAAAGAAUACU | AGUAUUCUUUAUUCUGCUG | 870 |
| 209 | 263 | AGCAGAAUAAAGAAUACUG | CAGUAUUCUUUAUUCUGCU | 871 |
| 210 | 264 | GCAGAAUAAAGAAUACUGC | GCAGUAUUCUUUAUUCUGC | 872 |
| 211 | 265 | CAGAAUAAAGAAUACUGCA | UGCAGUAUUCUUUAUUCUG | 873 |
| 212 | 266 | AGAAUAAAGAAUACUGCAG | CUGCAGUAUUCUUUAUUCU | 874 |
| 213 | 267 | GAAUAAAGAAUACUGCAGU | ACUGCAGUAUUCUUUAUUC | 875 |
| 214 | 268 | AAUAAAGAAUACUGCAGUA | UACUGCAGUAUUCUUUAUU | 876 |
| 215 | 269 | AUAAAGAAUACUGCAGUAA | UUACUGCAGUAUUCUUUAU | 877 |
| 216 | 270 | UAAAGAAUACUGCAGUAAA | UUUACUGCAGUAUUCUUUA | 878 |
| 217 | 271 | AAAGAAUACUGCAGUAAAG | CUUUACUGCAGUAUUCUUU | 879 |
| 218 | 272 | AAGAAUACUGCAGUAAAGA | UCUUUACUGCAGUAUUCUU | 880 |
| 219 | 273 | AGAAUACUGCAGUAAAGAA | UUCUUUACUGCAGUAUUCU | 881 |
| 220 | 274 | GAAUACUGCAGUAAAGAAG | CUUCUUUACUGCAGUAUUC | 882 |
| 221 | 275 | AAUACUGCAGUAAAGAAGG | CCUUCUUUACUGCAGUAUU | 883 |
| 222 | 276 | AUACUGCAGUAAAGAAGGC | GCCUUCUUUACUGCAGUAU | 884 |
| 223 | 277 | UACUGCAGUAAAGAAGGCC | GGCCUUCUUUACUGCAGUA | 885 |
| 224 | 278 | ACUGCAGUAAAGAAGGCCA | UGGCCUUCUUUACUGCAGU | 886 |
| 225 | 279 | CUGCAGUAAAGAAGGCCAC | GUGGCCUUCUUUACUGCAG | 887 |
| 226 | 280 | UGCAGUAAAGAAGGCCACA | UGUGGCCUUCUUUACUGCA | 888 |
| 227 | 281 | GCAGUAAAGAAGGCCACAU | AUGUGGCCUUCUUUACUGC | 889 |
| 228 | 282 | CAGUAAAGAAGGCCACAUA | UAUGUGGCCUUCUUUACUG | 890 |
| 229 | 283 | AGUAAAGAAGGCCACAUAC | GUAUGUGGCCUUCUUUACU | 891 |
| 230 | 284 | GUAAAGAAGGCCACAUACU | AGUAUGUGGCCUUCUUUAC | 892 |
| 231 | 285 | UAAAGAAGGCCACAUACUU | AAGUAUGUGGCCUUCUUUA | 893 |
| 232 | 286 | AAAGAAGGCCACAUACUUA | UAAGUAUGUGGCCUUCUUU | 894 |
| 233 | 287 | AAGAAGGCCACAUACUUAU | AUAAGUAUGUGGCCUUCUU | 895 |
| 234 | 288 | AGAAGGCCACAUACUUAUC | GAUAAGUAUGUGGCCUUCU | 896 |
| 235 | 289 | GAAGGCCACAUACUUAUCG | CGAUAAGUAUGUGGCCUUC | 897 |
| 236 | 290 | AAGGCCACAUACUUAUCGA | UCGAUAAGUAUGUGGCCUU | 898 |
| 237 | 291 | AGGCCACAUACUUAUCGAG | CUCGAUAAGUAUGUGGCCU | 899 |
| 238 | 292 | GGCCACAUACUUAUCGAGU | ACUCGAUAAGUAUGUGGCC | 900 |
| 239 | 293 | GCCACAUACUUAUCGAGUG | CACUCGAUAAGUAUGUGGC | 901 |
| 240 | 294 | CCACAUACUUAUCGAGUGU | ACACUCGAUAAGUAUGUGG | 902 |
| 241 | 295 | CACAUACUUAUCGAGUGUG | CACACUCGAUAAGUAUGUG | 903 |
| 242 | 296 | ACAUACUUAUCGAGUGUGG | CCACACUCGAUAAGUAUGU | 904 |
| 243 | 297 | CAUACUUAUCGAGUGUGGA | UCCACACUCGAUAAGUAUG | 905 |
| 244 | 298 | AUACUUAUCGAGUGUGGAG | CUCCACACUCGAUAAGUAU | 906 |
| 245 | 299 | UACUUAUCGAGUGUGGAGC | GCUCCACACUCGAUAAGUA | 907 |
| 246 | 300 | ACUUAUCGAGUGUGGAGCU | AGCUCCACACUCGAUAAGU | 908 |
| 247 | 301 | CUUAUCGAGUGUGGAGCUC | GAGCUCCACACUCGAUAAG | 909 |
| 248 | 302 | UUAUCGAGUGUGGAGCUCC | GGAGCUCCACACUCGAUAA | 910 |
| 249 | 303 | UAUCGAGUGUGGAGCUCCG | CGGAGCUCCACACUCGAUA | 911 |
| 250 | 334 | AAGCGCAGCGACCUGUCUA | UAGACAGGUCGCUGCGCUU | 912 |
| 251 | 339 | CAGCGACCUGUCUACUGCU | AGCAGUAGACAGGUCGCUG | 913 |
| 252 | 340 | AGCGACCUGUCUACUGCUG | CAGCAGUAGACAGGUCGCU | 914 |
| 253 | 341 | GCGACCUGUCUACUGCUGU | ACAGCAGUAGACAGGUCGC | 915 |
| 254 | 342 | CGACCUGUCUACUGCUGUG | CACAGCAGUAGACAGGUCG | 916 |
| 255 | 343 | GACCUGUCUACUGCUGUGA | UCACAGCAGUAGACAGGUC | 917 |
| 256 | 344 | ACCUGUCUACUGCUGUGAG | CUCACAGCAGUAGACAGGU | 918 |
| 257 | 345 | CCUGUCUACUGCUGUGAGU | ACUCACAGCAGUAGACAGG | 919 |
| 258 | 346 | CUGUCUACUGCUGUGAGUA | UACUCACAGCAGUAGACAG | 920 |
| 259 | 347 | UGUCUACUGCUGUGAGUAC | GUACUCACAGCAGUAGACA | 921 |
| 260 | 348 | GUCUACUGCUGUGAGUACC | GGUACUCACAGCAGUAGAC | 922 |
| 261 | 349 | UCUACUGCUGUGAGUACCC | GGGUACUCACAGCAGUAGA | 923 |
| 262 | 350 | CUACUGCUGUGAGUACCCU | AGGGUACUCACAGCAGUAG | 924 |
| 263 | 351 | UACUGCUGUGAGUACCCUU | AAGGGUACUCACAGCAGUA | 925 |
| 264 | 352 | ACUGCUGUGAGUACCCUUU | AAAGGGUACUCACAGCAGU | 926 |
| 265 | 353 | CUGCUGUGAGUACCCUUUU | AAAAGGGUACUCACAGCAG | 927 |
| 266 | 354 | UGCUGUGAGUACCCUUUUG | CAAAAGGGUACUCACAGCA | 928 |
| 267 | 355 | GCUGUGAGUACCCUUUUGG | CCAAAAGGGUACUCACAGC | 929 |
| 268 | 356 | CUGUGAGUACCCUUUUGGA | UCCAAAAGGGUACUCACAG | 930 |
| 269 | 357 | UGUGAGUACCCUUUUGGAG | CUCCAAAAGGGUACUCACA | 931 |
| 270 | 358 | GUGAGUACCCUUUUGGAGA | UCUCCAAAAGGGUACUCAC | 932 |
| 271 | 359 | UGAGUACCCUUUUGGAGAC | GUCUCCAAAAGGGUACUCA | 933 |
| 272 | 360 | GAGUACCCUUUUGGAGACG | CGUCUCCAAAAGGGUACUC | 934 |
| 273 | 361 | AGUACCCUUUUGGAGACGG | CCGUCUCCAAAAGGGUACU | 935 |
| 274 | 362 | GUACCCUUUUGGAGACGGG | CCCGUCUCCAAAAGGGUAC | 936 |
| 275 | 369 | UUUGGAGACGGGGUCUUUG | CAAAGACCCCGUCUCCAAA | 937 |
| 276 | 370 | UUGGAGACGGGGUCUUUGG | CCAAAGACCCCGUCUCCAA | 938 |
| 277 | 371 | UGGAGACGGGGUCUUUGGU | ACCAAAGACCCCGUCUCCA | 939 |
| 278 | 373 | GAGACGGGGUCUUUGGUGA | UCACCAAAGACCCCGUCUC | 940 |
| 279 | 374 | AGACGGGGUCUUUGGUGAC | GUCACCAAAGACCCCGUCU | 941 |
| 280 | 375 | GACGGGGUCUUUGGUGACU | AGUCACCAAAGACCCCGUC | 942 |
| 281 | 376 | ACGGGGUCUUUGGUGACUG | CAGUCACCAAAGACCCCGU | 943 |
| 282 | 377 | CGGGGUCUUUGGUGACUGU | ACAGUCACCAAAGACCCCG | 944 |
| 283 | 378 | GGGGUCUUUGGUGACUGUA | UACAGUCACCAAAGACCCC | 945 |
| 284 | 379 | GGGUCUUUGGUGACUGUAG | CUACAGUCACCAAAGACCC | 946 |
| 285 | 380 | GGUCUUUGGUGACUGUAGC | GCUACAGUCACCAAAGACC | 947 |
| 286 | 381 | GUCUUUGGUGACUGUAGCC | GGCUACAGUCACCAAAGAC | 948 |
| 287 | 382 | UCUUUGGUGACUGUAGCCG | CGGCUACAGUCACCAAAGA | 949 |
| 288 | 383 | CUUUGGUGACUGUAGCCGA | UCGGCUACAGUCACCAAAG | 950 |
| 289 | 384 | UUUGGUGACUGUAGCCGAG | CUCGGCUACAGUCACCAAA | 951 |
| 290 | 385 | UUGGUGACUGUAGCCGAGC | GCUCGGCUACAGUCACCAA | 952 |
| 291 | 386 | UGGUGACUGUAGCCGAGCA | UGCUCGGCUACAGUCACCA | 953 |
| 292 | 388 | GUGACUGUAGCCGAGCAGU | ACUGCUCGGCUACAGUCAC | 954 |
| 293 | 389 | UGACUGUAGCCGAGCAGUU | AACUGCUCGGCUACAGUCA | 955 |
| 294 | 390 | GACUGUAGCCGAGCAGUUC | GAACUGCUCGGCUACAGUC | 956 |
| 295 | 391 | ACUGUAGCCGAGCAGUUCC | GGAACUGCUCGGCUACAGU | 957 |
| 296 | 393 | UGUAGCCGAGCAGUUCCCU | AGGGAACUGCUCGGCUACA | 958 |
| 297 | 395 | UAGCCGAGCAGUUCCCUGU | ACAGGGAACUGCUCGGCUA | 959 |
| 298 | 396 | AGCCGAGCAGUUCCCUGUA | UACAGGGAACUGCUCGGCU | 960 |
| 299 | 397 | GCCGAGCAGUUCCCUGUAA | UUACAGGGAACUGCUCGGC | 961 |
| 300 | 398 | CCGAGCAGUUCCCUGUAAC | GUUACAGGGAACUGCUCGG | 962 |
| 301 | 399 | CGAGCAGUUCCCUGUAACG | CGUUACAGGGAACUGCUCG | 963 |
| 302 | 400 | GAGCAGUUCCCUGUAACGU | ACGUUACAGGGAACUGCUC | 964 |
| 303 | 401 | AGCAGUUCCCUGUAACGUA | UACGUUACAGGGAACUGCU | 965 |
| 304 | 402 | GCAGUUCCCUGUAACGUAU | AUACGUUACAGGGAACUGC | 966 |
| 305 | 403 | CAGUUCCCUGUAACGUAUG | CAUACGUUACAGGGAACUG | 967 |
| 306 | 404 | AGUUCCCUGUAACGUAUGU | ACAUACGUUACAGGGAACU | 968 |
| 307 | 405 | GUUCCCUGUAACGUAUGUG | CACAUACGUUACAGGGAAC | 969 |
| 308 | 406 | UUCCCUGUAACGUAUGUGA | UCACAUACGUUACAGGGAA | 970 |
| 309 | 407 | UCCCUGUAACGUAUGUGAG | CUCACAUACGUUACAGGGA | 971 |
| 310 | 408 | CCCUGUAACGUAUGUGAGA | UCUCACAUACGUUACAGGG | 972 |
| 311 | 409 | CCUGUAACGUAUGUGAGAA | UUCUCACAUACGUUACAGG | 973 |
| 312 | 410 | CUGUAACGUAUGUGAGAAA | UUUCUCACAUACGUUACAG | 974 |
| 313 | 411 | UGUAACGUAUGUGAGAAAU | AUUUCUCACAUACGUUACA | 975 |
| 314 | 412 | GUAACGUAUGUGAGAAAUU | AAUUUCUCACAUACGUUAC | 976 |
| 315 | 413 | UAACGUAUGUGAGAAAUUU | AAAUUUCUCACAUACGUUA | 977 |
| 316 | 414 | AACGUAUGUGAGAAAUUUC | GAAAUUUCUCACAUACGUU | 978 |
| 317 | 415 | ACGUAUGUGAGAAAUUUCC | GGAAAUUUCUCACAUACGU | 979 |
| 318 | 416 | CGUAUGUGAGAAAUUUCCG | CGGAAAUUUCUCACAUACG | 980 |
| 319 | 417 | GUAUGUGAGAAAUUUCCGC | GCGGAAAUUUCUCACAUAC | 981 |
| 320 | 418 | UAUGUGAGAAAUUUCCGCG | CGCGGAAAUUUCUCACAUA | 982 |
| 321 | 419 | AUGUGAGAAAUUUCCGCGG | CCGCGGAAAUUUCUCACAU | 983 |
| 322 | 420 | UGUGAGAAAUUUCCGCGGG | CCCGCGGAAAUUUCUCACA | 984 |
| 323 | 421 | GUGAGAAAUUUCCGCGGGC | GCCCGCGGAAAUUUCUCAC | 985 |
| 324 | 422 | UGAGAAAUUUCCGCGGGCU | AGCCCGCGGAAAUUUCUCA | 986 |
| 325 | 423 | GAGAAAUUUCCGCGGGCUG | CAGCCCGCGGAAAUUUCUC | 987 |
| 326 | 424 | AGAAAUUUCCGCGGGCUGG | CCAGCCCGCGGAAAUUUCU | 988 |
| 327 | 426 | AAAUUUCCGCGGGCUGGCU | AGCCAGCCCGCGGAAAUUU | 989 |
| 328 | 434 | GCGGGCUGGCUGAACUUUU | AAAAGUUCAGCCAGCCCGC | 990 |
| 329 | 435 | CGGGCUGGCUGAACUUUUG | CAAAAGUUCAGCCAGCCCG | 991 |
| 330 | 436 | GGGCUGGCUGAACUUUUGA | UCAAAAGUUCAGCCAGCCC | 992 |
| 331 | 437 | GGCUGGCUGAACUUUUGAA | UUCAAAAGUUCAGCCAGCC | 993 |
| 332 | 438 | GCUGGCUGAACUUUUGAAA | UUUCAAAAGUUCAGCCAGC | 994 |
| 333 | 439 | CUGGCUGAACUUUUGAAAG | CUUUCAAAAGUUCAGCCAG | 995 |
| 334 | 440 | UGGCUGAACUUUUGAAAGU | ACUUUCAAAAGUUCAGCCA | 996 |
| 335 | 441 | GGCUGAACUUUUGAAAGUG | CACUUUCAAAAGUUCAGCC | 997 |
| 336 | 442 | GCUGAACUUUUGAAAGUGA | UCACUUUCAAAAGUUCAGC | 998 |
| 337 | 443 | CUGAACUUUUGAAAGUGAG | CUCACUUUCAAAAGUUCAG | 999 |
| 338 | 444 | UGAACUUUUGAAAGUGAGC | GCUCACUUUCAAAAGUUCA | 1000 |
| 339 | 445 | GAACUUUUGAAAGUGAGCG | CGCUCACUUUCAAAAGUUC | 1001 |
| 340 | 446 | AACUUUUGAAAGUGAGCGG | CCGCUCACUUUCAAAAGUU | 1002 |
| 341 | 447 | ACUUUUGAAAGUGAGCGGG | CCCGCUCACUUUCAAAAGU | 1003 |
| 342 | 448 | CUUUUGAAAGUGAGCGGGA | UCCCGCUCACUUUCAAAAG | 1004 |
| 343 | 449 | UUUUGAAAGUGAGCGGGAA | UUCCCGCUCACUUUCAAAA | 1005 |
| 344 | 450 | UUUGAAAGUGAGCGGGAAG | CUUCCCGCUCACUUUCAAA | 1006 |
| 345 | 451 | UUGAAAGUGAGCGGGAAGA | UCUUCCCGCUCACUUUCAA | 1007 |
| 346 | 452 | UGAAAGUGAGCGGGAAGAU | AUCUUCCCGCUCACUUUCA | 1008 |
| 347 | 453 | GAAAGUGAGCGGGAAGAUG | CAUCUUCCCGCUCACUUUC | 1009 |
| 348 | 454 | AAAGUGAGCGGGAAGAUGC | GCAUCUUCCCGCUCACUUU | 1010 |
| 349 | 455 | AAGUGAGCGGGAAGAUGCA | UGCAUCUUCCCGCUCACUU | 1011 |
| 350 | 456 | AGUGAGCGGGAAGAUGCAG | CUGCAUCUUCCCGCUCACU | 1012 |
| 351 | 458 | UGAGCGGGAAGAUGCAGCA | UGCUGCAUCUUCCCGCUCA | 1013 |
| 352 | 464 | GGAAGAUGCAGCAGCGUGA | UCACGCUGCUGCAUCUUCC | 1014 |
| 353 | 465 | GAAGAUGCAGCAGCGUGAU | AUCACGCUGCUGCAUCUUC | 1015 |
| 354 | 466 | AAGAUGCAGCAGCGUGAUU | AAUCACGCUGCUGCAUCUU | 1016 |
| 355 | 467 | AGAUGCAGCAGCGUGAUUG | CAAUCACGCUGCUGCAUCU | 1017 |
| 356 | 468 | GAUGCAGCAGCGUGAUUGG | CCAAUCACGCUGCUGCAUC | 1018 |
| 357 | 469 | AUGCAGCAGCGUGAUUGGA | UCCAAUCACGCUGCUGCAU | 1019 |
| 358 | 470 | UGCAGCAGCGUGAUUGGAA | UUCCAAUCACGCUGCUGCA | 1020 |
| 359 | 471 | GCAGCAGCGUGAUUGGAAG | CUUCCAAUCACGCUGCUGC | 1021 |
| 360 | 472 | CAGCAGCGUGAUUGGAAGA | UCUUCCAAUCACGCUGCUG | 1022 |
| 361 | 473 | AGCAGCGUGAUUGGAAGAC | GUCUUCCAAUCACGCUGCU | 1023 |
| 362 | 474 | GCAGCGUGAUUGGAAGACA | UGUCUUCCAAUCACGCUGC | 1024 |
| 363 | 475 | CAGCGUGAUUGGAAGACAG | CUGUCUUCCAAUCACGCUG | 1025 |
| 364 | 476 | AGCGUGAUUGGAAGACAGC | GCUGUCUUCCAAUCACGCU | 1026 |
| 365 | 477 | GCGUGAUUGGAAGACAGCU | AGCUGUCUUCCAAUCACGC | 1027 |
| 366 | 478 | CGUGAUUGGAAGACAGCUG | CAGCUGUCUUCCAAUCACG | 1028 |
| 367 | 479 | GUGAUUGGAAGACAGCUGU | ACAGCUGUCUUCCAAUCAC | 1029 |
| 368 | 480 | UGAUUGGAAGACAGCUGUA | UACAGCUGUCUUCCAAUCA | 1030 |
| 369 | 481 | GAUUGGAAGACAGCUGUAC | GUACAGCUGUCUUCCAAUC | 1031 |
| 370 | 482 | AUUGGAAGACAGCUGUACA | UGUACAGCUGUCUUCCAAU | 1032 |
| 371 | 483 | UUGGAAGACAGCUGUACAC | GUGUACAGCUGUCUUCCAA | 1033 |
| 372 | 484 | UGGAAGACAGCUGUACACG | CGUGUACAGCUGUCUUCCA | 1034 |
| 373 | 485 | GGAAGACAGCUGUACACGU | ACGUGUACAGCUGUCUUCC | 1035 |
| 374 | 486 | GAAGACAGCUGUACACGUC | GACGUGUACAGCUGUCUUC | 1036 |
| 375 | 487 | AAGACAGCUGUACACGUCA | UGACGUGUACAGCUGUCUU | 1037 |
| 376 | 488 | AGACAGCUGUACACGUCAU | AUGACGUGUACAGCUGUCU | 1038 |
| 377 | 489 | GACAGCUGUACACGUCAUA | UAUGACGUGUACAGCUGUC | 1039 |
| 378 | 490 | ACAGCUGUACACGUCAUAG | CUAUGACGUGUACAGCUGU | 1040 |
| 379 | 491 | CAGCUGUACACGUCAUAGU | ACUAUGACGUGUACAGCUG | 1041 |
| 380 | 492 | AGCUGUACACGUCAUAGUG | CACUAUGACGUGUACAGCU | 1042 |
| 381 | 493 | GCUGUACACGUCAUAGUGG | CCACUAUGACGUGUACAGC | 1043 |
| 382 | 494 | CUGUACACGUCAUAGUGGG | CCCACUAUGACGUGUACAG | 1044 |
| 383 | 495 | UGUACACGUCAUAGUGGGC | GCCCACUAUGACGUGUACA | 1045 |
| 384 | 496 | GUACACGUCAUAGUGGGCC | GGCCCACUAUGACGUGUAC | 1046 |
| 385 | 497 | UACACGUCAUAGUGGGCCC | GGGCCCACUAUGACGUGUA | 1047 |
| 386 | 520 | GGUUGUGGGAAGAGCCAGU | ACUGGCUCUUCCCACAACC | 1048 |
| 387 | 521 | GUUGUGGGAAGAGCCAGUG | CACUGGCUCUUCCCACAAC | 1049 |
| 388 | 522 | UUGUGGGAAGAGCCAGUGG | CCACUGGCUCUUCCCACAA | 1050 |
| 389 | 532 | AGCCAGUGGGCCCGUAAUU | AAUUACGGGCCCACUGGCU | 1051 |
| 390 | 533 | GCCAGUGGGCCCGUAAUUU | AAAUUACGGGCCCACUGGC | 1052 |
| 391 | 534 | CCAGUGGGCCCGUAAUUUU | AAAAUUACGGGCCCACUGG | 1053 |
| 392 | 535 | CAGUGGGCCCGUAAUUUUG | CAAAAUUACGGGCCCACUG | 1054 |
| 393 | 536 | AGUGGGCCCGUAAUUUUGC | GCAAAAUUACGGGCCCACU | 1055 |
| 394 | 537 | GUGGGCCCGUAAUUUUGCU | AGCAAAAUUACGGGCCCAC | 1056 |
| 395 | 538 | UGGGCCCGUAAUUUUGCUG | CAGCAAAAUUACGGGCCCA | 1057 |
| 396 | 539 | GGGCCCGUAAUUUUGCUGA | UCAGCAAAAUUACGGGCCC | 1058 |
| 397 | 540 | GGCCCGUAAUUUUGCUGAG | CUCAGCAAAAUUACGGGCC | 1059 |
| 398 | 541 | GCCCGUAAUUUUGCUGAGC | GCUCAGCAAAAUUACGGGC | 1060 |
| 399 | 542 | CCCGUAAUUUUGCUGAGCC | GGCUCAGCAAAAUUACGGG | 1061 |
| 400 | 543 | CCGUAAUUUUGCUGAGCCU | AGGCUCAGCAAAAUUACGG | 1062 |
| 401 | 544 | CGUAAUUUUGCUGAGCCUA | UAGGCUCAGCAAAAUUACG | 1063 |
| 402 | 545 | GUAAUUUUGCUGAGCCUAG | CUAGGCUCAGCAAAAUUAC | 1064 |
| 403 | 546 | UAAUUUUGCUGAGCCUAGC | GCUAGGCUCAGCAAAAUUA | 1065 |
| 404 | 547 | AAUUUUGCUGAGCCUAGCG | CGCUAGGCUCAGCAAAAUU | 1066 |
| 405 | 548 | AUUUUGCUGAGCCUAGCGA | UCGCUAGGCUCAGCAAAAU | 1067 |
| 406 | 549 | UUUUGCUGAGCCUAGCGAC | GUCGCUAGGCUCAGCAAAA | 1068 |
| 407 | 550 | UUUGCUGAGCCUAGCGACA | UGUCGCUAGGCUCAGCAAA | 1069 |
| 408 | 551 | UUGCUGAGCCUAGCGACAC | GUGUCGCUAGGCUCAGCAA | 1070 |
| 409 | 554 | CUGAGCCUAGCGACACCUA | UAGGUGUCGCUAGGCUCAG | 1071 |
| 410 | 555 | UGAGCCUAGCGACACCUAC | GUAGGUGUCGCUAGGCUCA | 1072 |
| 411 | 556 | GAGCCUAGCGACACCUACU | AGUAGGUGUCGCUAGGCUC | 1073 |
| 412 | 557 | AGCCUAGCGACACCUACUG | CAGUAGGUGUCGCUAGGCU | 1074 |
| 413 | 559 | CCUAGCGACACCUACUGGA | UCCAGUAGGUGUCGCUAGG | 1075 |
| 414 | 560 | CUAGCGACACCUACUGGAA | UUCCAGUAGGUGUCGCUAG | 1076 |
| 415 | 561 | UAGCGACACCUACUGGAAG | CUUCCAGUAGGUGUCGCUA | 1077 |
| 416 | 562 | AGCGACACCUACUGGAAGC | GCUUCCAGUAGGUGUCGCU | 1078 |
| 417 | 564 | CGACACCUACUGGAAGCCU | AGGCUUCCAGUAGGUGUCG | 1079 |
| 418 | 565 | GACACCUACUGGAAGCCUA | UAGGCUUCCAGUAGGUGUC | 1080 |
| 419 | 566 | ACACCUACUGGAAGCCUAG | CUAGGCUUCCAGUAGGUGU | 1081 |
| 420 | 567 | CACCUACUGGAAGCCUAGU | ACUAGGCUUCCAGUAGGUG | 1082 |
| 421 | 568 | ACCUACUGGAAGCCUAGUA | UACUAGGCUUCCAGUAGGU | 1083 |
| 422 | 569 | CCUACUGGAAGCCUAGUAG | CUACUAGGCUUCCAGUAGG | 1084 |
| 423 | 570 | CUACUGGAAGCCUAGUAGA | UCUACUAGGCUUCCAGUAG | 1085 |
| 424 | 571 | UACUGGAAGCCUAGUAGAA | UUCUACUAGGCUUCCAGUA | 1086 |
| 425 | 572 | ACUGGAAGCCUAGUAGAAA | UUUCUACUAGGCUUCCAGU | 1087 |
| 426 | 573 | CUGGAAGCCUAGUAGAAAU | AUUUCUACUAGGCUUCCAG | 1088 |
| 427 | 574 | UGGAAGCCUAGUAGAAAUA | UAUUUCUACUAGGCUUCCA | 1089 |
| 428 | 575 | GGAAGCCUAGUAGAAAUAA | UUAUUUCUACUAGGCUUCC | 1090 |
| 429 | 576 | GAAGCCUAGUAGAAAUAAG | CUUAUUUCUACUAGGCUUC | 1091 |
| 430 | 577 | AAGCCUAGUAGAAAUAAGU | ACUUAUUUCUACUAGGCUU | 1092 |
| 431 | 578 | AGCCUAGUAGAAAUAAGUG | CACUUAUUUCUACUAGGCU | 1093 |
| 432 | 579 | GCCUAGUAGAAAUAAGUGG | CCACUUAUUUCUACUAGGC | 1094 |
| 433 | 580 | CCUAGUAGAAAUAAGUGGU | ACCACUUAUUUCUACUAGG | 1095 |
| 434 | 581 | CUAGUAGAAAUAAGUGGUG | CACCACUUAUUUCUACUAG | 1096 |
| 435 | 582 | UAGUAGAAAUAAGUGGUGG | CCACCACUUAUUUCUACUA | 1097 |
| 436 | 583 | AGUAGAAAUAAGUGGUGGG | CCCACCACUUAUUUCUACU | 1098 |
| 437 | 584 | GUAGAAAUAAGUGGUGGGA | UCCCACCACUUAUUUCUAC | 1099 |
| 438 | 585 | UAGAAAUAAGUGGUGGGAU | AUCCCACCACUUAUUUCUA | 1100 |
| 439 | 586 | AGAAAUAAGUGGUGGGAUG | CAUCCCACCACUUAUUUCU | 1101 |
| 440 | 587 | GAAAUAAGUGGUGGGAUGG | CCAUCCCACCACUUAUUUC | 1102 |
| 441 | 588 | AAAUAAGUGGUGGGAUGGA | UCCAUCCCACCACUUAUUU | 1103 |
| 442 | 589 | AAUAAGUGGUGGGAUGGAU | AUCCAUCCCACCACUUAUU | 1104 |
| 443 | 590 | AUAAGUGGUGGGAUGGAUA | UAUCCAUCCCACCACUUAU | 1105 |
| 444 | 591 | UAAGUGGUGGGAUGGAUAU | AUAUCCAUCCCACCACUUA | 1106 |
| 445 | 592 | AAGUGGUGGGAUGGAUAUC | GAUAUCCAUCCCACCACUU | 1107 |
| 446 | 593 | AGUGGUGGGAUGGAUAUCA | UGAUAUCCAUCCCACCACU | 1108 |
| 447 | 594 | GUGGUGGGAUGGAUAUCAU | AUGAUAUCCAUCCCACCAC | 1109 |
| 448 | 595 | UGGUGGGAUGGAUAUCAUG | CAUGAUAUCCAUCCCACCA | 1110 |
| 449 | 596 | GGUGGGAUGGAUAUCAUGG | CCAUGAUAUCCAUCCCACC | 1111 |
| 450 | 597 | GUGGGAUGGAUAUCAUGGA | UCCAUGAUAUCCAUCCCAC | 1112 |
| 451 | 598 | UGGGAUGGAUAUCAUGGAG | CUCCAUGAUAUCCAUCCCA | 1113 |
| 452 | 599 | GGGAUGGAUAUCAUGGAGA | UCUCCAUGAUAUCCAUCCC | 1114 |
| 453 | 600 | GGAUGGAUAUCAUGGAGAA | UUCUCCAUGAUAUCCAUCC | 1115 |
| 454 | 601 | GAUGGAUAUCAUGGAGAAG | CUUCUCCAUGAUAUCCAUC | 1116 |
| 455 | 602 | AUGGAUAUCAUGGAGAAGA | UCUUCUCCAUGAUAUCCAU | 1117 |
| 456 | 603 | UGGAUAUCAUGGAGAAGAA | UUCUUCUCCAUGAUAUCCA | 1118 |
| 457 | 604 | GGAUAUCAUGGAGAAGAAG | CUUCUUCUCCAUGAUAUCC | 1119 |
| 458 | 605 | GAUAUCAUGGAGAAGAAGU | ACUUCUUCUCCAUGAUAUC | 1120 |
| 459 | 606 | AUAUCAUGGAGAAGAAGUU | AACUUCUUCUCCAUGAUAU | 1121 |
| 460 | 607 | UAUCAUGGAGAAGAAGUUG | CAACUUCUUCUCCAUGAUA | 1122 |
| 461 | 608 | AUCAUGGAGAAGAAGUUGU | ACAACUUCUUCUCCAUGAU | 1123 |
| 462 | 609 | UCAUGGAGAAGAAGUUGUU | AACAACUUCUUCUCCAUGA | 1124 |
| 463 | 610 | CAUGGAGAAGAAGUUGUUG | CAACAACUUCUUCUCCAUG | 1125 |
| 464 | 611 | AUGGAGAAGAAGUUGUUGU | ACAACAACUUCUUCUCCAU | 1126 |
| 465 | 612 | UGGAGAAGAAGUUGUUGUU | AACAACAACUUCUUCUCCA | 1127 |
| 466 | 613 | GGAGAAGAAGUUGUUGUUU | AAACAACAACUUCUUCUCC | 1128 |
| 467 | 614 | GAGAAGAAGUUGUUGUUUU | AAAACAACAACUUCUUCUC | 1129 |
| 468 | 615 | AGAAGAAGUUGUUGUUUUG | CAAAACAACAACUUCUUCU | 1130 |
| 469 | 616 | GAAGAAGUUGUUGUUUUGG | CCAAAACAACAACUUCUUC | 1131 |
| 470 | 617 | AAGAAGUUGUUGUUUUGGA | UCCAAAACAACAACUUCUU | 1132 |
| 471 | 618 | AGAAGUUGUUGUUUUGGAU | AUCCAAAACAACAACUUCU | 1133 |
| 472 | 619 | GAAGUUGUUGUUUUGGAUG | CAUCCAAAACAACAACUUC | 1134 |
| 473 | 620 | AAGUUGUUGUUUUGGAUGA | UCAUCCAAAACAACAACUU | 1135 |
| 474 | 621 | AGUUGUUGUUUUGGAUGAU | AUCAUCCAAAACAACAACU | 1136 |
| 475 | 622 | GUUGUUGUUUUGGAUGAUU | AAUCAUCCAAAACAACAAC | 1137 |
| 476 | 623 | UUGUUGUUUUGGAUGAUUU | AAAUCAUCCAAAACAACAA | 1138 |
| 477 | 640 | UUUUAUGGCUGGUUACCUU | AAGGUAACCAGCCAUAAAA | 1139 |
| 478 | 641 | UUUAUGGCUGGUUACCUUG | CAAGGUAACCAGCCAUAAA | 1140 |
| 479 | 642 | UUAUGGCUGGUUACCUUGG | CCAAGGUAACCAGCCAUAA | 1141 |
| 480 | 643 | UAUGGCUGGUUACCUUGGG | CCCAAGGUAACCAGCCAUA | 1142 |
| 481 | 644 | AUGGCUGGUUACCUUGGGA | UCCCAAGGUAACCAGCCAU | 1143 |
| 482 | 645 | UGGCUGGUUACCUUGGGAU | AUCCCAAGGUAACCAGCCA | 1144 |
| 483 | 646 | GGCUGGUUACCUUGGGAUG | CAUCCCAAGGUAACCAGCC | 1145 |
| 484 | 647 | GCUGGUUACCUUGGGAUGA | UCAUCCCAAGGUAACCAGC | 1146 |
| 485 | 648 | CUGGUUACCUUGGGAUGAU | AUCAUCCCAAGGUAACCAG | 1147 |
| 486 | 649 | UGGUUACCUUGGGAUGAUC | GAUCAUCCCAAGGUAACCA | 1148 |
| 487 | 650 | GGUUACCUUGGGAUGAUCU | AGAUCAUCCCAAGGUAACC | 1149 |
| 488 | 651 | GUUACCUUGGGAUGAUCUA | UAGAUCAUCCCAAGGUAAC | 1150 |
| 489 | 652 | UUACCUUGGGAUGAUCUAC | GUAGAUCAUCCCAAGGUAA | 1151 |
| 490 | 653 | UACCUUGGGAUGAUCUACU | AGUAGAUCAUCCCAAGGUA | 1152 |
| 491 | 654 | ACCUUGGGAUGAUCUACUG | CAGUAGAUCAUCCCAAGGU | 1153 |
| 492 | 655 | CCUUGGGAUGAUCUACUGA | UCAGUAGAUCAUCCCAAGG | 1154 |
| 493 | 656 | CUUGGGAUGAUCUACUGAG | CUCAGUAGAUCAUCCCAAG | 1155 |
| 494 | 657 | UUGGGAUGAUCUACUGAGA | UCUCAGUAGAUCAUCCCAA | 1156 |
| 495 | 658 | UGGGAUGAUCUACUGAGAC | GUCUCAGUAGAUCAUCCCA | 1157 |
| 496 | 659 | GGGAUGAUCUACUGAGACU | AGUCUCAGUAGAUCAUCCC | 1158 |
| 497 | 660 | GGAUGAUCUACUGAGACUG | CAGUCUCAGUAGAUCAUCC | 1159 |
| 498 | 661 | GAUGAUCUACUGAGACUGU | ACAGUCUCAGUAGAUCAUC | 1160 |
| 499 | 662 | AUGAUCUACUGAGACUGUG | CACAGUCUCAGUAGAUCAU | 1161 |
| 500 | 663 | UGAUCUACUGAGACUGUGU | ACACAGUCUCAGUAGAUCA | 1162 |
| 501 | 664 | GAUCUACUGAGACUGUGUG | CACACAGUCUCAGUAGAUC | 1163 |
| 502 | 665 | AUCUACUGAGACUGUGUGA | UCACACAGUCUCAGUAGAU | 1164 |
| 503 | 666 | UCUACUGAGACUGUGUGAC | GUCACACAGUCUCAGUAGA | 1165 |
| 504 | 667 | CUACUGAGACUGUGUGACC | GGUCACACAGUCUCAGUAG | 1166 |
| 505 | 668 | UACUGAGACUGUGUGACCG | CGGUCACACAGUCUCAGUA | 1167 |
| 506 | 669 | ACUGAGACUGUGUGACCGG | CCGGUCACACAGUCUCAGU | 1168 |
| 507 | 670 | CUGAGACUGUGUGACCGGU | ACCGGUCACACAGUCUCAG | 1169 |
| 508 | 671 | UGAGACUGUGUGACCGGUA | UACCGGUCACACAGUCUCA | 1170 |
| 509 | 672 | GAGACUGUGUGACCGGUAU | AUACCGGUCACACAGUCUC | 1171 |
| 510 | 673 | AGACUGUGUGACCGGUAUC | GAUACCGGUCACACAGUCU | 1172 |
| 511 | 674 | GACUGUGUGACCGGUAUCC | GGAUACCGGUCACACAGUC | 1173 |
| 512 | 675 | ACUGUGUGACCGGUAUCCA | UGGAUACCGGUCACACAGU | 1174 |
| 513 | 676 | CUGUGUGACCGGUAUCCAU | AUGGAUACCGGUCACACAG | 1175 |
| 514 | 677 | UGUGUGACCGGUAUCCAUU | AAUGGAUACCGGUCACACA | 1176 |
| 515 | 678 | GUGUGACCGGUAUCCAUUG | CAAUGGAUACCGGUCACAC | 1177 |
| 516 | 679 | UGUGACCGGUAUCCAUUGA | UCAAUGGAUACCGGUCACA | 1178 |
| 517 | 680 | GUGACCGGUAUCCAUUGAC | GUCAAUGGAUACCGGUCAC | 1179 |
| 518 | 681 | UGACCGGUAUCCAUUGACU | AGUCAAUGGAUACCGGUCA | 1180 |
| 519 | 682 | GACCGGUAUCCAUUGACUG | CAGUCAAUGGAUACCGGUC | 1181 |
| 520 | 683 | ACCGGUAUCCAUUGACUGU | ACAGUCAAUGGAUACCGGU | 1182 |
| 521 | 684 | CCGGUAUCCAUUGACUGUA | UACAGUCAAUGGAUACCGG | 1183 |
| 522 | 685 | CGGUAUCCAUUGACUGUAG | CUACAGUCAAUGGAUACCG | 1184 |
| 523 | 686 | GGUAUCCAUUGACUGUAGA | UCUACAGUCAAUGGAUACC | 1185 |
| 524 | 687 | GUAUCCAUUGACUGUAGAG | CUCUACAGUCAAUGGAUAC | 1186 |
| 525 | 688 | UAUCCAUUGACUGUAGAGA | UCUCUACAGUCAAUGGAUA | 1187 |
| 526 | 689 | AUCCAUUGACUGUAGAGAC | GUCUCUACAGUCAAUGGAU | 1188 |
| 527 | 690 | UCCAUUGACUGUAGAGACU | AGUCUCUACAGUCAAUGGA | 1189 |
| 528 | 691 | CCAUUGACUGUAGAGACUA | UAGUCUCUACAGUCAAUGG | 1190 |
| 529 | 692 | CAUUGACUGUAGAGACUAA | UUAGUCUCUACAGUCAAUG | 1191 |
| 530 | 693 | AUUGACUGUAGAGACUAAA | UUUAGUCUCUACAGUCAAU | 1192 |
| 531 | 694 | UUGACUGUAGAGACUAAAG | CUUUAGUCUCUACAGUCAA | 1193 |
| 532 | 695 | UGACUGUAGAGACUAAAGG | CCUUUAGUCUCUACAGUCA | 1194 |
| 533 | 696 | GACUGUAGAGACUAAAGGG | CCCUUUAGUCUCUACAGUC | 1195 |
| 534 | 697 | ACUGUAGAGACUAAAGGGG | CCCCUUUAGUCUCUACAGU | 1196 |
| 535 | 729 | UUUGGCCCGCAGUAUUUUG | CAAAAUACUGCGGGCCAAA | 1197 |
| 536 | 730 | UUGGCCCGCAGUAUUUUGA | UCAAAAUACUGCGGGCCAA | 1198 |
| 537 | 731 | UGGCCCGCAGUAUUUUGAU | AUCAAAAUACUGCGGGCCA | 1199 |
| 538 | 732 | GGCCCGCAGUAUUUUGAUU | AAUCAAAAUACUGCGGGCC | 1200 |
| 539 | 733 | GCCCGCAGUAUUUUGAUUA | UAAUCAAAAUACUGCGGGC | 1201 |
| 540 | 734 | CCCGCAGUAUUUUGAUUAC | GUAAUCAAAAUACUGCGGG | 1202 |
| 541 | 735 | CCGCAGUAUUUUGAUUACC | GGUAAUCAAAAUACUGCGG | 1203 |
| 542 | 736 | CGCAGUAUUUUGAUUACCA | UGGUAAUCAAAAUACUGCG | 1204 |
| 543 | 737 | GCAGUAUUUUGAUUACCAG | CUGGUAAUCAAAAUACUGC | 1205 |
| 544 | 738 | CAGUAUUUUGAUUACCAGC | GCUGGUAAUCAAAAUACUG | 1206 |
| 545 | 739 | AGUAUUUUGAUUACCAGCA | UGCUGGUAAUCAAAAUACU | 1207 |
| 546 | 740 | GUAUUUUGAUUACCAGCAA | UUGCUGGUAAUCAAAAUAC | 1208 |
| 547 | 741 | UAUUUUGAUUACCAGCAAU | AUUGCUGGUAAUCAAAAUA | 1209 |
| 548 | 742 | AUUUUGAUUACCAGCAAUC | GAUUGCUGGUAAUCAAAAU | 1210 |
| 549 | 743 | UUUUGAUUACCAGCAAUCA | UGAUUGCUGGUAAUCAAAA | 1211 |
| 550 | 744 | UUUGAUUACCAGCAAUCAG | CUGAUUGCUGGUAAUCAAA | 1212 |
| 551 | 745 | UUGAUUACCAGCAAUCAGG | CCUGAUUGCUGGUAAUCAA | 1213 |
| 552 | 746 | UGAUUACCAGCAAUCAGGC | GCCUGAUUGCUGGUAAUCA | 1214 |
| 553 | 747 | GAUUACCAGCAAUCAGGCC | GGCCUGAUUGCUGGUAAUC | 1215 |
| 554 | 748 | AUUACCAGCAAUCAGGCCC | GGGCCUGAUUGCUGGUAAU | 1216 |
| 555 | 749 | UUACCAGCAAUCAGGCCCC | GGGGCCUGAUUGCUGGUAA | 1217 |
| 556 | 766 | CCCCAGGAAUGGUACUCCU | AGGAGUACCAUUCCUGGGG | 1218 |
| 557 | 767 | CCCAGGAAUGGUACUCCUC | GAGGAGUACCAUUCCUGGG | 1219 |
| 558 | 768 | CCAGGAAUGGUACUCCUCA | UGAGGAGUACCAUUCCUGG | 1220 |
| 559 | 769 | CAGGAAUGGUACUCCUCAA | UUGAGGAGUACCAUUCCUG | 1221 |
| 560 | 770 | AGGAAUGGUACUCCUCAAC | GUUGAGGAGUACCAUUCCU | 1222 |
| 561 | 771 | GGAAUGGUACUCCUCAACU | AGUUGAGGAGUACCAUUCC | 1223 |
| 562 | 772 | GAAUGGUACUCCUCAACUG | CAGUUGAGGAGUACCAUUC | 1224 |
| 563 | 773 | AAUGGUACUCCUCAACUGC | GCAGUUGAGGAGUACCAUU | 1225 |
| 564 | 774 | AUGGUACUCCUCAACUGCU | AGCAGUUGAGGAGUACCAU | 1226 |
| 565 | 775 | UGGUACUCCUCAACUGCUG | CAGCAGUUGAGGAGUACCA | 1227 |
| 566 | 776 | GGUACUCCUCAACUGCUGU | ACAGCAGUUGAGGAGUACC | 1228 |
| 567 | 777 | GUACUCCUCAACUGCUGUC | GACAGCAGUUGAGGAGUAC | 1229 |
| 568 | 778 | UACUCCUCAACUGCUGUCC | GGACAGCAGUUGAGGAGUA | 1230 |
| 569 | 779 | ACUCCUCAACUGCUGUCCC | GGGACAGCAGUUGAGGAGU | 1231 |
| 570 | 780 | CUCCUCAACUGCUGUCCCA | UGGGACAGCAGUUGAGGAG | 1232 |
| 571 | 781 | UCCUCAACUGCUGUCCCAG | CUGGGACAGCAGUUGAGGA | 1233 |
| 572 | 783 | CUCAACUGCUGUCCCAGCU | AGCUGGGACAGCAGUUGAG | 1234 |
| 573 | 784 | UCAACUGCUGUCCCAGCUG | CAGCUGGGACAGCAGUUGA | 1235 |
| 574 | 785 | CAACUGCUGUCCCAGCUGU | ACAGCUGGGACAGCAGUUG | 1236 |
| 575 | 786 | AACUGCUGUCCCAGCUGUA | UACAGCUGGGACAGCAGUU | 1237 |
| 576 | 787 | ACUGCUGUCCCAGCUGUAG | CUACAGCUGGGACAGCAGU | 1238 |
| 577 | 788 | CUGCUGUCCCAGCUGUAGA | UCUACAGCUGGGACAGCAG | 1239 |
| 578 | 789 | UGCUGUCCCAGCUGUAGAA | UUCUACAGCUGGGACAGCA | 1240 |
| 579 | 790 | GCUGUCCCAGCUGUAGAAG | CUUCUACAGCUGGGACAGC | 1241 |
| 580 | 791 | CUGUCCCAGCUGUAGAAGC | GCUUCUACAGCUGGGACAG | 1242 |
| 581 | 792 | UGUCCCAGCUGUAGAAGCU | AGCUUCUACAGCUGGGACA | 1243 |
| 582 | 793 | GUCCCAGCUGUAGAAGCUC | GAGCUUCUACAGCUGGGAC | 1244 |
| 583 | 794 | UCCCAGCUGUAGAAGCUCU | AGAGCUUCUACAGCUGGGA | 1245 |
| 584 | 795 | CCCAGCUGUAGAAGCUCUC | GAGAGCUUCUACAGCUGGG | 1246 |
| 585 | 796 | CCAGCUGUAGAAGCUCUCU | AGAGAGCUUCUACAGCUGG | 1247 |
| 586 | 797 | CAGCUGUAGAAGCUCUCUA | UAGAGAGCUUCUACAGCUG | 1248 |
| 587 | 798 | AGCUGUAGAAGCUCUCUAU | AUAGAGAGCUUCUACAGCU | 1249 |
| 588 | 799 | GCUGUAGAAGCUCUCUAUC | GAUAGAGAGCUUCUACAGC | 1250 |
| 589 | 800 | CUGUAGAAGCUCUCUAUCG | CGAUAGAGAGCUUCUACAG | 1251 |
| 590 | 801 | UGUAGAAGCUCUCUAUCGG | CCGAUAGAGAGCUUCUACA | 1252 |
| 591 | 802 | GUAGAAGCUCUCUAUCGGA | UCCGAUAGAGAGCUUCUAC | 1253 |
| 592 | 803 | UAGAAGCUCUCUAUCGGAG | CUCCGAUAGAGAGCUUCUA | 1254 |
| 593 | 804 | AGAAGCUCUCUAUCGGAGG | CCUCCGAUAGAGAGCUUCU | 1255 |
| 594 | 805 | GAAGCUCUCUAUCGGAGGA | UCCUCCGAUAGAGAGCUUC | 1256 |
| 595 | 806 | AAGCUCUCUAUCGGAGGAU | AUCCUCCGAUAGAGAGCUU | 1257 |
| 596 | 807 | AGCUCUCUAUCGGAGGAUU | AAUCCUCCGAUAGAGAGCU | 1258 |
| 597 | 808 | GCUCUCUAUCGGAGGAUUA | UAAUCCUCCGAUAGAGAGC | 1259 |
| 598 | 809 | CUCUCUAUCGGAGGAUUAC | GUAAUCCUCCGAUAGAGAG | 1260 |
| 599 | 810 | UCUCUAUCGGAGGAUUACU | AGUAAUCCUCCGAUAGAGA | 1261 |
| 600 | 811 | CUCUAUCGGAGGAUUACUA | UAGUAAUCCUCCGAUAGAG | 1262 |
| 601 | 812 | UCUAUCGGAGGAUUACUAC | GUAGUAAUCCUCCGAUAGA | 1263 |
| 602 | 813 | CUAUCGGAGGAUUACUACU | AGUAGUAAUCCUCCGAUAG | 1264 |
| 603 | 814 | UAUCGGAGGAUUACUACUU | AAGUAGUAAUCCUCCGAUA | 1265 |
| 604 | 815 | AUCGGAGGAUUACUACUUU | AAAGUAGUAAUCCUCCGAU | 1266 |
| 605 | 816 | UCGGAGGAUUACUACUUUG | CAAAGUAGUAAUCCUCCGA | 1267 |
| 606 | 817 | CGGAGGAUUACUACUUUGC | GCAAAGUAGUAAUCCUCCG | 1268 |
| 607 | 818 | GGAGGAUUACUACUUUGCA | UGCAAAGUAGUAAUCCUCC | 1269 |
| 608 | 819 | GAGGAUUACUACUUUGCAA | UUGCAAAGUAGUAAUCCUC | 1270 |
| 609 | 820 | AGGAUUACUACUUUGCAAU | AUUGCAAAGUAGUAAUCCU | 1271 |
| 610 | 821 | GGAUUACUACUUUGCAAUU | AAUUGCAAAGUAGUAAUCC | 1272 |
| 611 | 822 | GAUUACUACUUUGCAAUUU | AAAUUGCAAAGUAGUAAUC | 1273 |
| 612 | 823 | AUUACUACUUUGCAAUUUU | AAAAUUGCAAAGUAGUAAU | 1274 |
| 613 | 824 | UUACUACUUUGCAAUUUUG | CAAAAUUGCAAAGUAGUAA | 1275 |
| 614 | 825 | UACUACUUUGCAAUUUUGG | CCAAAAUUGCAAAGUAGUA | 1276 |
| 615 | 826 | ACUACUUUGCAAUUUUGGA | UCCAAAAUUGCAAAGUAGU | 1277 |
| 616 | 827 | CUACUUUGCAAUUUUGGAA | UUCCAAAAUUGCAAAGUAG | 1278 |
| 617 | 828 | UACUUUGCAAUUUUGGAAG | CUUCCAAAAUUGCAAAGUA | 1279 |
| 618 | 829 | ACUUUGCAAUUUUGGAAGA | UCUUCCAAAAUUGCAAAGU | 1280 |
| 619 | 830 | CUUUGCAAUUUUGGAAGAC | GUCUUCCAAAAUUGCAAAG | 1281 |
| 620 | 831 | UUUGCAAUUUUGGAAGACU | AGUCUUCCAAAAUUGCAAA | 1282 |
| 621 | 832 | UUGCAAUUUUGGAAGACUG | CAGUCUUCCAAAAUUGCAA | 1283 |
| 622 | 833 | UGCAAUUUUGGAAGACUGC | GCAGUCUUCCAAAAUUGCA | 1284 |
| 623 | 834 | GCAAUUUUGGAAGACUGCU | AGCAGUCUUCCAAAAUUGC | 1285 |
| 624 | 835 | CAAUUUUGGAAGACUGCUG | CAGCAGUCUUCCAAAAUUG | 1286 |
| 625 | 836 | AAUUUUGGAAGACUGCUGG | CCAGCAGUCUUCCAAAAUU | 1287 |
| 626 | 837 | AUUUUGGAAGACUGCUGGA | UCCAGCAGUCUUCCAAAAU | 1288 |
| 627 | 838 | UUUUGGAAGACUGCUGGAG | CUCCAGCAGUCUUCCAAAA | 1289 |
| 628 | 839 | UUUGGAAGACUGCUGGAGA | UCUCCAGCAGUCUUCCAAA | 1290 |
| 629 | 840 | UUGGAAGACUGCUGGAGAA | UUCUCCAGCAGUCUUCCAA | 1291 |
| 630 | 841 | UGGAAGACUGCUGGAGAAC | GUUCUCCAGCAGUCUUCCA | 1292 |
| 631 | 842 | GGAAGACUGCUGGAGAACA | UGUUCUCCAGCAGUCUUCC | 1293 |
| 632 | 843 | GAAGACUGCUGGAGAACAA | UUGUUCUCCAGCAGUCUUC | 1294 |
| 633 | 844 | AAGACUGCUGGAGAACAAU | AUUGUUCUCCAGCAGUCUU | 1295 |
| 634 | 845 | AGACUGCUGGAGAACAAUC | GAUUGUUCUCCAGCAGUCU | 1296 |
| 635 | 846 | GACUGCUGGAGAACAAUCC | GGAUUGUUCUCCAGCAGUC | 1297 |
| 636 | 847 | ACUGCUGGAGAACAAUCCA | UGGAUUGUUCUCCAGCAGU | 1298 |
| 637 | 848 | CUGCUGGAGAACAAUCCAC | GUGGAUUGUUCUCCAGCAG | 1299 |
| 638 | 849 | UGCUGGAGAACAAUCCACG | CGUGGAUUGUUCUCCAGCA | 1300 |
| 639 | 850 | GCUGGAGAACAAUCCACGG | CCGUGGAUUGUUCUCCAGC | 1301 |
| 640 | 851 | CUGGAGAACAAUCCACGGA | UCCGUGGAUUGUUCUCCAG | 1302 |
| 641 | 852 | UGGAGAACAAUCCACGGAG | CUCCGUGGAUUGUUCUCCA | 1303 |
| 642 | 853 | GGAGAACAAUCCACGGAGG | CCUCCGUGGAUUGUUCUCC | 1304 |
| 643 | 854 | GAGAACAAUCCACGGAGGU | ACCUCCGUGGAUUGUUCUC | 1305 |
| 644 | 855 | AGAACAAUCCACGGAGGUA | UACCUCCGUGGAUUGUUCU | 1306 |
| 645 | 856 | GAACAAUCCACGGAGGUAC | GUACCUCCGUGGAUUGUUC | 1307 |
| 646 | 857 | AACAAUCCACGGAGGUACC | GGUACCUCCGUGGAUUGUU | 1308 |
| 647 | 858 | ACAAUCCACGGAGGUACCC | GGGUACCUCCGUGGAUUGU | 1309 |
| 648 | 860 | AAUCCACGGAGGUACCCGA | UCGGGUACCUCCGUGGAUU | 1310 |
| 649 | 861 | AUCCACGGAGGUACCCGAA | UUCGGGUACCUCCGUGGAU | 1311 |
| 650 | 869 | AGGUACCCGAAGGCCGAUU | AAUCGGCCUUCGGGUACCU | 1312 |
| 651 | 870 | GUACCCGAAGGCCGAUUUG | AAAUCGGCCUUCGGGUACC | 1313 |
| 652 | 871 | UACCCGAAGGCCGAUUUGA | CAAAUCGGCCUUCGGGUAC | 1314 |
| 653 | 872 | ACCCGAAGGCCGAUUUGAA | UCAAAUCGGCCUUCGGGUA | 1315 |
| 654 | 873 | CCCGAAGGCCGAUUUGAAG | UUCAAAUCGGCCUUCGGGU | 1316 |
| 655 | 874 | CCGAAGGCCGAUUUGAAGC | CUUCAAAUCGGCCUUCGGG | 1317 |
| 656 | 875 | CGAAGGCCGAUUUGAAGCA | GCUUCAAAUCGGCCUUCGG | 1318 |
| 657 | 876 | GAAGGCCGAUUUGAAGCAG | UGCUUCAAAUCGGCCUUCG | 1319 |
| 658 | 877 | AAGGCCGAUUUGAAGCAGU | CUGCUUCAAAUCGGCCUUC | 1320 |
| 659 | 878 | AGGCCGAUUUGAAGCAGUG | ACUGCUUCAAAUCGGCCUU | 1321 |
| 660 | 879 | GGCCGAUUUGAAGCAGUGG | CACUGCUUCAAAUCGGCCU | 1322 |
| 661 | 880 | GCCGAUUUGAAGCAGUGGA | CCACUGCUUCAAAUCGGCC | 1323 |
| 662 | 881 | CCGAUUUGAAGCAGUGGAC | UCCACUGCUUCAAAUCGGC | 1324 |
| 663 | 882 | CGAUUUGAAGCAGUGGACC | GUCCACUGCUUCAAAUCGG | 1325 |
| 664 | 883 | GAUUUGAAGCAGUGGACCC | GGUCCACUGCUUCAAAUCG | 1326 |
| 665 | 884 | AUUUGAAGCAGUGGACCCA | GGGUCCACUGCUUCAAAUC | 1327 |
| 666 | 885 | UUUGAAGCAGUGGACCCAC | UGGGUCCACUGCUUCAAAU | 1328 |
| 667 | 886 | UUGAAGCAGUGGACCCACC | GUGGGUCCACUGCUUCAAA | 1329 |
| 668 | 887 | ACCCACCCUGUGCCCUUUU | GGUGGGUCCACUGCUUCAA | 1330 |
| 669 | 899 | ACCCUGUGCCCUUUUCCCA | AAAAGGGCACAGGGUGGGU | 1331 |
| 670 | 903 | CCCUGUGCCCUUUUCCCAU | UGGGAAAAGGGCACAGGGU | 1332 |
| 671 | 904 | CCUGUGCCCUUUUCCCAUA | AUGGGAAAAGGGCACAGGG | 1333 |
| 672 | 905 | CUGUGCCCUUUUCCCAUAU | UAUGGGAAAAGGGCACAGG | 1334 |
| 673 | 906 | UGUGCCCUUUUCCCAUAUA | AUAUGGGAAAAGGGCACAG | 1335 |
| 674 | 907 | GUGCCCUUUUCCCAUAUAA | UAUAUGGGAAAAGGGCACA | 1336 |
| 675 | 908 | UGCCCUUUUCCCAUAUAAA | UUAUAUGGGAAAAGGGCAC | 1337 |
| 676 | 909 | CCCAUAUAAAAUAAAUUAC | UUUAUAUGGGAAAAGGGCA | 1338 |
| 677 | 918 | GUACCCGAAGGCCGAUUUG | GUAAUUUAUUUUAUAUGGG | 1339 |

#indicates start position

### Example 2

### Porcine Circovirus Sequences

In addition to the example dsRNAs targeting the ref gene of Example 1, the complete genomes of several PCV1 are available for consideration. Targets can be selected as in Example 1.

AY193712.1: 47-985 PCV1 strain PCV1, complete
genome:
ATGCCAAGCAAGAAAAGCGGCCCGCAACCCCATAAGAGGTGGGTGTTC
ACCCTTAATAATCCTTCCGAGGAGGAGAAAAACAAAATACGGGAGCTT
CCAATCTCCCTTTTTGATTATTTTGTTTGCGGAGAGGAAGGTTTGGAA
GAGGGTAGAACTCCTCACCTCCAGGGGTTTGCGAATTTTGCTAAGAAG
CAGACTTTTAACAAGGTGAAGTGGTATTTTGGTGCCCGCTGCCACATC
GAGAAAGCGAAAGGAACCGACCAGCAGAATAAAGAATACTGCAGTAAA
GAAGGCCACATACTTATCGAGTGTGGAGCTCCGCGGAACCAGGGGAAG
CGCAGCGACCTGTCTACTGCTGTGAGTACCCTTTTGGAGACGGGGTCT
TTGGTGACTGTAGCCGAGCAGTTCCCTGTAACGTATGTGAGAAATTTC
CGCGGGCTGGCTGAACTTTTGAAAGTGAGCGGGAAGATGCAGCAGCGT
GATTGGAAGACAGCTGTACACGTCATAGTGGGCCCGCCCGGTTGTGGG
AAGAGCCAGTGGGCCCGTAATTTTGCTGAGCCTAGCGACACCTACTGG
AAGCCTAGTAGAAATAAGTGGTGGGATGGATATCATGGAGAAGAAGTT
GTTGTTTTGGATGATTTTTATGGCTGGTTACCTTGGGATGATCTACTG
AGACTGTGTGACCGGTATCCATTGACTGTAGAGACTAAAGGGGGTACT
GTTCCTTTTTTGGCCCGCAGTATTTTGATTACCAGCAATCAGGCCCCC
CAGGAATGGTACTCCTCAACTGCTGTCCCAGCTGTAGAAGCTCTCTAT
CGGAGGATTACTACTTTGCAATTTTGGAAGACTGCTGGAGAACAATCC
ACGGAGGTACCCGAAGGCCGATTTGAAGCAGTGGACCCACCCTGTGCC
CTTTTCCCATATAAAATAAATTACTGA
(SEQ ID NO: 1340)
EF533941.1: 819-1757 PCV1 strain HZ2006, complete
genome
ATGCCAAGCAAGAAAAGCGGCCCGCAACCCCATAAGAGGTGGGTGTTC
ACCCTTAATAATCCTTCCGAGGAGGAGAAAAACAAAATACGGGAGCTT
CCAATCTCCCTTTTTGATTATTTTGTTTGCGGAGAGGAAGGTTTGGAA
GAGGGTAGAACTCCTCACCTCCAGGGGTTTGCGAATTTTGCTAAGAAG
CAGACTTTTAACAAGGTGAAGTGGTATTTTGGTGCCCGCTGCCACATC
GAGAAAGCGAAAGGAACCGACCAGCAGAATAAAGAATACTGCAGTAAA
GAAGGCCACATACTTATCGAGTGTGGAGCTCCGCGGAACCAGGGGAAG
CGCAGCGACCTGTCTACTGCTGTGAGTACCCTTTTGGAGACGGGGTCT
TTGGTGACTGTAGCCGAGCAGTCCCCTGTAACGTATGTGAGAAATTTC
CGCGGGCTGGCTGAACTTTTGAAAGTGAGCGGGAAGATGCAGCAGCGT
GATTGGAAGACAGCTGTACACGTCATAGTGGGCCCGCCCGGTTGTGGG
AAGAGCCAGTGGGCCCGTAATTTTGCTGAGCCTAGCGACACCTACTGG
AAGCCTAGTAGAAATAAGTGGTGGGATGGATATCATGGAGAAGAAGTT
GTTGTTTTGGATGATTTTTATGGCTGGTTACCTTGGGATGATCTACTG
AGACTGTGTGACCGGTATCCATTGACTGTAGAGACTAAAGGGGGTACT
GTTCCTTTTTTGGCCCGCAGTATTTTGATTACCAGCAATCAGGCCCCC
CAGGAATGGTACTCCTCAACTGCTGTCCCAGCTGTAGAAGCTCTCTAT
CGGAGGATTACTACTTTGCAATTTTGGAAGACTGCTGGAGAACAATCC
ACGGAGGTACCCGAAGGCCGATTTGAAGCAGTGGACCCACCCTGTGCC
CTTTTCCCATATAAAATAAATTACTGA
(SEQ ID NO: 1341)
FJ475129.2: 47-985 PCV1 strain BJ-1, complete
genome
ATGCCAAGCAAGAAAAGCGGCCCGCAACCCCATAAGAGGTGGGTGTTC
ACCCTTAATAATCCTTCCGAGGAGGAGAAAAACAAAATACGGGAGCTT
CCAATCTCCCTTTTTGATTATTTTGTTTGCGGAGAGGAAGGTTTGGAA
GAGGGTAGAACTCCTCACCTCCAGGGGTTTGCGAATTTTGCTAAGAAG
CAGACTTTTAACAAGGTGAAGTGGTATTTTGGTGCCCGCTGCCACATC
GAGAAAGCGAAAGGAACCGACCAGCAGAATAAAGAATACTGCAGTAAA
GAAGGCCACATACTTATCGAGTGTGGAGCTCCGCGGAACCAGGGGAAG
CGCAGCGACCTGTCTACTGCTGTGAGTACCCTTTTGGAGACGGGGTCT
TTGGTGACTGTAGCCGAGCAGTTCCCTGTAACGTATGTGAGAAATTTC
CGCGGGCTGGCTGAACTTTTGAAAGTGAGCGGGAAGATGCAGCAGCGT
GATTGGAAGACAGCTGTACACGTCATAGTGGGCCCGCCCGGTTGTGGG
AAGAGCCAGTGGGCCCGTAATTTTGCTGAGCCTAGCGACACCTACTGG
AAGCCTAGTAGAAATAAGTGGTGGGATGGATATCATGGAGAAGAAGTT
GTTGTTTTGGATGATTTTTATGGCTGGTTACCTTGGGATGATCTACTG
AGACTGTGTGACCGGTATCCATTGACTGTAGAGACTAAAGGGGGTACT
GTTCCTTTTTTGGCCCGCAGTATTTTGATTACCAGCAATCAGGCCCCC
CAGGAATGGTACTCCTCAACTGCTGTCCCAGCTGTAGAAGCTCTCTAT
CGGAGGATTACTACTTTGCAATTTTGGAAGACTGCTGGAGAACAATCC
ACGGAGGTACCCGAAGGCCGATTTGAAGCAGTGGACCCACCCTGTGCC
CTTTTCCCATATAAAATAAATTACTGA
(SEQ ID NO: 1342)
GU371908.1: 47-985 PCV1 isolate Tian Jin,
complete genome
ATGCCAAGCAAGAAAAGCGGCCCGCAACCCCATAAGAGGTGGGTGTTC
ACCCTTAATAATCCTTCCGAGGAGGAGAAAAACAAAATACGGGAGCTT
CCAATCTCCCTTTTTGATTATTTTGTTTGCGGAGAGGAAGGTTTGGAA
GAGGGTAGAACTCCTCACCTCCAGGGGTTTGCGAATTTTGCTAAGAAG
CAGACTTTTAACAAGGTGAAGTGGTATTTTGGTGCCCGCTGCCACATC
GAGAAAGCGAAAGGAACCGACCAGCAGAATAAAGAATACTGCAGTAAA
GAAGGCCACATACTTATCGAGTGTGGAGCTCCGCGGAACCAGGGGAAG
CGCAGCGACCTGTCTACTGCTGTGAGTACCCTTTTGGAGACGGGGTCT
TTGGTGACTGTAGCCGAGCAGTTCCCTGTAACGTATGTGAGAAATTTC
CGCGGGTTGGCTGAACTTTTGAAAGTGAGCGGGAAGATGCAGCAGCGT
GATTGGAAGACAGCTGTACACGTCATAGTGGGCCCGCCCGGTTGTGGG
AAGAGCCAGTGGGCCCGTAATTTTGCTGAGCCTAGCGACACCTACTGG
AAGCCTAGTAGAAATAAGTGGTGGGATGGATATCATGGAGAAGAAGTT
GTTGTTTTGGATGATTTTTATGGCTGGTTACCTTGGGATGATCTACTG
AGACTGTGTGACCGGTATCCATTGACTGTAGAGACTAAAGGGGGTACT
GTTCCTTTTTTGGCCCGCAGTATTTTGATTACCAGCAATCAGGCCCCC
CAGGAATGGTACTCCTCAACTGCTGTCCCAGCTGTAGAAGCTCTCTAT
CGGAGGATTACTACTTTGCAATTTTGGAAGACTGCTGGAGAACAATCC
ACGGAGGTACCCGAAGGCCGATTTGAAGCAGTGGACCCACCCTGTGCC
CTTTTCCCATATAAAATAAATTACTGA
(SEQ ID NO: 1343)
GU722334.1: 47-985 PCV1 isolate Jiangsu,
complete genome
ATGCCAAGCAAGAAAAGCGGCCCGCAACCCCATAAGAGGTGGGTGTTC
ACCCTTAATAATCCTTCCGAGGAGGAGAAAAACAAAATACGGGAGCTT
CCAATCTCCCTTTTTGATTATTTTGTTTGCGGAGAGGAAGGTTTGGAA
GAGGGTAGAACTCCTCACCTCCAGGGGTTTGCGAATTTTGCTAAGAAG
CAGACTTTTAACAAGGTGAAGTGGTATTTTGGTGCCCGCTGCCACATC
GAGAAAGCGAAAGGAACCGACCACCGGAATAAAGAATACTGCAGTAAA
GAAGGCCACATACTTATCGAGTGTGGAGCTCCGCGGAACCAGGGGAAG
CGCAGCGACCTGTCTACTGCTGTGAGTACCCTTTTGGAGACGGGGTCT
TTGGTGACTGTAGCCGAGCAGTTCCCTGTAACGTATGTGAGAAATTTC
CGCGGGCTGGCTGAACTTTAGAAAGTGAGCGGGAAGATGCAGCAGCGT
GATTGGAAGACAGCTGTACACGTCATAGTGGGCCCGCCCGGTTGTGGG
AAGAGCCAGTGGGCCCGTAATTTTGCTGAGCCTAGCGACACCTACTGG
AAGCCTAGTAGAAATAAGTGGTGGGATGGATATCATGGAGAAGAAGTT
GTTGTTTTGGATGATTTTTATGGCTGGTTACCTTGGGATGATCTACTA
GAACTGTGTGACCGGTATCCATTGACTGTAGAGACTAAAGGCGGTACT
GTTCCTTTTTTGGCCCGCAGTATTTTGATTACCAGCAATCAGGCCCCC
CAGGAATGGTACTCCTCAACTGCTGTCCCAGCTGTAGAAGCTCTCTAT
CGGAGGATTACTACTTTGCAATTTTGGAAGACTGCTGGAGAACAATCC
ACGGAGGTACCCGAAGGCCGATTTGAAGCAGTGGACCCACCCTGTGCC
CTTTTCCCATATAAAATAAATTACTGA
(SEQ ID NO: 1344)

### Example 3

### Target Gene Vesivirus

TABLE 3
Sense and antisense strand sequences of dsRNA against vesivirus 2117

| SEQ ID | Sense (3′ to 5′) | AntiSense (5′ to 3′) | SEQ ID |
|---|---|---|---|
| 1445 | AUCUGGCUAGCUACUAUUC | GAAUAGUAGCUAGCCAGAU | 1500 |
| 1446 | GCUAGCUACUAUUCUACAG | CUGUAGAAUAGUAGCUAGC | 1501 |
| 1447 | UGCUUUCCGGAUUACUUAG | CUAAGUAAUCCGGAAAGCA | 1502 |
| 1448 | GCUUUCCGGAUUACUUAGA | UCUAAGUAAUCCGGAAAGC | 1503 |
| 1449 | UCCGGAUUACUUAGAUUCC | GGAAUCUAAGUAAUCCGGA | 1504 |
| 1450 | CCGGAUUACUUAGAUUCCC | GGGAAUCUAAGUAAUCCGG | 1505 |
| 1451 | GUAUAUCUACGAAGGCGAC | GUCGCCUUCGUAGAUAUAC | 1506 |
| 1452 | UAUAUCUACGAAGGCGACG | CGUCGCCUUCGUAGAUAUA | 1507 |
| 1453 | AUAUCUACGAAGGCGACGA | UCGUCGCCUUCGUAGAUAU | 1508 |
| 1454 | UAUCUACGAAGGCGACGAA | UUCGUCGCCUUCGUAGAUA | 1509 |
| 1455 | ACGAACCCUACAUCGUUCU | AGAACGAUGUAGGGUUCGU | 1510 |
| 1456 | GAACCCUACAUCGUUCUAA | UUAGAACGAUGUAGGGUUC | 1511 |
| 1457 | AACCCUACAUCGUUCUAAA | UUUAGAACGAUGUAGGGUU | 1512 |
| 1458 | ACCAAUUGGAGUCGUCAAC | GUUGACGACUCCAAUUGGU | 1513 |
| 1459 | CCAAUUGGAGUCGUCAACU | AGUUGACGACUCCAAUUGG | 1514 |
| 1460 | CAAUUGGAGUCGUCAACUU | AAGUUGACGACUCCAAUUG | 1515 |
| 1461 | CACUUUCACUUACCCUAUA | UAUAGGGUAAGUGAAAGUG | 1516 |
| 1462 | ACUUUCACUUACCCUAUAC | GUAUAGGGUAAGUGAAAGU | 1517 |
| 1463 | UACACCACCGUCAAUUGGU | ACCAAUUGACGGUGGUGUA | 1518 |
| 1464 | CUAUGGAGAUUCGCUUCAC | GUGAAGCGAAUCUCCAUAG | 1519 |
| 1465 | GCCGGAGUGAAACUAAAUA | UAUUUAGUUUCACUCCGGC | 1520 |
| 1466 | AUGGUUGGCCCGACUAUUA | UAAUAGUCGGGCCAACCAU | 1521 |
| 1467 | CGACUAUUACGGUCGAAAC | GUUUCGACCGUAAUAGUCG | 1522 |
| 1468 | GACUAUUACGGUCGAAACA | UGUUUCGACCGUAAUAGUC | 1523 |
| 1469 | ACUAUUACGGUCGAAACAU | AUGUUUCGACCGUAAUAGU | 1524 |
| 1470 | CUAUUACGGUCGAAACAUC | GAUGUUUCGACCGUAAUAG | 1525 |
| 1471 | UAUUACGGUCGAAACAUCA | UGAUGUUUCGACCGUAAUA | 1526 |
| 1472 | CUUUGGCGAGGUUAGUGAA | UUCACUAACCUCGCCAAAG | 1527 |
| 1473 | UUUGGCGAGGUUAGUGAAG | CUUCACUAACCUCGCCAAA | 1528 |
| 1474 | AACUGUUGUUCCUUACCGU | ACGGUAAGGAACAACAGUU | 1529 |
| 1475 | CGCUAAGAACUCUACAAUA | UAUUGUAGAGUUCUUAGCG | 1530 |
| 1476 | AGGUAAUUUCUACGUCGGA | UCCGACGUAGAAAUUACCU | 1531 |
| 1477 | AUUUCUACGUCGGAGGCAA | UUGCCUCCGACGUAGAAAU | 1532 |
| 1478 | UACGUCGGAGGCAACUUUA | UAAAGUUGCCUCCGACGUA | 1533 |
| 1479 | UACCCAACAAGGUAACGUG | CACGUUACCUUGUUGGGUA | 1534 |
| 1480 | AGGUAACGUGGUUGUCUAU | AUAGACAACCACGUUACCU | 1535 |
| 1481 | UGGUGGUAACUAUCCUAUU | AAUAGGAUAGUUACCACCA | 1536 |
| 1482 | AUGUUACUGAAUUUCCGGC | GCCGGAAAUUCAGUAACAU | 1537 |
| 1483 | UGUUACUGAAUUUCCGGCA | UGCCGGAAAUUCAGUAACA | 1538 |
| 1484 | GUUACUGAAUUUCCGGCAG | CUGCCGGAAAUUCAGUAAC | 1539 |
| 1485 | UUACUGAAUUUCCGGCAGC | GCUGCCGGAAAUUCAGUAA | 1540 |
| 1486 | CCACGAGUCUACAAUUCAC | GUGAAUUGUAGACUCGUGG | 1541 |
| 1487 | UUGCACAAGAUGUGUACGA | UCGUACACAUCUUGUGCAA | 1542 |
| 1488 | CUGGUAACUGGUUCGAUAU | AUAUCGAACCAGUUACCAG | 1543 |
| 1489 | UGGUAACUGGUUCGAUAUU | AAUAUCGAACCAGUUACCA | 1544 |
| 1490 | GGUAACUGGUUCGAUAUUG | CAAUAUCGAACCAGUUACC | 1545 |
| 1491 | GUAACUGGUUCGAUAUUGG | CCAAUAUCGAACCAGUUAC | 1546 |
| 1492 | UAACUGGUUCGAUAUUGGC | GCCAAUAUCGAACCAGUUA | 1547 |
| 1493 | AACUGGUUCGAUAUUGGCA | UGCCAAUAUCGAACCAGUU | 1548 |
| 1494 | GGUUCGAUAUUGGCAUAUC | GAUAUGCCAAUAUCGAACC | 1549 |
| 1495 | GUUCGAUAUUGGCAUAUCG | CGAUAUGCCAAUAUCGAAC | 1550 |
| 1496 | UUCGAUAUUGGCAUAUCGU | ACGAUAUGCCAAUAUCGAA | 1551 |
| 1497 | UCGAUAUUGGCAUAUCGUG | CACGAUAUGCCAAUAUCGA | 1552 |
| 1498 | CGAUAUUGGCAUAUCGUGU | ACACGAUAUGCCAAUAUCG | 1553 |
| 1499 | AUAUUGGCAUAUCGUGUGA | UCACACGAUAUGCCAAUAU | 1554 |

### Example 4

### Genes Targeting Sialic Acid Pathways in Avian Cells

Target genes can be those involved in reducing sialic acid from the host cell surface, which reduces virus binding, and therefore increases recovery of the virus in cell culture media. These targets include: solute carrier family 35 (CMP-sialic acid transporter) member A1 (SLC35A1), avian target gene in Table 8; UDP-N-acetylglucosamine 2-epimerase/N-acetylmannosamine kinase (GNE), avian target gene in Table 7; cytidine monophospho-N-acetylneuraminic acid synthetase (Cmas), avian target gene in Table 6; UDP-Gal:betaGlcNAc beta 1,4-galactosyltransferase (B4GalT1), avian target gene in Table 4; and UDP-Gal:betaGlcNAc beta 1,4-galactosyltransferase, polypeptide 6 (B4GalT6), avian target gene in Table 5.

TABLE 4
Chicken B4GALT1

| SEQ ID NO: | # | Sense (3′ to 5′) | AntiSense (5′ to 3′) | SEQ ID NO: |
|---|---|---|---|---|
| 1555 | 106 | CGUUUUGCUCCAAAGGACU | AGUCCUUUGGAGCAAAACG | 1579 |
| 1556 | 136 | CAGAAAGUAGCAAUCAUCA | UGAUGAUUGCUACUUUCUG | 1580 |
| 1557 | 222 | GCAGCAGCUAGAUUAUGGA | UCCAUAAUCUAGCUGCUGC | 1581 |
| 1558 | 226 | CAGCUAGAUUAUGGAGUGU | ACACUCCAUAAUCUAGCUG | 1582 |
| 1559 | 267 | CGAAGAAUUUAACCGUGCU | AGCACGGUUAAAUUCUUCG | 1583 |
| 1560 | 268 | GAAGAAUUUAACCGUGCUA | UAGCACGGUUAAAUUCUUC | 1584 |
| 1561 | 298 | GUAGGAUUCACGGAAGCUU | AAGCUUCCGUGAAUCCUAC | 1585 |
| 1562 | 373 | GAUGACAGGAACACCUACA | UGUAGGUGUUCCUGUCAUC | 1586 |
| 1563 | 387 | CUACAAGUGCUACAGCCAA | UUGGCUGUAGCACUUGUAG | 1587 |
| 1564 | 423 | CUCCAUGGAUAAAUUCGGA | UCCGAAUUUAUCCAUGGAG | 1588 |
| 1565 | 425 | CCAUGGAUAAAUUCGGAUU | AAUCCGAAUUUAUCCAUGG | 1589 |
| 1566 | 430 | GAUAAAUUCGGAUUUCGGU | ACCGAAAUCCGAAUUUAUC | 1590 |
| 1567 | 439 | GGAUUUCGGUUACCCUACA | UGUAGGGUAACCGAAAUCC | 1591 |
| 1568 | 440 | GAUUUCGGUUACCCUACAA | UUGUAGGGUAACCGAAAUC | 1592 |
| 1569 | 445 | CGGUUACCCUACAAUCAGU | ACUGAUUGUAGGGUAACCG | 1593 |
| 1570 | 460 | CAGUAUUUUGGAGGUGUGU | ACACACCUCCAAAAUACUG | 1594 |
| 1571 | 462 | GUAUUUUGGAGGUGUGUCU | AGACACACCUCCAAAAUAC | 1595 |
| 1572 | 479 | CUGCCUUGAGCAAAGAACA | UGUUCUUUGCUCAAGGCAG | 1596 |
| 1573 | 488 | GCAAAGAACAAUUCACGAA | UUCGUGAAUUGUUCUUUGC | 1597 |
| 1574 | 493 | GAACAAUUCACGAAGAUCA | UGAUCUUCGUGAAUUGUUC | 1598 |
| 1575 | 510 | CAAUGGGUUCCCAAACAAU | AUUGUUUGGGAACCCAUUG | 1599 |
| 1576 | 607 | GCUGUCAUUGGGAAAUGCA | UGCAUUUCCCAAUGACAGC | 1600 |
| 1577 | 669 | GGAGAGGUUUGACCGUAUU | AAUACGGUCAAACCUCUCC | 1601 |
| 1578 | 777 | GAUCACAGUGGAUAUCGGA | UCCGAUAUCCACUGUGAUC | 1602 |

#indicates start position

TABLE 5
Chicken B4GALT6

| SEQ ID NO: | # | Sense (3′ to 5′) | AntiSense (5′ to 3′) | SEQ ID NO: |
|---|---|---|---|---|
| 1603 | 379 | CGGCAUAGCAAAUACAUAU | AUAUGUAUUUGCUAUGCCG | 1627 |
| 1604 | 419 | GGUAUAAUGUUGAGAGAAA | UUUCUCUCAACAUUAUACC | 1628 |
| 1605 | 455 | CACAUGAUCAGAUUGUAUA | UAUACAAUCUGAUCAUGUG | 1629 |
| 1606 | 463 | CAGAUUGUAUACUAACAAA | UUUGUUAGUAUACAAUCUG | 1630 |
| 1607 | 530 | GACUGUGUUGCUCAAACAA | UUGUUUGAGCAACACAGUC | 1631 |
| 1608 | 609 | CUUACAUGAGAGGCCUUAU | AUAAGGCCUCUCAUGUAAG | 1632 |
| 1609 | 613 | CAUGAGAGGCCUUAUUGAU | AUCAAUAAGGCCUCUCAUG | 1633 |
| 1610 | 618 | GAGGCCUUAUUGAUGUAAA | UUUACAUCAAUAAGGCCUC | 1634 |
| 1611 | 640 | GAGUGAAAUUAGUUUUGAU | AUCAAAACUAAUUUCACUC | 1635 |
| 1612 | 751 | CAUCAUUCCUUUUCGUAAU | AUUACGAAAAGGAAUGAUG | 1636 |
| 1613 | 798 | GGCAUCUGAUACCGAUGUU | AACAUCGGUAUCAGAUGCC | 1637 |
| 1614 | 859 | GACAGGUACACAACCUUUU | AAAAGGUUGUGUACCUGUC | 1638 |
| 1615 | 869 | CAACCUUUUAAUCGUGCAA | UUGCACGAUUAAAAGGUUG | 1639 |
| 1616 | 969 | CUGAAAAUGACCGAAAUUA | UAAUUUCGGUCAUUUUCAG | 1640 |
| 1617 | 977 | GACCGAAAUUAUUACGGAU | AUCCGUAAUAAUUUCGGUC | 1641 |
| 1618 | 998 | GGAGAAAUGCCACGUCAUU | AAUGACGUGGCAUUUCUCC | 1642 |
| 1619 | 1083 | GACUGACAGUGGAACAAUU | AAUUGUUCCACUGUCAGUC | 1643 |
| 1620 | 1102 | CAAGAAGAUUAAUGGAUUU | AAAUCCAUUAAUCUUCUUG | 1644 |
| 1621 | 1177 | CUAUGCUGGAUACAACGUA | UACGUUGUAUCCAGCAUAG | 1645 |
| 1622 | 1181 | GCUGGAUACAACGUAACAA | UUGUUACGUUGUAUCCAGC | 1646 |
| 1623 | 1205 | GAGGGAGACUUAGGGAAAU | AUUUCCCUAAGUCUCCCUC | 1647 |
| 1624 | 1294 | CAGAGAACGUCAGUAUAUU | AAUAUACUGACGUUCUCUG | 1648 |
| 1625 | 1349 | CUUGUCAGUAGAUUGUAUA | UAUACAAUCUACUGACAAG | 1649 |
| 1626 | 1400 | GCUCCUAUUAGAGACUAUU | AAUAGUCUCUAAUAGGAGC | 1650 |

#indicates start position

TABLE 6
Chicken CMAS

| SEQ ID NO: | # | Sense (3′ to 5′) | AntiSense (5′ to 3′) | SEQ ID NO: |
|---|---|---|---|---|
| 1651 | 109 | CACAGUGUGUGGGUAUCUA | UAGAUACCCACACACUGUG | 1675 |
| 1652 | 117 | GUGGGUAUCUACAGACCAU | AUGGUCUGUAGAUACCCAC | 1676 |
| 1653 | 237 | GUUUCUUAACCAUCAUCAU | AUGAUGAUGGUUAAGAAAC | 1677 |
| 1654 | 246 | CCAUCAUCAUGAGGUCGAU | AUCGACCUCAUGAUGAUGG | 1678 |
| 1655 | 322 | GUAGCAAAUCUGAUUCAAA | UUUGAAUCAGAUUUGCUAC | 1679 |
| 1656 | 333 | GAUUCAAAAGGAGGGCUUU | AAAGCCCUCCUUUUGAAUC | 1680 |
| 1657 | 537 | CUACUUGCAGGGUGGUAAA | UUUACCACCCUGCAAGUAG | 1681 |
| 1658 | 548 | GUGGUAAAAUGGCCUACUA | UAGUAGGCCAUUUUACCAC | 1682 |
| 1659 | 585 | CAGUGUGGAUAUUGAUAUA | UAUAUCAAUAUCCACACUG | 1683 |
| 1660 | 589 | GUGGAUAUUGAUAUAGAUA | UAUCUAUAUCAAUAUCCAC | 1684 |
| 1661 | 627 | GCAAAGAGUAUUGAGCUUU | AAAGCUCAAUACUCUUUGC | 1685 |
| 1662 | 632 | GAGUAUUGAGCUUUGGAUA | UAUCCAAAGCUCAAUACUC | 1686 |
| 1663 | 634 | GUAUUGAGCUUUGGAUAUU | AAUAUCCAAAGCUCAAUAC | 1687 |
| 1664 | 681 | GCUAUUGGUUUGCAGUGUU | AACACUGCAAACCAAUAGC | 1688 |
| 1665 | 709 | CUGACAAAUGGUCGCAUUU | AAAUGCGACCAUUUGUCAG | 1689 |
| 1666 | 711 | GACAAAUGGUCGCAUUUAU | AUAAAUGCGACCAUUUGUC | 1690 |
| 1667 | 748 | GAAAUGGUCUCCUACGAUU | AAUCGUAGGAGACCAUUUC | 1691 |
| 1668 | 758 | CCUACGAUUAUAGAGAUAU | AUAUCUCUAUAAUCGUAGG | 1692 |
| 1669 | 759 | CUACGAUUAUAGAGAUAUU | AAUAUCUCUAUAAUCGUAG | 1693 |
| 1670 | 770 | GAGAUAUUGUUGGUAUCAA | UUGAUACCAACAAUAUCUC | 1694 |
| 1671 | 778 | GUUGGUAUCAAUCUAUUAA | UUAAUAGAUUGAUACCAAC | 1695 |
| 1672 | 864 | GCUGGGAUGUAUAGCAGAA | UUCUGCUAUACAUCCCAGC | 1696 |
| 1673 | 877 | GCAGAAGUUAAUGCAACAA | UUGUUGCAUUAACUUCUGC | 1697 |
| 1674 | 941 | GCUGGAAAGAGGUUGCUUA | UAAGCAACCUCUUUCCAGC | 1698 |

#indicates start position

TABLE 7
Chicken GNE

| SEQ ID | | | | SEQ ID |
|---|---|---|---|---|
| NO: | # | Sense (5′ to 3′) | Antisense (5′ to 3′) | NO: |
| 1699 | 309 | GUAACACUUAUCGCAUGAU | AUCAUGCGAUAAGUGUUAC | 1723 |
| 1700 | 446 | CGCCUGAAACCUGACAUAA | UUAUGUCAGGUUUCAGGCG | 1724 |
| 1701 | 564 | GGACCAUUGACGACUCUAU | AUAGAGUCGUCAAUGGUCC | 1725 |
| 1702 | 568 | CAUUGACGACUCUAUUAGA | UCUAAUAGAGUCGUCAAUG | 1726 |
| 1703 | 885 | CACUUGUUCUAUUCCCCAA | UUGGGGAAUAGAACAAGUG | 1727 |
| 1704 | 1026 | CUGGUUGUAUGAUUGGUAA | UUACCAAUCAUACAACCAG | 1728 |
| 1705 | 1251 | GAAUUUUGAAGUUCCUUAA | UUAAGGAACUUCAAAAUUC | 1729 |
| 1706 | 1403 | CGAGUAGCAAUUGUCAGUA | UACUGACAAUUGCUACUCG | 1730 |
| 1707 | 1785 | GAGGUGGAAUCAUUCACCA | UGGUGAAUGAUUCCACCUC | 1731 |
| 1708 | 1917 | CCUCUGGAAUAGCAUUACA | UGUAAUGCUAUUCCAGAGG | 1732 |
| 1709 | 1944 | CUAAGAAACUGCAUGACGA | UCGUCAUGCAGUUUCUUAG | 1733 |
| 1710 | 2233 | GGUGGUCUCAAAUCUAGCA | UGCUAGAUUUGAGACCACC | 1734 |
| 1711 | 2288 | GAUUAUACUACCCGUAGAA | UUCUACGGGUAGUAUAAUC | 1735 |
| 1712 | 2298 | CCCGUAGAAUAUACUAGGU | ACCUAGUAUAUUCUACGGG | 1736 |
| 1713 | 2451 | GCAUUUCCUAAUUGAGGUA | UACCUCAAUUAGGAAAUGC | 1737 |
| 1714 | 2843 | GACUUGGAGGAUACGGUUA | UAACCGUAUCCUCCAAGUC | 1738 |
| 1715 | 3164 | GUACAUAUGAGUAUAAAGA | UCUUUAUACUCAUAUGUAC | 1739 |
| 1716 | 3324 | CACAUUCAAGGUAUUCAUU | AAUGAAUACCUUGAAUGUG | 1740 |
| 1717 | 3345 | CCUUGGAGCAACUAACCUU | AAGGUUAGUUGCUCCAAGG | 1741 |
| 1718 | 3353 | CAACUAACCUUAAAGGCUU | AAGCCUUUAAGGUUAGUUG | 1742 |
| 1719 | 3367 | GGCUUGCCUUGUUAAGACA | UGUCUUAACAAGGCAAGCC | 1743 |
| 1720 | 3372 | GCCUUGUUAAGACAUAGUU | AACUAUGUCUUAACAAGGC | 1744 |
| 1721 | 3693 | CUGGCAUUUAACAACCUAA | UUAGGUUGUUAAAUGCCAG | 1745 |
| 1722 | 3719 | GCCCCAUAGUAAGGAUAUA | UAUAUCCUUACUAUGGGGC | 1746 |

# indicates start position

TABLE 8
Chicken SLC35A1

| SEQ ID | | | | SEQ ID |
|---|---|---|---|---|
| NO: | # | Sense (5′ to 3′) | Antisense (5′ to 3′) | NO: |
| 1747 | 220 | GGCGCAGAGCUCUACUUUU | AAAAGUAGAGCUCUGCGCC | 1771 |
| 1748 | 289 | GGCAUCUUGGCUAGAGAAU | AUUCUCUAGCCAAGAUGCC | 1772 |
| 1749 | 309 | GGGAAGUCUGGCAAGGUUA | UAACCUUGCCAGACUUCCC | 1773 |
| 1750 | 311 | GAAGUCUGGCAAGGUUAAU | AUUAACCUUGCCAGACUUC | 1774 |
| 1751 | 320 | CAAGGUUAAUAACAUCUUU | AAAGAUGUUAUUAACCUUG | 1775 |
| 1752 | 359 | GUCCCAAGGAAUUGCUAAA | UUUAGCAAUUCCUUGGGAC | 1776 |
| 1753 | 373 | CUAAAAUUAAGUGUUCCAU | AUGGAACACUUAAUUUUAG | 1777 |
| 1754 | 399 | GUACGCUGUCCAAAACAAU | AUUGUUUUGGACAGCGUAC | 1778 |
| 1755 | 422 | CUUUUGUGGCUCUUAGCAA | UUGCUAAGAGCCACAAAAG | 1779 |
| 1756 | 452 | CAGUCUACCAGGUGACGUA | UACGUCACCUGGUAGACUG | 1780 |
| 1757 | 460 | CAGGUGACGUACCAGUUAA | UUAACUGGUACGUCACCUG | 1781 |
| 1758 | 494 | CGUUAUGUACAGUCCUCAU | AUGAGGACUGUACAUAACG | 1782 |
| 1759 | 618 | GGAGCAGAAUCCAUGGCUA | UAGCCAUGGAUUCUGCUCC | 1783 |
| 1760 | 624 | GAAUCCAUGGCUAGGGUUU | AAACCCUAGCCAUGGAUUC | 1784 |
| 1761 | 645 | GGCCAUAACUAUUGCUGUA | UACAGCAAUAGUUAUGGCC | 1785 |
| 1762 | 659 | CUGUACUCUGUUCGGGAUU | AAUCCCGAACAGAGUACAG | 1786 |
| 1763 | 770 | CUUUGGUUGGCGUGUACAU | AUGUACACGCCAACCAAAG | 1787 |
| 1764 | 988 | CAGAUAACGGUUACCUUUA | UAAAGGUAACCGUUAUCUG | 1788 |
| 1765 | 1020 | CCUGGUGUGCGUUUCUAUU | AAUAGAAACGCACACCAGG | 1789 |
| 1766 | 1029 | CGUUUCUAUUUACUUCUAU | AUAGAAGUAAAUAGAAACG | 1790 |
| 1767 | 1078 | CAGCCCUCAGAGACUAAAA | UUUUAGUCUCUGAGGGCUG | 1791 |
| 1768 | 1130 | CUUGAAAAUGGACAGGCAA | UUGCCUGUCCAUUUUCAAG | 1792 |
| 1769 | 1200 | GCCUUAAGCUAUUUUCGAA | UUCGAAAAUAGCUUAAGGC | 1793 |
| 1770 | 1231 | GGAUAGAUUAAAAGCGGAA | UUCCGCUUUUAAUCUAUCC | 1794 |

# indicates start position

### Example 5

### Target Genes Associated with Cell Viability

As discussed herein, a gene associated with host cell viability can be targeted to improve the yield of biomaterial products in cell-based bioprocessing. Example target genes include Bak, Bax, LDH, and FN1.

TABLE 9
Sense and antisense strand sequences of dsRNA against hamster Bak.

| SEQ ID NO: | Sense (3′ to 5′) | AntiSense (5′ to 3′) | SEQ ID NO: |
|---|---|---|---|
| 1795 | ACAGAGGAGGUCUUUCGAA | UUCGAAAGACCUCCUCUGU | 1827 |
| 1796 | GUCUUUCGAAGCUAUGUUU | AAACAUAGCUUCGAAAGAC | 1828 |
| 1797 | UUUCCAUCUCCACCAUCAG | CUGAUGGUGGAGAUGGAAA | 1829 |
| 1798 | AUCUCCACCAUCAGGAACA | UGUUCCUGAUGGUGGAGAU | 1830 |
| 1799 | ACCAUCAGGAACAAGAGAC | GUCUCUUGUUCCUGAUGGU | 1831 |
| 1800 | ACCCCGAGAUGGACAAUUU | AAAUUGUCCAUCUCGGGGU | 1832 |
| 1801 | GAUGGACAAUUUGCUCCUA | UAGGAGCAAAUUGUCCAUC | 1833 |
| 1802 | CAAUUUGCUCCUAGAACCC | GGGUUCUAGGAGCAAAUUG | 1834 |
| 1803 | AGAACCCAACAGCAUCUUG | CAAGAUGCUGUUGGGUUCU | 1835 |
| 1804 | GCAGCUUGCUAUCAUUGGA | UCCAAUGAUAGCAAGCUGC | 1836 |
| 1805 | GCUAUCAUUGGAGAUGACA | UGUCAUCUCCAAUGAUAGC | 1837 |
| 1806 | AUGACAUUAACCGGAGAUA | UAUCUCCGGUUAAUGUCAU | 1838 |
| 1807 | UUAACCGGAGAUACGACAC | GUGUCGUAUCUCCGGUUAA | 1839 |
| 1808 | GAUACGACACAGAGUUCCA | UGGAACUCUGUGUCGUAUC | 1840 |
| 1809 | AGUUCCAGAAUUUACUGGA | UCCAGUAAAUUCUGGAACU | 1841 |
| 1810 | AUUUACUGGAGCAGCUGCA | UGCAGCUGCUCCAGUAAAU | 1842 |
| 1811 | GGAAUGCCUACGAACUCUU | AAGAGUUCGUAGGCAUUCC | 1843 |
| 1812 | ACGAACUCUUCACCAAGAU | AUCUUGGUGAAGAGUUCGU | 1844 |
| 1813 | CUCUUCACCAAGAUUGCCU | AGGCAAUCUUGGUGAAGAG | 1845 |
| 1814 | AGAUUGCCUCCAGCCUAUU | AAUAGGCUGGAGGCAAUCU | 1846 |
| 1815 | GCCUAUUUAAGAGCGGCAU | AUGCCGCUCUUAAAUAGGC | 1847 |
| 1816 | CCUGUAUGUCUACCAACGU | ACGUUGGUAGACAUACAGG | 1848 |
| 1817 | AUGUCUACCAACGUGGUUU | AAACCACGUUGGUAGACAU | 1849 |
| 1818 | UUUUGGCGGAUAUCAUACU | AGUAUGAUAUCCGCCAAAA | 1850 |
| 1819 | GAUAUCAUACUGCACCAUU | AAUGGUGCAGUAUGAUAUC | 1851 |
| 1820 | AUACUGCACCAUUGCAUCG | CGAUGCAAUGGUGCAGUAU | 1852 |
| 1821 | AGACCCAAUCCUGAUUGUG | CACAAUCAGGAUUGGGUCU | 1853 |
| 1822 | CCUGAUUGUGAUGACAAUU | AAUUGUCAUCACAAUCAGG | 1854 |
| 1823 | GAUGACAAUUCUUGGUGUG | CACACCAAGAAUUGUCAUC | 1855 |
| 1824 | UCUUGGUGUGGUUCUGUUG | CAACAGAACCACACCAAGA | 1856 |
| 1825 | CGUGGUACACAGAUUCUUC | GAAGAAUCUGUGUACCACG | 1857 |
| 1826 | UUCUUCAGAUCAUGACUCC | GGAGUCAUGAUCUGAAGAA | 1858 |

TABLE 10
Target gene: Gallus (chicken) BAK1

| SEQ ID | | | | SEQ ID |
|---|---|---|---|---|
| NO: | # | Sense (5′ to 3′) | Antisense (5′ to 3′) | NO: |
| 1859 | 8 | AAUGGAAUGGGAAGUUGGA | UCCAACUUCCCAUUCCAUU | 1880 |
| 1860 | 20 | AGUUGGAGCCUCUGGAUAU | AUAUCCAGAGGCUCCAACU | 1881 |
| 1861 | 40 | GGAAAGGAGAGACCUCGUU | AACGAGGUCUCUCCUUUCC | 1882 |
| 1862 | 41 | GAAAGGAGAGACCUCGUUU | AAACGAGGUCUCUCCUUUC | 1883 |
| 1863 | 146 | CUGCAAUGGAAGGGAACGA | UCGUUCCCUUCCAUUGCAG | 1884 |
| 1864 | 211 | AGGCCAUCACGAGAGAUCA | UGAUCUCUCGUGAUGGCCU | 1885 |
| 1865 | 216 | AUCACGAGAGAUCAAUGCA | UGCAUUGAUCUCUCGUGAU | 1886 |
| 1866 | 268 | UUCCGGAGCUACACCUUCU | AGAAGGUGUAGCUCCGGAA | 1887 |
| 1867 | 269 | UCCGGAGCUACACCUUCUA | UAGAAGGUGUAGCUCCGGA | 1888 |
| 1868 | 278 | ACACCUUCUACCGCUACCA | UGGUAGCGGUAGAAGGUGU | 1889 |
| 1869 | 413 | ACGACAUCAACAAGCGGUA | UACCGCUUGUUGAUGUCGU | 1890 |
| 1870 | 518 | UGUUCGACAGCGGCAUUAA | UUAAUGCCGCUGUCGAACA | 1891 |
| 1871 | 560 | UGGGUUUCGGUUACUGCAU | AUGCAGUAACCGAAACCCA | 1892 |
| 1872 | 565 | UUCGGUUACUGCAUGGCCA | UGGCCAUGCAGUAACCGAA | 1893 |
| 1873 | 716 | ACAAUGUUUACAUGAAGUA | UACUUCAUGUAAACAUUGU | 1894 |
| 1874 | 762 | GGUGGGGCAUUUAGUGGUA | UACCACUAAAUGCCCCACC | 1895 |
| 1875 | 765 | GGGGCAUUUAGUGGUACGA | AGCGUCGUACCACUAAAUG | 1896 |
| 1876 | 770 | AUUUAGUGGUACGACGCUU | AAGCGUCGUACCACUAAAU | 1897 |
| 1877 | 772 | UUAGUGGUACGACGCUUCU | AGAAGCGUCGUACCACUAA | 1898 |
| 1878 | 773 | UAGUGGUACGACGCUUCUU | AAGAAGCGUCGUACCACUA | 1899 |
| 1879 | 1110 | AAUGCAUUGCUGUGAGAGU | ACUCUCACAGCAAUGCAUU | 1900 |

# indicates start position

TABLE 11
dsRNA against hamster Bax.

| SEQ ID NO: | Sense (3′ to 5′) | AntiSense (5′ to 3′) | SEQ ID NO: |
|---|---|---|---|
| 1901 | AGCUCUGAGCAGAUCAUGA | UCAUGAUCUGCUCAGAGCU | 1933 |
| 1902 | GAGCAGAUCAUGAAGACAG | CUGUCUUCAUGAUCUGCUC | 1934 |
| 1903 | UUUGCUACAGGGUUUCAUC | GAUGAAACCCUGUAGCAAA | 1935 |
| 1904 | UGCUACAGGGUUUCAUCCA | UGGAUGAAACCCUGUAGCA | 1936 |
| 1905 | UUUCAUCCAGGAUCGAGCU | AGCUCGAUCCUGGAUGAAA | 1937 |
| 1906 | AUACACCUGAGCUGACCUU | AAGGUCAGCUCAGGUGUAU | 1938 |
| 1907 | AGGAUCCGUCUACCAAGAA | UUCUUGGUAGACGGAUCCU | 1939 |
| 1908 | CCGUCUACCAAGAAGUUGA | UCAACUUCUUGGUAGACGG | 1940 |
| 1909 | UACCAAGAAGUUGAGCGAG | CUCGCUCAACUUCUUGGUA | 1941 |
| 1910 | GAAUUGGAGAUGAGCUGGA | UCCAGCUCAUCUCCAAUUC | 1942 |
| 1911 | AUGAGCUGGACAGCAACAU | AUGUUGCUGUCCAGCUCAU | 1943 |
| 1912 | CUGCAGAGGAUGAUUGCUA | UAGCAAUCAUCCUCUGCAG | 1944 |
| 1913 | UGAUUGCUAAUGUGGAUAC | GUAUCCACAUUAGCAAUCA | 1945 |
| 1914 | AUACUAACUCCCCACGAGA | UCUCGUGGGGAGUUAGUAU | 1946 |
| 1915 | CAGCUGACAUGUUUGCUGA | UCAGCAAACAUGUCAGCUG | 1947 |
| 1916 | UGUUUGCUGAUGGCAACUU | AAGUUGCCAUCAGCAAACA | 1948 |
| 1917 | GUUGUUGCCCUUUUCUACU | AGUAGAAAAGGGCAACAAC | 1949 |
| 1918 | CUUUGCUAGCAAACUGGUG | CACCAGUUUGCUAGCAAAG | 1950 |
| 1919 | AAAGUGCCCGAGCUAAUCA | UGAUUAGCUCGGGCACUUU | 1951 |
| 1920 | CUAAUCAGAACCAUCAUGG | CCAUGAUGGUUCUGAUUAG | 1952 |
| 1921 | UAAUCAGAACCAUCAUGGG | CCCAUGAUGGUUCUGAUUA | 1953 |
| 1922 | UUGUCUGGAUCCAAGACCA | UGGUCUUGGAUCCAGACAA | 1954 |
| 1923 | AUGGCCUCCUCUCAUACUU | AAGUAUGAGAGGAGGCCAU | 1955 |
| 1924 | GACAGUGACUAUCUUUGUG | CACAAAGAUAGUCACUGUC | 1956 |
| 1925 | GUGACUAUCUUUGUGGCUG | CAGCCACAAAGAUAGUCAC | 1957 |
| 1926 | ACUCACGAUCUGGAAGAAC | GUUCUUCCAGAUCGUGAGU | 1958 |
| 1927 | AUCUGGAAGAACAUGGGCU | AGCCCAUGUUCUUCCAGAU | 1959 |
| 1928 | GUCUUGGACUAUUGUCUUU | AAAGACAAUAGUCCAAGAC | 1960 |
| 1929 | UUUGUAAUUAUUGGGAGGG | CCCUCCCAAUAAUUACAAA | 1961 |
| 1930 | GUAAUUAUUGGGAGGGGUG | CACCCCUCCCAAUAAUUAC | 1962 |
| 1931 | AGGGGACAAUAAACCUCAG | CUGAGGUUUAUUGUCCCCU | 1963 |
| 1932 | ACCUCAGGUCCACUUUAAA | UUUAAAGUGGACCUGAGGU | 1964 |

TABLE 12
Sense and antisense strand sequences of dsRNA against hamster LDH-A.

| SEQ ID NO: | Sense (3′ to 5′) | AntiSense (5′ to 3′) | SEQ ID NO: |
|---|---|---|---|
| 1965 | ACUCAAGGACCAGCUGAUU | AAUCAGCUGGUCCUUGAGU | 1997 |
| 1966 | CUACUUAAGGAAGAACAGA | UCUGUUCUUCCUUAAGUAG | 1998 |
| 1967 | GAUUACGAUUGUUGGGGUU | AACCCCAACAAUCGUAAUC | 1999 |
| 1968 | CAGUAUCCUCAUGAAGGAC | GUCCUUCAUGAGGAUACUG | 2000 |
| 1969 | ACUUAGCAGAUGAGCUUGC | GCAAGCUCAUCUGCUAAGU | 2001 |
| 1970 | CCCUUGUUGAUGUCAUGGA | UCCAUGACAUCAACAAGGG | 2002 |
| 1971 | UUUCCUCAGAACACCAAAA | UUUUGGUGUUCUGAGGAAA | 2003 |
| 1972 | AAGACUAUAGUGUGACAGC | GCUGUCACACUAUAGUCUU | 2004 |
| 1973 | CAAGCUGGUCAUUGUCACA | UGUGACAAUGACCAGCUUG | 2005 |
| 1974 | AACGAAACGUGAACAUCUU | AAGAUGUUCACGUUUCGUU | 2006 |
| 1975 | UCAUCAUUCCCAACGUUGU | ACAACGUUGGGAAUGAUGA | 2007 |
| 1976 | GAAAUACAGUCCAGACUGC | GCAGUCUGGACUGUAUUUC | 2008 |
| 1977 | UUCAAACCCAGUGGAUAUC | GAUAUCCACUGGGUUUGAA | 2009 |
| 1978 | UUAUUGGGAGUGGCUGCAA | UUGCAGCCACUCCCAAUAA | 2010 |
| 1979 | CCGUUAUCUGAUGGGAGAA | UUCUCCCAUCAGAUAACGG | 2011 |
| 1980 | UUCACCCACUAAGCUGUCA | UGACAGCUUAGUGGGUGAA | 2012 |
| 1981 | UGUGUGGAGUGGAGUGAAU | AUUCACUCCACUCCACACA | 2013 |
| 1982 | GAGUGGAGUGAAUGUAGCU | AGCUACAUUCACUCCACUC | 2014 |
| 1983 | UCUCCCUGAAGAAUCUGAA | UUCAGAUUCUUCAGGGAGA | 2015 |
| 1984 | ACAAGGAGCAGUGGAAUGA | UCAUUCCACUGCUCCUUGU | 2016 |
| 1985 | AAUGAGGUUCACAAGCAGG | CCUGCUUGUGAACCUCAUU | 2017 |
| 1986 | AGUGCCUAUGAGGUGAUCA | UGAUCACCUCAUAGGCACU | 2018 |
| 1987 | UCAAACUGAAGGGUUAUAC | GUAUAACCCUUCAGUUUGA | 2019 |
| 1988 | CCUGGGCCAUUGGUCUGUC | GACAGACCAAUGGCCCAGG | 2020 |
| 1989 | GAGAGCAUAAUGAAGAAUC | GAUUCUUCAUUAUGCUCUC | 2021 |
| 1990 | UGCAUCCCAUUUCUACCAU | AUGGUAGAAAUGGGAUGCA | 2022 |
| 1991 | AAGGGUCUCUAUGGAAUCA | UGAUUCCAUAGAGACCCUU | 2023 |
| 1992 | CUAUGGAAUCAAGGAUGAU | AUCAUCCUUGAUUCCAUAG | 2024 |
| 1993 | CUGGGACAAAAUGGAAUCU | AGAUUCCAUUUUGUCCCAG | 2025 |
| 1994 | UGUGAAGGUGACUCUAACU | AGUUAGAGUCACCUUCACA | 2026 |
| 1995 | GACUCUAACUUCUGAAGAG | CUCUUCAGAAGUUAGAGUC | 2027 |
| 1996 | CUUGAAGAAGAGUGCAGAU | AUCUGCACUCUUCUUCAAG | 2028 |

TABLE 13
dsRNA against hamster Bax, hamster Bak and hamster LDH-A.

| | | SEQ | | | SEQ | IC₅₀ |
|---|---|---|---|---|---|---|
| Duplex | Target | ID | Sense (3′ to 5′) | AntiSense (5′ to 3′) | ID | (nM) |
| A7 | Bax | 2029 | CCGUCUACCAAGAAGUUGAdT | UCAACUUCUUGGUAGACGGdT | 2044 | 0.38 |
| | | | dT | dT | | |
| B2 | Bax | 2030 | CAGCUGACAUGUUUGCUGAdT | UCAGCAAACAUGUCAGCUGdT | 2045 | 1.46 |
| | | | dT | dT | | |
| B4 | Bax | 2031 | GUUGUUGCCCUUUUCUACUdT | AGUAGAAAAGGGCAACAACdT | 2046 | 0.08 |
| | | | dT | dT | | |
| B11 | Bax | 2032 | GACAGUGACUAUCUUUGUGdT | CACAAAGAUAGUCACUGUCdT | 2047 | 0.22 |
| | | | dT | dT | | |
| C6 | Bax | 2033 | AGCUCUGAGCAGAUCAUGAdT | UCAUGAUCUGCUCAGAGCUdT | 2048 | 0.17 |
| | | | dT | dT | | |
| A2 | Bak | 2034 | GUCUUUCGAAGCUAUGUUUdT | AAACAUAGCUUCGAAAGACdT | 2049 | 0.07 |
| | | | dT | dT | | |
| A10 | Bak | 2035 | GCAGCUUGCUAUCAUUGGAdT | UCCAAUGAUAGCAAGCUGCdT | 2050 | 0.38 |
| | | | dT | dT | | |
| A11 | Bak | 2036 | GCUAUCAUUGGAGAUGACAdT | UGUCAUCUCCAAUGAUAGCdT | 2051 | 0.14 |
| | | | dT | dT | | |
| B9 | Bak | 2037 | GCCUAUUUAAGAGCGGCAUdT | AUGCCGCUCUUAAAUAGGCdT | 2052 | 0.08 |
| | | | dT | dT | | |
| C7 | Bak | 2038 | CGUGGUACACAGAUUCUUCdT | GAAGAAUCUGUGUACCACGdT | 2053 | 0.04 |
| | | | dT | dT | | |
| C10 | LDH | 2039 | CUACUUAAGGAAGAACAGAdT | UCUGUUCUUCCUUAAGUAGdT | 2054 | 0.06 |
| | | | dT | dT | | |
| D5 | LDH | 2040 | CAAGCUGGUCAUUGUCACAdT | UGUGACAAUGACCAGCUUGdT | 2055 | 0.06 |
| | | | dT | dT | | |
| D7 | LDH | 2041 | UCAUCAUUCCCAACGUUGUdT | ACAACGUUGGGAAUGAUGAdT | 2056 | 0.13 |
| | | | dT | dT | | |
| E2 | LDH | 2042 | GAGUGGAGUGAAUGUAGCUdT | AGCUACAUUCACUCCACUCdT | 2057 | 0.40 |
| | | | dT | dT | | |
| E4 | LDH | 2043 | ACAAGGAGCAGUGGAAUGAdT | UCAUUCCACUGCUCCUUGUdT | 2058 | 0.15 |
| | | | dT | dT | | |

TABLE 14
Target gene: Gallus (chicken) FN1

| SEQ ID | | | | SEQ ID |
|---|---|---|---|---|
| NO: | # | Sense (5′ to 3′) | Antisense (5′ to 3′ | NO: |
| 2059 | 173 | GGACGGUACUACCAGAUAA | UUAUCUGGUAGUACCGUCC | 2088 |
| 2060 | 174 | GACGGUACUACCAGAUAAA | UUUAUCUGGUAGUACCGUC | 2089 |
| 2061 | 257 | GGGUUUAACUGCGAAAGCA | UGCUUUCGCAGUUAAACCC | 2090 |
| 2062 | 258 | GGUUUAACUGCGAAAGCAA | UUGCUUUCGCAGUUAAACC | 2091 |
| 2063 | 325 | CCGAGUAGGGGAGACUUAU | AUAAGUCUCCCCUACUCGG | 2092 |
| 2064 | 832 | CCUGCAUACAACUAGCACU | AGUGCUAGUUGUAUGCAGG | 2093 |
| 2065 | 1465 | GUGGGAUAAACAGCACGAU | AUCGUGCUGUUUAUCCCAC | 2094 |
| 2066 | 1467 | GGGAUAAACAGCACGAUAU | AUAUCGUGCUGUUUAUCCC | 2095 |
| 2067 | 1700 | CAGGAUUCCGAAACUCGUA | UACGAGUUUCGGAAUCCUG | 2096 |
| 2068 | 1973 | CACUGGAAGGAAGCUACCA | UGGUAGCUUCCUUCCAGUG | 2097 |
| 2069 | 2256 | CUGGAUUCCGUGUUGAGUA | UACUCAACACGGAAUCCAG | 2098 |
| 2070 | 2765 | GUUUCUGACGUCAAGGUCA | UGACCUUGACGUCAGAAAC | 2099 |
| 2071 | 2921 | GGAACGACCUAUCUCUUUA | UAAAGAGAUAGGUCGUUCC | 2100 |
| 2072 | 3085 | CUCAGGAUACCGACUGACU | AGUCAGUCGGUAUCCUGAG | 2101 |
| 2073 | 3146 | GGCCCUUCUGCUACUAAGU | ACUUAGUAGCAGAAGGGCC | 2102 |
| 2074 | 3252 | GAGUCUUCACAACGUAUCA | UGAUACGUUGUGAAGACUC | 2103 |
| 2075 | 3827 | GACCUAAGCUUUGUUGACA | UGUCAACAAAGCUUAGGUC | 2104 |
| 2076 | 3868 | GAGGUGGACCCCGUUAAAU | AUUUAACGGGGUCCACCUC | 2105 |
| 2077 | 4291 | GGUGAGAGUCUACAGUGUU | AACACUGUAGACUCUCACC | 2106 |
| 2078 | 4373 | GGCCUUGACUUCUCGGAUA | UAUCCGAGAAGUCAAGGCC | 2107 |
| 2079 | 4434 | CCACCAUUACGGGCUAUAA | UUAUAGCCCGUAAUGGUGG | 2108 |
| 2080 | 4443 | CGGGCUAUAAGAUUCGGCA | UGCCGAAUCUUAUAGCCCG | 2109 |
| 2081 | 4444 | GGGCUAUAAGAUUCGGCAU | AUGCCGAAUCUUAUAGCCC | 2110 |
| 2082 | 4923 | CCCAGAUGCAAGUUACCGA | UCGGUAACUUGCAUCUGGG | 2111 |
| 2083 | 5874 | GCCCCGAUGUUAGGACUUA | UAAGUCCUAACAUCGGGGC | 2112 |
| 2084 | 5882 | GUUAGGACUUACACUAUCA | UGAUAGUGUAAGUCCUAAC | 2113 |
| 2085 | 6179 | GAACCAGGGACCGAAUACA | UGUAUUCGGUCCCUGGUUC | 2114 |
| 2086 | 7158 | CUCAUGAGACCACAUGCUA | UAGCAUGUGGUCUCAUGAG | 2115 |
| 2087 | 7647 | GACUUUUCUCACGAUUGGU | ACCAAUCGUGAGAAAAGUC | 2116 |

# indicates start position

TABLE 15
Target gene: Gallus (chicken) PTEN

| SEQ ID | | | | SEQ ID |
|---|---|---|---|---|
| NO: | # | Sense (5′-3′) | Antisense (5′-3′) | NO: |
| 2117 | 300 | UAUUCAGUAGUACGGAUUU | AAAUCCGUACUACUGAAUA | 2147 |
| 2118 | 310 | UACGGAUUUCUAUUAAGUU | AACUUAAUAGAAAUCCGUA | 2148 |
| 2119 | 672 | AGGAGUAUACCGGAACAAU | AUUGUUCCGGUAUACUCCU | 2149 |
| 2120 | 867 | UGACCAAUGGCUAAGUGAA | UUCACUUAGCCAUUGGUCA | 2150 |
| 2121 | 938 | GUGUAAUGAUAUGUGCAUA | UAUGCACAUAUCAUUACAC | 2151 |
| 2122 | 960 | GUUGCAUCGAGGCAAGUUU | AAACUUGCCUCGAUGCAAC | 2152 |
| 2123 | 986 | CUCAAGAAGCCCUAGAUUU | AAAUCUAGGGCUUCUUGAG | 2153 |
| 2124 | 1058 | GACGCUACGUGUACUACUA | UAGUAGUACACGUAGCGUC | 2154 |
| 2125 | 2084 | AUGUGUAUACGUAGGAAUU | AAUUCCUACGUAUACACAU | 2155 |
| 2126 | 2085 | UGUGUAUACGUAGGAAUUU | AAAUUCCUACGUAUACACA | 2156 |
| 2127 | 2323 | GUACCAUAUCCGGCUAUUU | AAAUAGCCGGAUAUGGUAC | 2157 |
| 2128 | 2324 | UACCAUAUCCGGCUAUUUA | UAAAUAGCCGGAUAUGGUA | 2158 |
| 2129 | 2356 | UUAGCAUGCUGCCGUAAUU | AAUUACGGCAGCAUGCUAA | 2159 |
| 2130 | 2642 | AUAAAAGUUGUCGGUGAUU | AAUCACCGACAACUUUUAU | 2160 |
| 2131 | 2748 | ACGCCAGGCAACAUCUUAA | UUAAGAUGUUGCCUGGCGU | 2161 |
| 2132 | 2752 | CAGGCAACAUCUUAACAUU | AAUGUUAAGAUGUUGCCUG | 2162 |
| 2133 | 2770 | UUCUUAUUAGGUCUUCAAU | AUUGAAGACCUAAUAAGAA | 2163 |
| 2134 | 2972 | GACCUUACACGUUGUAUUA | UAAUACAACGUGUAAGGUC | 2164 |
| 2135 | 3427 | AAAUUUACACUUGCUCGAU | AUCGAGCAAGUGUAAAUUU | 2165 |
| 2136 | 3558 | AGCAUUAAUGGUAAGGAUU | AAUCCUUACCAUUAAUGCU | 2166 |
| 2137 | 3631 | UUUGGGAUCCGUUGGUCAA | UUGACCAACGGAUCCCAAA | 2167 |
| 2138 | 3658 | AUCACAUUCACGAUUCUAA | UUAGAAUCGUGAAUGUGAU | 2168 |
| 2139 | 3664 | UUCACGAUUCUAACUUCUA | UAGAAGUUAGAAUCGUGAA | 2169 |
| 2140 | 3665 | UCACGAUUCUAACUUCUAA | UUAGAAGUUAGAAUCGUGA | 2170 |
| 2141 | 3812 | GACGUUAACUCUUACCCUU | AAGGGUAAGAGUUAACGUC | 2171 |
| 2142 | 4089 | UUUUGAAUGUUUCGAUGUU | AACAUCGAAACAUUCAAAA | 2172 |
| 2143 | 4315 | UAAUUAGCUUGAAACGCAU | AUGCGUUUCAAGCUAAUUA | 2173 |
| 2144 | 4469 | AUUGGUAGCCCCACUCAAA | UUUGAGUGGGGCUACCAAU | 2174 |
| 2145 | 4570 | GCAUCAGAUGUUUCGGCUU | AAGCCGAAACAUCUGAUGC | 2175 |
| 2146 | 5042 | CCCUUUAUCUCUUAGUGUU | AACACUAAGAGAUAAAGGG | 2176 |

# indicates start position

TABLE 16
Target gene: Gallus (chicken) LDHA

| SEQ ID | | | | SEQ ID |
|---|---|---|---|---|
| NO: | # | Sense (5′-3′) | Antisense (5′-3′) | NO: |
| 2177 | 281 | GGCAGAUGAACUUACCCUU | AAGGGUAAGUUCAUCUGCC | 2203 |
| 2178 | 283 | CAGAUGAACUUACCCUUGU | ACAAGGGUAAGUUCAUCUG | 2204 |
| 2179 | 456 | GGAGAAAGCCGUCUUAACU | AGUUAAGACGGCUUUCUCC | 2205 |
| 2180 | 457 | GAGAAAGCCGUCUUAACUU | AAGUUAAGACGGCUUUCUC | 2206 |
| 2181 | 464 | CCGUCUUAACUUGGUCCAA | UUGGACCAAGUUAAGACGG | 2207 |
| 2182 | 522 | GUGAAAUACAGUCCUGACU | AGUCAGGACUGUAUUUCAC | 2208 |
| 2183 | 542 | CAAGCUGCUGAUUGUCUCA | UGAGACAAUCAGCAGCUUG | 2209 |
| 2184 | 548 | GCUGAUUGUCUCAAACCCA | UGGGUUUGAGACAAUCAGC | 2210 |
| 2185 | 983 | GGGCAUGCACGGAAUAAAA | UUUUAUUCCGUGCAUGCCC | 2211 |
| 2186 | 1056 | GUAGUGAAGAUGAUCCUAA | UUAGGAUCAUCUUCACUAC | 2212 |
| 2187 | 1061 | GAAGAUGAUCCUAAAACCU | AGGUUUUAGGAUCAUCUUC | 2213 |
| 2188 | 1194 | CUUAAUGUUGCACUUUCCA | UGGAAAGUGCAACAUUAAG | 2214 |
| 2189 | 1230 | GGUGUAUGUAUCUCAACUA | UAGUUGAGAUACAUACACC | 2215 |
| 2190 | 1358 | CCACCUAGUAUUGUACAGU | ACUGUACAAUACUAGGUGG | 2216 |
| 2191 | 1380 | GUGGUGUGACAUCAAUCCA | UGGAUUGAUGUCACACCAC | 2217 |
| 2192 | 1389 | CAUCAAUCCAGCAUUGAAA | UUUCAAUGCUGGAUUGAUG | 2218 |
| 2193 | 1432 | GUUUAAACAUUAGCCCAGA | UCUGGGCUAAUGUUUAAAC | 2219 |
| 2194 | 1508 | CACAGGAUUGUCAAACUAU | AUAGUUUGACAAUCCUGUG | 2220 |
| 2195 | 1519 | CAAACUAUUUGACAAACCU | AGGUUUGUCAAAUAGUUUG | 2221 |
| 2196 | 1541 | CUGCCAGCUGAAUAGCUUA | UAAGCUAUUCAGCUGGCAG | 2222 |
| 2197 | 1569 | CUGUUGGUAGGUCCAAAUA | UAUUUGGACCUACCAACAG | 2223 |
| 2198 | 1599 | CUUCUUUGGAACUUGUGUA | UACACAAGUUCCAAAGAAG | 2224 |
| 2199 | 1606 | GGAACUUGUGUAUACCUUU | AAAGGUAUACACAAGUUCC | 2225 |
| 2200 | 1607 | GAACUUGUGUAUACCUUUU | AAAAGGUAUACACAAGUUC | 2226 |
| 2201 | 1649 | GUACUGUAUCAGACUAGUU | AACUAGUCUGAUACAGUAC | 2227 |
| 2202 | 1708 | GUUUAUAUAUGCAACAAGA | UCUUGUUGCAUAUAUAAAC | 2228 |

# indicates start position

### Example 6

### Targets Associated with Glycosylation

In addition to the target genes associated with sialic acid, as described herein, yield and/or qualities of a biological product can be optimized by targeting genes associated with glycosylation in the host cell.

TABLE 17
dsRNA against hamster fucosyltransferase (FUT8).

| SEQ ID NO: | Sense (3′ to 5′) | AntiSense (5′ to 3′) | SEQ ID NO: |
|---|---|---|---|
| 2229 | ACUCUGAAAAUGCGGGCAU | AUGCCCGCAUUUUCAGAGU | 2249 |
| 2230 | GGUUCCUGGCGUUGGAUUA | UAAUCCAACGCCAGGAACC | 2250 |
| 2231 | GGAUUAUGCUCAUUCUUUU | AAAAGAAUGAGCAUAAUCC | 2251 |
| 2232 | UUUAUAUAGGUGGUCAUUU | AAAUGACCACCUAUAUAAA | 2252 |
| 2233 | UAUAGGUGGUCAUUUGGUU | AACCAAAUGACCACCUAUA | 2253 |
| 2234 | GGUCAUUUGGUUCGAGAUA | UAUCUCGAACCAAAUGACC | 2254 |
| 2235 | GUCAUUUGGUUCGAGAUAA | UUAUCUCGAACCAAAUGAC | 2255 |
| 2236 | CUCUCCAAGAUUCUUGCAA | UUGCAAGAAUCUUGGAGAG | 2256 |
| 2237 | CAGGAAGAGUCCGUGUUUU | AAAACACGGACUCUUCCUG | 2257 |
| 2238 | GAGCUAAAGAGCUCUGGUU | AACCAGAGCUCUUUAGCUC | 2258 |
| 2239 | AAGGUCUAUCAUGACAGAU | AUCUGUCAUGAUAGACCUU | 2259 |
| 2240 | CUGGUCCAGCGGAGAAUAA | UUAUUCUCCGCUGGACCAG | 2260 |
| 2241 | GCACCCAGCGAACACUCAU | AUGAGUGUUCGCUGGGUGC | 2261 |
| 2242 | ACUGUGUUUAGACCUGUAA | UUACAGGUCUAAACACAGU | 2262 |
| 2243 | UGGCUUCAAACAUCCAGUU | AACUGGAUGUUUGAAGCCA | 2263 |
| 2244 | CAAAGACAAAGUACUCCAA | UUGGAGUACUUUGUCUUUG | 2264 |
| 2245 | AAGACAAAGUACUCCAAUU | AAUUGGAGUACUUUGUCUU | 2265 |
| 2246 | AGACAAAGUACUCCAAUUA | UAAUUGGAGUACUUUGUCU | 2266 |
| 2247 | GACAAAGUACUCCAAUUAU | AUAAUUGGAGUACUUUGUC | 2267 |
| 2248 | UGAUCCUGGAUAUACACUU | AAGUGUAUAUCCAGGAUCA | 2268 |

TABLE 18
dsRNA against hamster GDP-mannose 4,6-dehydratase (GMDS).

| SEQ ID NO: | Sense (3′ to 5′) | AntiSense (5′ to 3′) | SEQ ID NO: |
|---|---|---|---|
| 2269 | UCAUUUAAUACAGGUCGAA | UUCGACCUGUAUUAAAUGA | 2289 |
| 2270 | AUUUAAUACAGGUCGAAUU | AAUUCGACCUGUAUUAAAU | 2290 |
| 2271 | UACAGGUCGAAUUGAACAU | AUGUUCAAUUCGACCUGUA | 2291 |
| 2272 | GUCGAAUUGAACAUUUAUA | UAUAAAUGUUCAAUUCGAC | 2292 |
| 2273 | AUAUUGAAGGAAACAUGAA | UUCAUGUUUCCUUCAAUAU | 2293 |
| 2274 | GGAAACAUGAAGUUGCACU | AGUGCAACUUCAUGUUUCC | 2294 |
| 2275 | AGCUAAGAUUUACCUUGGA | UCCAAGGUAAAUCUUAGCU | 2295 |
| 2276 | CUAAGAUUUACCUUGGACA | UGUCCAAGGUAAAUCUUAG | 2296 |
| 2277 | UAAGAUUUACCUUGGACAA | UUGUCCAAGGUAAAUCUUA | 2297 |
| 2278 | GAGACUGGGGCCAUGCCAA | UUGGCAUGGCCCCAGUCUC | 2298 |
| 2279 | GGGCCAUGCCAAGGACUAU | AUAGUCCUUGGCAUGGCCC | 2299 |
| 2280 | UGUCAUAGCUACUGGGGAA | UUCCCCAGUAGCUAUGACA | 2300 |
| 2281 | ACCAUUGUGUGGGAAGGAA | UUCCUUCCCACACAAUGGU | 2301 |
| 2282 | GUGGGCAGAUGUAAAGAGA | UCUCUUUACAUCUGCCCAC | 2302 |
| 2283 | GAUGUAAAGAGACCGGCAA | UUGCCGGUCUCUUUACAUC | 2303 |
| 2284 | GAAGCCCCGCGUUGCCUUU | AAAGGCAACGCGGGGCUUC | 2304 |
| 2285 | UCACAUACUCAUUUUACUU | AAGUAAAAUGAGUAUGUGA | 2305 |
| 2286 | ACUUGAAAUUAUGUCACUA | UAGUGACAUAAUUUCAAGU | 2306 |
| 2287 | AAAUUAUGUCACUAGACAA | UUGUCUAGUGACAUAAUUU | 2307 |
| 2288 | GAGUCUUGAGAUUGUUUUU | AAAAACAAUCUCAAGACUC | 2308 |

### Example 7

### Gene Targets Associated with a Host Cell Immune Response

TABLE 19
Target gene: Gallus (chicken) IFNAR1

| SEQ ID | | | | SEQ ID |
|---|---|---|---|---|
| NO: | # | Sense (5′-3′) | Antisense (5′-3′) | NO: |
| 2309 | 205 | AAUACAAAUUUCACCCUAA | UUAGGGUGAAAUUUGUAUU | 2338 |
| 2310 | 430 | UCUCCAUGGUCUAGCAUUU | AAAUGCUAGACCAUGGAGA | 2339 |
| 2311 | 540 | UCCAGAAGCAAAUCAGGUU | AACCUGAUUUGCUUCUGGA | 2340 |
| 2312 | 673 | GCUCCAGAGACUACCUAUU | AACUUUCAAACAAUAGGUA | 2341 |
| 2313 | 684 | UACCUAUUGUUUGAAAGUU | AACUUUCAAACAAUAGGUA | 2342 |
| 2314 | 839 | UUUGGGAUAAUCACUAUAA | UUAUAGUGAUUAUCCCAAA | 2343 |
| 2315 | 843 | GGAUAAUCACUAUAACGAA | UUCGUUAUAGUGAUUAUCC | 2344 |
| 2316 | 866 | UGACCUAUACCGUACAGUA | UACUGUACGGUAUAGGUCA | 2345 |
| 2317 | 1059 | GUCUAAAGACGUAGAAGUA | UACUUCUACGUCUUUAGAC | 2346 |
| 2318 | 1071 | AGAAGUAGAUCCUCCGGUA | UACCGGAGGAUCUACUUCU | 2347 |
| 2319 | 1072 | GAAGUAGAUCCUCCGGUAA | UUACCGGAGGAUCUACUUC | 2348 |
| 2320 | 1181 | AAAUCAUGAGUGACCUUUA | UAAAGGUCACUCAUGAUUU | 2349 |
| 2321 | 1187 | UGAGUGACCUUUAUGAUUU | AAAUCAUAAAGGUCACUCA | 2350 |
| 2322 | 1407 | ACACUUACCUCUGAUCAUU | AAUGAUCAGAGGUAAGUGU | 2351 |
| 2323 | 1487 | UCCUGUAUCAAGUCUAUAA | UUAUAGACUUGAUACAGGA | 2352 |
| 2324 | 1600 | GUAGAAGAACCGGUAGAAA | UUUCUACCGGUUCUUCUAC | 2353 |
| 2325 | 1610 | CGGUAGAAAUUUGUUACAU | AUGUAACAAAUUUCUACCG | 2354 |
| 2326 | 1625 | ACAUAAUUGAGAGUAGGAU | AUCCUACUCUCAAUUAUGU | 2355 |
| 2327 | 1689 | UAAACAGAGCAGUCGAGAU | AUCUCGACUGCUCUGUUUA | 2356 |
| 2328 | 1701 | UCGAGAUUCAGGGAAUUAU | AUAAUUCCCUGAAUCUCGA | 2357 |
| 2329 | 1725 | CGACGAUAAUACCUCACAA | UUGUGAGGUAUUAUCGUCG | 2358 |
| 2330 | 1765 | CUAGGAAAUGACAUACUAU | AUAGUAUGUCAUUUCCUAG | 2359 |
| 2331 | 1766 | UAGGAAAUGACAUACUAUA | UAUAGUAUGUCAUUUCCUA | 2360 |
| 2332 | 1809 | UAUGGGACUUGUAGUAUAA | UUAUACUACAAGUCCCAUA | 2361 |
| 2333 | 1887 | UUGGGAUCCUCGUUCUGAA | UUCAGAACGAGGAUCCCAA | 2362 |
| 2334 | 1998 | ACACCAUUCUGACUGAAUA | UAUUCAGUCAGAAUGGUGU | 2363 |
| 2335 | 2054 | AGAAGAGAUCCGUAUGCUU | AAGCAUACGGAUCUCUUCU | 2364 |
| 2336 | 2078 | ACUUGAACCUGAUAACCUU | AAGGUUAUCAGGUUCAAGU | 2365 |
| 2337 | 2170 | GACAGAGUCCUUAGAUUAU | AUAAUCUAAGGACUCUGUC | 2366 |

# indicates start position

TABLE 20
Target gene: Gallus (chicken) CDKN2A

| SEQ ID | | | | SEQ ID |
|---|---|---|---|---|
| NO: | # | Sense (5′-3′) | Antisense (5′-3′) | NO: |
| 2367 | 868 | GUGUUGAUCUCAGUCCGAU | AUCGGACUGAGAUCAACAC | 2401 |
| 2368 | 895 | UAUUCUUAACCGGGCUGAU | AUCAGCCCGGUUAAGAAUA | 2402 |
| 2369 | 905 | CGGGCUGAUUGUAUUCGUU | AACGAAUACAAUCAGCCCG | 2403 |
| 2370 | 906 | GGGCUGAUUGUAUUCGUUA | UAACGAAUACAAUCAGCCC | 2404 |
| 2371 | 907 | GGCUGAUUGUAUUCGUUAU | AUAACGAAUACAAUCAGCC | 2405 |
| 2372 | 968 | AUGCUUUCAUAUAGCCGUU | AACGGCUAUAUGAAAGCAU | 2406 |
| 2373 | 972 | UUUCAUAUAGCCGUUAGAA | UUCUAACGGCUAUAUGAAA | 2407 |
| 2374 | 973 | UUCAUAUAGCCGUUAGAAA | UUUCUAACGGCUAUAUGAA | 2408 |
| 2375 | 986 | UAGAAAGAGAAACGGGCAU | AUGCCCGUUUCUCUUUCUA | 2409 |
| 2376 | 1060 | CAGAAAAUCUUACGAAGUU | AACUUCGUAAGAUUUUCUG | 2410 |
| 2377 | 1092 | AAUUAGGUUCUAGGAGCUA | UAGCUCCUAGAACCUAAUU | 2411 |
| 2378 | 1161 | UAGUCAGUUGAAGCGGUUU | AAACCGCUUCAACUGACUA | 2412 |
| 2379 | 1165 | CAGUUGAAGCGGUUUUGUU | AACAAAACCGCUUCAACUG | 2413 |
| 2380 | 1173 | GCGGUUUUGUUACUGCGAA | UUCGCAGUAACAAAACCGC | 2414 |
| 2381 | 1177 | UUUUGUUACUGCGAAGUUU | AAACUUCGCAGUAACAAAA | 2415 |
| 2382 | 1178 | UUUGUUACUGCGAAGUUUU | AAAACUUCGCAGUAACAAA | 2416 |
| 2383 | 1215 | AUCCAAAAGCUCUGUGUAA | UUACACAGAGCUUUUGGAU | 2417 |
| 2384 | 1216 | UCCAAAAGCUCUGUGUAAU | AUUACACAGAGCUUUUGGA | 2418 |
| 2385 | 1224 | CUCUGUGUAAUGAGUGUUU | AAACACUCAUUACACAGAG | 2419 |
| 2386 | 1313 | UGACCUUGGUCCACUGUAA | UUACAGUGGACCAAGGUCA | 2420 |
| 2387 | 1314 | GACCUUGGUCCACUGUAAA | UUUACAGUGGACCAAGGUC | 2421 |
| 2388 | 1413 | GAGGCAUCCGAUUCAACAA | UUGUUGAAUCGGAUGCCUC | 2422 |
| 2389 | 1419 | UCCGAUUCAACAACGAAAU | AUUUCGUUGUUGAAUCGGA | 2423 |
| 2390 | 1422 | GAUUCAACAACGAAAUGUA | UACAUUUCGUUGUUGAAUC | 2424 |
| 2391 | 1440 | AAAAUUGGACUGUUACGUU | AACGUAACAGUCCAAUUUU | 2425 |
| 2392 | 1452 | UUACGUUCCACUUAAAGUU | AACUUUAAGUGGAACGUAA | 2426 |
| 2393 | 1472 | AAGUGUAAAGGGAUCCGUU | AACGGAUCCCUUUACACUU | 2427 |
| 2394 | 1473 | AGUGUAAAGGGAUCCGUUA | UAACGGAUCCCUUUACACU | 2428 |
| 2395 | 1474 | GUGUAAAGGGAUCCGUUAU | AUAACGGAUCCCUUUACAC | 2429 |
| 2396 | 1483 | GAUCCGUUAUGCUCUUUUA | UAAAAGAGCAUAACGGAUC | 2430 |
| 2397 | 1484 | AUCCGUUAUGCUCUUUUAU | AUAAAAGAGCAUAACGGAU | 2431 |
| 2398 | 1485 | UCCGUUAUGCUCUUUUAUU | AAUAAAAGAGCAUAACGGA | 2432 |
| 2399 | 1486 | CCGUUAUGCUCUUUUAUUA | UAAUAAAAGAGCAUAACGG | 2433 |
| 2400 | 1487 | CGUUAUGCUCUUUUAUUAA | UUAAUAAAAGAGCAUAACG | 2434 |

# indicates start position

### Example 8

### Eliminating Latent or Adventitious Viruses from Bioprocessing Cell Culture

RNAi provides a mechanism to block cell culture infections by treating cells while in the bioreactor with siRNAs directed against the infecting latent or adventitious virus. In this manner, RNAi can be used to “cure” the cell bank-line or production cell-line by eliminating or substantially eliminating certain viral types from the contaminated cells.

For example, ROTARIX® (rotavirus vaccine, live, oral) is a liquid suspension of the live attenuated human rotavirus RIX4414 strain grow in bioprocessing-scale cultures of Vero cells. In March, 2010, preparations of ROTARIX® vaccine were found to be contaminated with PCV1 DNA. PVC1 is a single-stranded, ambisense, circular DNA of about 1800 to 2000 bp (see Examples 1 and 2 herein). Two mRNAs for the Rep and Cap proteins are encoded by the encapsidated viral DNA and the complementary DNA of the replicative intermediate DNA strand.

In establishing reagents and uptake conditions to remove PCV1 from cell cultures, Vero from frozen stocks are grown in liquid media. Uptake conditions are established with NDLs using FITC-labeled siRNA. Viability, efficiency, and cell growth are evaluated. The appropriate lipid formulation for RNAi delivery is identified. siRNAs for Vero cell targets and PCV1 are identified (+/− label (Cy3 or Alexa)). Example 1, herein, provides example targets of the PCV1 rep gene. Table 21, herein provides example targets of the PCV1 cap gene. Potent sets are identified, and the best dosing conditions in Vero cells grown in a bioprocessor are established. NDL/siRNA is optimized to maximize cellular uptake and minimize NDL/siRNA in cell culture media. MS analysis of media and cell extracts is compared, including NDL and siRNA analysis. Multiple siRNAs can be used, for example directed against pS-RepA, pS-RepB, and pS-RepC.

Vero cells are then treated prophalactically with “best” PCV1 siRNA(s) at different PCV1 multiplicities of infection (MOI). In test systems, siRNAs are added, then followed by PCV1 infection. Alternatively, Vero cells are infected with PCV1 at a low MOI, then PCV1 siRNA(s) are added. Infected Vero cells can also be passaged at minimal cell densities, and dosed with PCV1 siRNA with each passage. Viral titers may be increased to monitor efficacy of the system, which may be established in a high throughput, e.g., 96-well-plate at single cell densities, format. Additionally, Bax/Bak siRNAs can be included to increase cell density. Cells are sampled at different time points for qPCR, PERT (or other assay) analysis for viral DNA, with the goal of demonstrating that the PCV1 siRNA-treated Vero cells do not support viral replication.

Not to be bound by theory, because PCV1 does not integrate into host genome, blocking viral replication of PCV1 allows the virus to be eliminated from growing Vero cells. In other words, PCV1 replication can be blocked using siRNAs directed against PCV1 while sorting for Vero cells that do not contain PCV1. A PCV1 DNA-based sorting assay is then used that allows Vero cell-sorting to identify and expand those cells that do not contain virus. The sorting assays can be PCR-based, FRET-based, and/or include FACS sorting. Blocking viral replication while sorting for PCV1-free cells, then expanding these non-infected cells, allows for validation of virus removal from the cell bank.

Rotavirus-producing Vero cells can then treated prophalactically with the “best” PCV1 siRNA(s), using best dose concentration and frequency, for maintenance of the cell bank. The bioprocessing of the vaccine is carried out as usual, and tested for PCV1 DNA or viral proteins as discussed herein or otherwise known in the art. Thus, PCV1 siRNA treatment of ROTARIX® vaccine-producing Vero cell line can block PCV1 replication and eliminate any detectable virus from vaccine product.

TABLE 21
Target gene: PCV1 cap

| SEQ ID | | | | SEQ ID |
|---|---|---|---|---|
| NO: | # | Sense (5′ to 3′) | Antisense (5′ to 3′) | NO: |
| 1345 | 48 | GAGGGUCUUUUAGGAUAAA | UUUAUCCUAAAAGACCCUC | 1393 |
| 1346 | 50 | GGGUCUUUUAGGAUAAAUU | AAUUUAUCCUAAAAGACCC | 1394 |
| 1347 | 69 | CUCUGAAUUGUACAUAAAU | AUUUAUGUACAAUUCAGAG | 1395 |
| 1348 | 91 | CAGCCUUACCACAUAAUUU | AAAUUAUGUGGUAAGGCUG | 1396 |
| 1349 | 120 | CUGCAUUUUGGAGCGCAUA | UAUGCGCUCCAAAAUGCAG | 1397 |
| 1350 | 156 | CGACAUUGGUGUGGGUAUU | AAUACCCACACCAAUGUCG | 1398 |
| 1351 | 157 | GACAUUGGUGUGGGUAUUU | AAAUACCCACACCAAUGUC | 1399 |
| 1352 | 159 | CAUUGGUGUGGGUAUUUAA | UUAAAUACCCACACCAAUG | 1400 |
| 1353 | 181 | GAGCCACAGCUGGUUUCUU | AAGAAACCAGCUGUGGCUC | 1401 |
| 1354 | 183 | GCCACAGCUGGUUUCUUUU | AAAAGAAACCAGCUGUGGC | 1402 |
| 1355 | 184 | CCACAGCUGGUUUCUUUUA | UAAAAGAAACCAGCUGUGG | 1403 |
| 1356 | 187 | CAGCUGGUUUCUUUUAUUA | UAAUAAAAGAAACCAGCUG | 1404 |
| 1357 | 189 | GCUGGUUUCUUUUAUUAUU | AAUAAUAAAAGAAACCAGC | 1405 |
| 1358 | 211 | GUGGAACCAAUCAAUUGUU | AACAAUUGAUUGGUUCCAC | 1406 |
| 1359 | 227 | GUUUGGUCCAGCUCAGGUU | AACCUGAGCUGGACCAAAC | 1407 |
| 1360 | 256 | GUACCUGGAGUGGUAGGUA | UACCUACCACUCCAGGUAC | 1408 |
| 1361 | 294 | GGCGGGAGGAGUAGUUAAU | AUUAACUACUCCUCCCGCC | 1409 |
| 1362 | 295 | GCGGGAGGAGUAGUUAAUA | UAUUAACUACUCCUCCCGC | 1410 |
| 1363 | 296 | CGGGAGGAGUAGUUAAUAU | AUAUUAACUACUCCUCCCG | 1411 |
| 1364 | 297 | GGGAGGAGUAGUUAAUAUA | UAUAUUAACUACUCCUCCC | 1412 |
| 1365 | 341 | GGGGUUACAAAGUUGGCAU | AUGCCAACUUUGUAACCCC | 1413 |
| 1366 | 348 | CAAAGUUGGCAUCCAAGAU | AUCUUGGAUGCCAACUUUG | 1414 |
| 1367 | 377 | GACCCAACACCUCUUUGAU | AUCAAAGAGGUGUUGGGUC | 1415 |
| 1368 | 387 | CUCUUUGAUUAGAGGUGAU | AUCACCUCUAAUCAAAGAG | 1416 |
| 1369 | 426 | CAUAUUUAGCCUUUCUAAU | AUUAGAAAGGCUAAAUAUG | 1417 |
| 1370 | 436 | CUUUCUAAUACGGUAGUAU | AUACUACCGUAUUAGAAAG | 1418 |
| 1371 | 446 | CGGUAGUAUUGGAAAGGUA | UACCUUUCCAAUACUACCG | 1419 |
| 1372 | 498 | GGAACUGGCCGAUGUUGAA | UUCAACAUCGGCCAGUUCC | 1420 |
| 1373 | 499 | GAACUGGCCGAUGUUGAAU | AUUCAACAUCGGCCAGUUC | 1421 |
| 1374 | 511 | GUUGAAUUUGAGGUGGUUA | UAACCACCUCAAAUUCAAC | 1422 |
| 1375 | 520 | GAGGUGGUUAACAUUCCAA | UUGGAAUGUUAACCACCUC | 1423 |
| 1376 | 523 | GUGGUUAACAUUCCAAGAU | AUCUUGGAAUGUUAACCAC | 1424 |
| 1377 | 535 | CCAAGAUGGCUGCGAGUAU | AUACUCGCAGCCAUCUUGG | 1425 |
| 1378 | 543 | GCUGCGAGUAUCCUCCUUU | AAAGGAGGAUACUCGCAGC | 1426 |
| 1379 | 544 | CUGCGAGUAUCCUCCUUUU | AAAAGGAGGAUACUCGCAG | 1427 |
| 1380 | 546 | GCGAGUAUCCUCCUUUUAU | AUAAAAGGAGGAUACUCGC | 1428 |
| 1381 | 554 | CCUCCUUUUAUGGUGAGUA | UACUCACCAUAAAAGGAGG | 1429 |
| 1382 | 557 | CCUUUUAUGGUGAGUACAA | UUGUACUCACCAUAAAAGG | 1430 |
| 1383 | 558 | CUUUUAUGGUGAGUACAAA | UUUGUACUCACCAUAAAAG | 1431 |
| 1384 | 565 | GGUGAGUACAAAUUCUCUA | UAGAGAAUUUGUACUCACC | 1432 |
| 1385 | 579 | CUCUAGAAAGGCGGGAAUU | AAUUCCCGCCUUUCUAGAG | 1433 |
| 1386 | 610 | CUUUCGGCGCCAUCUGUAA | UUACAGAUGGCGCCGAAAG | 1434 |
| 1387 | 616 | GCGCCAUCUGUAACGGUUU | AAACCGUUACAGAUGGCGC | 1435 |
| 1388 | 665 | CUCCGGAGGAUGUUUCCAA | UUGGAAACAUCCUCCGGAG | 1436 |
| 1389 | 668 | CGGAGGAUGUUUCCAAGAU | AUCUUGGAAACAUCCUCCG | 1437 |
| 1390 | 700 | GGUCCUUCUUCUGCGGUAA | UUACCGCAGAAGAAGGACC | 1438 |
| 1391 | 729 | GCCACGUCAUCCUAUAAAA | UUUUAUAGGAUGACGUGGC | 1439 |
| 1392 | 754 | GAAGUGCGCUGCUGUAGUA | UACUACAGCAGCGCACUUC | 1440 |

# indicates start position

1. A method of isolating a cell that is substantially devoid of a target virus, comprising:
a. contacting a population of cells a portion of which comprises the target virus with an RNA effector molecule that inhibits the growth or replication of the target virus;
b. detecting the presence of the target virus in each cell; and,
c. isolating at least one cell that is substantially devoid of the target virus.

2. The method of claim 1, further comprising repeating steps a-c.

3. The method of claim 2, further comprising detecting the presence or absence of target viral nucleic acid or protein.

4. The method of claim 3, wherein the isolated cell is free of the target virus.

5. The method of claim 4, wherein steps a-c are repeated when target viral nucleic acid or protein is detected.

6. The method of claim 5, wherein the target viral nucleic acid is detected by a method selected from the group consisting of: polymerase chain reaction (PCR), reverse transcription polymerase chain reaction (RT-PCR), rapid amplification of cDNA ends (RACE), dot blot hybridization, Northern hybridization, and Southern hybridization.

7. The method of claim 5, wherein the target viral protein is detected by a method selected from the group consisting of: radioimmunoassay, competitive-binding assay, ELISA, Western blot, FACS, immunohistochemistry, immunoprecipitation, proteomics, mass spectrometry, electrophoresis, and immunofluoresence.

8. The method of claim 1, wherein the presence of the target virus of step b. is immunologically detected.

9. The method of claim 8, wherein the presence of the target virus is detected using an antibody against a capsid protein (Cap).

10. The method of claim 1, wherein the cell further comprises a second virus.

11. The method of claim 10, further comprising detecting the presence of the second virus.

12. The method of claim 11, wherein the presence of the second virus is detected by detection of viral nucleic acid or viral protein of the second virus.

13. The method of claim 12, wherein the second viral nucleic acid is detected by a method selected from the group consisting of: polymerase chain reaction (PCR), reverse transcription polymerase chain reaction (RT-PCR), rapid amplification of cDNA ends (RACE), dot blot hybridization, Northern hybridization, and Southern hybridization.

14. The method of claim 12, wherein the second viral protein is detected by a method selected from the group consisting of: radioimmunoassay, competitive-binding assay, ELISA, Western blot, FACS, immunohistochemistry, immunoprecipitation, proteomics, mass spectrometry, electrophoresis, and immunofluoresence.

15. The method of claim 10, wherein the second virus is rotavirus.

16. The method of claim 1, wherein the target virus is a non-integrating virus.

17. The method of claim 16, wherein the non-integrating virus is porcine circovirus.

18. The method of claim 1, wherein the RNA effector molecule is detectably labeled.

19. The method of claim 1, further comprising detecting the presence of the RNA effector molecule in the cell.

20. A cell that is substantially devoid of a target virus, obtained by the process of claim 1.

21. A method of producing an immunogenic agent, comprising:
(a) culturing the cell of claim 20 under conditions that allow expression of the immunogenic agent; and,
(b) isolating the immunogenic agent.

22. A method of preparing a virus stock that is substantially devoid of an unwanted target virus, comprising:
(a) contacting a virus stock that contains unwanted target virus with an agent that binds specifically with the unwanted target virus; and
(b) collecting the unbound virus stock.

23. The method of claim 22, further comprising repeating steps (a) and (b).

24. The method of claim 22, further comprising detecting the presence or absence of target viral nucleic acid or protein from the virus stock.

25. The method of claim 22, wherein the agent is immobilized.

26. The method of claim 22, wherein the agent is a receptor, an antibody, or an antigen binding fragment thereof.

27. The method of claim 26, wherein the agent is an antibody that binds to the porcine circovirus-1 (PCV-1).

28. The method of claim 27, wherein the antibody binds to the porcine circovirus-1 (PCV-1) capsid (Cap) protein.

29. The method of claim 26, wherein the agent is a receptor and the receptor is heparin.

30. The method of claim 22, wherein the agent is a receptor on a cell surface.

31. The method of claim 30, wherein the receptor on a cell surface is heparin.

32. The method of claim 22, further comprising contacting the cell with a RNA effector molecule that increases the presence of the receptor on the cell surface.

33. The method of claim 32, wherein the RNA effector molecule targets the heparanase gene.

34. The method of claim 30, wherein the cell does not have a receptor that binds the desired virus of the virus stock.

35. The method of claim 34, wherein the receptor is sialic acid.

36. The method of claim 30, wherein the cell is treated to have decreased level of cell receptor to the desired virus stock.

37. The method of claim 36, wherein the cell is treated by physical, chemical, or enzymatic means.

38. The method of claim 34, wherein the presence of a cell receptor to the desired viral stock has been decreased by contacting the cell with a RNA effector molecule that inhibits expression of the cell receptor.

39. The method of claim 30, further comprising
(a) contacting the unbound virus stock with a host cell;
(b) contacting the cell with an RNA effector molecule that inhibits growth or replication of the unwanted target virus; and
(c) isolating the virus stock from the medium.

40. The method of claim 39, wherein the host cell does not have a receptor for the unwanted target virus.

41. A virus stock that is substantially devoid of unwanted target virus prepared by the method of claim 22.

42. The virus stock of claim 41, wherein the virus stock is rotavirus.

43. The virus stock of claim 42, wherein the unwanted target virus is PCV1.

44. A biological product made using the virus stock of claim 41.

45. The biological product of claim 44, wherein the product is rotavirus vaccine.

46. A method of preparing a virus stock that is substantially devoid of an unwanted target virus, comprising:
(a) propagating a virus stock in a host cell in which expression of a receptor for a suspected viral contaminant has been inhibited; and
(b) collecting the virus stock.

47. The method of claim 46, wherein the receptor is heparin.

48. The method of claim 47, wherein the heparin is inhibited by contacting the host cell with a RNA effector molecule that targets an epimerase, a xylosyltransferase, a galactosyltransferase, a N-acetylglucosaminyl transferase, a glucuronosyl transferase, or a 2-O sulfotransferase gene.

49. The method of claim 46, further comprising contacting the host cell with a RNA effector molecule that inhibits the growth or replication of the unwanted target virus.

50. The method of claim 46, wherein the unwanted target virus is PCV1.

51. A virus stock that is substantially devoid of an unwanted target virus, prepared by the method of claim 46.

52. The virus stock of claim 51, wherein the virus stock is rotavirus.

53. A biological product made using the virus stock of claim 51.

54. The biological product of claim 53, wherein the product is rotavirus vaccine.

55. A virus stock that is substantially devoid of an unwanted target virus, prepared by the process comprising:
(a) contacting a virus stock that contains unwanted target virus with an agent that binds specifically with the unwanted target virus; and
(b) collecting the unbound virus stock.

56. A virus stock that is substantially devoid of an unwanted target virus, prepared by the process comprising:
(a) propagating a virus stock in a host cell in which expression of a receptor for a suspected viral contaminant has been inhibited; and
(b) collecting the stock virus.
